(19) United States
(12) Patent Application Publication
(10) Pub. No.: US 2010/0076015 A1
(43) Pub. Date: Mar. 25, 2010

(54) Aminopyridine Derivatives

(75) Inventors: David Michael Evans, Southampton (GB); Christine Elizabeth Allan, Southampton (GB); John Horton, Southampton (GB); David Philip Rooker, Southampton (GB)

Correspondence Address: WOODCOCK WASHBURN LLP, CIRA CENTRE, 12TH FLOOR, 2929 ARCH STREET, PHILADELPHIA, PA, 19104-2891, US

(73) Assignee: VANTIA LIMITED, Southampton (GB)

(21) Appl. No.: 12/431,114

(22) Filed: Apr. 28, 2009

Related U.S. Application Data
(60) Provisional application No. 61/071,431, filed on Apr. 29, 2008.

(30) Foreign Application Priority Data
Apr. 29, 2008 (GB) GB 0807828.9

(51) Int. Cl. A61K 31/47; C07D 213/02; C07D 401/12; C07D 217/00; A61K 31/44; A61K 31/4439; A61P 11/00; A61P 29/00

(52) U.S. Cl. 514/307; 546/309; 546/279.1; 546/146; 514/352; 514/343

(57) ABSTRACT

The present invention provides compounds of formula (I):

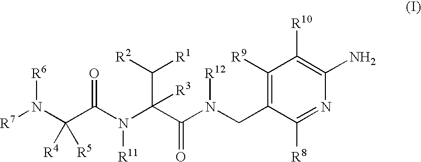

compositions comprising such compounds; the use of such compounds in therapy (such as asthma or COPD); and methods of treating patients with such compounds; wherein R¹-R¹¹ are as defined herein.

### CROSS REFERENCE TO RELATED APPLICATIONS

This application claims the benefit of U.S. patent application No. 61/071,431, filed Apr. 29, 2008, and Great Britain application number GB 0807828.9, filed Apr. 29, 2008, both of which are incorporated herein by reference in their entirety for all purposes.

This invention relates to aminopyridine derivatives and to processes for the preparation of, intermediates used in the preparation of, compositions containing and the uses of, such derivatives.

### BACKGROUND OF THE INVENTION

The aminopyridine derivatives of the present invention are inhibitors of tissue kallikrein and have a number of therapeutic applications, particularly in the treatment of inflammatory diseases such as asthma and chronic obstructive pulmonary disease (COPD).

The compounds of the invention are selective inhibitors of human tissue kallikrein (KLK1). In particular, they show an ability to inhibit KLK1 which is greater than their ability to inhibit other trypsin-like serine proteases.

Human tissue kallikrein, KLK1 (EC.3.4.21.35, also known as hK1, glandular kallikrein and urinary kallikrein) is a trypsin-like serine protease belonging to the kallikrein gene family of which there are 14 other members (including prostate specific antigen) (G. M. Yousef et al., Endocrine Rev., 2001, 22, 184). Other closely related trypsin-like serine proteases include plasma kallikrein, thrombin, trypsin and plasmin. Active KLK1 is a membrane-bound enzyme and is widely expressed. Strongest expression is observed in the pancreas, salivary gland, colon, kidney, lymph node, prostate, small intestine, stomach, thyroid gland and vagina. There is moderate expression of KLK1 in the lung, as well as expression in the saliva and its increased activity has also been detected in the sputum of patients following chronic lung injury.

KLK1 can liberate the kinins from kininogens by limited proteolysis, kallidin is released from low molecular weight kininogen whilst bradykinin is released from high molecular weight kininogen (K. D. Bhoola et al., Pharmacological Rev., 1992, 44, 1). Kinins such as kallidin (Lys-bradykinin) and bradykinin are potent mediators of inflammation. The actions of kinins are mediated by activation of two main bradykinin receptor subtypes, B1 and B2, both of which are members of the seven trans-membrane G protein-coupled receptor families. B1 receptors are involved in chronic responses and have low expression at basal levels but are upregulated following tissue injury and/or inflammation whilst B2 receptors are involved in acute responses and are constitutively expressed. KLK1 also activates the matrix metalloproteases (MMPs), pro-collagenase and pro-gelatinases and cleaves insulin-like growth factor binding protein-3 (J. A. Clements et al., Crit. Rev. Clin. Lab. Sci., 2004, 41, 265-312). There are also reports that KLK1 can directly activate the bradykinin receptors (C. Hecquet et al., Mol. Pharmacol., 2000, 39, 508-515).

Kinins have been shown to be important mediators in allergic inflammation such as asthma and hayfever (S. C. Chrstiansen et al., J. Clin. Invest., 1987, 79, 188-197) and that the enzyme chiefly responsible for the liberation of kinins in the airways of asthmatic subjects is KLK1 (S. C. Chrstiansen et al., Am. Rev. Respir. Dis., 1992, 145, 900-905). It has also been demonstrated that inflammatory cells release KLK1 (I. T. Lauredo et al., Am. J. Physiol. Lung Cell Mol. Physiol., 2004, 286, 734). Inhibition of KLK1 may be a novel approach for the treatment of asthma.

In addition KLK1 has been implicated in a number of other disease states including acute pancreatitis (T. Griesbacher, Pharmacology, 2000, 60, 113; T. Griesbacher et al., Br. J. Pharmacol., 2003, 139, 299), inflammatory bowel disease (A. Stadnicki, Digestive and Liver Disease, 2005, 37, 648; A. Stadnicki et al., Digestive Diseases and Science, 2003, 48, 615), arthritis (R. W. Colman, Immunopharmacology, 1999, 43, 103; R. J. Williams, Brit. J. Rheumatology, 1997, 36, 420).

High levels of circulating KLK1 induce chronic hypotension, Aprotinin a non-selective KLK1 inhibitor has been shown to suppress this (J. N. Sharma et al, Pharmacology, 1995, 50, 363; Q. Song et al., Immunopharmacology, 1996, 32, 105).

Antagonists of kinins (such as bradykinin receptor antagonists) have previously been investigated as potential therapeutic agents for the treatment of a number of inflammatory disorders (F. Marceau and D. Regoli, Nature Rev., Drug Discovery, 2004, 3, 845-852). In particular bradykinin B2 receptor antagonists have been investigated as potential treatments for airways disease (W. M. Abraham et al., Eur. J. Pharm., 2006, 533, 215).

There is also evidence that KLK1 plays a role in cancer (K. D. Bhoola et al., Curr. Opin. Invest. Drugs, 2007, 8, 462). KLK1 plays a role in increasing tumor invasiveness via activation of matrix metalloproteases, pro-collagenases and pro-gelatinases (K. D. Bhoola et al., Biol. Chem., 2001, 382, 77; H. Tschesche et al., Adv. Exp. Med. Biol., 1969, 247A, 545). Additionally KLK1 is indirectly involved in promoting proliferation through the liberation of mitogenic kinins (R. A. Roberts et al., J. Cell. Sci., 1989, 94, 527).

KLK1 is also involved in growth factor regulation and is implicated in processing of precursors of various growth factors e.g. EGF, NGF.

Endogenous inhibitors of KLK1 include the serpins, kallistatin, antiprotein C, α₁-antitrypsin, and α₁-antichymotrypsin. Aprotinin is also a potent non-selective KLK1 inhibitor. Low molecular weight inhibitors of KLK1 have previously been reported (M. Szelke et al., WO 199204371; M. Szelke et al., WO 199507291; C. Olivier et al., Peptides, 2000, 705; M. M. Staveski et al., WO 2003101941; M. Tokumasu et al., WO 2005095327; J. Burton et al., U.S. Pat. No. 5,464,820). KLK1 inhibitors have been reported to display activity in animal models of allergic inflammation (M. Szelke et al., Braz. J. Med. Biol. Res., 1994, 27, 1943; D. M. Evans et al., Immunopharmacology, 1996, 32, 117), citric acid induced cough (R. L. Featherstone et al., Lung, 1996, 174, 269) and acute pancreatitis (T. Griesbacher et al., Br. J. Pharmacol., 2002, 137, 692). KLK1 inhibitors have also been shown to be active in models of cancer (tumor cell migration in a matrigel invasion assay is inhibited in a dose-dependant manner by a KLK1 inhibitor) (W. C. Wolf et al., Am. J. Pathol, 2001, 159, 1797). A human KLK1 antibody that inhibits KLK1 with nanomolar potency has been shown to be active in an allergic sheep model of asthma. The antibody inhibited the late phase bronchoconstriction and completely blocked airway hyperresponsiveness (D. J. Sexton et al., WO 2006017538).

Hyaluronic acid which binds and inactivates KLK1 in vitro has been shown to block porcine pancreatic elastase induced bronchoconstriction in sheep (M. Scuri et al., Am. J. Respir. Crit. Care Med., 2001, 164, 1855).

Kallikrein-binding protein (KBP) is a serine protease inhibitor (serpin) which specifically binds to tissue kallikrein and inhibits kallikrein activity. KBP has been shown to inhibit retinal neovascularization and decrease vascular leakage by downregulation of vascular endothelial growth factor (VEGF) (G. Gao et al., Diabetologia, 2003, 46, 689) and to inhibit growth of gastric carcinoma by reducing VEGF production (L. Lu et al., Mol. Cancer. Ther., 2007, 6, 3297). VEGF has also been linked with blood-retinal barrier breakdown which is a hallmark of diabetic retinopathy (D. A. Antonettie et al., Diabetes, 1998, 47, 1953). VEGF has also been implicated in remodeling of airway vasculature in chronic inflammation (D. M. McDonald, Am. J. Respir. Crit. Care Med., 2001, 164, S39).

Selectivity with respect to the other members of the trypsin-like serine protease family, particularly plasma kallikrein, is an important issue. Inhibitors of tissue kallikrein displaying poor plasma kallikrein activity have previously been reported (M. Szelke et al., Brazilian J. Med. Biol. Res. 1994, 27, 1935 and D. M. Evans et al., Immunopharmacology, 1996, 32, 117), but there remains a need for further compounds that selectively inhibit tissue kallikrein. Several groups have disclosed synthetic inhibitors of plasma kallikrein. These include arginineketomethylene derivatives (WO 92/04371 and D. M. Evans et al., Immunopharmacology, 1996, 32, 115-116), noragmatine and agmatine derivatives (WO 95/07291, WO 94/29335), benzamidine derivatives (J. Sturzbecher et al., Brazilian J. Med. Biol. Res., 1994, 27, 1929-1934), boronic acid derivatives (U.S. Pat. No. 5,187,157) and aminomethylcyclohexanoyl derivatives (N. Teno et al., Chem. Pharm. Bull., 1993, 41, 1079-1090).

The compounds of the present invention, and their pharmaceutically acceptable salts, have the advantage that they are selective inhibitors of KLK1 (and so are likely to have reduced side effects). In addition, they may be more potent, they may be longer acting, they may have greater bioavailability or they may have other more desirable properties than the compounds of the prior art.

### SUMMARY OF THE INVENTION

In one aspect, the present invention provides compounds of formula (I):

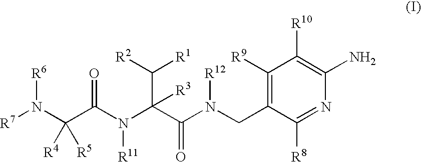

wherein:

R¹ and R² are independently selected from H, OH, (C₁-C₁₀)alkyl, (C₁-C₆)alkoxy, (C₂-C₆)alkenyl, (C₃-C₁₀)cycloalkyl, heterocycloalkyl, aryl, heteroaryl, aryl(C₁-C₄)alkyl- and heteroaryl(C₁-C₄)alkyl-;

R³ is selected from H, (C₁-C₁₀)alkyl and (C₂-C₆)alkenyl;

R⁴ and R⁵ are selected from H, (C₁-C₁₀)alkyl, (C₂-C₆)alkenyl, (C₃-C₁₀)cycloalkyl, heterocycloalkyl, aryl, heteroaryl, aryl(C₁-C₄)alkyl- and heteroaryl(C₁-C₄)alkyl-;

R⁶ and R⁷ are selected from H, (C₁-C₁₀)alkyl, (C₂-C₆)alkenyl, (C₃-C₁₀)cycloalkyl, heterocycloalkyl, aryl, heteroaryl, aryl(C₁-C₄)alkyl-, aryl(C₂-C₄)alkenyl-, heteroaryl(C₁-C₄)alkyl-, —SO₂(C₁-C₆)alkyl, —SO₂aryl and —SO₂aryl(C₁-C₄)alkyl;
or R⁶ and R⁷ together with the nitrogen atom to which they are attached may form a 4-7 membered N-containing ring, optionally containing one further heteroatom selected from N, O and S, and optionally substituted with 1 or 2 substituents independently selected from (C₁-C₆)alkyl, (C₁-C₆)alkoxy, halo, CN and hydroxyl, said N-containing ring may also optionally be fused to an aryl group;
or R⁴ and R⁶ together with the atoms to which they are attached may form a saturated or partially unsaturated 4-7 membered N-containing ring, optionally containing one further heteroatom selected from N, O and S, and optionally substituted on carbon with 1 or 2 substituents independently selected from (C₁-C₆)alkyl, (C₁-C₆)alkoxy, halo, CN and hydroxyl;
or R⁵ is absent and R⁴ and R⁶ together with the atoms to which they are attached may form a 5, 6, 9 or 10 membered mono- or bi-cylic N-containing aromatic ring, optionally containing one further heteroatom selected from N, O and S, and optionally substituted on carbon with 1, 2 or 3 substituents independently selected from (C₁-C₆)alkyl, (C₁-C₆)alkoxy, halo, CN, aryl, COOR¹⁴ and hydroxyl;
or R⁴ and R⁶ may together form a group according to formula II or formula III:

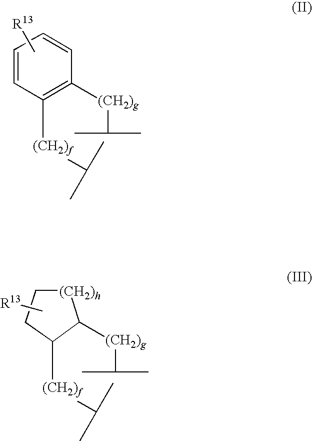

R⁸, R⁹ and R¹⁰ are independently selected from H, (C₁-C₁₀)alkyl, halogen, hydroxyl and (C₁-C₆)alkoxy;

R¹¹ is selected from H and (C₁-C₆)alkyl;

R¹² is selected from H and (C₁-C₆)alkyl;

R¹³ is selected from H, (C₁-C₆)alkyl, (C₁-C₆)alkoxy, OH, CN, CF₃, COOR¹⁴, halo and NR¹⁴R¹⁵;

R¹⁴ and R¹⁵ are independently selected from H and (C₁-C₆)alkyl;

f and g are independently selected from 0, 1, 2 and 3, such that f+g =1, 2 or 3;

h is selected from 1 and 2;

wherein:
alkyl may optionally be substituted with 1 or 2 substituents independently selected from (C₃-C₁₀)cycloalkyl, (C₁-C₆)alkoxy, OH, CN, CF₃, COOR¹⁴, halo and NR¹⁴R¹⁵;
alkenyl may optionally be substituted with 1 or 2 substituents independently selected from (C₃-C₁₀)cycloalkyl, (C₁-C₆)alkoxy, OH, CN, CF₃, COOR¹⁴, halo and NR¹⁴R¹⁵;
alkoxy may optionally be substituted with 1 or 2 substituents independently selected from (C₃-C₁₀)cycloalkyl, OH, CN, CF₃, COOR¹⁴, halo and NR¹⁴R¹⁵;
cycloalkyl is a non-aromatic mono- or bi-cylic hydrocarbon ring, optionally fused to an aryl group, wherein said cycloalkyl ring optionally contains, where possible, up to 2 double bonds; and wherein, unless otherwise stated, said cycloalkyl may optionally be substituted with 1 or 2 substituents independently selected from (C₁-C₆)alkyl, (C₁-C₆)alkoxy, OH, CN, CF₃, COOR¹⁴ , halo and NR¹⁴R¹⁵;
heterocycloalkyl is a C-linked or N-linked 3 to 10 membered non-aromatic, mono- or bi-cyclic ring, wherein said heterocycloalkyl ring contains, where possible, 1, 2 or 3 heteroatoms independently selected from N, NR¹⁴, S(O)_{q} and O; and said heterocycloalkyl ring optionally contains, where possible, 1 or 2 double bonds, and is optionally substituted on carbon with 1 or 2 substituents independently selected from (C₁-C₆)alkyl, (C₁-C₆)alkoxy, OH, CN, CF₃, halo, COOR¹⁴, NR¹⁴R¹⁵ and aryl;
aryl is a single or fused aromatic ring system containing 6 or 10 carbon atoms; wherein, unless otherwise stated, each occurrence of aryl may be optionally substituted with up to 5 substituents independently selected from (C₁-C₆)alkyl, (C₁-C₆)alkoxy, OH, halo, CN, COOR¹⁴, CF₃ and NR¹⁴R¹⁵;
heteroaryl is a 5, 6, 9 or 10 membered mono- or bi-cyclic aromatic ring, containing 1 or 2 N atoms and, optionally, an NR¹⁴ atom, or one NR¹⁴ atom and an S or an O atom, or one S atom, or one O atom; wherein, unless otherwise stated, said heteroaryl may be optionally substituted with 1, 2 or 3 substituents independently selected from (C₁-C₆)alkyl, (C₁-C₆)alkoxy, OH, halo, CN, COOR¹⁴, CF₃ and NR¹⁴R¹⁵;
q is 0, 1 or 2;

and tautomers, stereoisomers, pharmaceutically acceptable salts and solvates thereof.

In another aspect the present invention provides a prodrug of a compound of formula (I) as herein defined, or a pharmaceutically acceptable salt thereof.

In yet another aspect the present invention provides an N-oxide of a compound of formula (I) as herein defined, or a prodrug or pharmaceutically acceptable salt thereof.

It will be understood that certain compounds of the present invention may exist in solvated, for example hydrated, as well as unsolvated forms. It is to be understood that the present invention encompasses all such solvated forms.

In one subset of the compounds of formula (I):

R¹ is selected from (C₁-C₆)alkyl, (C₃-C₁₀)cycloalkyl, heterocycloalkyl, aryl and heteroaryl;

R² is selected from H, (C₁-C₆)alkyl, OH, (C₁-C₆)alkoxy, (C₃-C₁₀)cycloalkyl and aryl;

R³ is selected from H and (C₁-C₆)alkyl;

R⁴ is selected from H, (C₁-C₆)alkyl, (C₃-C₁₀)cycloalkyl, (C₃-C₁₀)cycloalkyl(C₁-C₄)alkyl-, aryl and aryl(C₁-C₄)alkyl-;

R⁵ is selected from H and (C₁-C₆)alkyl;

R⁶ is selected from H and (C₁-C₆)alkyl;

R⁷ is selected from H, (C₁-C₆)alkyl, (C₃-C₁₀)cycloalkyl, (C₃-C₁₀)cycloalkyl(C₁-C₄)alkyl-, aryl, heteroaryl, aryl(C₁-C₄)alkyl-, aryl(C₂-C₄)alkenyl-, heteroaryl(C₁-C₄)alkyl- and —SO₂(C₁-C₆)alkyl;
or R⁶ and R⁷ together with the nitrogen atom to which they are attached may form a 4-7 membered N-containing ring, optionally containing one further heteroatom selected from N, O and S, and optionally fused to an aryl group;
or R⁴ and R⁶ together with the atoms to which they are attached may form a 5 or 6 membered N-containing ring, optionally containing one carbon-carbon double bond, and optionally containing one further heteroatom selected from O and S, wherein said N-containing ring is optionally substituted on carbon with 1 or 2 substituents independently selected from (C₁-C₆)alkyl, halo, CN and hydroxyl;
or R⁵ is absent and R⁴ and R⁶ together with the atoms to which they are attached may form a 5, 6 or 9 membered mono- or bi-cyclic N-containing aromatic ring, optionally containing one further heteroatom selected from N and O, and optionally substituted on carbon with 1, 2 or 3 substituents independently selected from (C₁-C₆)alkyl, (C₁-C₆)alkoxy, halo, CN, aryl, COOR¹⁴ and hydroxyl;
or R⁴ and R⁶ may together form a group according to formula II or formula III:

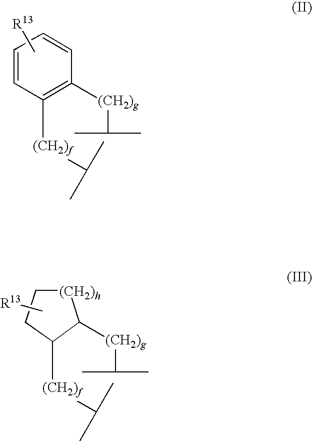

R⁸, R⁹ and R¹⁰ are indepently selected from H, (C₁-C₁₀)alkyl, halogen, hydroxyl and (C₁-C₆)alkoxy;

R¹¹ is selected from H and (C₁-C₁₀)alkyl;

R¹² is selected from H and (C₁-C₆)alkyl;

R¹³ is H;

f and g are independently selected from 0, 1, 2 and 3, such that f+g=1, 2 or 3;

h is selected from 1 and 2;

wherein alkyl, alkenyl, alkoxy, cycloalkyl, heterocycloalkyl, aryl and heteroaryl are as defined above;

and tautomers, stereoisomers, pharmaceutically acceptable salts and solvates thereof.

The present invention also comprises the following aspects and combinations thereof:

In one aspect, the present invention provides a compound of formula (I) wherein R¹ is selected from (C₁-C₁₀)alkyl, (C₃-C₁₀)cycloalkyl, aryl, heteroaryl and aryl(C₁-C₄)alkyl-.

In another aspect, the present invention provides a compound of formula (I) wherein R¹ is selected from (C₁-C₆)alkyl, (C₅-C₁₀)cycloalkyl, aryl and heteroaryl.

In a further aspect, the present invention provides a compound of formula (I) wherein R¹ is selected from (C₅-C₁₀)cycloalkyl, aryl and heteroaryl.

In a yet further aspect, the present invention provides a compound of formula (I) wherein R¹ is optionally substituted phenyl. Optional substituents are selected from those defined above for ‘aryl’.

In one aspect, the present invention provides a compound of formula (I) wherein R² is selected from H, (C₁-C₆)alkyl, OH, (C₁-C₆)alkoxy, (C₃-C₁₀)cycloalkyl and aryl.

In another aspect, the present invention provides a compound of formula (I) wherein R² is selected from H, (C₁-C₆)alkyl, OH, (C₁-C₆)alkoxy and (C₃-C₁₀)cycloalkyl.

In yet another aspect, the present invention provides a compound of formula (I) wherein R² is selected from H, OH and (C₄-C₆)cycloalkyl.

In a further aspect, the present invention provides a compound of formula (I) wherein R² is H.

In one aspect, the present invention provides a compound of formula (I) wherein R³ is selected from H and (C₁-C₆)alkyl.

In another aspect, the present invention provides a compound of formula (I) wherein R³ is H.

In one aspect, the present invention provides a compound of formula (I) wherein R³ is H and the carbon atom to which R³ is attached is chiral and has an (S) configuration.

In another aspect, the present invention provides a compound of formula (I) wherein R³ is H and the carbon atom to which R³ is attached is chiral and has an (R) configuration.

In one aspect, the present invention provides a compound of formula (I) wherein R⁴ is selected from H, (C₁-C₁₀)alkyl, (C₃-C₁₀)cyclo alkyl, (C₃-C₁₀)cycloalkyl(C₁-C₄)alkyl-, aryl, heteroaryl, heteroaryl(C₁-C₄)alkyl- and aryl(C₁-C₄)alkyl-.

In another aspect, the present invention provides a compound of formula (I) wherein R⁴ is selected from (C₁-C₁₀)alkyl, (C₃-C₁₀)cycloalkyl, aryl, heteroaryl, heteroaryl(C₁-C₄)alkyl- and aryl(C₁-C₄)alkyl-.

In a further aspect, the present invention provides a compound of formula (I) wherein R⁴ is selected from H, (C₁-C₁₀)alkyl, (C₃-C₁₀)cycloalkyl, aryl and aryl(C₁-C₄)alkyl-.

In a yet further aspect, the present invention provides a compound of formula (I) wherein R⁴ is selected from H and (C₁-C₆)alkyl, (C₄-C₆)cycloalkyl, optionally substituted phenyl and optionally substituted phenyl(C₁-C₄)alkyl-. Optional substituents are selected from those defined above for ‘aryl’.

In one aspect, the present invention provides a compound of formula (I) wherein R⁵ is selected from H and (C₁-C₆)alkyl.

In another aspect, the present invention provides a compound of formula (I) wherein R⁵ is H.

In one aspect, the present invention provides a compound of formula (I) wherein one of R⁴ and R⁵ is H and the other of R⁴ and R⁵ is not H, and the carbon atom to which R⁴ and R⁵ is attached is chiral and has an (R) configuration.

In another aspect, the present invention provides a compound of formula (I) wherein one of R⁴ and R⁵ is H and the other of R⁴ and R⁵ is not H, and the carbon atom to which R⁴ and R⁵ is attached is chiral and has an (S) configuration.

In one aspect, the present invention provides a compound of formula (I) wherein R³ is H and the carbon atom to which R³ is attached is chiral and has an (R) configuration, and wherein one of R⁴ and R⁵ is H and the other of R⁴ and R⁵ is not H, and the carbon atom to which R⁴ and R⁵ are attached is chiral and has an (S) configuration.

In another aspect, the present invention provides a compound of formula (I) wherein R³ is H and the carbon atom to which R³ is attached is chiral and has an (S) configuration, and wherein one of R⁴ and R⁵ is H and the other of R⁴ and R⁵ is not H, and the carbon atom to which R⁴ and R⁵ are attached is chiral and has an (R) configuration.

In one aspect, the present invention provides compounds of formula (I) wherein R⁶ is selected from H, (C₁-C₁₀)alkyl, (C₃-C₁₀)cyclo alkyl, (C₃-C₁₀)cycloalkyl(C₁-C₄)alkyl-, aryl, aryl(C₁-C₄)alkyl- and —SO₂(C₁-C₆)alkyl.

In another aspect, the present invention provides compounds of formula (I) wherein R⁶ is selected from H and (C₁-C₆)alkyl.

In a further aspect, the present invention provides compounds of formula (I) wherein R⁶ is H.

In one aspect, the present invention provides compounds of formula (I) wherein R⁷ is selected from H, (C₁-C₁₀)alkyl, (C₃-C₁₀)cyclo alkyl, (C₃-C₁₀)cycloalkyl(C₁-C₄)alkyl-, aryl, heteroaryl, aryl(C₁-C₄)alkyl-, aryl(C₂-C₄)alkenyl-, heteroaryl(C₁-C₄)alkyl-, —SO₂(C₁-C₆)alkyl and —SO₂aryl.

In another aspect, the present invention provides compounds of formula (I) wherein R⁷ is selected from H, (C₁-C₆)alkyl, (C₃-C₁₀)cycloalkyl, aryl, heteroaryl, aryl(C₁-C₄)alkyl-, aryl(C₂-C₄)alkenyl-, heteroaryl(C₁-C₄)alkyl- and —SO₂(C₁-C₆)alkyl.

In a further aspect, the present invention provides compounds of formula (I) wherein R⁷ is selected from H, (C₁-C₆)alkyl, aryl, aryl(C₁-C₄)alkyl-, aryl(C₂-C₄)alkenyl-, heteroaryl(C₁-C₄)alkyl- and —SO₂(C₁-C₆)alkyl.

In a yet further aspect, the present invention provides compounds of formula (I) wherein R⁷ is selected from H, (C₁-C₆)alkyl, phenyl and phenyl(C₁-C₄)alkyl-.

In a still further aspect, the present invention provides compounds of formula (I) wherein R⁷ is selected from H and (C₁-C₆)alkyl.

In one aspect, the present invention provides compounds of formula (I) wherein R⁶ and R⁷ together with the nitrogen atom to which they are attached may form a 5-6 membered N-containing ring, optionally containing one further heteroatom selected from N, O and S, and optionally substituted with 1 or 2 substituents independently selected from (C₁-C₆)alkyl, (C₁-C₆)alkoxy, halo, CN and hydroxyl, said N-containing ring may also optionally be fused to an aryl group.

In another aspect, the present invention provides compounds of formula (I) wherein R⁶ and R⁷ together with the nitrogen atom to which they are attached may form a 5-6 membered N-containing ring, optionally containing one further heteroatom selected from O, and optionally substituted with 1 or 2 substituents independently selected from (C₁-C₆)alkyl, (C₁-C₆)alkoxy, halo, CN and hydroxyl, said N-containing ring may also optionally be fused to an aryl group.

In a further aspect, the present invention provides compounds of formula (I) wherein R⁶ and R⁷ together with the nitrogen atom to which they are attached may form a 5-6 membered N-containing ring, optionally containing one further heteroatom selected from O, and optionally fused to a phenyl group.

In a yet further aspect, the present invention provides compounds of formula (I) wherein R⁶ and R⁷ together with the nitrogen atom to which they are attached may form a 5-6 membered N-containing ring, optionally containing one further heteroatom selected from O, and optionally substituted with 1 or 2 substituents independently selected from (C₁-C₆)alkyl, (C₁-C₆)alkoxy, halo, CN and hydroxyl.

In a yet further aspect still, the present invention provides compounds of formula (I) wherein R⁶ and R⁷ together with the nitrogen atom to which they are attached may form a group selected from pyrolidinyl, tetrahydroquinolinyl, tetrahydroisoquinolinyl, piperidinyl and morpholinyl, wherein each of said groups may optionally be substituted with 1, 2 or 3 substituents selected from (C₁-C₆)alkyl, (C₁-C₆)alkoxy, halo and hydroxyl.

In one aspect, the present invention provides compounds of formula (I) wherein R⁴ and R⁶ together with the atoms to which they are attached may form a saturated or unsaturated 4-7 membered N-containing ring, optionally containing one further heteroatom selected from N, O and S, and optionally substituted on carbon with 1 or 2 substituents independently selected from (C₁-C₆)alkyl, (C₁-C₆)alkoxy, halo, CN and hydroxyl;

In another aspect, the present invention provides compounds of formula (I) wherein R⁴ and R⁶ together with the atoms to which they are attached may form a 4-7 membered N-containing ring, optionally containing one carbon-carbon double bond, optionally containing one further heteroatom selected from N, O and S, and optionally substituted on carbon with 1 or 2 substituents independently selected from (C₁-C₆)alkyl, (C₁-C₆)alkoxy, halo, CN and hydroxyl. Typically in this aspect, R⁵ is H and the carbon to which R⁴ and R⁵ are attached is chiral and has an (R) configuration.

In yet another aspect, the present invention provides compounds of formula (I) wherein R⁴ and R⁶ together with the atoms to which they are attached may form a 5 or 6 membered N-containing ring, optionally containing one carbon-carbon double bond, and optionally containing one further heteroatom selected from O and S, wherein said N-containing ring is optionally substituted on carbon with 1 or 2 substituents independently selected from (C₁-C₆)alkyl, halo, CN and hydroxyl. Typically in this aspect, R⁵ is H and the carbon to which R⁴ and R⁵ are attached is chiral and has an (R) configuration.

In a further aspect, the present invention provides compounds of formula (I) wherein R⁵ is absent and R⁴ and R⁶ together with the atoms to which they are attached may form a 5, 6 or 9 membered N-containing mono- or bi-cyclic aromatic ring, optionally containing one further heteroatom selected from N and O, and optionally substituted on carbon with 1, 2 or 3 substituents independently selected from (C₁-C₆)alkyl, (C₁-C₆)alkoxy, halo, aryl, COOR¹⁴ and hydroxyl.

In a yet further aspect, the present invention provides compounds of formula (I) wherein R⁴ and R⁶ together with the atoms to which they are attached may form a group selected from pyrolidinyl, thiazolidinyl, tetrahydroisoquinolinyl, dihydro-1H-pyrrolyl, piperidinyl, pyrrolyl, imidazolyl, pyrazolyl, indolyl and benzimidazolyl, wherein each of said groups may optionally be substituted with 1, 2 or 3 substituents selected from (C₁-C₆)alkyl, (C₁-C₆)alkoxy, halo, aryl, COOR¹⁴ and hydroxyl.

In one aspect, the present invention provides compounds of formula (I) wherein R⁴ and R⁶ may together form a group according to formula II or formula III:

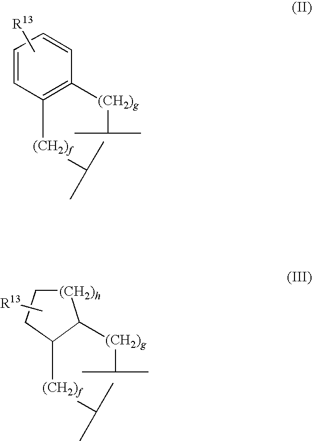

wherein R¹³ is H and f and g are independently selected from 0, 1, 2 and 3, such that f+g=1, 2 or 3; and h is selected from 1 and 2.

In one aspect, the present invention provides compounds of formula (I) wherein R⁸, R⁹ and R¹⁰ are independently selected from H, (C₁-C₁₀)alkyl and halogen.

In another aspect, the present invention provides compounds of formula (I) wherein R⁸, R⁹ and R¹⁰ are independently selected from H and (C₁-C₆)alkyl.

In a further aspect, the present invention provides compounds of formula (I) wherein R⁸, R⁹ and R¹⁰ are independently selected from H and methyl.

In a yet further aspect, the present invention provides compounds of formula (I) wherein one of R⁸, R⁹ and R¹⁰ is methyl and the other two are H.

In a yet further aspect still, the present invention provides compounds of formula (I) wherein R⁸, R⁹ and R¹⁰ are H.

In one aspect, the present invention provides compounds of formula (I) wherein R¹¹ is H.

In one aspect, the present invention provides compounds of formula (I) wherein R¹² is H.

In one aspect, the present invention provides a compound of formula (I) selected from:

(R)-2-Amino-3-methyl-pentanoic acid {(S)-1-[(6-amino-pyridin-3-ylmethyl)-carbamoyl]-2-naphthalen-1-yl-ethyl}-amide;

(R)-2-Amino-3-methyl-pentanoic acid [(S)-1-[(6-amino-pyridin-3-ylmethyl)-carbamoyl]-2-(decahydro-naphthalen-1-yl)-ethyl]-amide;

(R)-3-Methyl-2-methylamino-pentanoic acid [(S)-1-[(6-amino-pyridin-3-ylmethyl)-carbamoyl]-2-(3,4-dichloro-phenyl)-ethyl]-amide;

(R)-Pyrrolidine-2-carboxylic acid [(S)-1-[(6-amino-pyridin-3-ylmethyl)-carbamoyl]-2-(3,4-dichloro-phenyl)-ethyl]-amide;

(R)-1-Methyl-pyrrolidine-2-carboxylic acid [(S)-1-[(6-amino-pyridin-3-ylmethyl)-carbamoyl]-2-(3,4-difluoro-phenyl)-ethyl]-amide;

(R)-1-Isopropyl-pyrrolidine-2-carboxylic acid [(S)-1-[(6-amino-pyridin-3-ylmethyl)-carbamoyl]-2-(3,4-difluoro-phenyl)-ethyl]-amide;

(R)-1-Benzyl-pyrrolidine-2-carboxylic acid [(S)-1-[(6-amino-pyridin-3-ylmethyl)-carbamoyl]-2-(3,4-dichloro-phenyl)-ethyl]-amide;

(R)-2-(Methanesulfonyl-methyl-amino)-3-methyl-pentanoic acid [(S)-1-[(6-amino-pyridin-3-ylmethyl)-carbamoyl]-2-(3,4-dichloro-phenyl)-ethyl]-amide;

(R)-1-Methyl-pyrrolidine-2-carboxylic acid {(S)-1-[(6-amino-2-methyl-pyridin-3-ylmethyl)-carbamoyl]-2-naphthalen-1-yl-ethyl}-amide;

(S)—N-(6-Amino-pyridin-3-ylmethyl)-2-(2-diethylamino-acetylamino)-3-(3,4-difluoro-phenyl)-propionamide;

(S)-2-Amino-3-methyl-pentanoic acid {(R)-1-[(6-amino-pyridin-3-ylmethyl)-carbamoyl]-2,2-dicyclohexyl-ethyl}-amide;

(S)-2-Amino-3-methyl-pentanoic acid [(S)-1-[(6-amino-pyridin-3-ylmethyl)-carbamoyl]-2-(decahydro-naphthalen-1-yl)-ethyl]-amide;

(R)-2-Amino-N—[(S)-1-[(6-amino-pyridin-3-ylmethyl)carbamoyl]-2-(decahydro-naphthalen-1-yl)-ethyl]-3-(4-chloro-phenyl)-propionamide;

(R)-2-Amino-N—[(S)-1-[(6-amino-pyridin-3-ylmethyl)-carbamoyl]-2-(decahydro-naphthalen-1-yl)-ethyl]-3-methyl-butyramide;

(R)-Pyrrolidine-2-carboxylic acid [(S)-1-[(6-amino-pyridin-3-ylmethyl)-carbamoyl]-2-(decahydro-naphthalen-1-yl)-ethyl]-amide;

(R)-2-Amino-4-methyl-pentanoic acid [(S)-1-[(6-amino-pyridin-3-ylmethyl)-carbamoyl]-2-(decahydro-naphthalen-1-yl)-ethyl]-amide;

(S)-2-(2-Amino-acetylamino)-N-(6-amino-pyridin-3-ylmethyl)-3-(decahydro-naphthalen-1-yl)-propionamide;

(R)-2-Amino-N—[(S)-1-[(6-amino-pyridin-3-ylmethyl)-carbamoyl]-2-(decahydro-naphthalen-1-yl)-ethyl]-propionamide;

(S)—N-(6-Amino-pyridin-3-ylmethyl)-3-(decahydro-naphthalen-1-yl)-2-(2-methylamino-acetylamino)-propionamide;

(R)-2-Amino-3-methyl-pentanoic acid {(S)-1-[(6-amino-pyridin-3-ylmethyl)-carbamoyl]-2-cyclohexyl-ethyl}-amide;

(R)-3-Methyl-2-methylamino-pentanoic acid {(S)-1-[(6-amino-pyridin-3-ylmethyl)-carbamoyl]-2-cyclohexyl-ethyl}-amide;

(R)-2-Amino-N—{(S)-1-[(6-amino-pyridin-3-ylmethyl)-carbamoyl]-2-naphthalen-1-yl-ethyl}-3-methyl-butyramide;

(R)-Pyrrolidine-2-carboxylic acid {(S)-1-[(6-amino-pyridin-3-ylmethyl)-carbamoyl]-2-naphthalen-1-yl-ethyl}-amide;

(R)-2-Amino-3-methyl-pentanoic acid [(S)-1-[(6-amino-pyridin-3-ylmethyl)-carbamoyl]-2-(2-chloro-phenyl)-ethyl]-amide;

(R)-2-Amino-3-methyl-pentanoic acid [(S)-1-[(6-amino-pyridin-3-ylmethyl)-carbamoyl]-2-(2-trifluoromethyl-phenyl)-ethyl]-amide;

(R)-2-Amino-3-methyl-pentanoic acid {(S)-1-[(6-amino-pyridin-3-ylmethyl)-carbamoyl]-2-phenyl-ethyl}-amide;

(R)-2-Amino-3-methyl-pentanoic acid {(S)-1-[(6-amino-pyridin-3-ylmethyl)-carbamoyl]-2-m-tolyl-ethyl}-amide;

(R)-2-Amino-3-methyl-pentanoic acid {(S)-1-[(6-amino-pyridin-3-ylmethyl)-carbamoyl]-2-o-tolyl-ethyl}-amide;

(R)-2-Amino-3-methyl-pentanoic acid [(S)-1-[(6-amino-pyridin-3-ylmethyl)-carbamoyl]-2-(3-fluoro-phenyl)-ethyl]-amide;

(R)-2-Amino-3-methyl-pentanoic acid [(S)-1-[(6-amino-pyridin-3-ylmethyl)-carbamoyl]-2-(3-trifluoromethyl-phenyl)-ethyl]-amide;

(R)-2-Amino-3-methyl-pentanoic acid [(S)-1-[(6-amino-pyridin-3-ylmethyl)-carbamoyl]-2-(3-cyano-phenyl)-ethyl]-amide;

(R)-2-Amino-3-methyl-pentanoic acid [(S)-1-[(6-amino-pyridin-3-ylmethyl)-carbamoyl]-2-(3,4-difluoro-phenyl)-ethyl]-amide;

(R)-2-Amino-3-methyl-pentanoic acid [(S)-1-[(6-amino-pyridin-3-ylmethyl)-carbamoyl]-2-(4-fluoro-phenyl)-ethyl]-amide;

(R)-2-Amino-3-methyl-pentanoic acid {(S)-1-[(6-amino-pyridin-3-ylmethyl)-carbamoyl]-2-naphthalen-2-yl-ethyl}-amide;

(R)-2-Amino-3-methyl-pentanoic acid [(S)-1-[(6-amino-pyridin-3-ylmethyl)-carbamoyl]-2-(3,4-dichloro-phenyl)-ethyl]-amide;

(R)-2-Amino-3-methyl-pentanoic acid [(S)-1-[(6-amino-pyridin-3-ylmethyl)-carbamoyl]-2-(4-chloro-phenyl)-ethyl]-amide;

(R)-2-Amino-3-methyl-pentanoic acid [(S)-1-[(6-amino-pyridin-3-ylmethyl)-carbamoyl]-2-(4-hydroxy-phenyl)-ethyl]-amide;

(R)-2-Amino-3-methyl-pentanoic acid [(S)-1-[(6-amino-pyridin-3-ylmethyl)-carbamoyl]-2-(2,4,5-trifluoro-phenyl)-ethyl]-amide;

(R)-2-Amino-3-methyl-pentanoic acid [(S)-1-[(6-amino-pyridin-3-ylmethyl)-carbamoyl]-2-(3,5-difluoro-phenyl)-ethyl]-amide;

(R)-2-Amino-3-methyl-pentanoic acid [(S)-1-[(6-amino-pyridin-3-ylmethyl)-carbamoyl]-2-(1H-indol-3-yl)-ethyl]-amide;

(R)-2-Amino-3-methyl-pentanoic acid {(S)-1-[(6-amino-pyridin-3-ylmethyl)-carbamoyl]-2-benzo[b]thiophen-3-yl-ethyl}-amide;

(R)-3-Methyl-2-methylamino-pentanoic acid [(S)-1-[(6-amino-pyridin-3-ylmethyl)-carbamoyl]-2-(3-trifluoromethyl-phenyl)-ethyl]-amide;

(R)-3-Methyl-2-methylamino-pentanoic acid [(S)-1-[(6-amino-pyridin-3-ylmethyl)-carbamoyl]-2-(3,4-difluoro-phenyl)-ethyl]-amide;

(R)-3-Methyl-2-methylamino-pentanoic acid [(S)-1-[(6-amino-pyridin-3-ylmethyl)-carbamoyl]-2-(4-fluoro-phenyl)-ethyl]-amide;

(R)-3-Methyl-2-methylamino-pentanoic acid [(S)-1-[(6-amino-pyridin-3-ylmethyl)-carbamoyl]-2-(3-chloro-phenyl)-ethyl]-amide;

(R)-3-Methyl-2-methylamino-pentanoic acid [(S)-1-[(6-amino-pyridin-3-ylmethyl)-carbamoyl]-2-(4-chloro-phenyl)-ethyl]-amide;

(R)-3-Methyl-2-methylamino-pentanoic acid [(S)-1-[(6-amino-pyridin-3-ylmethyl)-carbamoyl]-2-(4-hydroxy-phenyl)-ethyl]-amide;

(R)-3-Methyl-2-methylamino-pentanoic acid [(S)-1-[(6-amino-pyridin-3-ylmethyl)-carbamoyl]-2-(2,4,5-trifluoro-phenyl)-ethyl]-amide;

(R)-3-Methyl-2-methylamino-pentanoic acid [(S)-1-[(6-amino-pyridin-3-ylmethyl)-carbamoyl]-2-(3,5-difluoro-phenyl)-ethyl]-amide;

(R)-3-Methyl-2-methylamino-pentanoic acid {(S)-1-[(6-amino-pyridin-3-ylmethyl)-carbamoyl]-2-pentafluorophenyl-ethyl}-amide;

(R)-3-Methyl-2-methylamino-pentanoic acid {(S)-1-[(6-amino-pyridin-3-ylmethyl)-carbamoyl]-2-benzo[b]thiophen-3-yl-ethyl}-amide;

(R)-3-Methyl-2-methylamino-pentanoic acid [(S)-1-[(6-amino-pyridin-3-ylmethyl)-carbamoyl]-2-(4-tert-butyl-phenyl)-ethyl]-amide;

(R)-3-Methyl-2-methylamino-pentanoic acid [(S)-1-[(6-amino-pyridin-3-ylmethyl)-carbamoyl]-2-(3,4-difluoro-phenyl)-ethyl]-amide;

(R)-2-Amino-N—[(S)-1-[(6-amino-pyridin-3-ylmethyl)-carbamoyl]-2-(3,4-dichloro-phenyl)-ethyl]-3-phenyl-propionamide;

(R)-Pyrrolidine-2-carboxylic acid [(S)-1-[(6-amino-pyridin-3-ylmethyl)-carbamoyl]-2-(3,4-dichloro-phenyl)-ethyl]-amide;

(S)-Thiazolidine-4-carboxylic acid [(S)-1-[(6-amino-pyridin-3-ylmethyl)-carbamoyl]-2-(3,4-dichloro-phenyl)-ethyl]-amide;

(S)-2-((R)-2-Amino-2-phenyl-acetylamino)-N-(6-amino-pyridin-3-ylmethyl)-3-(3,4-dichloro-phenyl)-propionamide;

(S)-2-((S)-2-Amino-2-phenyl-acetylamino)-N-(6-amino-pyridin-3-ylmethyl)-3-(3,4-dichloro-phenyl)-propionamide;

(R)-2-Amino-N—[(S)-1-[(6-amino-pyridin-3-ylmethyl)-carbamoyl]-2-(3,4-dichloro-phenyl)-ethyl]-3-(4-chloro-phenyl)-propionamide;

(R)—N—[(S)-1-[(6-Amino-pyridin-3-ylmethyl)-carbamoyl]-2-(3,4-dichloro-phenyl)-ethyl]-2-methylamino-3-phenyl-propionamide;

(R)-2-Amino-N—[(S)-1-[(6-amino-pyridin-3-ylmethyl)-carbamoyl]-2-(3,4-dichloro-phenyl)-ethyl]-3,3-dimethyl-butyramide;

(R)—N—[(S)-1-[(6-Amino-pyridin-3-ylmethyl)-carbamoyl]-2-(3,4-dichloro-phenyl)-ethyl]-2-methylamino-propionamide;

(R)-Piperidine-2-carboxylic acid [(S)-1-[(6-amino-pyridin-3-ylmethyl)-carbamoyl]-2-(3,4-dichloro-phenyl)-ethyl]-amide;

(R)-1,2,3,4-Tetrahydro-isoquinoline-3-carboxylic acid [(S)-1-[(6-amino-pyridin-3-ylmethyl)-carbamoyl]-2-(3,4-dichloro-phenyl)-ethyl]-amide;

(R)-2,5-Dihydro-1H-pyrrole-2-carboxylic acid [(S)-1-[(6-amino-pyridin-3-ylmethyl)-carbamoyl]-2-(3,4-dichloro-phenyl)-ethyl]-amide;

(R)-4,4-Difluoro-pyrrolidine-2-carboxylic acid [(S)-1-[(6-amino-pyridin-3-ylmethyl)-carbamoyl]-2-(3,4-dichloro-phenyl)-ethyl]-amide;

(R)-2-Amino-N—[(S)-1-[(6-amino-pyridin-3-ylmethyl)-carbamoyl]-2-(3,4-dichloro-phenyl)-ethyl]-4-phenyl-butyramide;

(R)-2-Amino-N—[(S)-1-[(6-amino-pyridin-3-ylmethyl)-carbamoyl]-2-(3,4-dichloro-phenyl)-ethyl]-3-cyclohexyl-propionamide;

(S)—N-(6-Amino-pyridin-3-ylmethyl)-3-(3,4-dichloro-phenyl)-2-(2-methylamino-acetylamino)-propionamide;

(R)-2-Amino-N—[(S)-1-[(6-amino-pyridin-3-ylmethyl)-carbamoyl]-2-(3-trifluoromethyl-phenyl)-ethyl]-3-(4-chloro-phenyl)-propionamide;

(R)-Pyrrolidine-2-carboxylic acid [(S)-1-[(6-amino-pyridin-3-ylmethyl)-carbamoyl]-2-(4-fluoro-phenyl)-ethyl]-amide;

(R)-Pyrrolidine-2-carboxylic acid [(S)-1-[(6-amino-pyridin-3-ylmethyl)-carbamoyl]-2-(3,4-difluoro-phenyl)-ethyl]-amide;

(R)-Piperidine-2-carboxylic acid [(S)-1-[(6-amino-pyridin-3-ylmethyl)-carbamoyl]-2-(4-fluoro-phenyl)-ethyl]-amide;

(R)-Piperidine-2-carboxylic acid [(S)-1-[(6-amino-pyridin-3-ylmethyl)-carbamoyl]-2-(3,4-difluoro-phenyl)-ethyl]-amide;

(R)-Piperidine-2-carboxylic acid {(S)-1-[(6-amino-pyridin-3-ylmethyl)-carbamoyl]-2-naphthalen-1-ylethyl}-amide;

(R)-Piperidine-2-carboxylic acid [(S)-1-[(6-amino-pyridin-3-ylmethyl)-carbamoyl]-2-(2-chloro-phenyl)-ethyl]-amide;

(R)-Piperidine-2-carboxylic acid [(S)-1-[(6-amino-pyridin-3-ylmethyl)-carbamoyl]-2-(3-chloro-phenyl)-ethyl]-amide;

(R)-Piperidine-2-carboxylic acid {(S)-1-[(6-amino-pyridin-3-ylmethyl)-carbamoyl]-2-benzo[b]thiophen-3-yl-ethyl}-amide;

(S)—N-(6-Amino-pyridin-3-ylmethyl)-3-(3,4-difluoro-phenyl)-2-(2-methylamino-acetylamino)-propionamide;

(R)-2-Amino-N—[(S)-1-[(6-amino-pyridin-3-ylmethyl)-carbamoyl]-2-(3,4-difluoro-phenyl)-ethyl]-3-phenyl-propionamide;

(R)—N—[(S)-1-[(6-Amino-pyridin-3-ylmethyl)carbamoyl]-2-(3,4-difluoro-phenyl)-ethyl]-2-methylamino-3-phenyl-propionamide;

(S)-3-Methyl-2-methylamino-pentanoic acid [(S)-1-[(6-amino-pyridin-3-ylmethyl)-carbamoyl]-2-(3,4-difluoro-phenyl)-ethyl]-amide;

(S)-2-Amino-3-methyl-pentanoic acid [(S)-1-[(6-amino-pyridin-3-ylmethyl)-carbamoyl]-2-(3,4-difluoro-phenyl)-ethyl]-amide;

(R)—N—[(S)-1-[(6-Amino-pyridin-3-ylmethyl)-carbamoyl]-2-(3,4-difluoro-phenyl)-ethyl]-2-methylamino-propionamide;

(S)—N—[(S)-1-[(6-Amino-pyridin-3-ylmethyl)-carbamoyl]-2-(3,4-difluoro-phenyl)-ethyl]-2-methylamino-propionamide;

N—[(S)-1-[(6-Amino-pyridin-3-ylmethyl)-carbamoyl]-2-(3,4-difluoro-phenyl)-ethyl]-2-methyl-2-methylamino-propionamide;

(R)—N—[(S)-1-[(6-Amino-pyridin-3-ylmethyl)-carbamoyl]-2-(4-fluoro-phenyl)-ethyl]-2-methylamino-propionamide;

(S)—N—[(S)-1-[(6-Amino-pyridin-3-ylmethyl)-carbamoyl]-2-(4-fluoro-phenyl)-ethyl]-2-methylamino-propionamide;

(R)—N—[(S)-1-[(6-Amino-pyridin-3-ylmethyl)-carbamoyl]-2-(4-fluoro-phenyl)-ethyl]-3-methyl-2-methylamino-butyramide;

(R)-1-Methyl-pyrrolidine-2-carboxylic acid [(S)-1-[(6-amino-pyridin-3-ylmethyl)-carbamoyl]-2-(3,4-dichloro-phenyl)-ethyl]-amide;

(R)-1-Methyl-pyrrolidine-2-carboxylic acid [(S)-1-[(6-amino-pyridin-3-ylmethyl)-carbamoyl]-2-(3-trifluoromethyl-phenyl)-ethyl]-amide;

(R)-1-Methyl-pyrrolidine-2-carboxylic acid {(S)-1-[(6-amino-pyridin-3-ylmethyl)-carbamoyl]-2-naphthalen-1-yl-ethyl}-amide;

(R)-1-Methyl-pyrrolidine-2-carboxylic acid [(S)-1-[(6-amino-pyridin-3-ylmethyl)-carbamoyl]-2-(4-chloro-phenyl)-ethyl]-amide;

(R)-1-Methyl-pyrrolidine-2-carboxylic acid [(S)-1-[(6-amino-pyridin-3-ylmethyl)-carbamoyl]-2-(2,4,5-trifluoro-phenyl)-ethyl]-amide;

(R)-1-Methyl-pyrrolidine-2-carboxylic acid {(S)-1-[(6-amino-pyridin-3-ylmethyl)-carbamoyl]-2-benzo[b]thiophen-3-yl-ethyl}-amide;

(R)-1-Methyl-pyrrolidine-2-carboxylic acid [(S)-1-[(6-amino-pyridin-3-ylmethyl)-carbamoyl]-2-(3,5-difluoro-phenyl)-ethyl]-amide;

(R)-1-Methyl-pyrrolidine-2-carboxylic acid {(S)-1-[(6-amino-pyridin-3-ylmethyl)-carbamoyl]-2-pentafluorophenyl-ethyl}-amide;

(R)-1-Methyl-pyrrolidine-2-carboxylic acid [(S)-1-[(6-amino-pyridin-3-ylmethyl)-carbamoyl]-2-(4-fluoro-phenyl)-ethyl]-amide;

(R)-1-Methyl-pyrrolidine-2-carboxylic acid [(S)-1-[(6-amino-pyridin-3-ylmethyl)-carbamoyl]-2-(3-fluoro-phenyl)-ethyl]-amide;

(R)-1-Methyl-pyrrolidine-2-carboxylic acid [(S)-1-[(6-amino-pyridin-3-ylmethyl)-carbamoyl]-2-(4-methoxy-phenyl)-ethyl]-amide;

(R)-1-Methyl-pyrrolidine-2-carboxylic acid {(S)-1-[(6-amino-pyridin-3-ylmethyl)-carbamoyl]-2-phenyl-ethyl}-amide;

(R)-1-Methyl-pyrrolidine-2-carboxylic acid [(S)-1-[(6-amino-pyridin-3-ylmethyl)-carbamoyl]-2-(4-tert-butyl-phenyl)-ethyl]-amide;

(R)-1-Methyl-pyrrolidine-2-carboxylic acid [(S)-1-[(6-amino-pyridin-3-ylmethyl)-carbamoyl]-2-(3-chloro-phenyl)-ethyl]-amide;

(R)-1-Methyl-pyrrolidine-2-carboxylic acid {(S)-1-[(6-amino-pyridin-3-ylmethyl)-carbamoyl]-2-o-tolyl-ethyl}-amide;

(R)-1-Methyl-pyrrolidine-2-carboxylic acid {(S)-1-[(6-amino-pyridin-3-ylmethyl)-carbamoyl]-2-m-tolyl-ethyl}-amide;

(R)-1-Methyl-pyrrolidine-2-carboxylic acid {(S)-1-[(6-amino-pyridin-3-ylmethyl)-carbamoyl]-2-p-tolyl-ethyl}-amide;

(R)-1-Methyl-pyrrolidine-2-carboxylic acid [(S)-1-[(6-amino-pyridin-3-ylmethyl)-carbamoyl]-2-(3-methoxy-phenyl)-ethyl]-amide;

(R)-1-Methyl-pyrrolidine-2-carboxylic acid [(S)-1-[(6-amino-pyridin-3-ylmethyl)-carbamoyl]-2-(2-fluoro-phenyl)-ethyl]-amide;

(R)-1-Methyl-pyrrolidine-2-carboxylic acid [(S)-1-[(6-amino-pyridin-3-ylmethyl)-carbamoyl]-2-(2-chloro-phenyl)-ethyl]-amide;

(R)-1-Methyl-pyrrolidine-2-carboxylic acid [(S)-1-[(6-amino-pyridin-3-ylmethyl)-carbamoyl]-2-(2,4-dichloro-phenyl)-ethyl]-amide;

(R)-1-Methyl-pyrrolidine-2-carboxylic acid [(S)-1-[(6-amino-pyridin-3-ylmethyl)-carbamoyl]-2-(2-trifluoromethyl-phenyl)-ethyl]-amide;

(R)-1-Methyl-pyrrolidine-2-carboxylic acid {(S)-1-[(6-amino-pyridin-3-ylmethyl)-carbamoyl]-2-pyridin-4-yl-ethyl}-amide;

(R)-1-Methyl-pyrrolidine-2-carboxylic acid [(S)-1-[(6-amino-pyridin-3-ylmethyl)-carbamoyl]-2-(1H-indol-3-yl)-ethyl]-amide;

(R)-1-Methyl-pyrrolidine-2-carboxylic acid {(S)-1-[(6-amino-pyridin-3-ylmethyl)-carbamoyl]-2-quinolin-2-yl-ethyl}-amide;

(R)-1-Methyl-pyrrolidine-2-carboxylic acid {(S)-1-[(6-amino-pyridin-3-ylmethyl)-carbamoyl]-2-cyclohexyl-ethyl}-amide;

(S)-1-Methyl-pyrrolidine-2-carboxylic acid [(S)-1-[(6-amino-pyridin-3-ylmethyl)-carbamoyl]-2-(3,4-difluoro-phenyl)-ethyl]-amide;

(R)-1-Isopropyl-pyrrolidine-2-carboxylic acid [(S)-1-[(6-amino-pyridin-3-ylmethyl)-carbamoyl]-2-(3,4-dichloro-phenyl)-ethyl]-amide;

(R)-1-Isopropyl-pyrrolidine-2-carboxylic acid {(S)-1-[(6-amino-pyridin-3-ylmethyl)-carbamoyl]-2-naphthalen-1-yl-ethyl}-amide;

(R)-1-Isopropyl-pyrrolidine-2-carboxylic acid [(S)-1-[(6-amino-pyridin-3-ylmethyl)-carbamoyl]-2-(4-fluoro-phenyl)-ethyl]-amide;

(R)-1-Isopropyl-pyrrolidine-2-carboxylic acid [(S)-1-[(6-amino-pyridin-3-ylmethyl)-carbamoyl]-2-(3-chloro-phenyl)-ethyl]-amide;

(R)-1-Isopropyl-pyrrolidine-2-carboxylic acid {(S)-1-[(6-amino-pyridin-3-ylmethyl)-carbamoyl]-2-o-tolyl-ethyl}-amide;

(R)-1-Isopropyl-pyrrolidine-2-carboxylic acid {(S)-1-[(6-amino-pyridin-3-ylmethyl)-carbamoyl]-2-m-tolyl-ethyl}-amide;

(R)-1-Isopropyl-pyrrolidine-2-carboxylic acid {(S)-1-[(6-amino-pyridin-3-ylmethyl)-carbamoyl]-2-p-tolyl-ethyl}-amide;

(R)-1-Isopropyl-pyrrolidine-2-carboxylic acid [(S)-1-[(6-amino-pyridin-3-ylmethyl)-carbamoyl]-2-(3-methoxy-phenyl)-ethyl]-amide;

(R)-1-Isopropyl-pyrrolidine-2-carboxylic acid [(S)-1-[(6-amino-pyridin-3-ylmethyl)-carbamoyl]-2-(2-fluoro-phenyl)-ethyl]-amide;

(R)-1-Isopropyl-pyrrolidine-2-carboxylic acid [(S)-1-[(6-amino-pyridin-3-ylmethyl)-carbamoyl]-2-(2-chloro-phenyl)-ethyl]-amide;

(R)-1-Isopropyl-pyrrolidine-2-carboxylic acid [(S)-1-[(6-amino-pyridin-3-ylmethyl)-carbamoyl]-2-(2,4-dichloro-phenyl)-ethyl]-amide;

(R)-1-Isopropyl-pyrrolidine-2-carboxylic acid [(S)-1-[(6-amino-pyridin-3-ylmethyl)-carbamoyl]-2-(2-trifluoromethyl-phenyl)-ethyl]-amide;

(R)-1-Isopropyl-pyrrolidine-2-carboxylic acid {(S)-1-[(6-amino-pyridin-3-ylmethyl)-carbamoyl]-2-pyridin-4-yl-ethyl}-amide;

(R)-1-Isopropyl-pyrrolidine-2-carboxylic acid [(S)-1-[(6-amino-pyridin-3-ylmethyl)-carbamoyl]-2-(1H-indol-3-yl)-ethyl]-amide;

(R)-1-Ethyl-pyrrolidine-2-carboxylic acid [(S)-1-[(6-amino-pyridin-3-ylmethyl)-carbamoyl]-2-(3,4-difluoro-phenyl)-ethyl]-amide;

(R)-1-Propyl-pyrrolidine-2-carboxylic acid [(S)-1-[(6-amino-pyridin-3-ylmethyl)-carbamoyl]-2-(3,4-difluoro-phenyl)-ethyl]-amide

(R)-1-Isobutyl-pyrrolidine-2-carboxylic acid [(S)-1-[(6-amino-pyridin-3-ylmethyl)-carbamoyl]-2-(3,4-difluoro-phenyl)-ethyl]-amide;

{(R)-2-[(S)-1-[(6-Amino-pyridin-3-ylmethyl)-carbamoyl]-2-(3,4-difluoro-phenyl)-ethylcarbamoyl]-pyrrolidin-1-yl}-acetic acid methyl ester;

{(R)-2-[(S)-1-[(6-Amino-pyridin-3-ylmethyl)-carbamoyl]-2-(3,4-difluoro-phenyl)-ethylcarbamoyl]-pyrrolidin-1-yl}-acetic acid;

(R)-1-Ethyl-pyrrolidine-2-carboxylic acid [(S)-1-[(6-amino-pyridin-3-ylmethyl)-carbamoyl]-2-(4-fluoro-phenyl)-ethyl]-amide;

(R)-1-Ethyl-pyrrolidine-2-carboxylic acid {(S)-1-[(6-amino-pyridin-3-ylmethyl)-carbamoyl]-2-m-tolyl-ethyl}-amide;

(R)-1-Ethyl-pyrrolidine-2-carboxylic acid [(S)-1-[(6-amino-pyridin-3-ylmethyl)-carbamoyl]-2-(3-chloro-phenyl)-ethyl]-amide;

(R)-1-Propyl-pyrrolidine-2-carboxylic acid [(S)-1-[(6-amino-pyridin-3-ylmethyl)-carbamoyl]-2-(4-fluoro-phenyl)-ethyl]-amide;

(R)-1-Benzyl-pyrrolidine-2-carboxylic acid [(S)-1-[(6-amino-pyridin-3-ylmethyl)-carbamoyl]-2-(3,4-difluoro-phenyl)-ethyl]-amide;

(R)-1-Benzyl-pyrrolidine-2-carboxylic acid [(S)-1-[(6-amino-pyridin-3-ylmethyl)-carbamoyl]-2-(3,4-dichloro-phenyl)-ethyl]-amide;

(R)-1-(4-Chloro-benzyl)-pyrrolidine-2-carboxylic acid [(S)-1-[(6-amino-pyridin-3-ylmethyl)-carbamoyl]-2-(3,4-difluoro-phenyl)-ethyl]-amide;

(R)-1-(3-Chloro-benzyl)-pyrrolidine-2-carboxylic acid [(S)-1-[(6-amino-pyridin-3-ylmethyl)-carbamoyl]-2-(3,4-difluoro-phenyl)-ethyl]-amide;

(R)-1-Methyl-piperidine-2-carboxylic acid [(S)-1-[(6-amino-pyridin-3-ylmethyl)-carbamoyl]-2-(3,4-difluoro-phenyl)-ethyl]-amide;

(R)-1-Methyl-piperidine-2-carboxylic acid [(S)-1-[(6-amino-pyridin-3-ylmethyl)-carbamoyl]-2-(4-fluoro-phenyl)-ethyl]-amide;

(R)-1-Isopropyl-piperidine-2-carboxylic acid [(S)-1-[(6-amino-pyridin-3-ylmethyl)-carbamoyl]-2-(3,4-difluoro-phenyl)-ethyl]-amide;

(R)-1-Isopropyl-piperidine-2-carboxylic acid [(S)-1-[(6-amino-pyridin-3-ylmethyl)-carbamoyl]-2-(4-fluoro-phenyl)-ethyl]-amide;

(2R,4R)-4-Hydroxy-1-methyl-pyrrolidine-2-carboxylic acid [(S)-1-[(6-amino-pyridin-3-ylmethyl)-carbamoyl]-2-(3,4-difluoro-phenyl)-ethyl]-amide;

(R)-2-Methyl-1,2,3,4-tetrahydro-isoquinoline-3-carboxylic acid [(S)-1-[(6-amino-pyridin-3-ylmethyl)-carbamoyl]-2-(3,4-difluoro-phenyl)-ethyl]-amide;

(S)—N-(6-Amino-pyridin-3-ylmethyl)-3-(3,4-dichloro-phenyl)-2-[2-(isopropyl-methyl-amino)-acetylamino]-propionamide;

(R)—N—[(S)-1-[(6-Amino-pyridin-3-ylmethyl)-carbamoyl]-2-(3,4-dichloro-phenyl)-ethyl]-2-(isopropyl-methyl-amino)-propionamide;

(S)—N-(6-Amino-pyridin-3-ylmethyl)-2-[2-(benzyl-methyl-amino)-acetylamino]-3-(3,4-dichloro-phenyl)-propionamide;

(S)—N-(6-Amino-pyridin-3-ylmethyl)-3-(3,4-dichloro-phenyl)-2-(2-dimethylamino-acetylamino)-propionamide;

(S)—N-(6-Amino-pyridin-3-ylmethyl)-3-(3,4-dichloro-phenyl)-2-[2-(isobutyl-methyl-amino)-acetylamino]-propionamide;

(R)-2-(Isopropyl-methyl-amino)-3-methyl-pentanoic acid [(S)-1-[(6-amino-pyridin-3-ylmethyl)-carbamoyl]-2-(3,4-dichloro-phenyl)-ethyl]-amide;

(S)—N-(6-Amino-pyridin-3-ylmethyl)-3-(3,4-difluoro-phenyl)-2-[2-(isopropyl-methyl-amino)-acetylamino]-propionamide;

(S)—N-(6-Amino-pyridin-3-ylmethyl)-3-(3,4-difluoro-phenyl)-2-(2-diisopropylamino-acetylamino)-propionamide;

(S)—N-(6-Amino-pyridin-3-ylmethyl)-3-(3,4-difluoro-phenyl)-2-(2-dipropylamino-acetylamino)-propionamide;

(S)—N-(6-Amino-pyridin-3-ylmethyl)-3-(3,4-difluoro-phenyl)-2-(2-diisobutylamino-acetylamino)-propionamide;

(S)—N-(6-Amino-pyridin-3-ylmethyl)-3-(3,4-difluoro-phenyl)-2-(2-phenethylamino-acetylamino)-propionamide;

(S)—N-(6-Amino-pyridin-3-ylmethyl)-3-(3,4-difluoro-phenyl)-2-[2-(methyl-phenethyl-amino)-acetylamino]-propionamide;

(S)—N-(6-Amino-pyridin-3-ylmethyl)-3-(3,4-difluoro-phenyl)-2-{2-[methyl-((E)-3-phenyl-allyl)-amino]-acetylamino}-propionamide;

(S)—N-(6-Amino-pyridin-3-ylmethyl)-2-{2-[(4-chloro-benzyl)-methyl-amino]-acetylamino}-3-(3,4-difluoro-phenyl)-propionamide;

(S)—N-(6-Amino-pyridin-3-ylmethyl)-2-{2-[(3-chloro-benzyl)-methyl-amino]-acetylamino}-3-(3,4-difluoro-phenyl)-propionamide;

(S)—N-(6-Amino-pyridin-3-ylmethyl)-2-{2-[(2-chloro-benzyl)-methyl-amino]-acetylamino}-3-(3,4-difluoro-phenyl)-propionamide;

(S)—N-(6-Amino-pyridin-3-ylmethyl)-3-(3,4-difluoro-phenyl)-2-{2-[(4-methoxy-benzyl)-methyl-amino]-acetylamino}-propionamide;

(S)—N-(6-Amino-pyridin-3-ylmethyl)-2-[2-(butyl-methyl-amino)-acetylamino]-3-(3,4-difluoro-phenyl)-propionamide;

(S)—N-(6-Amino-pyridin-3-ylmethyl)-3-(3,4-difluoro-phenyl)-2-[2-(isobutyl-methyl-amino)-acetylamino]-propionamide;

(S)—N-(6-Amino-pyridin-3-ylmethyl)-2-[2-(cyclohexylmethyl-methyl-amino)-acetylamino]-3-(3,4-difluoro-phenyl)-propionamide;

(S)—N-(6-Amino-pyridin-3-ylmethyl)-3-(3,4-difluoro-phenyl)-2-(2-piperidin-1-yl-acetylamino)-propionamide;

(S)—N-(6-Amino-pyridin-3-ylmethyl)-3-(3,4-difluoro-phenyl)-2-(2-morpholin-4-yl-acetylamino)-propionamide;

(S)—N-(6-Amino-pyridin-3-ylmethyl)-3-(3,4-difluoro-phenyl)-2-(2-3,4-dihydro-1H-isoquinolin-2-yl-acetylamino)-propionamide;

(S)—N-(6-Amino-pyridin-3-ylmethyl)-3-(3,4-difluoro-phenyl)-2-(2-3,4-dihydro-2H-quinolin-1-yl-acetylamino)-propionamide;

(S)—N-(6-Amino-pyridin-3-ylmethyl)-3-(3,4-difluoro-phenyl)-2-[2-(methyl-phenyl-amino)-acetylamino]-propionamide;

(R)—N—[(S)-1-[(6-Amino-pyridin-3-ylmethyl)-carbamoyl]-2-(3,4-difluoro-phenyl)-ethyl]-2-(isopropyl-methyl-amino)-propionamide;

(S)—N—[(S)-1-[(6-Amino-pyridin-3-ylmethyl)-carbamoyl]-2-(3,4-difluoro-phenyl)-ethyl]-2-(isopropyl-methyl-amino)-propionamide;

(S)—N—[(S)-1-[(6-Amino-pyridin-3-ylmethyl)-carbamoyl]-2-(3,4-difluoro-phenyl)-ethyl]-2-(isobutyl-methyl-amino)-propionamide;

(R)-2-Dimethylamino-3-methyl-pentanoic acid [(S)-1-[(6-amino-pyridin-3-ylmethyl)-carbamoyl]-2-(3,4-difluoro-phenyl)-ethyl]-amide;

(R)—N—[(S)-1-[(6-Amino-pyridin-3-ylmethyl)-carbamoyl]-2-(3,4-difluoro-phenyl)-ethyl]-2-dimethylamino-3,3-dimethyl-butyramide;

(R)—N—[(S)-1-[(6-Amino-pyridin-3-ylmethyl)-carbamoyl]-2-(4-fluoro-phenyl)-ethyl]-2-(isopropyl-methyl-amino)-propionamide;

(S)—N—[(S)-1-[(6-Amino-pyridin-3-ylmethyl)-carbamoyl]-2-(4-fluoro-phenyl)-ethyl]-2-(isopropyl-methyl-amino)-propionamide;

(R)-2-Dimethylamino-3-methyl-pentanoic acid [(S)-1-[(6-amino-pyridin-3-ylmethyl)-carbamoyl]-2-(4-fluoro-phenyl)-ethyl]-amide;

(R)—N—[(S)-1-[(6-Amino-pyridin-3-ylmethyl)-carbamoyl]-2-(4-fluoro-phenyl)-ethyl]-2-dimethylamino-3,3-dimethyl-butyramide;

(R)—N—[(S)-1-[(6-Amino-pyridin-3-ylmethyl)-carbamoyl]-2-(3,4-difluoro-phenyl)-ethyl]-2-isopropylamino-3-phenyl-propionamide;

(R)—N—[(S)-1-[(6-Amino-pyridin-3-ylmethyl)-carbamoyl]-2-(3,4-difluoro-phenyl)-ethyl]-2-dimethylamino-3-phenyl-propionamide;

(R)-2-Amino-N—[(S)-1-[(6-amino-2-methyl-pyridin-3-ylmethyl)-carbamoyl]-2-(decahydro-naphthalen-1-yl)-ethyl]-3-methyl-butyramide;

(R)-2-Amino-3-methyl-pentanoic acid [(S)-1-[(6-amino-2-methyl-pyridin-3-ylmethyl)-carbamoyl]-2-(decahydro-naphthalen-1-yl)-ethyl]-amide;

(2R)-3-Methyl-2-methylamino-pentanoic acid [(R)-1-[(6-amino-2-methyl-pyridin-3-ylmethyl)-carbamoyl]-2-(3,4-dichloro-phenyl)-ethyl]-amide;

(R)-Pyrrolidine-2-carboxylic acid [(R)-1-[(6-amino-2-methyl-pyridin-3-ylmethyl)-carbamoyl]-2-(3,4-dichloro-phenyl)-ethyl]-amide;

(R)-1-Methyl-pyrrolidine-2-carboxylic acid [(R)-1-[(6-amino-2-methyl-pyridin-3-ylmethyl)-carbamoyl]-2-(3,4-dichloro-phenyl)-ethyl]-amide;

(S)-Thiazolidine-4-carboxylic acid [(R)-1-[(6-amino-2-methyl-pyridin-3-ylmethyl)-carbamoyl]-2-(3,4-dichloro-phenyl)-ethyl]-amide;

(R)-4,4-Difluoro-pyrrolidine-2-carboxylic acid [(S)-1-[(6-amino-2-methyl-pyridin-3-ylmethyl)-carbamoyl]-2-(3,4-dichloro-phenyl)-ethyl]-amide;

(S)—N—[(R)-1-[(6-Amino-2-methyl-pyridin-3-ylmethyl)-carbamoyl]-2-(3,4-dichloro-phenyl)-ethyl]-2-methylamino-3-phenyl-propionamide;

(R)-2-Amino-N—[(S)-1-[(6-amino-2-methyl-pyridin-3-ylmethyl)-carbamoyl]-2-(3,4-dichloro-phenyl)-ethyl]-3-(4-fluoro-phenyl)-propionamide;

(R)-2-Amino-N—[(S)-1-[(6-amino-2-methyl-pyridin-3-ylmethyl)-carbamoyl]-2-(3,4-dichloro-phenyl)-ethyl]-3-(4-chloro-phenyl)-propionamide;

(R)-2-Amino-N—[(S)-1-[(6-amino-2-methyl-pyridin-3-ylmethyl)-carbamoyl]-2-(3,4-dichloro-phenyl)-ethyl]-3-pyridin-3-yl-propionamide;

(R)—N-(6-Amino-2-methyl-pyridin-3-ylmethyl)-2-((S)-2-amino-2-phenyl-acetylamino)-3-(3,4-dichloro-phenyl)-propionamide;

(S)-2-((R)-2-Amino-2-cyclohexyl-acetylamino)-N-(6-amino-2-methyl-pyridin-3-ylmethyl)-3-(3,4-dichloro-phenyl)-propionamide;

(R)-2-Amino-N—[(S)-1-[(6-amino-2-methyl-pyridin-3-ylmethyl)-carbamoyl]-2-(3,4-dichloro-phenyl)-ethyl]-3,3-dimethyl-butyramide;

(R)-2-Amino-N—[(S)-1-[(6-amino-2-methyl-pyridin-3-ylmethyl)-carbamoyl]-2-(3,4-dichloro-phenyl)-ethyl]-3-cyclohexyl-propionamide;

(R)-Piperidine-2-carboxylic acid [(S)-1-[(6-amino-2-methyl-pyridin-3-ylmethyl)-carbamoyl]-2-(3,4-dichloro-phenyl)-ethyl]-amide;

(R)-1,2,3,4-Tetrahydro-quinoline-2-carboxylic acid [(S)-1-[(6-amino-2-methyl-pyridin-3-ylmethyl)-carbamoyl]-2-(3,4-dichloro-phenyl)-ethyl]-amide;

(R)-3-Methyl-2-methylamino-pentanoic acid {(S)-1-[(6-amino-2-methyl-pyridin-3-ylmethyl)-carbamoyl]-2-naphthalen-1-yl-ethyl}-amide;

(R)-1-Methyl-pyrrolidine-2-carboxylic acid [(S)-1-[(6-amino-2-methyl-pyridin-3-ylmethyl)-carbamoyl]-2-(4-fluoro-phenyl)-ethyl]-amide;

(R)-1-Methyl-pyrrolidine-2-carboxylic acid [(S)-1-[(6-amino-2-methyl-pyridin-3-ylmethyl)-carbamoyl]-2-(3,4-difluoro-phenyl)-ethyl]-amide;

(R)-1-Methyl-pyrrolidine-2-carboxylic acid [(S)-1-[(6-amino-2,4-dimethyl-pyridin-3-ylmethyl)-carbamoyl]-2-(3,4-difluoro-phenyl)-ethyl]-amide;

(R)-3-Methyl-2-methylamino-pentanoic acid [(S)-1-[(6-amino-2,4-dimethyl-pyridin-3-ylmethyl)-carbamoyl]-2-(3,4-difluoro-phenyl)-ethyl]-amide;

(R)-2-Amino-3-methyl-pentanoic acid [(S)-1-[(6-amino-2,4-dimethyl-pyridin-3-ylmethyl)-carbamoyl]-2-(decahydro-naphthalen-1-yl)-ethyl]-amide;

(R)-2-Amino-3-methyl-pentanoic acid {(S)-1-[(6-amino-5-methyl-pyridin-3-ylmethyl)-carbamoyl]-2-naphthalen-1-yl-ethyl}-amide;

(R)-1-Methanesulfonyl-pyrrolidine-2-carboxylic acid [(S)-1-[(6-amino-pyridin-3-ylmethyl)-carbamoyl]-2-(3,4-dichloro-phenyl)-ethyl]-amide;

(R)-2-Methanesulfonylamino-3-methyl-pentanoic acid [(S)-1-[(6-amino-pyridin-3-ylmethyl)-carbamoyl]-2-(3,4-dichloro-phenyl)-ethyl]-amide;

(R)—N—[(S)-1-[(6-Amino-pyridin-3-ylmethyl)-carbamoyl]-2-(3,4-dichloro-phenyl)-ethyl]-2-(methanesulfonyl-methyl-amino)-propionamide;

(R)-2-(Methanesulfonyl-methyl-amino)-3-methyl-pentanoic acid {(S)-1-[(6-amino-pyridin-3-ylmethyl)-carbamoyl]-2-benzo[b]thiophen-3-yl-ethyl}-amide;

(R)-2-Amino-3-methyl-pentanoic acid {(R)-1-[(6-amino-pyridin-3-ylmethyl)-carbamoyl]-2,2-dicyclohexyl-ethyl}-amide;

(S)-Pyrrolidine-2-carboxylic acid {(R)-1-[(6-amino-pyridin-3-ylmethyl)-carbamoyl]-2,2-dicyclohexyl-ethyl}-amide;

(S)-2-Amino-N—{(R)-1-[(6-amino-pyridin-3-ylmethyl)-carbamoyl]-2,2-dicyclohexyl-ethyl}-3-phenyl-propionamide;

(S)-2-Amino-N—{(R)-1-[(6-amino-pyridin-3-ylmethyl)-carbamoyl]-2,2-dicyclohexyl-ethyl}-3,3-dimethyl-butyramide;

(S)—N—{(R)-1-[(6-Amino-pyridin-3-ylmethyl)-carbamoyl]-2,2-dicyclohexyl-ethyl}-2-methylamino-propionamide;

(S)-3-Methyl-2-methylamino-pentanoic acid {(R)-1-[(6-amino-pyridin-3-ylmethyl)-carbamoyl]-2,2-dicyclohexyl-ethyl}-amide;

(R)-2-(2-Amino-acetylamino)-N-(6-amino-pyridin-3-ylmethyl)-3,3-dicyclohexyl-propionamide;

(R)—N-(6-Amino-pyridin-3-ylmethyl)-3,3-dicyclohexyl-2-(2-methylamino-acetylamino)-propionamide;

(R)—N-(6-Amino-pyridin-3-ylmethyl)-3,3-dicyclohexyl-2-(2-dimethylamino-acetylamino)-propionamide;

(S)-1-Methyl-pyrrolidine-2-carboxylic acid {(R)-1-[(6-amino-pyridin-3-ylmethyl)-carbamoyl]-2,2-dicyclohexyl-ethyl}-amide;

(S)-2-Amino-3-methyl-pentanoic acid {(1R,2R)-1-[(6-amino-pyridin-3-ylmethyl)-carbamoyl]-2-hydroxy-2-phenyl-ethyl}-amide;

(R)-1-Methyl-pyrrolidine-2-carboxylic acid {(S)-1-[(6-amino-pyridin-3-ylmethyl)-carbamoyl]-2-phenyl-ethyl}-methyl-amide;

3-Methyl-1H-pyrrole-2-carboxylic acid [(S)-1-[(6-amino-pyridin-3-ylmethyl)-carbamoyl]-2-(3,4-difluoro-phenyl)-ethyl]-amide;

1H-Pyrrole-2-carboxylic acid [(S)-1-[(6-amino-pyridin-3-ylmethyl)-carbamoyl]-2-(decahydro-naphthalen-1-yl)-ethyl]-amide;

1H-Pyrrole-2-carboxylic acid {(S)-1-[(6-amino-pyridin-3-ylmethyl)-carbamoyl]-2-naphthalen-1-yl-ethyl}-amide;

1H-Pyrrole-2-carboxylic acid [(S)-1-[(6-amino-pyridin-3-ylmethyl)-carbamoyl]-2-(3-trifluoromethyl-phenyl)-ethyl]-amide;

3,4,5-Trimethyl-1H-pyrrole-2-carboxylic acid [(S)-1-[(6-amino-pyridin-3-ylmethyl)-carbamoyl]-2-(3-trifluoromethyl-phenyl)-ethyl]-amide;

3,5-Dimethyl-1H-pyrrole-2-carboxylic acid {(S)-1-[(6-amino-pyridin-3-ylmethyl)-carbamoyl]-2-naphthalen-1-yl-ethyl}-amide;

3,5-Dimethyl-1H-pyrrole-2-carboxylic acid [(S)-1-[(6-amino-pyridin-3-ylmethyl)-carbamoyl]-2-(3-trifluoromethyl-phenyl)-ethyl]-amide;

3,5-Dimethyl-1H-pyrrole-2-carboxylic acid [(S)-1-[(6-amino-pyridin-3-ylmethyl)-carbamoyl]-2-(3,4-dichloro-phenyl)-ethyl]-amide;

3,5-Dimethyl-1H-pyrrole-2-carboxylic acid {(S)-1-[(6-amino-pyridin-3-ylmethyl)-carbamoyl]-2-m-tolyl-ethyl}-amide;

3,5-Dimethyl-1H-pyrrole-2-carboxylic acid [(S)-1-[(6-amino-pyridin-3-ylmethyl)-carbamoyl]-2-(3-chloro-phenyl)-ethyl]-amide;

3,5-Dimethyl-1H-pyrrole-2-carboxylic acid [(S)-1-[(6-amino-pyridin-3-ylmethyl)-carbamoyl]-2-(3-fluoro-phenyl)-ethyl]-amide;

3,5-Dimethyl-1H-pyrrole-2-carboxylic acid {(S)-1-[(6-amino-pyridin-3-ylmethyl)-carbamoyl]-2-phenyl-ethyl}-amide;

3,5-Dimethyl-1H-pyrrole-2-carboxylic acid {(S)-1-[(6-amino-pyridin-3-ylmethyl)-carbamoyl]-2-cyclohexyl-ethyl}-amide;

3,5-Dimethyl-1H-pyrrole-2-carboxylic acid [(S)-1-[(;6-amino-pyridin-3-ylmethyl)-carbamoyl]-2-(2-chloro-phenyl)-ethyl]-amide;

3,5-Dimethyl-1H-pyrrole-2-carboxylic acid [(S)-1-[(6-amino-pyridin-3-ylmethyl)-carbamoyl]-2-(2-trifluoromethyl-phenyl)-ethyl]-amide;

3,5-Dimethyl-1H-pyrrole-2-carboxylic acid [(S)-1-[(6-amino-pyridin-3-ylmethyl)-carbamoyl]-2-(1H-indol-3-yl)-ethyl]-amide;

3,5-Dimethyl-1H-pyrrole-2-carboxylic acid [(S)-1-[(6-amino-pyridin-3-ylmethyl)-carbamoyl]-2-(4-fluoro-phenyl)-ethyl]-amide;

3,5-Dimethyl-1H-pyrrole-2-carboxylic acid [(S)-1-[(6-amino-pyridin-3-ylmethyl)-carbamoyl]-2-(3,4-difluoro-phenyl)-ethyl]-amide;

3,5-Dimethyl-1H-pyrrole-2-carboxylic acid {(S)-1-[(6-amino-pyridin-3-ylmethyl)-carbamoyl]-2-o-tolyl-ethyl}-amide;

3,5-Dimethyl-1H-pyrrole-2-carboxylic acid [(S)-1-[(6-amino-pyridin-3-ylmethyl)-carbamoyl]-2-(2,4,5-trifluoro-phenyl)-ethyl]-amide;

3,5-Dimethyl-1H-pyrrole-2-carboxylic acid [(S)-1-[(6-amino-pyridin-3-ylmethyl)-carbamoyl]-2-(4-chloro-phenyl)-ethyl]-amide;

3,5-Dimethyl-1H-pyrrole-2-carboxylic acid [(S)-1-[(6-amino-pyridin-3-ylmethyl)-carbamoyl]-2-(4-hydroxy-phenyl)-ethyl]-amide;

3,5-Dimethyl-1H-pyrrole-2-carboxylic acid [(S)-1-[(6-amino-pyridin-3-ylmethyl)-carbamoyl]-2-(2,4,5-trifluoro-phenyl)-ethyl]-amide;

3,5-Dimethyl-1H-pyrrole-2-carboxylic acid [(S)-1-[(6-amino-pyridin-3-ylmethyl)-carbamoyl]-2-(3,5-difluoro-phenyl)-ethyl]-amide;

3,5-Dimethyl-1H-pyrrole-2-carboxylic acid {(S)-1-[(6-amino-pyridin-3-ylmethyl)-carbamoyl]-2-benzo[b]thiophen-3-yl-ethyl}-amide;

3,5-Dimethyl-1H-pyrrole-2-carboxylic acid {(S)-1-[(6-amino-pyridin-3-ylmethyl)-carbamoyl]-2-pentafluorophenyl-ethyl}-amide;

3,5-Dimethyl-1H-pyrrole-2-carboxylic acid {(S)-1-[(6-amino-pyridin-3-ylmethyl)-carbamoyl]-2-biphenyl-4-yl-ethyl}-amide;

3,5-Dimethyl-1H-pyrrole-2-carboxylic acid {(S)-1-[(6-amino-pyridin-3-ylmethyl)-carbamoyl]-2-p-tolyl-ethyl}-amide;

3,5-Dimethyl-1H-pyrrole-2-carboxylic acid [(S)-1-[(6-amino-pyridin-3-ylmethyl)-carbamoyl]-2-(4-methoxy-phenyl)-ethyl]-amide;

3,5-Dimethyl-1H-pyrrole-2-carboxylic acid [(S)-1-[(6-amino-pyridin-3-ylmethyl)-carbamoyl]-2-(4-ethoxy-phenyl)-ethyl]-amide;

3,5-Dimethyl-1H-pyrrole-2-carboxylic acid [(S)-1-[(6-amino-pyridin-3-ylmethyl)-carbamoyl]-2-(4-tert-butyl-phenyl)-ethyl]-amide;

3,5-Dimethyl-1H-pyrrole-2-carboxylic acid {(S)-1-[(6-amino-pyridin-3-ylmethyl)-carbamoyl]-2-quinolin-2-yl-ethyl}-amide;

3-Methyl-1H-pyrrole-2-carboxylic acid [(S)-1-[(6-amino-pyridin-3-ylmethyl)-carbamoyl]-2-(4-trifluoromethyl-phenyl)-ethyl]-amide;

4-Methyl-1H-pyrrole-2-carboxylic acid [(S)-1-[(6-amino-pyridin-3-ylmethyl)-carbamoyl]-2-(3-chloro-phenyl)-ethyl]-amide;

4-Methyl-1H-pyrrole-2-carboxylic acid [(S)-1-[(6-amino-pyridin-3-ylmethyl)-carbamoyl]-2-(4-fluoro-phenyl)-ethyl]-amide;

3-Methyl-1H-pyrrole-2-carboxylic acid [(S)-1-[(6-amino-pyridin-3-ylmethyl)-carbamoyl]-2-(4-fluoro-phenyl)-ethyl]-amide;

3-Methyl-1H-pyrrole-2-carboxylic acid [(S)-1-[(6-amino-pyridin-3-ylmethyl)-carbamoyl]-2-(2-chloro-phenyl)-ethyl]-amide;

3-Methyl-1H-pyrrole-2-carboxylic acid [(S)-1-[(6-amino-pyridin-3-ylmethyl)-carbamoyl]-2-(2,5-dichloro-phenyl)-ethyl]-amide;

3-Methyl-1H-pyrrole-2-carboxylic acid [(S)-1-[(6-amino-pyridin-3-ylmethyl)-carbamoyl]-2-(3,4-dichloro-phenyl)-ethyl]-amide;

3-Methyl-1H-pyrrole-2-carboxylic acid [(S)-1-[(6-amino-pyridin-3-ylmethyl)-carbamoyl]-2-(2-fluoro-phenyl)-ethyl]-amide;

3-Methyl-1H-pyrrole-2-carboxylic acid [(S)-1-[(6-amino-pyridin-3-ylmethyl)-carbamoyl]-2-(3-chloro-phenyl)-ethyl]-amide;

3-Methyl-1H-pyrrole-2-carboxylic acid [(S)-1-[(6-amino-pyridin-3-ylmethyl)-carbamoyl]-2-(4-chloro-phenyl)-ethyl]-amide;

3-Methyl-1H-pyrrole-2-carboxylic acid {(S)-1-[(6-amino-pyridin-3-ylmethyl)-carbamoyl]-2-m-tolyl-ethyl}-amide;

3-Methyl-1H-pyrrole-2-carboxylic acid [(S)-1-[(6-amino-pyridin-3-ylmethyl)-carbamoyl]-2-(3-fluoro-phenyl)-ethyl]-amide;

3-Methyl-1H-pyrrole-2-carboxylic acid [(S)-1-[(6-amino-pyridin-3-ylmethyl)-carbamoyl]-2-(3,5-difluoro-phenyl)-ethyl]-amide;

3-Methyl-1H-pyrrole-2-carboxylic acid [(S)-1-[(6-amino-pyridin-3-ylmethyl)-carbamoyl]-2-(3-trifluoromethyl-phenyl)-ethyl]-amide;

3-Methyl-1H-pyrrole-2-carboxylic acid [(S)-1-[(6-amino-pyridin-3-ylmethyl)-carbamoyl]-2-(4-trifluoromethyl-phenyl)-ethyl]-amide;

3-Methyl-1H-pyrrole-2-carboxylic acid {(S)-1-[(6-amino-pyridin-3-ylmethyl)-carbamoyl]-2-cyclohexyl-ethyl}-amide;

5-[(S)-1-[(6-Amino-pyridin-3-ylmethyl)-carbamoyl]-2-(3-trifluoromethyl-phenyl)-ethylcarbamoyl]-1H-pyrrole-2-carboxylic acid methyl ester;

5-[(S)-1-[(6-Amino-pyridin-3-ylmethyl)-carbamoyl]-2-(3-trifluoromethyl-phenyl)-ethylcarbamoyl]-1H-pyrrole-2-carboxylic acid;

5-[(S)-1-[(6-Amino-pyridin-3-ylmethyl)-carbamoyl]-2-(3-trifluoromethyl-phenyl)-ethylcarbamoyl]-4-methyl-1H-pyrrole-2-carboxylic acid;

3,5-Dimethyl-1H-pyrrole-2-carboxylic acid [(S)-1-[(6-amino-2-methyl-pyridin-3-ylmethyl)-carbamoyl]-2-(3-trifluoromethyl-phenyl)-ethyl]-amide;

3,5-Dimethyl-1H-pyrrole-2-carboxylic acid {(S)-1-[(6-amino-2-methyl-pyridin-3-ylmethyl)-carbamoyl]-2-naphthalen-1-yl-ethyl}-amide;

3,5-Dimethyl-1H-pyrrole-2-carboxylic acid [(S)-1-[(6-amino-2-methyl-pyridin-3-ylmethyl)-carbamoyl]-2-(3-chloro-phenyl)-ethyl]-amide;

3,5-Dimethyl-1H-pyrrole-2-carboxylic acid [(S)-1-[(6-amino-2-methyl-pyridin-3-ylmethyl)-carbamoyl]-2-(3,4-dichloro-phenyl)-ethyl]-amide;

3,5-Dimethyl-1H-pyrrole-2-carboxylic acid {(S)-1-[(6-amino-2-methyl-pyridin-3-ylmethyl)-carbamoyl]-2-m-tolyl-ethyl}-amide;

3,5-Dimethyl-1H-pyrrole-2-carboxylic acid [(S)-1-[(6-amino-2-methyl-pyridin-3-ylmethyl)-carbamoyl]-2-(2-trifluoromethyl-phenyl)-ethyl]-amide;

3,5-Dimethyl-1H-pyrrole-2-carboxylic acid [(S)-1-[(6-amino-2-methyl-pyridin-3-ylmethyl)-carbamoyl]-2-(1H-indol-3-yl)-ethyl]-amide;

3,5-Dimethyl-1H-pyrrole-2-carboxylic acid [(S)-1-[(6-amino-2-methyl-pyridin-3-ylmethyl)-carbamoyl]-2-(3,4-difluoro-phenyl)-ethyl]-amide;

3,5-Dimethyl-1H-pyrrole-2-carboxylic acid [(S)-1-[(6-amino-2-methyl-pyridin-3-ylmethyl)-carbamoyl]-2-(4-fluoro-phenyl)-ethyl]-amide;

3-Methyl-1H-pyrrole-2-carboxylic acid [(S)-1-[(6-amino-2-methyl-pyridin-3-ylmethyl)-carbamoyl]-2-(3,4-difluoro-phenyl)-ethyl]-amide;

1H-Imidazole-2-carboxylic acid [(S)-1-[(6-amino-pyridin-3-ylmethyl)-carbamoyl]-2-(4-fluoro-phenyl)-ethyl]-amide;

1H-Imidazole-2-carboxylic acid [(S)-1-[(6-amino-pyridin-3-ylmethyl)-carbamoyl]-2-(3-fluoro-phenyl)-ethyl]-amide;

1H-Imidazole-2-carboxylic acid {(S)-1-[(6-amino-pyridin-3-ylmethyl)-carbamoyl]-2-naphthalen-1-yl-ethyl}-amide;

1-Methyl-1H-imidazole-2-carboxylic acid [(S)-1-[(6-amino-pyridin-3-ylmethyl)-carbamoyl]-2-(4-fluoro-phenyl)-ethyl]-amide;

2,5-Dimethyl-2H-pyrazole-3-carboxylic acid [(S)-1-[(6-amino-pyridin-3-ylmethyl)-carbamoyl]-2-(4-fluoro-phenyl)-ethyl]-amide;

5-p-Tolyl-1H-pyrrole-2-carboxylic acid {(S)-1-[(6-amino-pyridin-3-ylmethyl)-carbamoyl]-2-naphthalen-1-yl-ethyl}-amide;

5-p-Tolyl-1H-pyrrole-2-carboxylic acid {(S)-1-[(6-amino-pyridin-3-ylmethyl)-carbamoyl]-2-benzo[b]thiophen-3-yl-ethyl}-amide;

5-p-Tolyl-1H-pyrrole-2-carboxylic acid [(S)-1-[(6-amino-pyridin-3-ylmethyl)-carbamoyl]-2-(3,4-difluoro-phenyl)-ethyl]-amide;

5-p-Tolyl-1H-pyrrole-2-carboxylic acid [(S)-1-[(6-amino-pyridin-3-ylmethyl)-carbamoyl]-2-(2-chloro-phenyl)-ethyl]-amide;

5-p-Tolyl-1H-pyrrole-2-carboxylic acid [(S)-1-[(6-amino-pyridin-3-ylmethyl)-carbamoyl]-2-(3,4-dichloro-phenyl)-ethyl]-amide;

5-p-Tolyl-1H-pyrrole-2-carboxylic acid [(S)-1-[(6-amino-pyridin-3-ylmethyl)-carbamoyl]-2-(3-chloro-phenyl)-ethyl]-amide;

1H-Indole-2-carboxylic acid {(S)-1-[(6-amino-pyridin-3-ylmethyl)-carbamoyl]-2-naphthalen-1-yl-ethyl}-amide;

1H-Benzoimidazole-2-carboxylic acid {(S)-1-[(6-amino-pyridin-3-ylmethyl)-carbamoyl]-2-naphthalen-1-yl-ethyl}-amide;

5-Methoxy-1H-indole-2-carboxylic acid {(S)-1-[(6-amino-pyridin-3-ylmethyl)-carbamoyl]-2-naphthalen-1-yl-ethyl}-amide;

5-Fluoro-1H-indole-2-carboxylic acid {(S)-1-[(6-amino-pyridin-3-ylmethyl)-carbamoyl]-2-naphthalen-1-yl-ethyl}-amide;

5-Hydroxy-1H-indole-2-carboxylic acid {(S)-1-[(6-amino-pyridin-3-ylmethyl)-carbamoyl]-2-naphthalen-1-yl-ethyl}-amide;

1H-Indole-2-carboxylic acid {(S)-1-[(6-amino-2-methyl-pyridin-3-ylmethyl)-carbamoyl]-2-naphthalen-1-yl-ethyl}-amide;

5-Methoxy-1H-indole-2-carboxylic acid {(S)-1-[(6-amino-2-methyl-pyridin-3-ylmethyl)-carbamoyl]-2-naphthalen-1-yl-ethyl}-amide;

5-Fluoro-1H-indole-2-carboxylic acid {(S)-1-[(6-amino-2-methyl-pyridin-3-ylmethyl)-carbamoyl]-2-naphthalen-1-yl-ethyl}-amide;

1-Methyl-1H-indole-2-carboxylic acid {(S)-1-[(6-amino-pyridin-3-ylmethyl)-carbamoyl]-2-naphthalen-1-yl-ethyl}-amide;

1H-Indole-2-carboxylic acid [(S)-1-[(6-amino-pyridin-3-ylmethyl)-carbamoyl]-2-(3-trifluoromethyl-phenyl)-ethyl]-amide;

5-Methoxy-1H-indole-2-carboxylic acid [(S)-1-[(6-amino-pyridin-3-ylmethyl)-carbamoyl]-2-(3-trifluoromethyl-phenyl)-ethyl]-amide;

1H-Indole-2-carboxylic acid [(S)-1-[(6-amino-2-methyl-pyridin-3-ylmethyl)-carbamoyl]-2-(3-trifluoromethyl-phenyl)-ethyl]-amide;

5-Methoxy-1H-indole-2-carboxylic acid [(S)-1-[(6-amino-2-methyl-pyridin-3-ylmethyl)-carbamoyl]-2-(3-trifluoromethyl-phenyl)-ethyl]-amide;

1H-Indole-2-carboxylic acid [(S)-1-[(6-amino-pyridin-3-ylmethyl)-carbamoyl]-2-(3-fluoro-phenyl)-ethyl]-amide;

5-Methoxy-1H-indole-2-carboxylic acid [(S)-1-[(6-amino-pyridin-3-ylmethyl)-carbamoyl]-2-(3-fluoro-phenyl)-ethyl]-amide;

1H-Indole-2-carboxylic acid [(S)-1-[(6-amino-pyridin-3-ylmethyl)-carbamoyl]-2-(3-chloro-phenyl)-ethyl]-amide;

5-Methoxy-1H-indole-2-carboxylic acid [(S)-1-[(6-amino-pyridin-3-ylmethyl)-carbamoyl]-2-(3-chloro-phenyl)-ethyl]-amide;

1H-Indole-2-carboxylic acid [(R)-1-[(6-amino-2-methyl-pyridin-3-ylmethyl)-carbamoyl]-2-(3-chloro-phenyl)-ethyl]-amide;

5-Methoxy-1H-indole-2-carboxylic acid [(S)-1-[(6-amino-2-methyl-pyridin-3-ylmethyl)-carbamoyl]-2-(3-chloro-phenyl)-ethyl]-amide;

1H-Indole-2-carboxylic acid {(S)-1-[(6-amino-pyridin-3-ylmethyl)-carbamoyl]-2-m-tolyl-ethyl}-amide;

5-Methoxy-1H-indole-2-carboxylic acid {(S)-1-[(6-amino-pyridin-3-ylmethyl)-carbamoyl]-2-m-tolyl-ethyl}-amide;

1H-Indole-2-carboxylic acid {(S)-1-[(6-amino-2-methyl-pyridin-3-ylmethyl)-carbamoyl]-2-m-tolyl-ethyl}-amide;

1H-Indole-2-carboxylic acid [(S)-1-[(6-amino-2-methyl-pyridin-3-ylmethyl)-carbamoyl]-2-(3,4-dichloro-phenyl)-ethyl]-amide; 5-Methoxy-1H-indole-2-carboxylic acid [(S)-1-[(6-amino-2-methyl-pyridin-3-ylmethyl)-carbamoyl]-2-(3,4-dichloro-phenyl)-ethyl]-amide;

1H-Indole-2-carboxylic acid [(S)-1-[(6-amino-2-methyl-pyridin-3-ylmethyl)-carbamoyl]-2-(2-trifluoromethyl-phenyl)-ethyl]-amide;

1H-Indole-2-carboxylic acid [(S)-1-[(6-amino-pyridin-3-ylmethyl)-carbamoyl]-2-(3,5-difluoro-phenyl)-ethyl]-amide;

1H-Indole-2-carboxylic acid [(S)-1-[(6-amino-pyridin-3-ylmethyl)-carbamoyl]-2-(2,4,5-trifluoro-phenyl)-ethyl]-amide;

1H-Indole-2-carboxylic acid [(S)-1-[(6-amino-pyridin-3-ylmethyl)-carbamoyl]-2-(3,4-difluoro-phenyl)-ethyl]-amide;

5-Fluoro-1H-indole-2-carboxylic acid [(S)-1-[(6-amino-pyridin-3-ylmethyl)-carbamoyl]-2-(3,4-difluoro-phenyl)-ethyl]-amide;

5-Methoxy-1H-indole-2-carboxylic acid [(S)-1-[(6-amino-pyridin-3-ylmethyl)-carbamoyl]-2-(3,4-difluoro-phenyl)-ethyl]-amide;

1H-Benzoimidazole-2-carboxylic acid [(S)-1-[(6-amino-pyridin-3-ylmethyl)-carbamoyl]-2-(3,4-difluoro-phenyl)-ethyl]-amide;

1H-Indole-2-carboxylic acid {(S)-1-[(6-amino-pyridin-3-ylmethyl)-carbamoyl]-2-pentafluorophenyl-ethyl}-amide;

1H-Indole-2-carboxylic acid [(S)-1-[(6-amino-pyridin-3-ylmethyl)-carbamoyl]-2-(4-fluoro-phenyl)-ethyl]-amide;

5-Fluoro-1H-indole-2-carboxylic acid [(S)-1-[(6-amino-pyridin-3-ylmethyl)-carbamoyl]-2-(4-fluoro-phenyl)-ethyl]-amide;

5-Methoxy-1H-indole-2-carboxylic acid [(S)-1-[(6-amino-pyridin-3-ylmethyl)-carbamoyl]-2-(4-fluoro-phenyl)-ethyl]-amide;

1H-Benzoimidazole-2-carboxylic acid [(S)-1-[(6-amino-pyridin-3-ylmethyl)-carbamoyl]-2-(4-fluoro-phenyl)-ethyl]-amide;

1H-Indole-2-carboxylic acid [(S)-1-[(6-amino-pyridin-3-ylmethyl)-carbamoyl]-2-(4-methoxy-phenyl)-ethyl]-amide;

1H-Indole-2-carboxylic acid {(S)-1-[(6-amino-pyridin-3-ylmethyl)-carbamoyl]-2-benzo[b]thiophen-3-yl-ethyl}-amide;

1H-Indole-2-carboxylic acid [(S)-1-[(6-amino-2-methyl-pyridin-3-ylmethyl)-carbamoyl]-2-(1H-indol-3-yl)-ethyl]-amide;

5-Methoxy-1H-indole-2-carboxylic acid [(S)-1-[(6-amino-2-methyl-pyridin-3-ylmethyl)-carbamoyl]-2-(1H-indol-3-yl)-ethyl]-amide;

1H-Indole-2-carboxylic acid {(S)-1-[(6-amino-pyridin-3-ylmethyl)-carbamoyl]-2-cyclohexyl-ethyl}-amide;

(S)-3-Methyl-2-methylamino-pentanoic acid {(S)-1-[(6-amino-pyridin-3-ylmethyl)-carbamoyl]-2-cyclohexyl-ethyl}-amide;

(R)—N—[(S)-1-[(6-Amino-pyridin-3-ylmethyl)-carbamoyl]-2-(3,4-dichloro-phenyl)-ethyl]-3-methyl-2-methylamino-butyramide;

(R)—N—[(S)-1-[(6-Amino-pyridin-3-ylmethyl)-carbamoyl]-2-(3-chloro-phenyl)-ethyl]-3-methyl-2-methylamino-butyramide;

(R)—N—[(S)-1-[(6-Amino-pyridin-3-ylmethyl)-carbamoyl]-2-(3-chloro-phenyl)-ethyl]-2-methylamino-3-phenyl-propionamide;

(S)—N—[(S)-1-[(6-Amino-pyridin-3-ylmethyl)-carbamoyl]-2-(3,4-difluoro-phenyl)-ethyl]-2-(cyclohexylmethyl-methyl-amino)-propionamide;

(S)—N—[(S)-1-[(6-Amino-pyridin-3-ylmethyl)-carbamoyl]-2-(3,4-difluoro-phenyl)-ethyl]-2-dimethylamino-propionamide;

(S)—N—[(S)-1-[(6-Amino-pyridin-3-ylmethyl)-carbamoyl]-2-(3,4-difluoro-phenyl)-ethyl]-2-(ethyl-methyl-amino)-propionamide;

(S)—N—[(S)-1-[(6-Amino-pyridin-3-ylmethyl)-carbamoyl]-2-(3,4-difluoro-phenyl)-ethyl]-2-(methyl-propyl-amino)-propionamide;

(S)—N—[(S)-1-[(6-Amino-pyridin-3-ylmethyl)-carbamoyl]-2-(3,4-difluoro-phenyl)-ethyl]-2-(butyl-methyl-amino)-propionamide;

(S)—N—[(S)-1-[(6-Amino-pyridin-3-ylmethyl)-carbamoyl]-2-(3,4-difluoro-phenyl)-ethyl]-2-(isopropyl-methyl-amino)-3-methyl-butyramide;

(S)-2-(Isopropyl-methyl-amino)-3-methyl-pentanoic acid [(S)-1-[(6-amino-pyridin-3-ylmethyl)-carbamoyl]-2-(3,4-difluoro-phenyl)-ethyl]-amide;

(S)—N—[(S)-1-[(6-Amino-pyridin-3-ylmethyl)-carbamoyl]-2-(3,4-difluoro-phenyl)-ethyl]-2-diisopropylamino-propionamide;

(S)—N—[(S)-1-[(6-Amino-pyridin-3-ylmethyl)-carbamoyl]-2-(3,4-difluoro-phenyl)-ethyl]-2-dimethylamino-3-phenyl-propionamide;

(S)—N—[(S)-1-[(6-Amino-pyridin-3-ylmethyl)-carbamoyl]-2-(3,4-difluoro-phenyl)-ethyl]-2-(ethyl-methyl-amino)-3-phenyl-propionamide;

(S)-2-(Isopropyl-methyl-amino)-3-methyl-pentanoic acid [(S)-1-[(6-amino-pyridin-3-ylmethyl)-carbamoyl]-2-(3,4-difluoro-phenyl)-ethyl]-amide;

N—[(S)-1-[(6-Amino-pyridin-3-ylmethyl)-carbamoyl]-2-(3,4-difluoro-phenyl)-ethyl]-2-piperidin-1-yl-propionamide;

(S)—N—[(S)-1-[(6-Amino-pyridin-3-ylmethyl)-carbamoyl]-2-(3,4-difluoro-phenyl)-ethyl]-2-diethylamino-propionamide;

(R)—N—[(S)-1-[(6-Amino-pyridin-3-ylmethyl)-carbamoyl]-2-(3,4-difluoro-phenyl)-ethyl]-2-pyrrolidin-1-yl-propionamide;

(S)—N—[(S)-1-[(6-Amino-pyridin-3-ylmethyl)-carbamoyl]-2-(3,4-difluoro-phenyl)-ethyl]-2-pyrrolidin-1-yl-propionamide;

(R)-2-Dimethylamino-3-methyl-pentanoic acid [(S)-1-[(6-amino-pyridin-3-ylmethyl)-carbamoyl]-2-(3,4-difluoro-phenyl)-ethyl]-amide;

(R)—N—[(S)-1-[(6-Amino-pyridin-3-ylmethyl)-carbamoyl]-2-(3,4-difluoro-phenyl)-ethyl]-2-dimethylamino-3-methyl-butyramide;

(S)—N—[(S)-1-[(6-Amino-pyridin-3-ylmethyl)-carbamoyl]-2-(3,4-difluoro-phenyl)-ethyl]-3-hydroxy-2-(isopropyl-methyl-amino)-propionamide;

(S)—N—[(S)-1-[(6-Amino-pyridin-3-ylmethyl)-carbamoyl]-2-(3,4-difluoro-phenyl)-ethyl]-2-(ethyl-methyl-amino)-3-hydroxy-propionamide;

(S)—N—[(S)-1-[(6-Amino-pyridin-3-ylmethyl)-carbamoyl]-2-(3,4-difluoro-phenyl)-ethyl]-2-diethylamino-3-hydroxy-propionamide;

(R)—N—[(S)-1-[(6-Amino-pyridin-3-ylmethyl)-carbamoyl]-2-(3,4-difluoro-phenyl)-ethyl]-2-diethylamino-3-hydroxy-propionamide;

(S)—N—[(S)-1-[(6-Amino-pyridin-3-ylmethyl)-carbamoyl]-2-(3,4-difluoro-phenyl)-ethyl]-3-diethylamino-succinamic acid methyl ester;

(S)—N-(6-Amino-pyridin-3-ylmethyl)-3-(3,4-difluoro-phenyl)-2-[2-(2,6-dimethyl-piperidin-1-yl)-acetylamino]-propionamide;

(S)—N-(6-Amino-pyridin-3-ylmethyl)-3-(3,4-difluoro-phenyl)-2-(2-pyrrolidin-1-acetylamino)-propionamide;

(S)—N—[(S)-1-[(6-Amino-pyridin-3-ylmethyl)-carbamoyl]-2-(4-fluoro-phenyl)-ethyl]-2-(ethyl-methyl-amino)-propionamide;

(S)—N—[(S)-1-[(6-Amino-pyridin-3-ylmethyl)-carbamoyl]-2-(4-fluoro-phenyl)-ethyl]-2-(methyl-propyl-amino)-propionamide;

(S)—N—[(S)-1-[(6-Amino-pyridin-3-ylmethyl)-carbamoyl]-2-(4-fluoro-phenyl)-ethyl]-2-(butyl-methyl-amino)-propionamide;

(S)—N—[(S)-1-[(6-Amino-pyridin-3-ylmethyl)-carbamoyl]-2-(4-fluoro-phenyl)-ethyl]-2-(isopropyl-methyl-amino)-3-methyl-butyramide;

(S)-2-(Isopropyl-methyl-amino)-3-methyl-pentanoic acid [(S)-1-[(6-amino-pyridin-3-ylmethyl)-carbamoyl]-2-(4-fluoro-phenyl)-ethyl]-amide;

(S)—N—[(S)-1-[(6-Amino-pyridin-3-ylmethyl)-carbamoyl]-2-(4-fluoro-phenyl)-ethyl]-2-diisopropylamino-propionamide;

(S)—N—[(S)-1-[(6-Amino-pyridin-3-ylmethyl)-carbamoyl]-2-(4-fluoro-phenyl)-ethyl]-2(isopropyl-methyl-amino)-3-phenyl-propionamide;

(R)—N—[(S)-1-[(6-Amino-pyridin-3-ylmethyl)-carbamoyl]-2-(4-fluoro-phenyl)-ethyl]-2-piperidin-1-yl-propionamide;

(S)—N—[(S)-1-[(6-Amino-pyridin-3-ylmethyl)-carbamoyl]-2-(4-fluoro-phenyl)-ethyl]-2-piperidin-1-yl-propionamide;

(S)—N—[(S)-1-[(6-Amino-pyridin-3-ylmethyl)-carbamoyl]-2-(4-fluoro-phenyl)-ethyl]-2-diethylamino-propionamide;

(R)-2-Dimethylamino-3-methyl-pentanoic acid [(S)-1-[(6-amino-pyridin-3-ylmethyl)-carbamoyl]-2-(3-fluoro-phenyl)-ethyl]-amide;

(S)—N-(6-Amino-pyridin-3-ylmethyl)-2-[2-(2,6-dimethyl-piperidin-1-yl)-acetylamino]-3-(4-fluoro-phenyl)-propionamide;

(S)—N-(6-Amino-pyridin-3-ylmethyl)-3-(4-fluoro-phenyl)-2-(2-piperidin-1-yl-acetylamino)-propionamide;

(S)—N-(6-Amino-pyridin-3-ylmethyl)-2-(2-diethylamino-acetylamino)-3-(4-fluoro-phenyl)-propionamide;

(S)—N—{(S)-1-[(6-Amino-pyridin-3-ylmethyl)-carbamoyl]-2-cyclohexyl-ethyl}-2-(isopropyl-methyl-amino)-propionamide;

(S)-1-Methyl-pyrrolidine-2-carboxylic acid {(S)-1-[(6-amino-pyridin-3-ylmethyl)-carbamoyl]-2-cyclohexyl-ethyl}-amide;

(S)—N—{(S)-1-[(6-Amino-pyridin-3-ylmethyl)-carbamoyl]-2-cyclohexyl-ethyl}-2-(methyl-propyl-amino)-propionamide;

(S)—N—{(S)-1-[(6-Amino-pyridin-3-ylmethyl)-carbamoyl]-2-cyclohexyl-ethyl}-2-(ethyl-methyl-amino)-propionamide;

(S)—N—{(S)-1-[(6-Amino-pyridin-3-ylmethyl)-carbamoyl]-2-cyclohexyl-ethyl}-2-dipropylaminopropionamide;

(S)—N—{(S)-1-[(6-Amino-pyridin-3-ylmethyl)-carbamoyl]-2-cyclohexyl-ethyl}-2-dimethylamino-3-hydroxy-propionamide;

(S)—N—{(S)-1-[(6-Amino-pyridin-3-ylmethyl)-carbamoyl]-2-cyclohexyl-ethyl}-2-(ethyl-methyl-amino)-3-hydroxy-propionamide;

(S)—N—{(S)-1-[(6-Amino-pyridin-3-ylmethyl)-carbamoyl]-2-cyclohexyl-ethyl}-3-hydroxy-2-(isopropyl-methyl-amino)-propionamide;

(S)—N—{(S)-1-[(6-Amino-pyridin-3-ylmethyl)-carbamoyl]-2-cyclohexyl-ethyl}-2-diethylamino-3-hydroxy-propionamide;

(S)—N—{(S)-1-[(6-Amino-pyridin-3-ylmethyl)-carbamoyl]-2-cyclohexyl-ethyl}-2-diethylamino-propionamide;

(S)—N—{(S)-1-[(6-Amino-pyridin-3-ylmethyl)-carbamoyl]-2-cyclohexyl-ethyl}-2-dimethylamino-propionamide;

(S)—N—(6-Amino-pyridin-3-ylmethyl)-3-cyclohexyl-2-[2-(2,6-dimethyl-piperidin-1-yl)-acetylamino]-propionamide;

(S)—N-(6-Amino-pyridin-3-ylmethyl)-3-cyclohexyl-2-(2-diisopropylamino-acetylamino)-propionamide;

(S)—N-(6-Amino-pyridin-3-ylmethyl)-3-(decahydro-naphthalen-1-yl)-2-(2-diisopropylamino-acetylamino)-propionamide;

(S)—N—[(S)-1-[(6-Amino-pyridin-3-ylmethyl)-carbamoyl]-2-(decahydro-naphthalen-1-yl)-ethyl]-2-(isopropyl-methyl-amino)-propionamide;

(R)-1-Methyl-pyrrolidine-2-carboxylic acid [(S)-1-[(6-amino-pyridin-3-ylmethyl)-carbamoyl]-2-(decahydro-naphthalen-1-yl)-ethyl]-amide;

(R)—N—[(S)-1-[(6-Amino-pyridin-3-ylmethyl)-carbamoyl]-2-(decahydro-naphthalen-1-yl)-ethyl]-2-dimethylamino-3-phenyl-propionamide;

(R)-2-Dimethylamino-3-methyl-pentanoic acid [(S)-1-[(6-amino-pyridin-3-ylmethyl)-carbamoyl]-2-(3,4-dichloro-phenyl)-ethyl]-amide;

(R)—N—[(S)-1-[(6-Amino-pyridin-3-ylmethyl)-carbamoyl]-2-(3,4-dichloro-phenyl)-ethyl]-2-dimethylamino-3-methyl-butyramide;

(R)—N—[(S)-1-[(6-Amino-pyridin-3-ylmethyl)-carbamoyl]-2-(3,4-dichloro-phenyl)-ethyl]-2-dimethylamino-3-phenyl-propionamide;

(S)—N—[(S)-1-[(6-Amino-pyridin-3-ylmethyl)-carbamoyl]-2-(3,4-dichloro-phenyl)-ethyl]-2-(isopropyl-methyl-amino)-propionamide;

(R)—N—[(S)-1-[(6-Amino-pyridin-3-ylmethyl)-carbamoyl]-2-(3,4-dichloro-phenyl)-ethyl]-2-piperidin-1-yl-propionamide;

(S)—N—[(S)-1-[(6-Amino-pyridin-3-ylmethyl)-carbamoyl]-2-(3,4-dichloro-phenyl)-ethyl]-2-piperidin-1-yl-propionamide;

(S)—N-(6-Amino-pyridin-3-ylmethyl)-3-(3,4-dichloro-phenyl)-2-(2-piperidin-1-yl-acetylamino)-propionamide;

(R)-2-Dimethylamino-3-methyl-pentanoic acid [(S)-1-[(6-amino-pyridin-3-ylmethyl)-carbamoyl]-2-(3-chloro-phenyl)-ethyl]-amide;

(R)—N—[(S)-1-[(6-Amino-pyridin-3-ylmethyl)-carbamoyl]-2-(3-chloro-phenyl)-ethyl]-2-dimethylamino-3-methyl-butyramide;

(R)—N—[(S)-1-[(6-Amino-pyridin-3-ylmethyl)-carbamoyl]-2-(3-chloro-phenyl)-ethyl]-2-dimethylamino-3-phenyl-propionamide;

(S)—N—[(S)-1-[(6-Amino-pyridin-3-ylmethyl)-carbamoyl]-2-(3-chloro-phenyl)-ethyl]-2-(ethyl-methyl-amino)-propionamide;

(S)—N—[(S)-1-[(6-Amino-pyridin-3-ylmethyl)-carbamoyl]-2-(3-chloro-phenyl)-ethyl]-2-(isopropyl-methyl-amino)-propionamide;

(S)—N—[(S)-1-[(6-Amino-pyridin-3-ylmethyl)-carbamoyl]-2-(3-chloro-phenyl)-ethyl]-2-diethylamino-propionamide;

N—[(S)-1-[(6-Amino-pyridin-3-ylmethyl)-carbamoyl]-2-(3-chloro-phenyl)-ethyl]-2-piperidin-1-yl-propionamide;

(S)—N-(6-Amino-pyridin-3-ylmethyl)-3-(3-chloro-phenyl)-2-(2-piperidin-1-yl-acetylamino)-propionamide;

(S)—N—{(S)-1-[(6-Amino-pyridin-3-ylmethyl)-carbamoyl]-2-m-tolyl-ethyl}-2-(ethyl-methyl-amino)-propionamide;

(S)—N—{(S)-1-[(6-Amino-pyridin-3-ylmethyl)-carbamoyl]-2-m-tolyl-ethyl}-2-(isopropyl-methyl-amino)-propionamide;

(S)—N—{(S)-1-[(6-Amino-pyridin-3-ylmethyl)-carbamoyl]-2-m-tolyl-ethyl}-2-diethylamino-propionamide;

(S)—N—[(S)-1-[(6-Amino-pyridin-3-ylmethyl)-carbamoyl]-2-(3-trifluoromethyl-phenyl)-ethyl]-2-(ethyl-methyl-amino)-propionamide;

(S)—N—[(S)-1-[(6-Amino-pyridin-3-ylmethyl)-carbamoyl]-2-(3-trifluoromethyl-phenyl)-ethyl]-2-(isopropyl-methyl-amino)-propionamide;

(S)—N—[(S)-1-[(6-Amino-pyridin-3-ylmethyl)-carbamoyl]-2-(3-trifluoromethyl-phenyl)-ethyl]-2-diethylamino-propionamide;

N—[(S)-1-[(6-Amino-pyridin-3-ylmethyl)-carbamoyl]-2-(3-trifluoromethyl-phenyl)-ethyl]-2-piperidin-1-yl-propionamide;

(S)—N-(6-Amino-pyridin-3-ylmethyl)-2-(2-piperidin-1-yl-acetylamino)-3-(3-trifluoromethyl-phenyl)-propionamide;

(R)—N—[(S)-1-[(6-Amino-pyridin-3-ylmethyl)-carbamoyl]-2-(4-trifluoromethyl-phenyl)-ethyl]-2-piperidin-1-yl-propionamide;

(S)—N—[(S)-1-[(6-Amino-pyridin-3-ylmethyl)-carbamoyl]-2-(4-trifluoromethyl-phenyl)-ethyl]-2-piperidin-1-yl-propionamide;

(S)—N-(6-Amino-pyridin-3-ylmethyl)-2-(2-piperidin-1-yl-acetylamino)-3-(4-trifluoromethyl-phenyl)-propionamide;

(R)-2-Dimethylamino-3-methyl-pentanoic acid [(S)-1-[(6-amino-pyridin-3-ylmethyl)-carbamoyl]-2-(3,5-difluoro-phenyl)-ethyl]-amide;

N—[(S)-1-[(6-Amino-pyridin-3-ylmethyl)-carbamoyl]-2-(3,5-difluoro-phenyl)-ethyl]-2-piperidin-1-yl-propionamide;

(S)—N-(6-Amino-pyridin-3-ylmethyl)-3-(4-chloro-phenyl)-2-[2-(2,6-dimethyl-piperidin-1-yl)-acetylamino]-propionamide;

(S)-2-[(S)-2-(Isopropyl-methyl-amino)-propionylamino]-4-methyl-pentanoic acid (6-amino-pyridin-3-ylmethyl)-amide;

(S)—N-(6-Amino-2-methyl-pyridin-3-ylmethyl)-2-(2-diethylamino-acetylamino)-3-(4-fluoro-phenyl)-propionamide;

(S)—N-(6-Amino-2-methyl-pyridin-3-ylmethyl)-3-(3,4-difluoro-phenyl)-2-[2-(2,6-dimethyl-piperidin-1-yl)-acetylamino]-propionamide;

(S)—N-(6-Amino-2-methyl-pyridin-3-ylmethyl)-3-(3,4-difluoro-phenyl)-2-(2-piperidin-1-yl-acetylamino)-propionamide;

(R)—N—[(S)-1-[(6-Amino-2-methyl-pyridin-3-ylmethyl)-carbamoyl]-2-(3,4-difluoro-phenyl)-ethyl]-2-piperidin-1-yl-propionamide;

(S)—N—[(S)-1-[(6-Amino-2-methyl-pyridin-3-ylmethyl)-carbamoyl]-2-(3,4-difluoro-phenyl)-ethyl]-2-piperidin-1-yl-propionamide;

(S)—N—{(R)-1-[(6-Amino-pyridin-3-ylmethyl)-carbamoyl]-2,2-dicyclohexyl-ethyl}-2-(isopropyl-methyl-amino)-propionamide;

(S)—N—{(R)-1-[(6-Amino-pyridin-3-ylmethyl)-carbamoyl]-2,2-dicyclohexyl-ethyl}-2-dimethylamino-propionamide;

(S)—N—{(R)-1-[(6-Amino-pyridin-3-ylmethyl)-carbamoyl]-2-cyclohexyl-2-hydroxy-ethyl}-2-(isopropyl-methyl-amino)-propionamide;

(R)—N-(6-Amino-pyridin-3-ylmethyl)-3-cyclohexyl-2-[(S)-2-(isopropyl-methyl-amino)-propionylamino]-butyramide;

(S)—N—{(S)-1-[(6-Amino-pyridin-3-ylmethyl)-carbamoyl]-2-cyclohexyl-ethyl}-2-(isopropyl-methyl-amino)-N-methyl-propionamide;

S)—N—{(S)-1-[(6-Amino-pyridin-3-ylmethyl)-carbamoyl]-2-cyclohexyl-ethyl}-2-diethylamino-N-methyl-propionamide;

(S)—N—{(S)-1-[(6-Amino-pyridin-3-ylmethyl)-carbamoyl]-2-cyclohexyl-ethyl}-2-dipropylamino-propionamide;

and tautomers, stereoisomers, pharmaceutically acceptable salts and solvates thereof.

In another aspect, the present invention provides a compound of formula (I) selected from:

(R)-1-Methyl-pyrrolidine-2-carboxylic acid {(S)-1-[(6-amino-pyridin-3-ylmethyl)-carbamoyl]-2-naphthalen-1-yl-ethyl}-amide;

(R)-1-Methyl-pyrrolidine-2-carboxylic acid [(S)-1-[(6-amino-pyridin-3-ylmethyl)-carbamoyl]-2-(3-chloro-phenyl)-ethyl]-amide;

(R)-1-Methyl-pyrrolidine-2-carboxylic acid [(S)-1-[(6-amino-pyridin-3-ylmethyl)-carbamoyl]-2-(3,4-difluoro-phenyl)-ethyl]-amide;

(R)-3-Methyl-2-methylamino-pentanoic acid [(S)-1-[(6-amino-pyridin-3-ylmethyl)-carbamoyl]-2-(3,4-difluoro-phenyl)-ethyl]-amide;

(R)-1-Methyl-pyrrolidine-2-carboxylic acid [(S)-1-[(6-amino-pyridin-3-ylmethyl)-carbamoyl]-2-(3,5-difluoro-phenyl)-ethyl]-amide;

(R)-1-Methyl-pyrrolidine-2-carboxylic acid [(S)-1-[(6-amino-pyridin-3-ylmethyl)-carbamoyl]-2-(4-fluoro-phenyl)-ethyl]-amide;

(R)-1-Methyl-pyrrolidine-2-carboxylic acid [(S)-1-[(6-amino-pyridin-3-ylmethyl)-carbamoyl]-2-(3-fluoro-phenyl)-ethyl]-amide;

(R)-1-Isopropyl-pyrrolidine-2-carboxylic acid [(S)-1-[(6-amino-pyridin-3-ylmethyl)-carbamoyl]-2-(3,4-difluoro-phenyl)-ethyl]-amide;

(R)-1-Isopropyl-pyrrolidine-2-carboxylic acid [(S)-1-[(6-amino-pyridin-3-ylmethyl)-carbamoyl]-2-(4-fluoro-phenyl)-ethyl]-amide;

(R)-1-Ethyl-pyrrolidine-2-carboxylic acid [(S)-1-[(6-amino-pyridin-3-ylmethyl)-carbamoyl]-2-(3,4-difluoro-phenyl)-ethyl]-amide;

(R)-1-Propyl-pyrrolidine-2-carboxylic acid [(S)-1-[(6-amino-pyridin-3-ylmethyl)-carbamoyl]-2-(3,4-difluoro-phenyl)-ethyl]-amide;

(R)-1-Isobutyl-pyrrolidine-2-carboxylic acid [(S)-1-[(6-amino-pyridin-3-ylmethyl)-carbamoyl]-2-(3,4-difluoro-phenyl)-ethyl]-amide;

(R)-1-Ethyl-pyrrolidine-2-carboxylic acid [(S)-1-[(6-amino-pyridin-3-ylmethyl)-carbamoyl]-2-(4-fluoro-phenyl)-ethyl]-amide;

(S)—N-(6-Amino-pyridin-3-ylmethyl)-3-(3,4-difluoro-phenyl)-2-(2-diisopropylamino-acetylamino)-propionamide;

(R)-1-Methyl-piperidine-2-carboxylic acid [(S)-1-[(6-amino-pyridin-3-ylmethyl)-carbamoyl]-2-(3,4-difluoro-phenyl)-ethyl]-amide;

(R)-1-Methyl-piperidine-2-carboxylic acid [(S)-1-[(6-amino-pyridin-3-ylmethyl)-carbamoyl]-2-(4-fluoro-phenyl)-ethyl]-amide;

(S)—N—[(S)-1-[(6-Amino-pyridin-3-ylmethyl)-carbamoyl]-2-(3,4-difluoro-phenyl)-ethyl]-2-(isopropyl-methyl-amino)-propionamide;

(R)-2-Dimethylamino-3-methyl-pentanoic acid [(S)-1-[(6-amino-pyridin-3-ylmethyl)-carbamoyl]-2-(3,4-difluoro-phenyl)-ethyl]-amide;

(R)—N—[(S)-1-[(6-Amino-pyridin-3-ylmethyl)-carbamoyl]-2-(3,4-difluoro-phenyl)-ethyl]-2-dimethylamino-3,3-dimethyl-butyramide;

(R)-1-Methyl-pyrrolidine-2-carboxylic acid [(S)-1-[(6-amino-2-methyl-pyridin-3-ylmethyl)-carbamoyl]-2-(4-fluoro-phenyl)-ethyl]-amide;

(R)-1-Methyl-pyrrolidine-2-carboxylic acid [(S)-1-[(6-amino-2-methyl-pyridin-3-ylmethyl)-carbamoyl]-2-(3,4-difluoro-phenyl)-ethyl]-amide;

(S)-1-Methyl-pyrrolidine-2-carboxylic acid {(R)-1-[(6-amino-pyridin-3-ylmethyl)-carbamoyl]-2,2-dicyclohexyl-ethyl}-amide;

3-Methyl-1H-pyrrole-2-carboxylic acid [(S)-1-[(6-amino-pyridin-3-ylmethyl)-carbamoyl]-2-(3,4-difluoro-phenyl)-ethyl]-amide;

3,5-Dimethyl-1H-pyrrole-2-carboxylic acid [(S)-1-[(6-amino-pyridin-3-ylmethyl)-carbamoyl]-2-(3-fluoro-phenyl)-ethyl]-amide;

3,5-Dimethyl-1H-pyrrole-2-carboxylic acid [(S)-1-[(6-amino-pyridin-3-ylmethyl)-carbamoyl]-2-(4-fluoro-phenyl)-ethyl]-amide;

3,5-Dimethyl-1H-pyrrole-2-carboxylic acid [(S)-1-[(6-amino-pyridin-3-ylmethyl)-carbamoyl]-2-(3,4-difluoro-phenyl)-ethyl]-amide;

3-Methyl-1H-pyrrole-2-carboxylic acid [(S)-1-[(6-amino-pyridin-3-ylmethyl)-carbamoyl]-2-(4-fluoro-phenyl)-ethyl]-amide;

3,5-Dimethyl-1H-pyrrole-2-carboxylic acid [(S)-1-[(6-amino-2-methyl-pyridin-3-ylmethyl)-carbamoyl]-2-(3-trifluoromethyl-phenyl)-ethyl]-amide;

(R)—N—[(S)-1-[(6-Amino-pyridin-3-ylmethyl)-carbamoyl]-2-(3,4-dichloro-phenyl)-ethyl]-3-methyl-2-methylamino-butyramide;

(S)—N—[(S)-1-[(6-Amino-pyridin-3-ylmethyl)-carbamoyl]-2-(3,4-difluoro-phenyl)-ethyl]-2-(ethyl-methyl-amino)-propionamide;

(S)—N—[(S)-1-[(6-Amino-pyridin-3-ylmethyl)-carbamoyl]-2-(3,4-difluoro-phenyl)-ethyl]-2-diethylamino-propionamide;

(S)—N—{(S)-1-[(6-Amino-pyridin-3-ylmethyl)-carbamoyl]-2-cyclohexyl-ethyl}-2-(isopropyl-methyl-amino)-propionamide;

(S)—N—{(S)-1-[(6-Amino-pyridin-3-ylmethyl)-carbamoyl]-2-cyclohexyl-ethyl}-2-diethylamino-propionamide;

(S)—N—[(S)-1-[(6-Amino-pyridin-3-ylmethyl)-carbamoyl]-2-(decahydro-naphthalen-1-yl)-ethyl]-2-(isopropyl-methyl-amino)-propionamide;

(R)-2-Dimethylamino-3-methyl-pentanoic acid [(S)-1-[(6-amino-pyridin-3-ylmethyl)-carbamoyl]-2-(3,4-dichloro-phenyl)-ethyl]-amide;

(R)—N—[(S)-1-[(6-Amino-pyridin-3-ylmethyl)-carbamoyl]-2-(3,4-dichloro-phenyl)-ethyl]-2-dimethylamino-3-methyl-butyramide;

(S)—N—[(S)-1-[(6-Amino-pyridin-3-ylmethyl)-carbamoyl]-2-(3,4-dichloro-phenyl)-ethyl]-2-(isopropyl-methyl-amino)-propionamide;

(S)—N—[(S)-1-[(6-Amino-pyridin-3-ylmethyl)-carbamoyl]-2-(3-chloro-phenyl)-ethyl]-2-(isopropyl-methyl-amino)-propionamide;

(S)—N—{(S)-1-[(6-Amino-pyridin-3-ylmethyl)-carbamoyl]-2-m-tolyl-ethyl}-2-(isopropyl-methyl-amino)-propionamide;

(S)—N—[(S)-1-[(6-Amino-pyridin-3-ylmethyl)-carbamoyl]-2-(3-trifluoromethyl-phenyl)-ethyl]-2-(isopropyl-methyl-amino)-propionamide;

and tautomers, stereoisomers, pharmaceutically acceptable salts and solvates thereof.

The skilled person will appreciate that each of the compounds identified above, or identified in the Examples provided herein below, taken alone or with any combination of the other identified compounds represents an independent aspect of the invention.

Therapeutic Applications

As previously mentioned, the compounds of the present invention have a number of therapeutic applications, particularly in the treatment of inflammatory diseases such as asthma and COPD, by virtue of their ability to inhibit KLK1.

In particular, the compounds of the present invention may be used for the treatment of respiratory disorders involving airways inflammation e.g. asthma (allergic and non-allergic) including exacerbations resulting from asthma and chronic obstructive pulmonary disease (COPD). Such compounds may also be used to treat other forms of allergic inflammation including allergic rhinitis (hayfever), rhino-conjunctivitis, rhinorrhoea, urticaria, excess lung mucus production and ascites build-up.

Other inflammatory disorders that may be treated with the compounds of the present invention include, multiple sclerosis, arthritis, rheumatoid arthritis, osteopathic arthritis, osteoarthritis, rhinitis, sinusitis, inflammatory bowel disease (such as Crohn's disease and ulcerative colitis), immune mediated diabetes, acute pancreatitis and interstitial cystitis, thermal injury, crush injury, conjunctivitis, periodontal disease, chronic prostate inflammation, chronic recurrent parotitis, inflammatory skin disorders (e.g. psoriasis, eczema), hepatic cirrhosis, spinal cord trauma and SIRS (systemic inflammatory response syndrome); smooth muscle spasm (e.g. asthma, angina), RDS (respiratory distress syndrome); hypotension (e.g. shock due to haemorrhage, septicaemia or anaphylaxis, carcinoid syndrome, dumping syndrome); oedema (e.g. burns, brain trauma, angioneurotic oedema whether or not as a result or treatment with inhibitors of angiotensin converting enzyme); pain and irritation (e.g. burns, wounds, cuts, rashes, stings, insect bites), migraine; male contraceptive agents by virtue of inhibition of prostate kallikrein; prevention of excessive blood loss during surgical procedures.

The compounds of the present invention may also be used to treat disorders that can be a response to the release of an inflammatory mediator (e.g. cough).

The compounds of the present invention may also be used to treat disorders involving regulation of growth factors (e.g. vascular endothelial growth factor (VEGF)) which may involve increased vascular permeability (e.g. diabetic retinopathy and septic shock).

The compounds of the present invention may be used to treat a neoplastic disorder (e.g. metastatic pancreatic adenocarcinomas, tumour angiogenesis) in particular they may be used to reduce angiogenesis associated with neoplasms e.g. cancer and tumour growth and to modulate angiogenesis and other processes associated with neoplasia and tumour growth and in particular may be used to block tumour angiogenesis and/or cancer cell invasion and metastasis.

Asthma

Asthma is a chronic lung condition that may be classified as allergic (intrinsic) or non-allergic (extrinsic). Patients with asthma experience difficulty breathing as a result of narrowing or obstruction of the airway, making it more difficult to move air in and out. This narrowing can result from airway inflammation and bronchoconstriction, Symptoms of asthma include, for example, wheezing, shortness of breath, bronchoconstriction, airway hyperreactivity, decreased lung capacity, fibrosis, airway inflammation and mucus production. A further symptom of asthma is exacerbations resulting from the original asthma attack which account for a significant morbidity from the disease. A KLK1 inhibitor can be used to ameliorate or prevent at least one symptom of asthma. A KLK1 inhibitor can also be administered in conjunction with another agent for treating asthma e.g. inhaled steroids, an oral steroid, a long acting beta-agonist, a leukotriene modifier, cromolyn sodium and nedocromil, theophylline and an anti-IgE antibody.

Allergic Rhinitis

Allergic rhinitis or “hay fever” involves an allergic reaction to pollen from grasses, trees, and weeds. When pollen is inhaled by an individual suffering from allergic rhinitis, antibody production and histamine release is triggered. Symptoms of allergic rhinitis include but are not limited to coughing, headache, itching of the eyes, mouth, throat, or nose, sneezing, nasal congestion, wheezing, sore throat, and watery eyes. The symptoms associated with hay fever vary significantly from person to person, and allergic rhinitis may be associated with other conditions such as asthma.

Chronic Obstructive Pulmonary Disease (COPD)

Chronic obstructive pulmonary disease (COPD) is a disease involving inflammation of the airways. Emphysema, along with chronic bronchitis, is part of COPD. It is a serious lung disease and is progressive, usually occurring in elderly patients. COPD causes over-inflation of structures in the lungs known as alveoli or air sacs. The walls of the alveoli break down resulting in a decrease in the respiratory ability of the lungs. Patients with this disease may first experience shortness of breath and cough. The KLK1 inhibitor can be used to ameliorate at least one symptom of COPD.

Cough

Cough can be caused by inflammation of the upper respiratory tract (throat and windpipe) due to a viral infection. Viral infections include; the common cold, flu, laryngitis, and bronchitis. These viral infections can also spread to the lower respiratory tract (bronchi) to cause a cough. A cough is a symptom of many illnesses and conditions including: asthma, bronchitis, influenza and whooping cough (pertussis) and may also result as a side effect from use of certain drugs such as ACE inhibitors. Individuals who smoke often have a smoker's cough, a loud, hacking cough which often results in the expiration of phlegm. The KLK1 inhibitor can be used to ameliorate or prevent at least one symptom of cough.

Pancreatitis

Pancreatitis is an inflammation of the pancreas. There are two types:

Acute pancreatitis—the inflammation comes on quickly over a few hours, and will usually go away leaving no permanent damage, although it can be fatal if complications occur (5% of cases).

Chronic pancreatitis—this condition often starts with bouts of acute pancreatitis, and eventually becomes a permanent condition. The pancreas becomes constantly inflamed. The KLK1 inhibitor can be used to ameliorate or prevent at least one symptom of pancreatitis.

Rheumatoid Arthritis (RA)

This order is characterised by inflammation in the lining of the joints and/or other internal organs. It is typically chronic, but can include flare-ups. Symptoms include, inflammation of joints, swelling, difficulty moving, pain and fever. A KLK1 inhibitor may be used to ameliorate or prevent at least one symptom of rheumatoid arthritis. A KLK1 inhibitor can be administered with another agent for treating rheumatoid arthritis, such as NSAIDs and aspirin, analgesics and corticosteroids which help reduce joint pain, stiffness and swelling.

Osteoarthritis

Osteoarthritis is a degenerative joint disease. It is characterised by the breakdown of cartilage in the joint, thus causing bones to rub against each other, causing pain and loss of movement. A KLK1 inhibitor can be used to ameliorate or prevent at least one symptom of osteoarthritis. A KLK1 inhibitor can be administered with another agent for treating rheumatoid arthritis, such as a corticosteroid or an NSAID.

Angiogenesis-Associated and Neoplastic Disorders

In one embodiment, a KLK1 inhibitor may be administered to a subject to modulate angiogenesis or other processes associated with neoplasia and tumour growth.

For example a KLK1 inhibitor may be used to reduce angiogenesis (e.g. uncontrolled or unwanted angiogenesis) such as angiogenesis associated with vascular malformations and cardiovascular disorders (e.g. atherosclerosis, restenosis and arteriovenous malformations), chronic inflammatory diseases (e.g. diabetes mellitus, inflammatory bowel disease, psoriasis and rheumatoid arthritis), dermatological disorders (e.g. arterial ulcers, systemic vasculitis and scleroderma) or ocular disorders (e.g. blindness caused by neovascular disease, neovascular glaucoma, corneal neovascularization, trachoma, diabetic retinopathy and myopic degeneration).

In particular, a KLK1 inhibitor can be used to reduce angiogenesis associated with neoplasia, e.g., cancer and tumour growth, e.g., growth of a benign, malignant, or metastatic tumour.

Examples of cancerous disorders include, but are not limited to, solid tumours, soft tissue tumours and metastatic lesions. Examples include sarcomas, adenocarcinomas and carcinomas of various organ systems such as those affecting lung, breast, lymphoid, gastrointestinal (e.g. colon) and genitourinary tract (e.g. renal urothelial cells), pharynx, prostate, ovary as well as adenocarcinomas which include malignancies such as most colon cancers, rectal cancer, renal-cell carcinoma, liver cancer, non-small cell carcinoma of the lung, pharynx, cancer of the small intestine, cancer of the esophagus and others.

Exemplary solid tumours that can be treated include: fibrosarcoma, myxosarcoma, liposarcoma, chrondrosarcoma, osteogenic sarcoma, chordoma, lymphanangioendotheliosarcoma, synovioma, mesothelioma, Ewing's tumour, leiomyosaarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, heptoma, bile duct carcinoma, choriocarcinoma, seminoma, embryonal carcinoma, cervical cancer, testicular tumour, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemagioblastoma, acoustic neuroma, oligodendroglioma, meningioma, melanoma, neuroblastoma and retinoblastoma.

The KLK1 inhibitor can also be used to treat a carcinoma, e.g. a malignancy of epithelial or endocrine tissues including respiratory system carcinomas, gastrointestinal system carcinomas, genitourinary system carcinomas and melanoma. Exemplary carcinoma include adenocarcinoma, carcinomas of tissue of the cervix, lung, prostate, breast, head and neck, colon and ovary.

The KLK1 inhibitor can also be used to treat sarcomas, e.g. malignant tumours of mesenchchymal derivation.

The KLK1 inhibitor can be administered in combination with another agent for treating neoplastic and/or metastatic disorders. Examples of such other agents include:

(i) other antiangiogenic agents (e.g. linomide, angiostatin, razoxane);

(ii) cytostatic agents such as antiestrogens(e.g. tamoxifan, toremifene, raloxifene), progestogens(e.g. megestrol acetate), aromatase inhibitors (e.g. anastrozole, letrozole), antiprogestogens, antiandrogens(e.g. flutamide, nilutamide, bicalutamide), anti-invasion agents (e.g. metalloproteinase inhibitors such as marimastat and inhibitors of urokinase plasminogen activator receptor function).

(iii) antiproliferative/antineoplastic drugs and combinations thereof, as used in medical oncology, such as antimetabolites (e.g. fluoropyrimidines like 5-fluorouracil, purine and adenosine analogues, cytosine arabinoside); Intercalating antitumour antibiotics (e.g. anthracyclines like doxorubicin, daunomycin, epirubicin); platinum derivatives (e.g. cisplatin, carboplatin)alkylating agents (e.g. chlorambucil, cyclophosphamide); antmitotic agents (e.g. vinca alkaloids lsuch as vincristine and taxoids like TAXOL® (paclitaxel), TAXOTERE® (docetaxel, topoisomerase inhibitors (e.g. epipodophyllotoxins such as etoposide and teniposide) and proteasome inhibitors such as VELCADE® (bortezomib).

Accordingly, the present invention provides a compound of formula (I) for use in therapy.

The present invention also provides for the use of a compound of formula (I) in the manufacture of a medicament for the treatment or prevention of a disease or condition in which KLK1 activity is implicated. Diseases or conditions in which KLK1 activity is implicated include inflammation, respiratory disorders, disorders involving regulation of growth factors and neoplastic disorders. Specific examples of such diseases and conditions include those listed above.

The present invention also provides a compound of formula (I) for the treatment or prevention of a disease or condition in which KLK1 activity is implicated. Diseases or conditions in which KLK1 activity is implicated include inflammation, respiratory disorders, disorders involving regulation of growth factors and neoplastic disorders. Specific examples of such diseases and conditions include those listed above.

The present invention also provides a method of treatment of a disease or condition in which KLK1 activity is implicated comprising administration to a subject in need thereof a therapeutically effective amount of a compound of formula (I). Diseases or conditions in which KLK1 activity is implicated include inflammation, respiratory disorders, disorders involving regulation of growth factors and neoplastic disorders. Specific examples of such diseases and conditions include those listed above.

In one aspect, the disease or condition in which KLK1 activity is implicated is selected from an inflammatory or respiratory disorder or condition selected from asthma (allergic and non-allergic), chronic obstructive pulmonary disease (COPD), allergic rhinitis (hayfever), cough, exacerbations resulting from asthma and chronic obstructive pulmonary disease (COPD), multiple sclerosis, arthritis, rheumatoid arthritis, osteopathic arthritis, osteoarthritis, rhinitis, sinusitis, inflammatory bowel disease (such as Crohn's disease and ulcerative colitis), immune mediated diabetes, acute pancreatitis and interstitial cystitis, conjunctivitis, periodontal disease, chronic prostate inflammation, chronic recurrent parotitis, inflammatory skin disorders (e.g. psoriasis, eczema), and SIRS (systemic inflammatory response syndrome); smooth muscle spasm (e.g. asthma, angina), RDS (respiratory distress syndrome) , rhino-conjunctivitis, rhinorrhoea, urticaria or a neoplastic disorder.

In another aspect, the disease or condition in which KLK1 activity is implicated is a respiratory disorder selected from asthma (allergic and non-allergic), chronic obstructive pulmonary disease (COPD), allergic rhinitis (hayfever), cough, exacerbations resulting from asthma and chronic obstructive pulmonary disease (COPD),

In a further aspect, the disease or condition in which KLK1 activity is implicated is a respiratory disorder selected from asthma (allergic and non-allergic) and cough.

### DEFINITIONS

The term “alkyl” includes saturated hydrocarbon residues including:
linear groups up to 10 atoms (C₁-C₁₀), or of up to 6 atoms (C₁-C₆), or of up to 4 atoms (C₁-C₄). Examples of such alkyl groups include, but are not limited, to C₁-methyl, C₂-ethyl, C₃-propyl and C₄-n-butyl.
branched groups of between 3 and 10 atoms (C₃-C₁₀), or of up to 7 atoms (C₃-C₇), or of up to 4 atoms (C₃-C₄). Examples of such alkyl groups include, but are not limited to, C₃-iso-propyl, C₄-sec-butyl, C₄-iso-butyl, C₄-tert-butyl and C₅-neo-pentyl.

each optionally substituted as stated above.

The term “alkenyl” includes monounsaturated hydrocarbon residues including:
linear groups of between 2 and 6 atoms (C₂-C₆). Examples of such alkenyl groups include, but are not limited to, C₂-vinyl, C₃-1-propenyl, C₃-allyl, C₄-2-butenyl
branched groups of between 3 and 8 atoms (C₃-C₈). Examples of such alkenyl groups include, but are not limited to, C₄-2-methyl-2-propenyl and C₆-2,3-dimethyl-2-butenyl.

each optionally substituted as stated above.

The term “alkoxy” includes O-linked hydrocarbon residues including:
linear groups of between 1 and 6 atoms (C₁-C₆), or of between 1 and 4 atoms (C₁-C₄). Examples of such alkoxy groups include, but are not limited to, C₁-methoxy, C₂-ethoxy, C₃-n-propoxy and C₄-n-butoxy.
branched groups of between 3 and 6 atoms (C₃-C₆) or of between 3 and 4 atoms (C₃-C₄). Examples of such alkoxy groups include, but are not limited to, C₃-iso-propoxy, and C₄-sec-butoxy and tert-butoxy.

each optionally substituted as stated above.

Unless otherwise stated, halo is selected from Cl, F, Br and I.

Cycloalkyl is as defined above. Conveniently cycloalkyl groups may contain from 4 to 10 carbon atoms, or from 5 to 10 carbon atoms, or from 4 to 6 carbon atoms. Examples of suitable monocyclic cycloalkyl groups include cyclopropyl, cyclobutyl, cyclopentyl, cyclohexyl, cycloheptyl, cyclopentene, cyclopenta-1,3-diene, cyclohexene and cyclohexa-1,4-diene (optionally substituted as stated above). Examples of suitable bicyclic cycloalkyl groups include decahydronaphthalene, octahydro-1H-indene (optionally substituted as stated above). Examples of suitable cycloalkyl groups, when fused with aryl, include indanyl and 1,2,3,4-tetrahydronaphthyl (optionally substituted as stated above).

Heterocycloalkyl is as defined above. Examples of suitable heterocycloalkyl groups include oxiranyl, aziridinyl, azetidinyl, tetrahydrofuranyl, pyrrolidinyl, tetrahydropyranyl, piperidinyl, N-methylpiperidinyl, morpholinyl, N-methyl morpholinyl, thiomorpholinyl, thiomorpholinyl-1-oxide, thiomorpholinyl-1,1-dioxide, piperazinyl, N-methylpiperazinyl, azepinyl oxazepinyl, diazepinyl, and 1,2,3,4-tetrahydropyridinyl (optionally substituted as stated above).

Aryl is as defined above. Typically, aryl will be optionally substituted with 1, 2 or 3 substituents. Optional substituents are seleted from those stated above. Examples of suitable aryl groups include phenyl and naphthyl (each optionally substituted as stated above).

Heteroaryl is as defined above. Examples of suitable heteroaryl groups include thienyl, furanyl, pyrrolyl, pyrazolyl, imidazoyl, oxazolyl, isoxazolyl, thiazolyl, isothiazolyl, triazolyl, oxadiazolyl, thiadiazolyl, tetrazolyl, pyridinyl, pyridazinyl, pyrimidinyl, pyrazinyl, indolyl, benzimidazolyl, benzotriazolyl, quinolinyl and isoquinolinyl (optionally substituted as stated above).

The term “C-linked”, such as in “C-linked heterocycloalkyl”, means that the heterocycloalkyl group is joined to the remainder of the molecule via a ring carbon atom.

The term “N-linked”, such as in “N-linked heterocycloalkyl”, means that the heterocycloalkyl group is joined to the remainder of the molecule via a ring nitrogen atom.

The term “O-linked”, such as in “O-linked hydrocarbon residue”, means that the hydrocarbon residue is joined to the remainder of the molecule via an oxygen atom.

In groups such as aryl(C₁-C₄)alkyl- and —SO₂(C₁-C₆)alkyl, “—” denotes the point of attachment of the group to the remainder of the molecule.

“Pharmaceutically acceptable salt” means a physiologically or toxicologically tolerable salt and includes, when appropriate, pharmaceutically acceptable base addition salts and pharmaceutically acceptable acid addition salts. For example (i) where a compound of the invention contains one or more acidic groups, for example carboxy groups, pharmaceutically acceptable base addition salts that can be formed include sodium, potassium, calcium, magnesium and ammonium salts, or salts with organic amines, such as, diethylamine, N-methyl-glucamine, diethanolamine or amino acids (e.g. lysine) and the like; (ii) where a compound of the invention contains a basic group, such as an amino group, pharmaceutically acceptable acid addition salts that can be formed include hydrochlorides, hydrobromides, sulfates, phosphates, acetates, citrates, lactates, tartrates, mesylates, succinates, oxalates, phosphates, esylates, tosylates, benzenesulfonates, naphthalenedisulphonates, maleates, fumarates, hippurates, xinafoates, p-acetamidobenzoates, dihydroxybenzoates, hydroxynaphthoates, succinates, ascorbates, oleates, bisulfates and the like.

Hemisalts of acids and bases can also be formed, for example, hemisulfate and hemicalcium salts.

For a review of suitable salts, see “Handbook of Pharmaceutical Salts: Properties, Selection and Use” by Stahl and Wermuth (Wiley-VCH, Weinheim, Germany, 2002).

“Prodrug” refers to a compound which is convertible in vivo by metabolic means (e.g. by hydrolysis, reduction or oxidation) to a compound of the invention. Suitable groups for forming pro-drugs are described in ‘The Practice of Medicinal Chemistry, 2^{nd} Ed. pp 561-585 (2003) and in F. J. Leinweber, Drug Metab. Res., 1987, 18, 379.

The compounds of the invention can exist in both unsolvated and solvated forms. The term ‘solvate’ is used herein to describe a molecular complex comprising the compound of the invention and a stoichiometric amount of one or more pharmaceutically acceptable solvent molecules, for example, ethanol. The term ‘hydrate’ is employed when the solvent is water.

Where compounds of the invention exist in one or more geometrical, optical, enantiomeric, diastereomeric and tautomeric forms, including but not limited to cis- and trans-forms, E- and Z-forms, R-, S- and meso-forms, keto-, and enol-forms. Unless otherwise stated a reference to a particular compound includes all such isomeric forms, including racemic and other mixtures thereof. Where appropriate such isomers can be separated from their mixtures by the application or adaptation of known methods (e.g. chromatographic techniques and recrystallisation techniques). Where appropriate such isomers can be prepared by the application or adaptation of known methods (e.g. asymmetric synthesis).

Typical configurations of the compounds of formula (I) include:

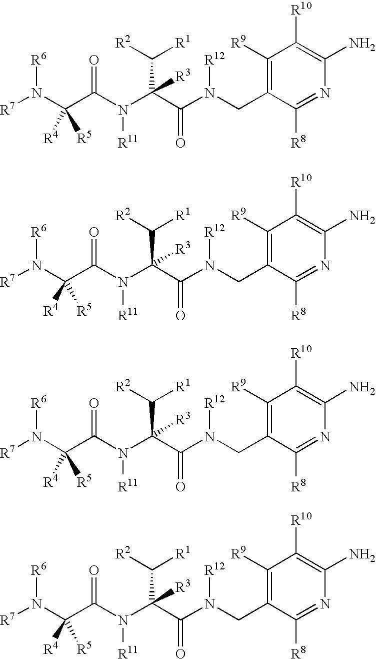

Other typical configurations of the compounds of formula (I) include:

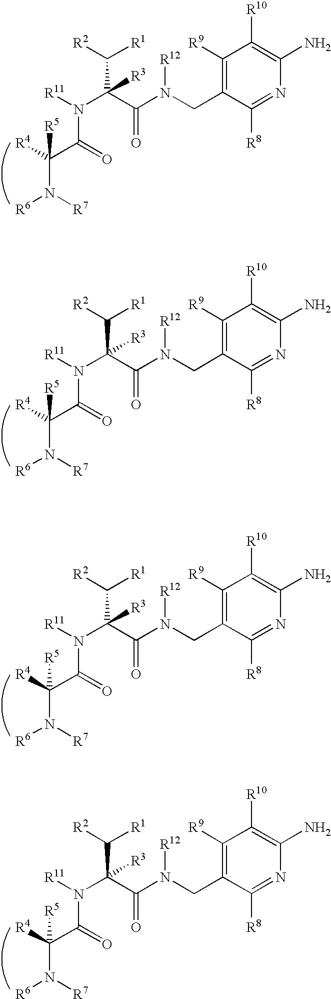

In the context of the present invention, references herein to “treatment” include references to curative, palliative and prophylactic treatment.

General Methods

The compounds of formula (I) should be assessed for their biopharmaceutical properties, such as solubility and solution stability (across pH), permeability, etc., in order to select the most appropriate dosage form and route of administration for treatment of the proposed indication.

Compounds of the invention intended for pharmaceutical use may be administered as crystalline or amorphous products. They may be obtained, for example, as solid plugs, powders, or films by methods such as precipitation, crystallization, freeze drying, spray drying, evaporative drying, melt congealing and extrusion. Conventional drying processes including static/dynamic oven, infrared, microwave or radio frequency drying may be used to assist in the formation of the above crystalline and amorphous products.

They may be administered alone or in combination with one or more other compounds of the invention or in combination with one or more other drugs (or as any combination thereof). Generally, they will be administered as a formulation in association with one or more pharmaceutically acceptable excipients. The term ‘excipient’ is used herein to describe any ingredient other than the compound(s) of the invention which may impart either a functional (i.e., drug release rate controlling) and/or a non-functional (i.e., processing aid or diluent) characteristic to the formulations. The choice of excipient will to a large extent depend on factors such as the particular mode of administration, the effect of the excipient on solubility and stability, and the nature of the dosage form.

Pharmaceutical compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Mack Publishing Company, 1995).

Accordingly, the present invention provides a pharmaceutical composition comprising a compound of formula (I) and a pharmaceutically acceptable carrier, diluent or excipient.

The compounds of the invention may be administered orally. Oral administration may involve swallowing, so that the compound enters the gastrointestinal tract, and/or buccal, lingual, or sublingual administration by which the compound enters the blood stream directly from the mouth.

Formulations suitable for oral administration include solid plugs, solid microparticulates, semi-solid and liquid (including multiple phases or dispersed systems) such as tablets; soft or hard capsules containing multi- or nano-particulates, liquids, emulsions or powders; lozenges (including liquid-filled); chews; gels; fast dispersing dosage forms; films; ovules; sprays; and buccal/mucoadhesive patches.

Formulations suitable for oral administration may also be designed to deliver the compounds of formula (I) in an immediate release manner or in a rate-sustaining manner, wherein the release profile can be delayed, pulsed, controlled, sustained, or delayed and sustained or modified in such a manner which optimises the therapeutic efficacy of the said compounds. Means to deliver compounds in a rate-sustaining manner are known in the art and include slow release polymers that can be formulated with the said compounds to control their release.

Examples of rate-sustaining polymers include degradable and non-degradable polymers that can be used to release the said compounds by diffusion or a combination of diffusion and polymer erosion. Examples of rate-sustaining polymers include hydroxypropyl methylcellulose, hydroxypropyl cellulose, methyl cellulose, ethyl cellulose, sodium carboxymethyl cellulose, polyvinyl alcohol, polyvinyl pyrrolidone, xanthum gum, polymethacrylates, polyethylene oxide and polyethylene glycol.

Liquid (including multiple phases and dispersed systems) formulations include emulsions, suspensions, solutions, syrups and elixirs. Such formulations may be presented as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Liang and Chen, Expert Opinion in Therapeutic Patents, 2001, 11(6), 981-986.

The formulation of tablets is discussed in Pharmaceutical Dosage Forms: Tablets, Vol. 1, by H. Lieberman and L. Lachman (Marcel Dekker, New York, 1980).

The compounds of the invention may also be administered directly into the blood stream, into subcutaneous tissue, into muscle, or into an internal organ. Suitable means for parenteral administration include intravenous, intraarterial, intraperitoneal, intrathecal, intraventricular, intraurethral, intrasternal, intracranial, intramuscular, intrasynovial and subcutaneous. Suitable devices for parenteral administration include needle (including microneedle) injectors, needle-free injectors and infusion techniques.

Parenteral formulations are typically aqueous or oily solutions. Where the solution is aqueous, excipients such as sugars (including but restricted to glucose, manitol, sorbitol, etc.) salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

Parenteral formulations may include implants derived from degradable polymers such as polyesters (i.e., polylactic acid, polylactide, polylactide-co-glycolide, polycapro-lactone, polyhydroxybutyrate), polyorthoesters and polyanhydrides. These formulations may be administered via surgical incision into the subcutaneous tissue, muscular tissue or directly into specific organs.

The preparation of parenteral formulations under sterile conditions, for example, by lyophilisation, may readily be accomplished using standard pharmaceutical techniques well known to those skilled in the art.

The solubility of compounds of formula (I) used in the preparation of parenteral solutions may be increased by the use of appropriate formulation techniques, such as the incorporation of co-solvents and/or solubility-enhancing agents such as surfactants, micelle structures and cyclodextrins.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler, as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane, or as nasal drops. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

Formulations for inhaled/intranasal administration may be formulated to be immediate and/or modified release using, for example, PGLA. Modified release formulations include delayed-, sustained-, pulsed-, controlled-, targeted and programmed release.

Inasmuch as it may desirable to administer a combination of active compounds, for example, for the purpose of treating a particular disease or condition, it is within the scope of the present invention that two or more pharmaceutical compositions, at least one of which contains a compound of formula (I), may conveniently be combined in the form of a kit suitable for coadministration of the compositions.

Thus the kit of the invention comprises two or more separate pharmaceutical compositions, at least one of which contains a compound of formula (I) in accordance with the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

In the present case, compounds of the present invention may be conveniently combined with an additional therapeutic agent or agents.

For example, as mentioned above, a KLK1 inhibitor can also be administered in conjunction with another agent for treating asthma e.g. inhaled steroids, an oral steroid, a long acting beta-agonist, a leukotriene modifier, cromolyn sodium and nedocromil, theophylline and an anti-IgE antibody.

If a combination of active agents is administered, then they may be administered simultaneously, separately or sequentially.

For administration to human patients, the total daily dose of the compounds of the invention is typically in the range 0.01 mg and 1000 mg, or between 0.1 mg and 250 mg, or between 1 mg and 50 mg depending, of course, on the mode of administration. The total daily dose may be administered in single or divided doses and may, at the physician's discretion, fall outside of the typical range given herein. These dosages are based on an average human subject having a weight of about 60 kg to 70 kg. The physician will readily be able to determine doses for subjects whose weight falls outside this range, such as infants and the elderly.

Synthetic Methods

The compounds of the present invention can be prepared according to the procedures of the following schemes and examples, using appropriate materials, and are further exemplified by the specific examples provided herein below. Moreover, by utilising the procedures described herein, one of ordinary skill in the art can readily prepare additional compounds that fall within the scope of the present invention claimed herein. The compounds illustrated in the examples are not, however, to be construed as forming the only genus that is considered as the invention. The examples further illustrate details for the preparation of the compounds of the present invention. Those skilled in the art will readily understand that known variations of the conditions and processes of the following preparative procedures can be used to prepare these compounds.

The compounds of the invention may be isolated in the form of their pharmaceutically acceptable salts, such as those described previously herein above.

It may be necessary to protect reactive functional groups (e.g. hydroxy, amino, thio or carboxy) in intermediates used in the preparation of compounds of the invention to avoid their unwanted participation in a reaction leading to the formation of the compounds. Conventional protecting groups, for example those described by T. W. Greene and P. G. M. Wuts in “Protective groups in organic chemistry” John Wiley and Sons, 4^{th} Edition, 2006, may be used. For example, a common amino protecting group suitable for use herein is tert-butoxy carbonyl (Boc), which is readily removed by treatment with an acid such as trifluoroacetic acid or hydrogen chloride in an organic solvent such as dichloromethane. Alternatively the amino protecting group may be a benzyloxycarbonyl (Z) group which can be removed by hydrogenation with a palladium catalyst under a hydrogen atmosphere or 9-fluorenylmethyloxycarbonyl (Fmoc) group which can be removed by solutions of secondary organic amines such as diethylamine or piperidine in an organic solvents. Carboxyl groups are typically protected as esters such as methyl, ethyl, benzyl or tert-butyl which can all be removed by hydrolysis in the presence of bases such as lithium or sodium hydroxide. Benzyl protecting groups can also be removed by hydrogenation with a palladium catalyst under a hydrogen atmosphere whilst tert-butyl groups can also be removed by trifluoroacetic acid. Alternatively a trichloroethyl ester protecting group is removed with zinc in acetic acid. A common hydroxy protecting group suitable for use herein is a methyl ether, deprotection conditions comprise refluxing in 48% aqueous HBr for 1-24 hours, or by stirring with borane tribromide in dichloromethane for 1-24 hours. Alternatively where a hydroxy group is protected as a benzyl ether, deprotection conditions comprise hydrogenation with a palladium catalyst under a hydrogen atmosphere.

In the following schemes:

R¹-R⁷ and R¹¹ are as previously defined for the compounds of formula (I);

PG₁, PG₂ or PG₃ is a suitable protecting group;

R²⁰ is H, (C₁-C₁₀)alkyl, halogen, hydroxyl or (C₁-C₆)alkoxy;

R²¹ is H, (C₁-C₆)alkyl, (C₃-C₁₀)cycloalkyl, (C₃-C₁₀)cycloalkyl(C₁-C₄)alkyl-, aryl, heteroaryl, aryl(C₁-C₄)alkyl-, aryl(C₂-C₄)alkenyl-, or heteroaryl(C₁-C₄)alkyl-;

R²² is H, (C₁-C₆)alkyl, (C₃-C₁₀)cycloalkyl, (C₃-C₁₀)cycloalkyl(C₁-C₄)alkyl-, aryl, heteroaryl, aryl(C₁-C₄)alkyl-, aryl(C₂-C₄)alkenyl-, or heteroaryl(C₁-C₄)alkyl-; and

R²³ is H, (C₁-C₆)alkyl, (C₃-C₁₀)cycloalkyl, (C₃-C₁₀)cycloalkyl(C₁-C₄)alkyl-, aryl or aryl(C₁-C₄)alkyl-.

The compounds according to general formula I can be prepared using conventional synthetic methods. In a typical first step, 5-aminomethyl-pyridin-2-ylamine or substituted 5-aminomethyl-pyridin-2-ylamine (3) is prepared by reduction of the corresponding nitrile (2). This can be achieved either by direct reduction of the nitrile by hydrogenation in a suitable solvent such as methanol in the presence of a suitable catalyst such as palladium on charcoal in the presence of an acid such as hydrochloric acid or reduction with a suitable borohydride in the presence of a suitable transition metal such as cobalt or nickel chloride in a suitable solvent such as methanol at room temperature; or by trapping out of the tert-butoxycarbonyl (Boc) protected amine(4) (using, for example, the method as described in S. Caddick et al., Tetrahedron Lett., 2000, 41, 3513) which is then subsequently deprotected by standard means described previously to give the amine(3).

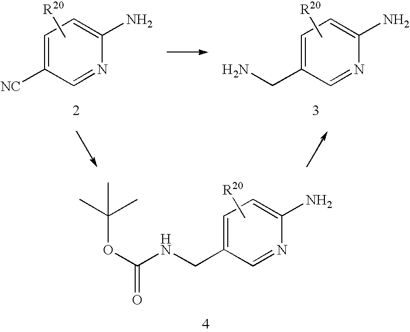

Alternatively amine (3) can be prepared from the corresponding acid (5) via a primary amide (6). Typically, acid (5) is treated with ammonia in the presence of a suitable coupling reagent in a suitable solvent such as dichloromethane and DMF at room temperature. The resulting amide (6) is then reduced with a reducing agent such as lithium aluminium hydride in a suitable solvent such as tetrahydrofuran at room temperature to yield amine (3).

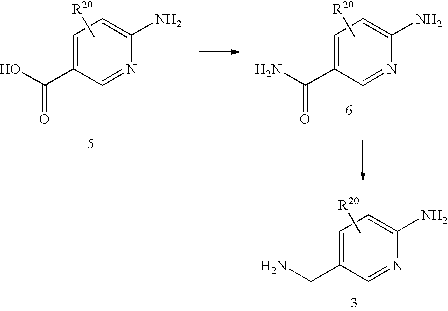

In a typical second step, the amine (3) is coupled using standard peptide coupling conditions to an alpha amino acid (7) suitably amino-protected with a standard protecting group such as tert-butyloxycarbonyl (Boc), benzyloxycarbonyl (Z) or 9-fluorenylmethyloxycarbonyl (Fmoc). The use of such groups is well known in the art. Where R²¹ has a reactive functional group such as an amine or a carboxylic acid, this group will also be protected. Standard peptide coupling methods include the reaction of acids with amines in the presence of hydroxybenzotriazole and carbodiimide such as water soluble carbodiimide, or 2-(1H-benzotriazole-1-yl)-1,1,3,3-tetramethylaminium hexafluorophosphate or benzotriazole-1-yl-oxy-tris-pyrrolidino-phosphoium hexaffluorophosphate or bromo-trispyrolidino-phosphoium hexafluorophosphate in the presence of organic bases such as triethylamine, diisopropylethylamine or N-methylmorpholine.

### Error! Objects cannot be Created from Editing Field Codes

The protecting group of (8) is removed using standard methods described previously to yield the amine (9).

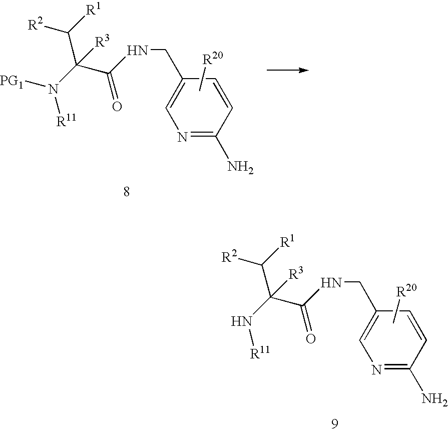

The amine (9) is coupled using the standard peptide coupling conditions described previously to an alpha amino acid (10) suitably amino-protected with a suitable protecting group such as tert-butyloxycarbonyl (Boc), benzyloxycarbonyl (Z) or 9-fluorenylmethyloxycarbonyl (Fmoc). Where R¹ or R² has a reactive functional group such as an amine or a carboxylic acid, this group will also be protected. The protecting group of the resulting protected dipeptide derivative (11) is removed using the standard methods described previously to give the amine (12).

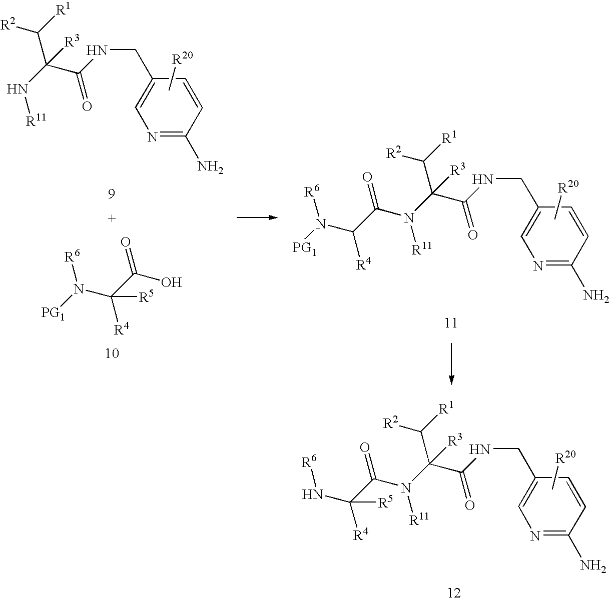

The amine (12) is further derivatised by reductive alkylation with a suitable aldehyde or ketone to yield the alkylated amine (13). Typically, amine (12) is allowed to react with the aldehyde or ketone in the presence of a suitable reducing agent such as sodium cyanoborohydride or sodium acetoxyborohydride in a suitable solvent such as methanol, at room temperature.

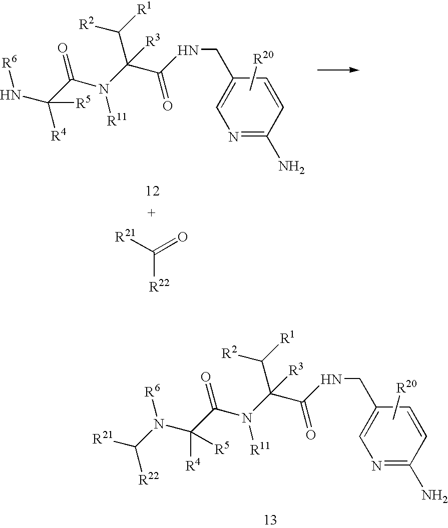

Compound 15 can also be prepared by coupling the alkylated alpha amino acid (14) with the amine (9) using standard peptide coupling conditions described previously.

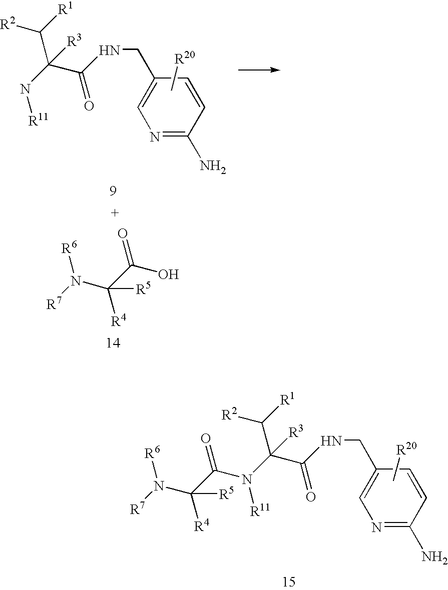

Alkylated alpha amino acids (17) can be prepared by the reductive alkylation of the parent alpha amino acid in which the carboxyl group is unprotected (16) or in which it is protected as an ester with a standard protecting group such as a methyl, tert-butyl or trichloroethyl ester (18), following alkylation this protecting group is removed using standard methods described previously. Typical conditions for carrying out the reductive alkylation are described above.

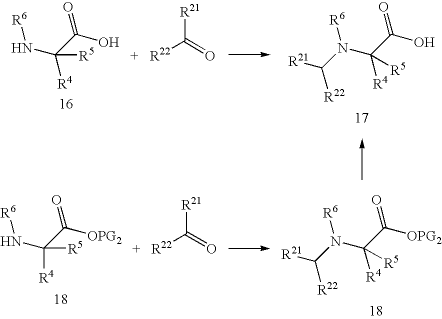

Alternatively the alpha amino acid (14) may be prepared from the corresponding bromoacetic acid derivative, suitably carboxyl-protected with a standard protecting group, such as a methyl, tert-butyl, trichloroethyl ester (19) by reaction with the required amine followed by the deprotection using standard methods. Typically, bromoacetic acid derivative (19) is allowed to react with the amine in the presence of a base such as diisopropylethylamine or potassium or sodium carbonate in a suitable solvent such as acetonitrile or tetrahydrofuran at room temperature.

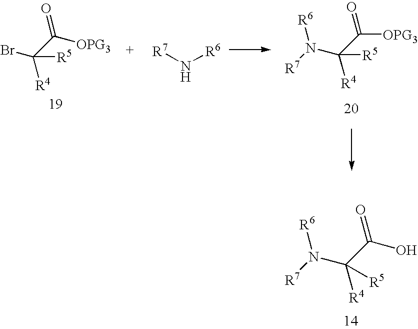

Compound (11) can also be synthesised from the dipeptide (22) suitably amino-protected with a standard protecting group such as tert-butyloxycarbonyl (Boc), benzyloxycarbonyl (Z) or 9-fluorenylmethyloxycarbonyl (Fmoc). Such a dipeptide can be prepared from two alpha amino acids one of which is amino-protected with a standard protecting group such as tert-butyloxycarbonyl (Boc), benzyloxycarbonyl (Z) or 9-fluorenylmethyloxycarbonyl (Fmoc) whilst the other is carboxyl-protected with a standard protecting group such as an ester such as a methyl, tert-butyl, trichloroethyl ester. The carboxyl protecting group of (21) is removed by standard methods described previously following the coupling reaction. The amide bond forming reactions may be carried out using the standard peptide coupling conditions described above.

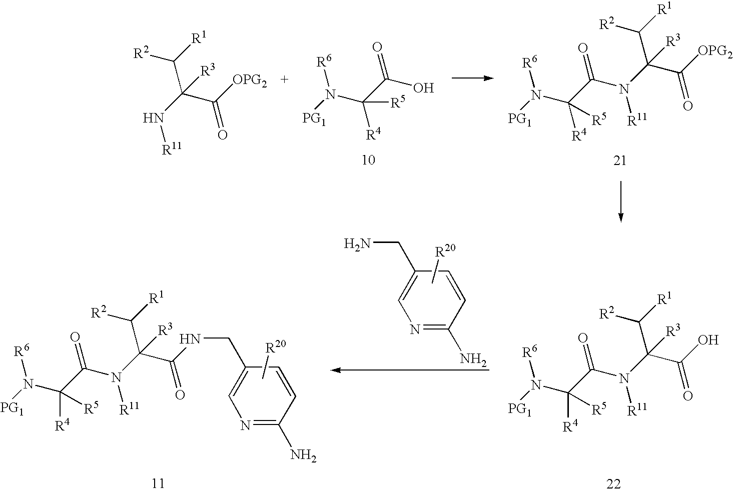

The amine 12 can be further derivatised by reaction with a sulphonyl chloride in the presence of a base such as triethylamine or diisopropylamine to yield the sulphonamide (23).

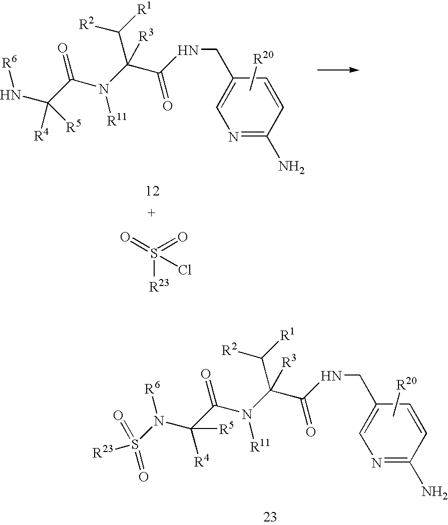

The present invention also encompasses intermediate compounds that have utility in the synthesis of the compounds of formula (I). Accordingly, one aspect of the present invention provides an intermediate compound selected from the group including:

{(S)-1-[(6-Amino-pyridin-3-ylmethyl)-carbamoyl]-2-naphthalen-1-yl-ethyl}-carbamic acid tert-butyl ester;

(S)-2-Amino-N-(6-amino-pyridin-3-ylmethyl)-3-naphthalen-1-yl-propionamide ditrifluoroacetate;

((R)-1-{(S)-1-[(6-Amino-pyridin-3-ylmethyl)-carbamoyl]-2-naphthalen-1-yl-ethylcarbamoyl}-2-methyl-butyl)-carbamic acid tert-butyl ester;

[(S)-1-[(6-Amino-pyridin-3-ylmethyl)-carbamoyl]-2-(decahydro-naphthalen-1-yl)-ethyl]-carbamic acid tert-butyl ester;

(S)-2-Amino-N-(6-amino-pyridin-3-ylmethyl)-3-(decahydro-naphthalen-1-yl)-propionamide dihydrochloride;

{(R)-1-[(S)-1-[(6-Amino-pyridin-3-ylmethyl)-carbamoyl]-2-(decahydro-naphthalen-1-yl)-ethylcarbamoyl]-2-methyl-butyl}-carbamic acid tert-butyl ester;

{(S)-1-[(S)-1-[(6-Amino-pyridin-3-ylmethyl)-carbamoyl]-2-(3,4-dichloro-phenyl)-ethylcarbamoyl]-2-methyl-butyl}-carbamic acid tert-butyl ester;

(S)-2-Amino-N-(6-amino-pyridin-3-ylmethyl)-3-(3,4-dichloro-phenyl)-propionamide dihydrochloride;

{(R)-1-[(S)-1-[(6-Amino-pyridin-3-ylmethyl)-carbamoyl]-2-(3,4-dichloro-phenyl)-ethylcarbamoyl]-2-methyl-butyl}-methyl-carbamic acid tert-butyl ester;

(R)-2-[(S)-1-[(6-Amino-pyridin-3-ylmethyl)-carbamoyl]-2-(3,4-dichloro-phenyl)-ethylcarbamoyl]-pyrrolidine-1-carboxylic acid tert-butyl ester;

[(S)-1-[(6-Amino-pyridin-3-ylmethyl)-carbamoyl]-2-(3,4-difluoro-phenyl)-ethyl]-carbamic acid tert-butyl ester;

(S)-2-Amino-N-(6-amino-pyridin-3-ylmethyl)-3-(3,4-difluoro-phenyl)-propionamide dihydrochloride;

{(S)-1-[(6-Amino-2-methyl-pyridin-3-ylmethyl)-carbamoyl]-2-naphthalen-1-yl-ethyl}-carbamic acid tert-butyl ester;

(S)-2-Amino-N-(6-amino-2-methyl-pyridin-3-ylmethyl)-3-naphthalen-1-yl-propionamide dihydrochloride;

{(R)-1-[(6-Amino-pyridin-3-ylmethyl)-carbamoyl]-2,2-dicyclohexyl-ethyl}-carbamic acid tert-butyl ester;

(R)-2-Amino-N-(6-amino-pyridin-3-ylmethyl)-3,3-dicyclohexyl-propionamide ditrifluoroacetate; and

((S)-1-{(R)-1-[(6-Amino-pyridin-3-ylmethyl)-carbamoyl]-2,2-dicyclohexyl-ethylcarbamoyl}-2-methyl-butyl)-carbamic acid tert-butyl ester.

In one aspect, the present invention provides a process for the preparation of a compound of formula (I),

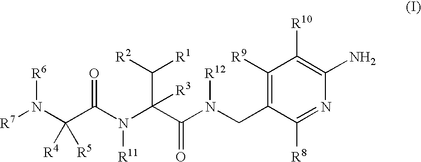

comprising the reaction of a compound of formula (IV):

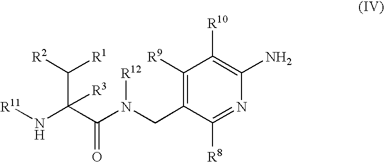

with a compound of formula (V):

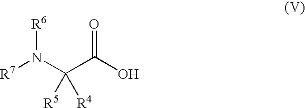

under standard peptide coupling conditions; wherein R¹-R¹² are as previously defined for the compounds of formula (I).

Standard peptide coupling conditions include the reaction of acids with amines in the presence of hydroxybenzotriazole and carbodiimide such as water soluble carbodiimide, or 2-(1H-benzotriazole-1-yl)-1,1,3,3-tetramethylaminium hexafluorophosphate or benzotriazole-1-yl-oxy-tris-pyrrolidino-phosphoium hex affluorophosphate or bromo-trispyrolidino-phophoium hexafluorophosphate in the presence of organic bases such as triethylamine, diisopropylethylamine or N-methylmorpholine. These reactions are typically carried out in solvents such as dichloromethane and dimethylformamide.

### EXAMPLES

The invention is illustrated by the following non-limiting examples in which the following abbreviations and definitions are used:

| | |
|---|---|
| DMF | N,N-Dimethylformamide |
| EtOAc | Ethyl Acetate |
| hrs | Hours |
| HBTU | 2-(1H-Benzotriazole-1-yl)-1,1,3,3-tetramethyluronium |
| | hexafluorophosphate |
| HOBt | Hydroxybenzotriazole |
| Ile | Isoleucine |
| LCMS | Liquid chromatography mass spectrometry |
| Me | Methyl |
| MeCN | Acetonitrile |
| MeOH | Methanol |
| Min | Minutes |
| MS | Mass spectrum |
| Nal | Napthylalanine |
| NMR | Nuclear magnetic resonance spectrum - NMR spectra were |
| | recorded at a frequency of 270 or 400 MHz unless otherwise |
| | indicated |
| Pet. Ether | Petroleum ether fraction boiling at 60-80° C. |
| Phe | Phenylalanine |
| Pro | Proline |
| PyBOP ® | Benzotriazole-1-yl-oxy-tris-pyrrolidino-phosphoium |
| | hexafluorophosphate |
| THF | Tetrahydrofuran |
| TFA | Trifluoroacetic acid |

All reactions were carried out under an atmosphere of nitrogen unless specified otherwise.

¹H NMR spectra were recorded on a Jeol EX 270 (270 MHz) or Brucker Avance III (400 MHz) spectrometer with reference to deuterium solvent and at room temperature. Molecular ions were obtained using LCMS which was carried out using a Chromolith Speedrod RP-18e column, 50×4.6 mm, with a linear gradient 10% to 90% 0.1% HCO₂H/MeCN into 0.1% HCO₂H/H₂O over 11 min, flow rate 1.5 mL/min. Data was collected using a Thermofinnigan Surveyor MSQ mass spectrometer with electospray ionisation in conjunction with a Thermofinnigan Surveyor LC system.

Chemical names were generated using the Autonom software provided as part of the ISIS draw package from MDL Information Systems.

Where products were purified by flash chromatography, ‘silica’ refers to silica gel for chromatography, 0.035 to 0.070 mm (220 to 440 mesh) (e.g. Merck silica gel 60), and an applied pressure of nitrogen up to 10 p.s.i accelerated column elution. Reverse phase preparative HPLC purifications were carried out using a Waters 2525 binary gradient pumping system at flow rates of typically 20 ml/min using a Waters 2996 photodiode array detector.

All solvents and commercial reagents were used as received.

### Example 1

### (R)-2-Amino-3-methyl-pentanoic acid {(S)-1-[(6-amino-pyridin-3-ylmethyl)-carbamoyl]-2-naphthalen-1-yl-ethyl}-amide

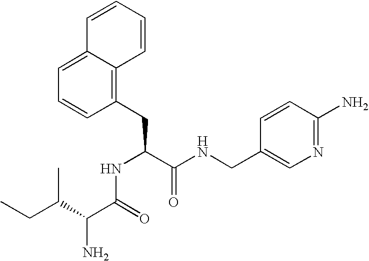

### A. (6-Amino-pyridin-3-ylmethyl)-carbamic acid tert-butyl ester

2-Amino-5-cyanopyridine (2.0 g, 16.8 mmol) was dissolved in methanol (100 ml). This solution was cooled to 0° C. Nickel (II) chloride hexahydrate (0.4 g, 1.67 mmol) and di-tertbutyl dicarbonate (7.33 g, 33.6 mmol) were added followed by sodium borohydride (4.49 g, 117 mmol) portionwise. The reaction mixture was stirred at 0° C. to room temp for 18 hrs. The MeOH was removed by evaporation. The residue was dissolved in EtOAc (100 ml), washed with sat NaHCO₃ (1×50 mls), water (1×50 mls), brine (1×50 mls), dried (Na₂SO4) and evaporated in vacuo to give a brown oil. Purified by flash chromatography, eluant 3% MeOH, 97% CHCl₃ to give an orange oil identified as the title compound.

Yield=2.74 g, 12.25 mmol, 73%

[M+H]⁺=224.1

### B. 5-Aminomethyl-pyridin-2-ylamine dihydrochloride

(6-Amino-pyridin-3-ylmethyl)-carbamic acid tert-butyl ester (2.74 g, 12.25 mmol) was dissolved in 4M HCl/dioxan (50 mls). After one hour at room temp the solvent was removed in vacuo to give a pale yellow solid identified as the title compound.

Yield=2.3 g, 12.1 mmol, 99%

[M+H]⁺=124.1

### C. {(S)-1-[(6-Amino-pyridin-3-ylmethyl)-carbamoyl]-2-naphthalen-1-yl-ethyl}-carbamic acid tert-butyl ester

Boc-1Nal-OH (1.0 g, 2.66 mmol) was dissolved in CH₂Cl₂ (30 mls). Triethylamine (0.805 g, 7.96 mmol) and HBTU (1.21 g, 3.18 mmol) was added followed by 5-aminomethyl-pyridin-2-ylamine dihydrochloride (0.52 g, 2.66 mmol). After 3 hours at room temperature the reaction mixture was diluted with CHCl₃ (50 mls), this solution was washed with sat. NaHCO₃ (1×20 mls), water (1×20 mls), brine (1×20 mls), dried (Na₂SO₄) and evaporated in vacuo. The residue was purified by flash chromatography (silica), eluent 3% MeOH, 97% CHCl₃, fractions combined and evaporated in vacuo to give a colourless oil identified as the title compound.

Yield=1.18 g, 2.15 mmol, 81%

[M+H]⁺=421.27

### D. (S)-2-Amino-N-(6-amino-pyridin-3-ylmethyl)-3-naphthalen-1-yl-propionamide ditrifluoroacetate

{(S)-1-[(6-Amino-pyridin-3-ylmethyl)-carbamoyl]-2-naphthalen-1-yl-ethyl}-carbamic acid tert-butyl ester (1.18 g, 2.81 mmol) was treated with trifluoroacetic acid (30 mls). After 1 hour at room temperature the solvent was removed in vacuo giving a pale brown solid identified as the title compound.

Yield=1.53 g, 2.8 mmol, 99%

[M+H]⁺=321.1

### E. ((R)-1-{(S)-1-[(6-Amino-pyridin-3-ylmethyl)-carbamoyl]-2-naphthalen-1-yl-ethylcarbamoyl}-2-methyl-butyl)-carbamic acid tert-butyl ester

(S)-2-Amino-N-(6-amino-pyridin-3-ylmethyl)-3-naphthalen-1-yl-propionamide ditrifluoroacetate (120 mg, 0.22 mmol) was dissolved in CH₂Cl₂ (20 mls) and DMF (2 mls). This solution was cooled to 0° C. Boc-DIle-OH (65 mg, 0.28 mmol) was added followed by HOBt (65 mg, 0.48 mmol) and water soluble carbodiimide (62 mg, 0.31 mmol). After 15 mins triethylamine (49 mg, 0.49 mmol) was added. After 18 hrs 0° C. to room temperature the reaction mixture was diluted with CHCl₃ (50 mls), this solution was washed with sat. NaHCO₃ (1×20 mls), water (1×20 mls), brine (1×20 mls), dried (Na₂SO₄) and evaporated in vacuo. The residue was purified by flash chromatography (silica), eluent 3% MeOH, 97% CHCl₃, fractions combined and evaporated in vacuo to give a colourless oil identified as the title compound.

Yield=98 mg, 0.18 mmol, 81%

[M+H]⁺=534.3

### F. (R)-2-Amino-3-methyl-pentanoic acid {(S)-1-[(6-amino-pyridin-3-ylmethyl)-carbamoyl]-2-naphthalen-1-yl-ethyl}-amide ditrifluoroacetate

((R)-1-{(S)-1-[(6-Amino-pyridin-3-ylmethyl)-carbamoyl]-2-naphthalen-1-yl-ethylcarbamoyl}-2-methyl-butyl)-carbamic acid tert-butyl ester (89 mg, 0.16 mmol) was treated with trifluoroacetic acid (30 mls). After 1 hour at room temperature the solvent was removed in vacuo and the residue purified by Prep HPLC. (19×250 mm Sunfire C-18 Column) 10 to 90% 0.1% TFA/MeCN into 0.1%TFA/H₂O over 35 min at 20 ml/min. Fractions combined and freeze dried to give a white solid identified as the title compound.

Yield 65 mg, 0.125 mmol, 78%

[M+H]⁺=434.2

¹H NMR: (270 MHz) (CD₃OD) 0.75 (6H, t, J=6.9 Hz), 1.1-1.2 (1H, m), 1.7-1.8 (1H, m), 3.31-3.37 (3H, m), 3.66-3.72 (3H, m), 4.14-4.26 (2H, m), 4.82-4.90 (5H, m), 6.87 (1H, d, J=8.4 Hz), 7.39 (2H, s), 7.49-7.61 (3H, m), 7.76-7.79 (1H, m), 7.86-7.89 (1H, m), 8.16-8.19 (1H, m), 8.65 (1H,s, br).

### Example 2

### (R)-2-Amino-3-methyl-pentanoic acid [(S)-1-[(6-amino-pyridin-3-ylmethyl)-carbamoyl]-2-(decahydro-naphthalen-1-yl)-ethyl]-amide

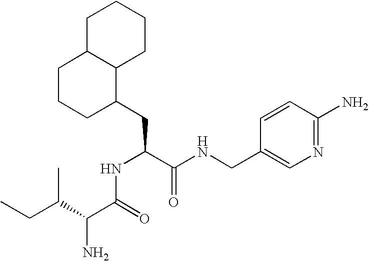

### A. (S)-2-tert-Butoxycarbonylamino-3-(decahydro-naphthalen-1-yl)-propionic acid

Boc-1-napthylalanine (6.0 g, 19.053 mmol) was dissolved in methanol (150 mls). This solution was hydrogenated over 5% Rh on carbon (100 mg) at 70 psi and room temperature. After 2 days the catalyst was filtered off through celite and the residue washed with MeOH (100 mls). The combined filtrates were evaporated in vacuo to give a pale yellow oil identified as the title compound.

Yield=6.15 g, 19.05 mmol, 100%

### B. [(S)-1-[(6-Amino-pyridin-3-ylmethyl)-carbamoyl]-2-(decahydro-naphthalen-1-yl)-ethyl]-carbamic acid tert-butyl ester

(S)-2-tert-Butoxycarbonylamino-3-(decahydro-naphthalen-1-yl)-propionic acid (800 mg, 2.43 mmol) was dissolved in CH₂Cl₂ (60 mls) and DMF (62 mls). This solution was cooled to 0° C. 5-Aminomethyl-pyridin-2-ylamine dihydrochloride (760 mg, 3.86 mmol) was added followed by HOBt (680 mg, 5.0 mmol) and water soluble carbodiimide (590 mg, 2.95 mmol). After 15 mins triethylamine (115 mg, 1.13 mmol) was added. After 18 hrs 0° C. to room temperature the reaction mixture was diluted with CHCl₃ (50 mls), this solution was washed with sat. NaHCO₃ (1×20 mls), water (1×20 mls), brine (1×20 mls), dried (Na₂SO₄) and evaporated in vacuo. The residue was purified by flash chromatography (silica), eluent 3% MeOH, 97% CHCl₃, fractions combined and evaporated in vacuo to give a yellow oil identified as the title compound.

Yield=310 mg, 0.72 mmol, 30%

### C. (S)-2-Amino-N-(6-amino-pyridin-3-ylmethyl)-3-(decahydro-naphthalen-1-yl)-propionamide dihydrochloride

[(S)-1-[(6-Amino-pyridin-3-ylmethyl)-carbamoyl]-2-(decahydro-naphthalen-1-yl)-ethyl]-carbamic acid tert-butyl ester (310 mg, 0.72 mmol) was treated with 4M HCl in dioxan (30 mls). After 1 hour at room temperature the solvent was removed in vacuo giving a pale brown solid identified as the title compound.

Yield=290 mg, 0.72 mmol, 100%

### D. {(R)-1-[(S)-1-[(6-Amino-pyridin-3-ylmethyl)-carbamoyl]-2-(decahydro-naphthalen-1-yl)-ethylcarbamoyl]-2-methyl-butyl}-carbamic acid tert-butyl ester

(S)-2-Amino-N-(6-amino-pyridin-3-ylmethyl)-3-(decahydro-naphthalen-1-yl)-propionamide dihydrochloride (82 mg, 0.22 mmol) was dissolved in CH₂Cl₂ (20 mls) and DMF (2 mls). This solution was cooled to 0° C. Boc-DIle-OH (62 mg, 0.27 mmol) was added followed by HOBt (61 mg, 0.45 mmol) and water soluble carbodiimide (54 mg, 0.27 mmol). After 15 mins triethylamine (49 mg, 0.49 mmol) was added. After 18 hrs 0° C. to room temperature the reaction mixture was diluted with CHCl₃ (50 mls), this solution was washed with sat. NaHCO₃ (1×20 mls), water (1×20 mls), brine (1×20 mls), dried (Na₂SO₄) and evaporated in vacuo. The residue was purified by flash chromatography (silica), eluent 3% MeOH, 97% CHCl₃, fractions combined and evaporated in vacuo to give a yellow oil identified as the title compound.

Yield=55 mg, 0.10 mmol, 46%

### E. (R)-2-Amino-3-methyl-pentanoic acid [(S)-1-[(6-amino-pyridin-3-ylmethyl)-carbamoyl]-2-(decahydro-naphthalen-1-yl)-ethyl]-amide ditrifluoroacetate

{(R)-1-[(S)-1-[(6-Amino-pyridin-3-ylmethyl)-carbamoyl]-2-(decahydro-naphthalen-1-yl)-ethylcarbamoyl]-2-methyl-butyl}-carbamic acid tert-butyl ester (45 mg, 0.083 mmol) was treated with trifluoroacetic acid (30 mls). After 1 hour at room temperature the solvent was removed in vacuo and the residue purified by Prep HPLC (19×250 mm Sunfire C-18 Column). 10 to 90% 0.1% TFA/MeCN into 0.1% TFA/H₂O over 35 min at 20 ml/min. Fractions combined and freeze dried to give a white solid identified as the title compound.

Yield 27 mg, 0.04 mmol, 48%

[M+H]⁺=444.3

¹H NMR: (270 MHz) (CD₃OD) 0.96-1.05 (6H, m), 1.25-1.78 (28H, m), 3.76-3.86 (1H, m), 4.26-4.28 (3H, m), 6.97 (1H, d, J=9Hz), 7.74 (1H, s), 7.80-7.90 (1H, m)

### Example 3

### (R)-3-Methyl-2-methylamino-pentanoic acid [(S)-1-[(6-amino-pyridin-3-ylmethyl)-carbamoyl]-2-(3,4-dichloro-phenyl)-ethyl]-amide

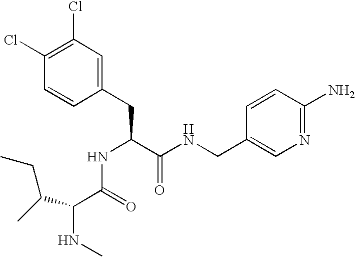

### A. 5-Aminomethyl-pyridin-2-ylamine dihydrochloride

6-Amino-3-pyridinecarbonitrile (12.5 g, 104 mmol) was dissolved (250 mls), 6M HCl (35 mls, 210 mmol) was added. 10% Pd/C (2.5 g) was added. The reaction mixture was shaken at 10 psi for 18 hours after which time the catalyst was filtered off through celite and the residue washed with methanol (200 mls) and water (20 mls). The combined filtrates were evaporated in vacuo to give a white solid. Recrystallised from MeOH/diethyl ether to give a white solid identified as the title compound

Yield=15.52 g, 79.2 mmol, 75%

[M+H]⁺=124.17

### B. {(S)-1-[(S)-1-[(6-Amino-pyridin-3-ylmethyl)-carbamoyl]-2-(3,4-dichloro-phenyl)-ethylcarbamoyl]-2-methyl-butyl}-carbamic acid tert-butyl ester

Boc-3,4-dichloro-Phe-OH (1.71 g, 5.1 mmol) was dissolved in CH₂Cl₂ (30 mls) and DMF (3 mls). This solution was cooled to 0° C. 5-Aminomethyl-pyridin-2-ylamine dihydrochloride (1.0 g, 5.1 mmol) was added followed by HOBt (827 mg, 6.1 mmol) and water soluble carbodiimide (978 mg, 5.1 mmol). After 15 mins diisopropylethylamine (1.98 g, 15.3 mmol) was added. After 5 hrs 0° C. to room temperature the reaction mixture was diluted with CHCl₃ (50 mls), this solution was washed with sat. NaHCO₃ (1×20 mls), water (1×20 mls), brine (1×20 mls), dried (Na₂SO₄) and evaporated in vacuo. The residue was purified by flash chromatography (silica), eluent 5% MeOH, 95% CHCl₃, fractions combined and evaporated in vacuo to give a yellow oil identified as the title compound.

Yield=1.44 g, 3.28 mmol, 64%

[M+H]⁺=439.20

### C. (S)-2-Amino-N-(6-amino-pyridin-3-ylmethyl)-3-(3,4-dichloro-phenyl)-propionamide dihydrochloride

{(S)-1-[(S)-1-[(6-Amino-pyridin-3-ylmethyl)-carbamoyl]-2-(3,4-dichloro-phenyl)-ethylcarbamoyl]-2-methyl-butyl}-carbamic acid tert-butyl ester (1.44 g, 3.28 mmol) was treated with 4M HCl in dioxan (50 mls). After 1 hour at room temperature the solvent was removed in vacuo giving a pale brown solid identified as the title compound.

Yield=1.3 g, 3.15 mmol, 96%

[M+H]⁺=339.0, 340.9

### D. {(R)-1-[(S)-1-[(6-Amino-pyridin-3-ylmethyl)-carbamoyl]-2-(3,4-dichloro-phenyl)-ethylcarbamoyl]-2-methyl-butyl}-methyl-carbamic acid tert-butyl ester

(S)-2-Amino-N-(6-amino-pyridin-3-ylmethyl)-3-(3,4-dichloro-phenyl)-propionamide dihydrochloride (1.3 g, 3.3 mmol) was dissolved in CH₂Cl₂ (20 mls) and DMF (2 mls). This solution was cooled to 0° C. Boc-N-Me-DIle-OH (811 mg, 3.3 mmol) was added followed by HOBt (536 mg, 3.9 mmol) and water soluble carbodiimide (633 mg, 3.3 mmol). After 15 mins diisopropylethylamine (1.3 g, 9.9 mmol) was added. After 5 hrs 0° C. to room temperature the reaction mixture was diluted with CHCl₃ (50 mls), this solution was washed with sat. NaHCO₃ (1×20 mls), water (1×20 mls), brine (1×20 mls), dried (Na₂SO₄) and evaporated in vacuo. The residue was purified by flash chromatography (silica), eluent 4% MeOH, 96% CHCl₃, fractions combined and evaporated in vacuo to give a yellow oil identified as the title compound.

Yield=983 mg, 1.74 mmol, 53%

[M+H]⁺=566.25, 568.26

### E. (R)-3-Methyl-2-methylamino-pentanoic acid [(S)-1-[(6-amino-pyridin-3-ylmethyl)-carbamoyl]-2-(3,4-dichloro-phenyl)-ethyl]-amide dihydrochloride

{(R)-1-[(S)-1-[(6-Amino-pyridin-3-ylmethyl)-carbamoyl]-2-(3,4-dichloro-phenyl)-ethylcarbamoyl]-2-methyl-butyl}-methyl-carbamic acid tert-butyl ester (983 mg, 1.74 mmol) was treated with 4M HCl in dioxan (30 mls). After 1 hour at room temperature the solvent was removed in vacuo, azetroped from toluene and the residue freeze dried from water to give a white solid identified as the title compound.

Yield 880 mg, 1.63 mmol, 94%

[M+H]⁺=466.01

¹H NMR: (270 MHz) (CD₃OD) 0.75-0.90 (7H, m), 1.20-1.40 (1H, m), 1.70-1.90 (1H, m), 2.62 (3H, s), 2.85-3.00 (1H, m), 3.15-3.25 (1H, m), 3.30-3.35 (2H, m), 3.65-3.75 (1H, m), 4.20-4.40 (2H, m), 4.60-4.70 (1H, m), 4.90-5.10 (2H, m), 6.90-7.10 (1H, m), 7.20-7.30 (1H, m), 7.40-7.50 (2H, m), 7.70-7.90 (2H, m), 8.75-8.85 (1H, m).

### Example 4

### (R)-Pyrrolidine-2-carboxylic acid [(S)-1-[(6-amino-pyridin-3-ylmethyl)-carbamoyl]-2-(3,4-dichloro-phenyl)-ethyl]-amide

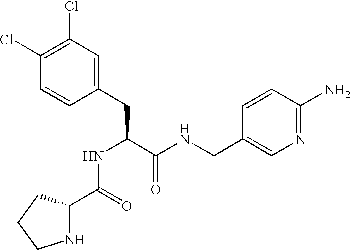

### A. (R)-2-[(S)-1-[(6-Amino-pyridin-3-ylmethyl)-carbamoyl]-2-(3,4-dichloro-phenyl)-ethylcarbamoyl]-pyrrolidine-1-carboxylic acid tert-butyl ester

Boc-DPro-OH (238 mg, 1.11 mmol) was dissolved in CH₂Cl₂ (30 mls). Triethylamine (323 mg, 3.32 mmol) and HBTU (419 mg, 1.11 mmol) was added followed by (S)-2-amino-N-(6-amino-pyridin-3-ylmethyl)-3-(3,4-dichloro-phenyl)-propionamide dihydrochloride (435 mg, 1.11 mmol). After 3 hours at room temperature the reaction mixture was diluted with CHCl₃ (50 mls), this solution was washed with sat. NaHCO₃ (1×20 mls), water (1×20 mls), brine (1×20 mls), dried (Na₂SO₄) and evaporated in vacuo. The residue was purified by flash chromatography (silica), eluent 5% MeOH, 99% CHCl₃, fractions combined and evaporated in vacuo to give a yellow oil identified as the title compound.

Yield=263 mg, 0.49 mmol, 44%

[M+H]⁺=536.2

### B. (R)-Pyrrolidine-2-carboxylic acid [(S)-1-[(6-amino-pyridin-3-ylmethyl)-carbamoyl]-2-(3,4-dichloro-phenyl)-ethyl]-amide dihydrochloride

(R)-2-[(S)-1-[(6-Amino-pyridin-3-ylmethyl)-carbamoyl]-2-(3,4-dichloro-phenyl)-ethylcarbamoyl]-pyrrolidine-1-carboxylic acid tert-butyl ester (263 mg, 0.49 mmol) was treated with 4M HCl in dioxan (30 mls). After 1 hour at room temperature the solvent was removed in vacuo, azetroped from toluene and the residue freeze dried from water to give a white solid identified as the title compound.

Yield 232 mg, 0.46 mmol, 93%

[M+H]⁺=436.0, 438.1

¹H NMR: (270 MHz) (CD₃OD) 1.60-2.10 (3H, m), 2.20-2.40 (1H, m), 2.80-3.50 (5H, m), 4.10-4.35 (3H, m), 4.55-4.85 (1H, m), 4.80-5.10 (3H, m), 6.90-7.00 (1H, m), 7.10-7.25 (1H, m), 7.30-7.50 (2H, m), 7.70-7.90 (2H, m), 8.70-8.90 (1H, m)

### Example 5

### (R)-1-Methyl-pyrrolidine-2-carboxylic acid [(S)-1-[(6-amino-pyridin-3-ylmethyl)-carbamoyl]-2-(3,4-difluoro-phenyl)-ethyl]-amide

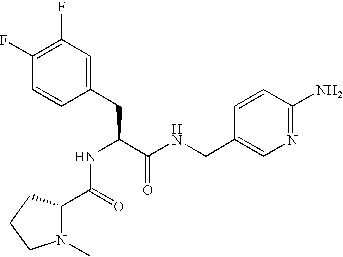

### A. (R)-1-Methyl-pyrrolidine-2-carboxylic acid (N-Me-DPro-OH)

H-DPro-OH (10.0 g, 86.9 mmol) was dissolved in methanol (200 mls), formaldehyde (37% by weight solution, 7 mls) was added followed by 10% Pd/C (5 g). The reaction mixture was shaken at 15 psi for 18 hours. After this time the catalyst was filtered off through Celite and the residue washed with MeOH (100 mls). The combined filtrates were evaporated in vacuo to give a white solid which was recystallised from MeOH/diethyl ether to give a white crystalline solid identified as the title compound

Yield=10.72 g, 83 mmol, 96%

[M+H]⁺=130.17

### B. [(S)-1-[(6-Amino-pyridin-3-ylmethyl)-carbamoyl]-2-(3,4-difluoro-phenyl)-ethyl]-carbamic acid tert-butyl ester

Boc-3,4-difluoro-Phe-OH (5.0 g, 16.6 mmol) was dissolved in CH₂Cl₂(100 mls) and DMF (10 mls). This solution was cooled to 0° C. 5-Aminomethyl-pyridin-2-ylamine dihydrochloride (3.58 g, 18.2 mmol) was added followed by HOBt (2.69 g, 19.9 mmol) and water soluble carbodiimide (3.18 g, 16.6 mmol). After 15 mins diisopropylethylamine (6.44 g, 49.8 mmol) was added. After 5 hrs 0° C. to room temperature the reaction mixture was diluted with CHCl₃ (150 mls), this solution was washed with sat. NaHCO₃ (1×50 mls), water (1×50 mls), brine (1×50 mls), dried (Na₂SO₄) and evaporated in vacuo. The residue was purified by flash chromatography (silica), eluent 5% MeOH, 95% CHCl₃, fractions combined and evaporated in vacuo to give a yellow oil identified as the title compound.

Yield=5.79 g, 14.2 mmol, 88%

[M+H]⁺=407.16

### C. (S)-2-Amino-N-(6-amino-pyridin-3-ylmethyl)-3-(3,4-difluoro-phenyl)-propionamide dihydrochloride

[(S)-1-[(6-Amino-pyridin-3-ylmethyl)-carbamoyl]-2-(3,4-difluoro-phenyl)-ethyl]-carbamic acid tert-butyl ester (5.79 g, 14.2 mmol) was treated with 4M HCl in dioxan (50 mls). After 1 hour at room temperature the solvent was removed in vacuo giving a pale brown solid identified as the title compound.

Yield=5.4 g, 14.2 mmol, 100%

[M+H]⁺=307.05

### D. (R)-1-Methyl-pyrrolidine-2-carboxylic acid [(S)-1-[(6-amino-pyridin-3-ylmethyl)-carbamoyl]-2-(3,4-difluoro-phenyl)-ethyl]-amide dihydrochloride

((S)-2-Amino-N-(6-amino-pyridin-3-ylmethyl)-3-(3,4-difluoro-phenyl)-propionamide dihydrochloride (5.55 g, 14.6 mmol) was dissolved in CH₂Cl₂ (100 mls) and DMF (10 mls). This solution was cooled to 0° C. N-Me-DPro-OH (1.89 g, 14.63 mmol) was added followed by HOBt (2.37 g, 17.5 mmol) and water soluble carbodiimide (3.93 g, 20.5 mmol). After 15 mins diisopropylethylamine (5.67 g, 43.9 mmol) was added. After 5 hrs 0° C. to room temperature the reaction mixture was diluted with CHCl₃ (150 mls), this solution was washed with sat. NaHCO₃ (1×20 mls), water (1×50 mls), brine (1×50 mls), dried (Na₂SO₄) and evaporated in vacuo. The residue was purified by flash chromatography (silica), eluent 7% MeOH, 93% CHCl₃, fractions combined and evaporated in vacuo to give a white solid. This solid was dissolved in 4M HCl in dioxan (100 mls), after 30 mins the solvent was removed in vacuo and the residue freeze dried from water and MeCN to yield a white solid identified as the title compound.

Yield=2.9 g, 5.91 mmol, 40%

[M+H]⁺=418.06 ¹H NMR: (270 MHz) (CD₃OD) 1.60-2.25 (3H, m), 2.40-2.60 (1H, m), 2.90 (3H, s), 3.10-3.20 (2H, m), 3.25-3.35 (2H, m), 3.60-3.75 (1H, m), 4.10-4.35 (3H, m), 4.60-4.80 (1H, m), 4.90-5.10 (2H, m), 6.90-7.30 (4H, m), 7.70-7.90 (2H, m), 8.75-8.90 (1H, m)

### Example 6

### (R)-1-Isopropyl-pyrrolidine-2-carboxylic acid [(S)-1-[(6-amino-pyridin-3-ylmethyl)-carbamoyl]-2-(3,4-difluoro-phenyl)-ethyl]-amide

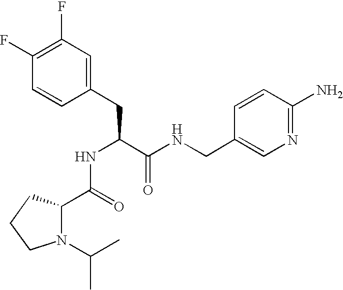

### A. (R)-1-Isopropyl-pyrrolidine-2-carboxylic acid

H-DPro-OH (5.0 g, 43.3 mmol) was dissolved in methanol (200 mls), acetone (3.78 g, 58.1 mmol) was added followed by 10% Pd/C (2.5 g). The reaction mixture was shaken at 15 psi for 18 hours. After this time the catalyst was filtered off through celite and the residue washed with MeOH (100 mls). The combined filtrates were evaporated in vacuo to give a white solid which was recystallised from MeOH/diethyl ether to give a white crystalline solid identified as the title compound

Yield=5.747 g, 33.4 mmol, 77%

[M+H]⁺=158.14

### B. (R)-1-Isopropyl-pyrrolidine-2-carboxylic acid [(S)-1-[(6-amino-pyridin-3-ylmethyl)-carbamoyl]-2-(3,4-difluoro-phenyl)-ethyl]-amide ditrifluoroacetate

(R)-1-Isopropyl-pyrrolidine-2-carboxylic acid (187 mg, 1.19 mmol) was dissolved in CH₂Cl₂(30 mls). Triethylamine (601 mg, 5.95 mmol) and HBTU (451 mg, 1.19 mmol) was added followed by ((S)-2-amino-N-(6-amino-pyridin-3-ylmethyl)-3-(3,4-difluoro-phenyl)-propionamide dihydrochloride (451 mg, 1.19 mmol). After 3 hours at room temperature the reaction mixture was diluted with CHCl₃ (50 mls), this solution was washed with sat. NaHCO₃ (1×20 mls), water (1×20 mls), brine (1×20 mls), dried (Na₂SO₄) and evaporated in vacuo. The residue was purified by Prep HPLC (19×250 mm Sunfire C-18 Column). 10 to 90% 0.1% TFA/MeCN into 0.1% TFA/H₂O over 35 min at 20 ml/min. Fractions combined and freeze dried to give a white solid identified as the title compound.

Yield=63 mg, 0.094 mmol, 8%

[M+H]⁺=446.13

¹H NMR: (270 MHz) (CD₃OD) 1.15-1.21 (6H, m) 1.70 (1H, br, s) 1.95 (1H, br, s) 2.31 (1H, br, s) 2.80-3.07 (3H, m) 3.42-3.52 (2H, m) 4.12-4.18 (3H, m) 4.50-4.56 (1H, m) 4.87 (5H, br, s) 6.85-7.08 (4H, m) 7.64-7.70 (2H, m)

### Example 7

### (R)-1-Benzyl-pyrrolidine-2-carboxylic acid [(S)-1-[(6-amino-pyridin-3-ylmethyl)-carbamoyl]-2-(3,4-dichloro-phenyl)-ethyl]-amide

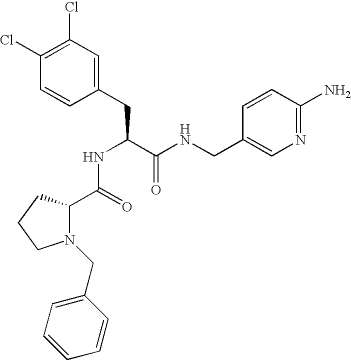

### A. (R)-1-Benzyl-pyrrolidine-2-carboxylic acid [(S)-1-[(6-amino-pyridin-3-ylmethyl)-carbamoyl]-2-(3,4-dichloro-phenyl)-ethyl]-amide ditrifluoroacetate

(R)-Pyrrolidine-2-carboxylic acid [(S)-1-[(6-amino-pyridin-3-ylmethyl)-carbamoyl]-2-(3,4-dichloro-phenyl)-ethyl]-amide dihydrochloride (50 mg, 0.075 mmol) was taken up in methanol/acetic acid (9:1, 10 mls). Benzaldehyde (7.9 mg, 0.075 mmol) was added and the solution stirred at room temp for 1 hour. Sodium cyanoborohydride (9.45 mg, 0.15 mmol) was added. After 18 hours at room temperature the solvent was removed in vacuo and the residue dissolved in CHCl₃ (50 mls), this solution was washed with sat. NaHCO₃ (1×20 mls), water (1×20 mls), brine (1×20 mls), dried (Na₂SO₄) and evaporated in vacuo. The residue was purified by Prep HPLC (19×250 mm Sunfire C-18 Column). 10 to 90% 0.1% TFA/MeCN into 0.1% TFA/H₂O over 35 min at 20 ml/min. Fractions combined and freeze dried to give a white solid identified as the title compound.

Yield=15 mg, 0.02 mmol, 26%

[M+H]⁺=526.06

¹H NMR: (270 MHz) (CD₃OD) 1.60-2.60 (4H, m), 2.80-2.90 (1H, m), 3.00-3.10 (1H, m), 3.20-3.40 (3H, m), 3.45-3.60 (1H, m), 4.05-4.40 (5H, m), 4.50-4.60 (1H, m), 4.75-4.95 (1H, m), 6.90-7.00 (1H, m), 7.05-7.10 (1H, m), 7.30-7.50 (7H, m), 7.65-7.80 (2H, m), 8.60-8.70 (1H, m)

### Example 8

### (R)-2-(Methanesulfonyl-methyl-amino)-3-methyl-pentanoic acid [(S)-1-[(6-amino-pyridin-3-ylmethyl)-carbamoyl]-2-(3,4-dichloro-phenyl)-ethyl]-amide

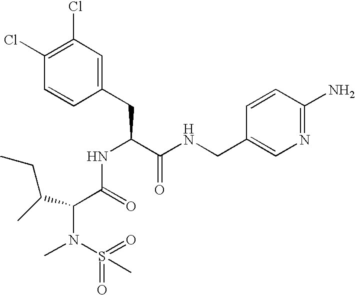

### A. (R)-2-(Methanesulfonyl-methyl-amino)-3-methyl-pentanoic acid [(S)-1-[(6-amino-pyridin-3-ylmethyl)-carbamoyl]-2-(3,4-dichloro-phenyl)-ethyl]-amide trifluoroacetate

(R)-3-Methyl-2-methylamino-pentanoic acid [(S)-1-[(6-amino-pyridin-3-ylmethyl)-carbamoyl]-2-(3,4-dichloro-phenyl)-ethyl]-amide ditrifluoroacetate (80 mg, 0.15 mmol) was dissolved in CH₂Cl₂ (30 mls). This solution was cooled to 0° C. triethylamine (30 mg, 0.30 mmol) was added followed by methanesulphonyl chloride (17 mg, 0.15 mmol). After 1 hr at 0° C. the reaction mixture was diluted with CHCl₃ (150 mls), this solution was washed with sat. NaHCO₃ (1×20 mls), water (1×50 mls), brine (1×50 mls), dried (Na₂SO₄) and evaporated in vacuo. The residue was purified by Prep HPLC (19×250 mm Sunfire C-18 Column). 10 to 90% 0.1% TFA/MeCN into 0.1% TFA/H₂O over 35 min at 20 ml/min. Fractions combined and freeze dried to give a white solid identified as the title compound.

Yield 10 mg, 0.015 mmol, 10%

[M+H]⁺=544.01

¹H NMR: (270 MHz) (CD₃OD) 0.54 (3H, d, J=6.70 Hz), 0.75-1.10 (5H, m), 1.45-1.60 (1H, m), 1.70-1.90 (1H, m), 2.86 (3H, s), 2.87 (3H, s), 3.10-3.30 (3H, m), 3.84 (1H, d, J=10.88 Hz), 4.10-4.40 (2H, m), 4.50-4.70 (1H, m), 6.95 (1H, d, J=9.15 Hz), 7.15-7.30 (1H, m), 7.35-7.50 (2H, m), 7.70-7.80 (2H, m), 8.35-8.50 (1H, m), 8.60-8.70 (1H, m)

### Example 9

### (R)-1-Methyl-pyrrolidine-2-carboxylic acid [(S)-1-[(6-amino-2-methyl-pyridin-3-ylmethyl)-carbamoyl]-2-naphthalen-1-yl-ethyl]-amide

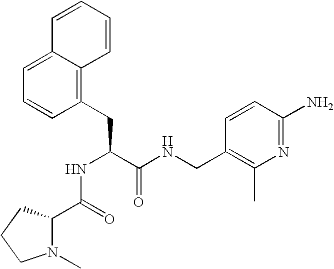

### A. 5-Aminomethyl-6-methyl-pyridin-2-ylamine

6-Amino-2-methylnicotinonitrile (2.0 g, 15.03 mmol) was dissolved in methanol (150 ml). This solution was cooled to 0° C. Cobalt chloride hexahydrate (8.0 g, 33.7 mmol) was added followed by sodium borohydride (6.4 g, 168 mmol) portionwise. The reaction mixture was stirred at 0° C. to room temp for 3 hrs and 3M HCl (100 ml) was added. The MeOH was removed by evaporation. The aqueous residue was washed with EtOAc (100 ml), basified with ammonium hydroxide and extracted with CHCl₃ (3×150 ml). The combined organic extracts were washed with water (1×50 mls), brine (1×50 mls), dried (Na₂SO₄) and evaporated in vacuo to give a brown oil identified as the title compound.

Yield=820 mg, 5.99 mmol, 40%

### B {(S)-1-[(6-Amino-2-methyl-pyridin-3-ylmethyl)-carbamoyl]-2-naphthalen-1-yl-ethyl}-carbamic acid tert-butyl ester

Boc-1Nal-OH (1.5 g, 4.76 mmol) was dissolved in CH₂Cl₂ (30 mls). Triethylamine (0.95 g, 9.5 mmol) and HBTU (1.98 g, 5.23 mmol) was added followed by 5-aminomethyl-pyridin-2-ylamine dihydrochloride (0.83 g, 4.76 mmol). After 3 hours at room temperature the reaction mixture was diluted with CHCl₃ (50 mls), this solution was washed with sat. NaHCO₃ (1×20 mls), water (1×20 mls), brine (1×20 mls), dried (Na₂SO₄) and evaporated in vacuo. The residue was purified by flash chromatography (silica), eluent 3% MeOH, 97% CHCl₃, fractions combined and evaporated in vacuo to give a colourless oil identified as the title compound.

Yield=1.5 g, 3.44 mmol, 72%

[M+H]⁺=435.29

### C. (S)-2-Amino-N-(6-amino-2-methyl-pyridin-3-ylmethyl)-3-naphthalen-1-yl-propionamide dihydrochloride

{(S)-1-[(6-Amino-2-methyl-pyridin-3-ylmethyl)-carbamoyl]-2-naphthalen-1-yl-ethyl}-carbamic acid tert-butyl ester (1.5 g, 3.44 mmol) was treated with 4M HCl in dioxan (30 mls). After 1 hour at room temperature the solvent was removed in vacuo giving a pale brown solid identified as the title compound.

Yield=969 mg, 2.38 mmol, 69%

[M+H]⁺=334.1

### D. (R)-1-Methyl-pyrrolidine-2-carboxylic acid {(S)-1-[(6-amino-2-methyl-pyridin-3-ylmethyl)-carbamoyl]-2-naphthalen-1-yl-ethyl}-amide ditrifluoroacetate

N-Me-DPro-OH (66 mg, 0.51 mmol) was dissolved in CH₂Cl₂ (30 mls). Triethylamine (110 mg, 1.1 mmol) and HBTU (211 mg, 0.56 mmol) was added followed by (S)-2-amino-N-(6-amino-2-methyl-pyridin-3-ylmethyl)-3-naphthalen-1-yl-propionamide dihydrochloride (200 mg, 0.51 mmol). After 3 hours at room temperature the reaction mixture was diluted with CHCl₃ (50 mls), this solution was washed with sat. NaHCO₃ (1×20 mls), water (1×20 mls), brine (1×20 mls), dried (Na₂SO₄) and evaporated in vacuo. The residue was purified by Prep HPLC (19×250 mm Sunfire C-18 Column). 10 to 90% 0.1% TFA/MeCN into 0.1% TFA/H₂O over 35 min at 20 ml/min. Fractions combined and freeze dried to give a white solid identified as the title compound.

Yield=118 mg, 0.27 mmol, 54%

[M+H]⁺=446.02

¹H NMR: (400 MHz) (DMSO-d⁶) 1.8-2.0 (1H, m), 2.2-2.3 (3H, m), 2.5-2.6 (2H, m), 2.7-2.8 (3H, m), 3.0-3.3 (1H, m), 3.3-3.8 (6H, m), 3.7-4.3 (3H, m), 4.7-4.8 (1H, m), 6.7-6.9 (1H, d, J=8 Hz), 7.1-7.2 (1H, m), (7.3-7.4 (2H, m), 7.7-8.0 (3H, m), 8.2-8.3 (1H, m), 8.7 (1H, m), 9.1-9.3 (1H, m), 9.4-9.6 (1H, m)

### Example 10

### (S)—N-(6-Amino-pyridin-3-ylmethyl)-2-(2-diethylamino-acetylamino)-3-(3,4-difluoro-phenyl)-propionamide

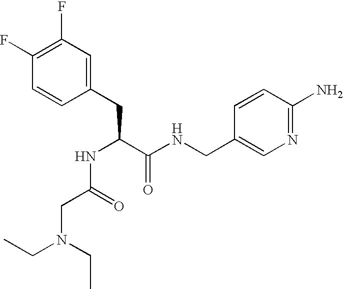

### A. Diethylamino-acetic acid tert-butyl ester

Diethylamine (177 mg, 2.42 mmol) and potassium carbonate (401 mg, 2.90 mmol) were added to a solution of tert-butylbromoacetate (470 mg, 2.42 mmol) in acetonitrile (10 mls). After stirring at room temperature for 18 hours the solvent was removed in vacuo and the residue taken up in ethyl acetate (50 mls). This solution was washed with water (1×20 mls), brine (1×20 mls), dried (Na₂SO₄) and evaporated in vacuo giving a brown oil identified as the title compound.

Yield=240 mg, 1.28 mmol, 53%

[M+H]⁺=188.5

### B. Diethylamino-acetic acid trifluoroacetate

Diethylamino-acetic acid tert-butyl ester (240 mg, 1.28 mmol) was dissolved in CH₂Cl₂ (4 mls). Trifluoroacetic acid (4 mls) was added. After 6 hours at room temperature the solvent was removed in vacuo giving a brown oil identified as the title compound.

Yield=310 mg, 1.26 mmol, 99%

[M+H]⁺=132.66

### C. (S)—N-(6-Amino-pyridin-3-ylmethyl)-2-(2-diethylamino-acetylamino)-3-(3,4-difluoro-phenyl)-propionamide ditrifluoroacetate

((S)-2-Amino-N-(6-amino-pyridin-3-ylmethyl)-3-(3,4-difluoro-phenyl)-propionamide dihydrochloride (155 mg, 0.41 mmol) was dissolved in CH₂Cl₂ (20 mls) and DMF (2 mls). This solution was cooled to 0° C. Diethylamino-acetic acid trifluoroacetate (100 mg, 0.41 mmol) was added followed by HOBt (66 mg, 0.49 mmol) and water soluble carbodiimide (86 mg, 0.45 mmol). After 15 mins triethylamine (200 mg, 2.04 mmol) was added. After 4 hrs 0° C. to room temperature the reaction mixture was diluted with CHCl₃ (150 mls), this solution was washed with sat. NaHCO₃ (1×20 mls), water (1×20 mls), brine (1×20 mls), dried (Na₂SO₄) and evaporated in vacuo. The residue was purified by flash chromatography (silica), eluent 10% MeOH, 90% CH₂Cl₂, fractions combined and evaporated in vacuo to give a colourless oil. The residue was further purified by Prep HPLC. (19×250 mm Sunfire C-18 Column) 0 to 60% 0.1% TFA/MeCN into 0.1% TFA/H₂O over 35 min at 20 ml/min. Fractions combined and freeze dried to give a white solid identified as the title compound.

Yield=60 mg, 0.09 mmol, 23%

[M+H]⁺=418.06

¹H NMR: (400 MHz) (CD₃OD) 1.26 (6H, t, J=6.9 Hz), 2.91-2.97 (1H, m), 3.10-3.21 (5H, m), 3.86-4.00 (2H, m), 4.22-4.34 (2H, m), 4.63-4.68 (1H, m), 6.97 (1H, d, J=9.2 Hz), 7.02-7.05 (1H, m), 7.12-7.23 (2H, m), 7.73 (1H, s), 7.77 (1H, dd, J=9.2, 2.0 Hz), 8.74 (1H, t, J=5.8 Hz).

### Example 11

### (S)-2-Amino-3-methyl-pentanoic acid [(R)-1-[(6-amino-pyridin-3-ylmethyl)-carbamoyl]-2,2-dicyclohexyl-ethyl]-amide

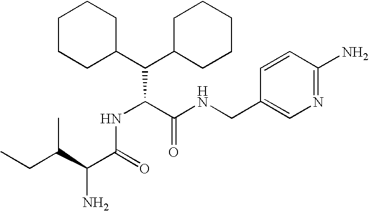

### A. (R)-2-tert-Butoxycarbonylamino-3,3-dicyclohexyl-propionic acid

Boc-D-3,3-Diphenylalanine (4.86 g, 14.06 mmol) was dissolved in methanol (200 mls). This solution was hydrogenated over 5% Rh on carbon (500 mg) at 60 psi and room temperature. After 2 days at room temperature further 5% Rh on carbon (500 mg) was added and hydrogenation continued at 60 psi and room temperature for a further 3 days. After this time the catalyst was filtered off through celite and the residue washed with MeOH (100 mls). The combined filtrates were evaporated in vacuo to give a foamy white solid identified as the title compound,

Yield=4.95 g, 14 mmol, 100%

[M+H]⁺=354.28

### B. {(R)-1-[(6-Amino-pyridin-3-ylmethyl)-carbamoyl]-2,2-dicyclohexyl-ethyl}-carbamic acid tert-butyl ester

(R)-2-tert-Butoxycarbonylamino-3,3-dicyclohexyl-propionic acid (995 mg, 2.82 mmol) was dissolved in CH₂Cl₂ (30 mls). Triethylamine (712 mg, 7.04 mmol) and HBTU (1.07 g, 2.81 mmol) was added followed by 5-aminomethyl-pyridin-2-ylamine dihydrochloride (460 mg, 2.32 mmol). After 3 hours at room temperature the reaction mixture was diluted with CHCl₃ (50 mls), this solution was washed with sat. NaHCO₃ (1×20 mls), water (1×20 mls), brine (1×20 mls), dried (Na₂SO₄) and evaporated in vacuo. The residue was purified by flash chromatography (silica), eluent 3% MeOH, 97% CHCl₃, fractions combined and evaporated in vacuo to give a colourless oil identified as the title compound.

Yield=872 mg, 1.90 mmol, 81%

[M+H]⁺=495.39

### C. (R)-2-Amino-N-(6-amino-pyridin-3-ylmethyl)-3,3-dicyclohexyl-propionamide ditrifluoroacetate

{(R)-1-[(6-Amino-pyridin-3-ylmethyl)-carbamoyl]-2,2-dicyclohexyl-ethyl}-carbamic acid tert-butyl ester (872 mg, 1.90 mmol) was dissolved in TFA (30 mls). After one hour at room temperature the solvent was removed to give a pale orange identified as the title compound.

Yield=1.105 g, 1.88 mmol, 99%

[M+H]⁺=359.30

### D. ((S)-1-[(R)-1-[(6-Amino-pyridin-3-ylmethyl)-carbamoyl]-2,2-dicyclohexyl-ethylcarbamoyl]-2-methyl-butyl)-carbamic acid tert-butyl ester

(R)-2-Amino-N-(6-amino-pyridin-3-ylmethyl)-3,3-dicyclohexyl-propionamide ditrifluoroacetate (90 mg, 0.158 mmol) was dissolved in CH₂Cl₂ (20 mls) and DMF (2 mls). This solution was cooled to 0° C. Boc-Ile-OH (42 mg, 0.187 mmol) was added followed by HOBt (47 mg, 0.31 mmol) and water soluble carbodiimide (35 mg, 0.18 mmol). After 15 mins triethylamine (31 mg, 0.31 mmol) was added. After 18 hrs 0° C. to room temperature the reaction mixture was diluted with CHCl₃ (50 mls), this solution was washed with sat. NaHCO₃ (1×20 mls), water (1×20 mls), brine (1×20 mls), dried (Na₂SO₄) and evaporated in vacuo. The residue was purified by flash chromatography (silica), eluent 4% MeOH, 96% CHCl₃, fractions combined and evaporated in vacuo to give a colourless oil identified as the title compound.

Yield=73 mg, 0.13 mmol, 83%

[M+H]⁺=572.6

### E. (S)-2-Amino-3-methyl-pentanoic acid {(R)-1-[(6-amino-pyridin-3-ylmethyl)-carbamoyl]-2,2-dicyclohexyl-ethyl}-amide ditrifluoroacetate

((S)-1-{(R)-1-[(6-Amino-pyridin-3-ylmethyl)-carbamoyl]-2,2-dicyclohexyl-ethylcarbamoyl}-2-methyl-butyl)-carbamic acid tert-butyl ester (65 mg, 0.11 mmol) was treated with TFA (20 mls). After one hour at room temp the solvent was evaporated in vacuo to give a colourless oil identified as the title compound. Freeze dried from water to give white solid. The residue was further purified by Prep HPLC. (19×250 mm Sunfire C-18 Column) 10 to 90% 0.1% TFA/MeCN into 0.1% TFA/H₂O over 35 min at 20 ml/min. Fractions combined and freeze dried to give a white solid identified as the title compound.

Yield 15 mg, 0.021 mmol, 19%

[M+H]⁺=472.4

¹H NMR: (270 MHz) (CD₃OD) 0.95-1.25 (18H, m), 1.52-1.70 (14H, m), 3.94 (1H, d, J=4.2 Hz), 4.19-4.27 (2H, m), 4.58 (1H, d, J=7.2 Hz), 4.90 (5H, s), 6.98 (1H, d, J=9.19 Hz), 7.79 (1H, d, J=1.5 Hz), 7.91 (1H, dd, J=2.9 Hz, 9.2Hz), 8.70-8.90 (1H, m)

TABLE 1
Compounds were synthesised as described for Examples 1 to 4

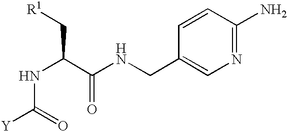

| Example | | Stereochemistry | | Mol | |
|---|---|---|---|---|---|
| No | Y | of Y | R¹ | Wt | [M + H]⁺ |
| 12 | 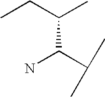 | S | 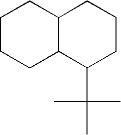 | 443.33 | 444.4 |
| 13 | 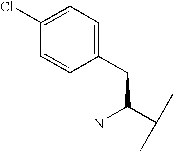 | R | 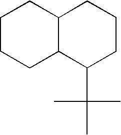 | 511.27 | 512.5, 514.5 |
| 14 | 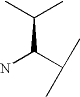 | R | 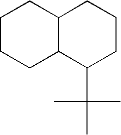 | 429.31 | 430.4 |
| 15 | 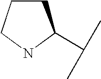 | R | 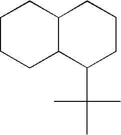 | 427.29 | 428.3 |
| 16 | 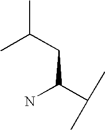 | R | 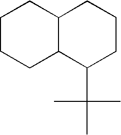 | 443.33 | 444.2 |
| 17 | 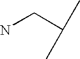 | | 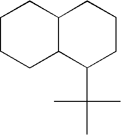 | 387.26 | 388.3 |
| 18 | 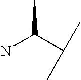 | R | 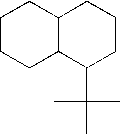 | 401.28 | 402.3 |
| 19 | 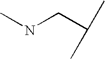 | | 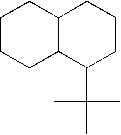 | 401.28 | 402.3 |
| 20 | 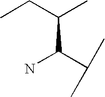 | R | 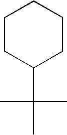 | 389.28 | 390.3 |
| 21 | 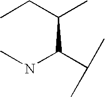 | R | 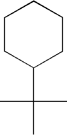 | 403.29 | 404.32 |
| 22 | 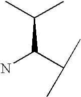 | R | 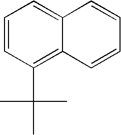 | 419.23 | 420.2 |
| 23 | 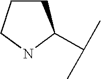 | R | 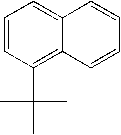 | 417.22 | 418.00 |
| 24 | 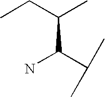 | R | 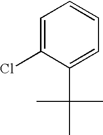 | 417.19 | 418.2, 420.2 |
| 25 | 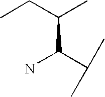 | R | 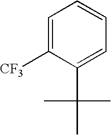 | 451.22 | 452.2 |
| 26 | 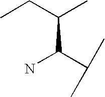 | R | 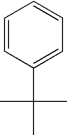 | 383.23 | 384.2 |
| 27 | 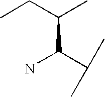 | R | 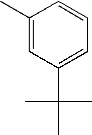 | 397.25 | 398.3 |
| 28 | 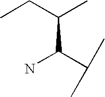 | R | 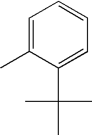 | 397.25 | 398.3 |
| 29 | 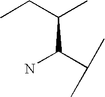 | R | 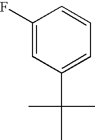 | 401.22 | 402.3 |
| 30 | 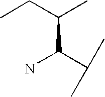 | R | 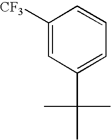 | 451.22 | 452.2 |
| 31 | 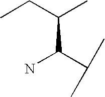 | R | 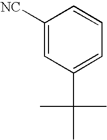 | 408.23 | 409.3 |
| 32 | 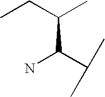 | R | 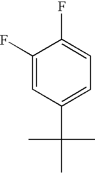 | 419.21 | 420.4 |
| 33 | 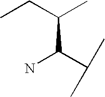 | R | 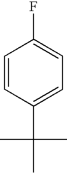 | 401.22 | 402.1 |
| 34 | 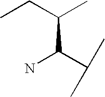 | R | 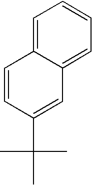 | 433.25 | 434.2 |
| 35 | 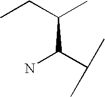 | R | 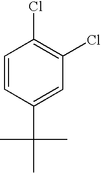 | 451.15 | 452.4, 454.3 |
| 36 | 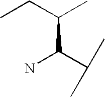 | R | 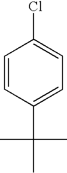 | 417.19 | 418.1, 420.2 |
| 37 | 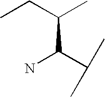 | R | 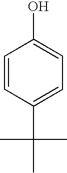 | 399.23 | 400.1 |
| 38 | 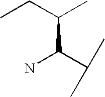 | R | 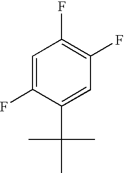 | 437.20 | 438.1 |
| 39 | 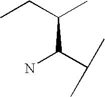 | R | 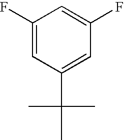 | 419.21 | 420.26 |
| 40 | 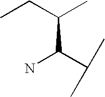 | R | 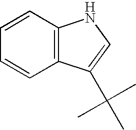 | 422.24 | 423.2 |
| 41 | 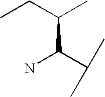 | R | 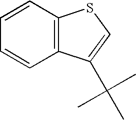 | 439.20 | 440.24 |
| 42 | 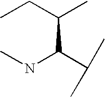 | R | 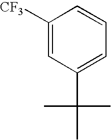 | 465.24 | 466.2 |
| 43 | 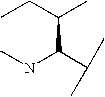 | R | 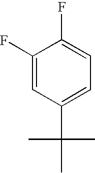 | 433.23 | 434.28 |
| 44 | 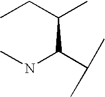 | R | 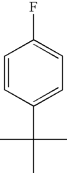 | 415.24 | 416.00 |
| 45 | 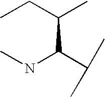 | R | 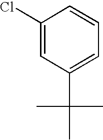 | 431.21 | 432.1 |
| 46 | 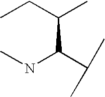 | R | 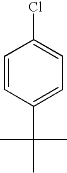 | 431.21 | 432.2, 434.2 |
| 47 | 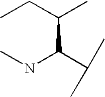 | R | 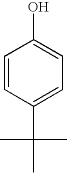 | 413.24 | 414.1 |
| 48 | 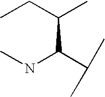 | R | 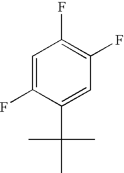 | 451.22 | 452.2 |
| 49 | 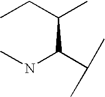 | R | 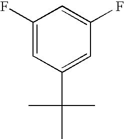 | 433.23 | 434.20 |
| 50 | 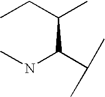 | R | 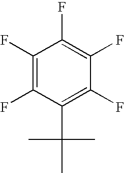 | 487.20 | 488.15 |
| 51 | 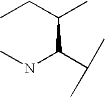 | R | 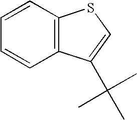 | 453.22 | 454.04 |
| 52 | 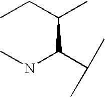 | R | 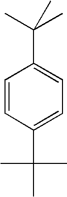 | 453.31 | 454.35 |
| 53 | 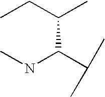 | S | 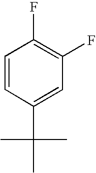 | 433.23 | 434.04 |
| 54 | 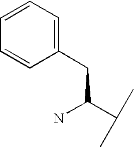 | R | 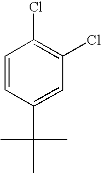 | 485.14 | 486.1, 488.2 |
| 55 | 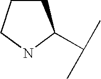 | R | 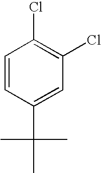 | 435.12 | 435.9, 438.0 |
| 56 | 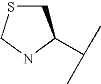 | S | 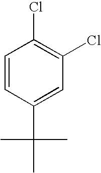 | 453.08 | 454.05 |
| 57 | 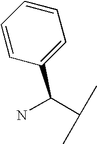 | R | 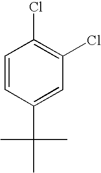 | 471.12 | 472.1, 474.0 |
| 58 | 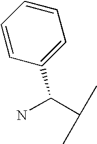 | S | 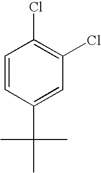 | 471.12 | 472.1, 474.0 |
| 59 | 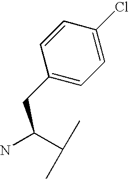 | R | 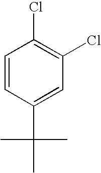 | 519.10 | 520.18, 522.18 |
| 60 | 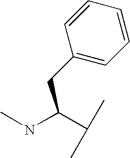 | R | 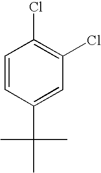 | 499.15 | 500.20 |
| 61 | 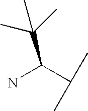 | R | 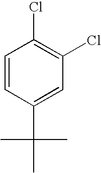 | 451.15 | 452.19 |
| 62 | 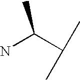 | R | 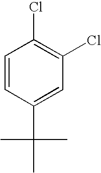 | 423.12 | 423.9, 425.9 |
| 63 | 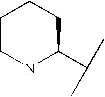 | R | 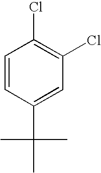 | 449.14 | 450.12, 452.12 |
| 64 | 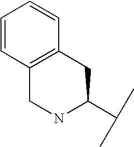 | R | 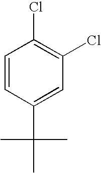 | 497.14 | 498.1, 500.1 |
| 65 | 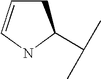 | R | 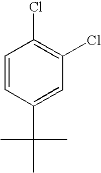 | 433.11 | 433.9, 460.0 |
| 66 | 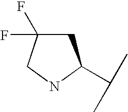 | R | 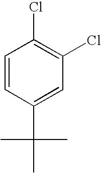 | 471.10 | 472.1, 474.1 |
| 67 | 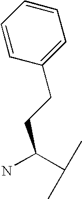 | R | 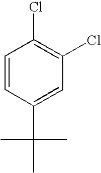 | 499.15 | 500.06 |
| 68 | 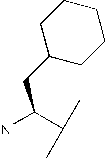 | R | 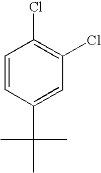 | 491.19 | 492.2, 494.2 |
| 69 | 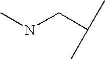 | | 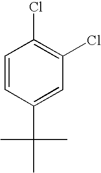 | 409.11 | 409.96 |
| 70 | 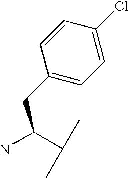 | R | 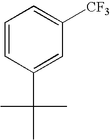 | 519.16 | 519.95, 521.99 |
| 71 | 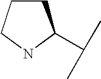 | R | 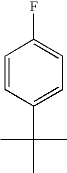 | 385.19 | 385.95 |
| 72 | 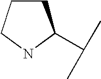 | R | 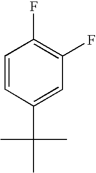 | 403.18 | 404.54 |
| 73 | 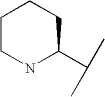 | R | 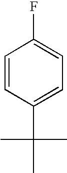 | 399.21 | 400.09 |
| 74 | 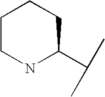 | R | 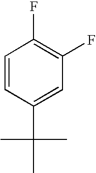 | 417.20 | 418.19 |
| 75 | 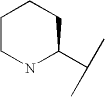 | R | 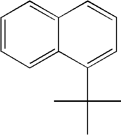 | 431.23 | 432.20 |
| 76 | 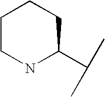 | R | 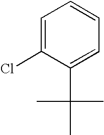 | 415.18 | 416.11 |
| 77 | 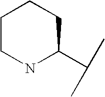 | R | 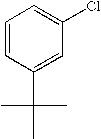 | 415.18 | 416.17 |
| 78 | 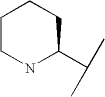 | R | 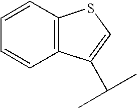 | 437.19 | 438.19 |
| 79 | 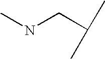 | | 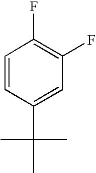 | 377.17 | 378.48 |
| 80 | 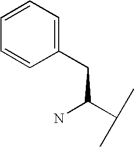 | R | 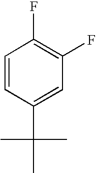 | 453.20 | 454.26 |
| 81 | 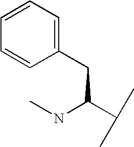 | R | 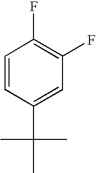 | 467.21 | 468.26 |
| 82 | 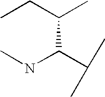 | S | 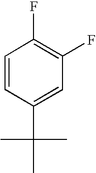 | 433.23 | 434.04 |
| 83 | 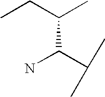 | S | 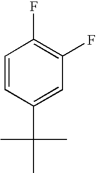 | 419.21 | 420.28 |
| 84 | 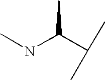 | R | 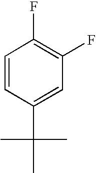 | 391.18 | 392.15 |
| 85 | 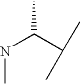 | S | 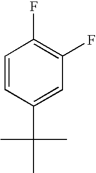 | 391.18 | 392.15 |
| 86 | 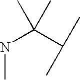 | R | 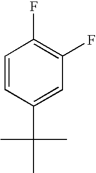 | 405.20 | 406.45 |
| 87 | 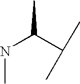 | R | 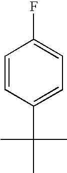 | 373.19 | 374.15 |
| 88 | 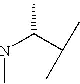 | S | 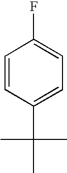 | 373.19 | 374.55 |
| 89 | 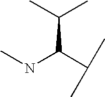 | R | 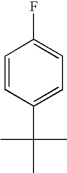 | 401.22 | 402.09 |

TABLE 2
Compounds were synthesised as described for Examples 1 to 7 and 9 and 10

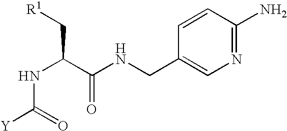

| Example | | Stereochemistry | | Mol | |
|---|---|---|---|---|---|
| No | Y | of Y | R¹ | Wt | [M + H]+ |
| 90 | 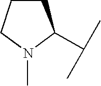 | R | 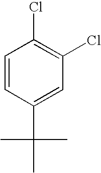 | 449.14 | 450.01 |
| 91 | 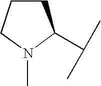 | R | 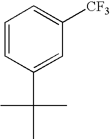 | 449.20 | 450.10 |
| 92 | 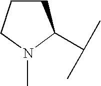 | R | 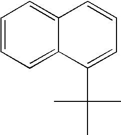 | 431.23 | 432.24 |
| 93 | 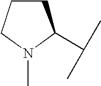 | R | 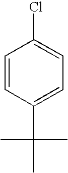 | 415.18 | 416.1, 418.20 |
| 94 | 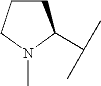 | R | 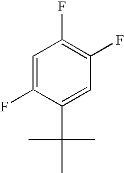 | 435.19 | 436.1 |
| 95 | 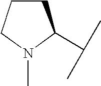 | R | 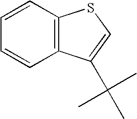 | 437.19 | 438.03 |
| 96 | 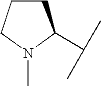 | R | 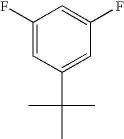 | 417.20 | 418.22 |
| 97 | 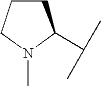 | R | 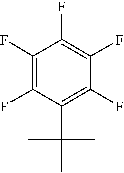 | 471.17 | 472.15 |
| 98 | 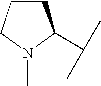 | R | 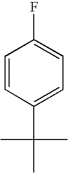 | 399.21 | 400.07 |
| 99 | 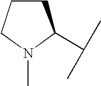 | R | 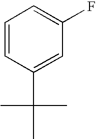 | 399.21 | 400.09 |
| 100 | 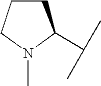 | R | 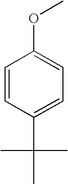 | 411.23 | 412.11 |
| 101 | 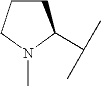 | R | 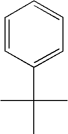 | 381.22 | 382.16 |
| 102 | 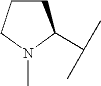 | R | 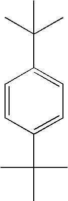 | 437.28 | 438.19 |
| 103 | 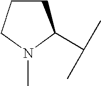 | R | 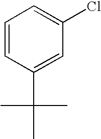 | 415.18 | 416.52 |
| 104 | 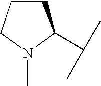 | R | 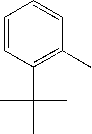 | 395.23 | 396.55 |
| 105 | 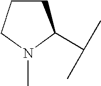 | R | 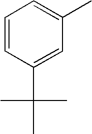 | 395.23 | 396.19 |
| 106 | 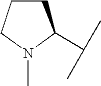 | R | 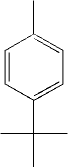 | 395.23 | 396.20 |
| 107 | 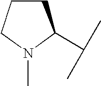 | R | 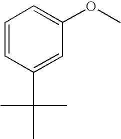 | 411.23 | 412.91 |
| 108 | 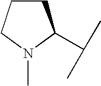 | R | 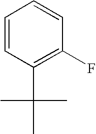 | 399.21 | 400.12 |
| 109 | 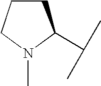 | R | 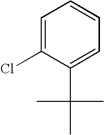 | 415.18 | 416.12, 418.10 |
| 110 | 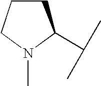 | R | 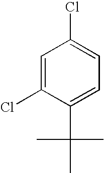 | 449.14 | 450.14, 452.10 |
| 111 | 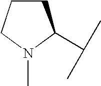 | R | 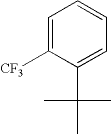 | 449.48 | 450.53 |
| 112 | 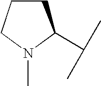 | R | 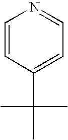 | 382.47 | 383.56 |
| 113 | 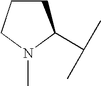 | R | 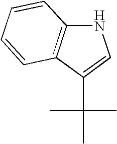 | 420.52 | 421.54 |
| 114 | 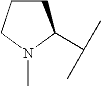 | R | 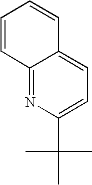 | 432.52 | 433.1 |
| 115 | 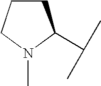 | R | 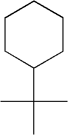 | 387.26 | 388.18 |
| 116 | 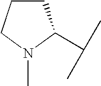 | S | 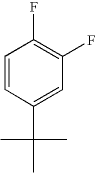 | 417.20 | 418.08 |
| 117 | 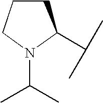 | R | 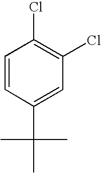 | 477.17 | 478.10 |
| 118 | 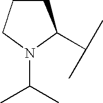 | R | 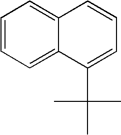 | 459.26 | 460.16 |
| 119 | 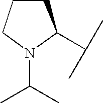 | R | 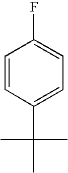 | 427.24 | 428.20 |
| 120 | 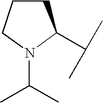 | R | 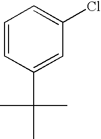 | 443.21 | 444.45 |
| 121 | 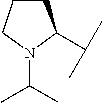 | R | 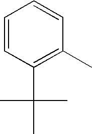 | 423.26 | 424.60 |
| 122 | 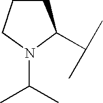 | R | 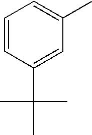 | 423.26 | 424.20 |
| 123 | 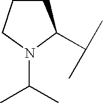 | R | 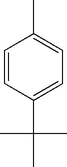 | 423.26 | 424.21 |
| 124 | 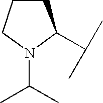 | R | 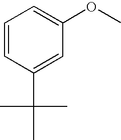 | 439.26 | 440.21 |
| 125 | 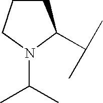 | R | 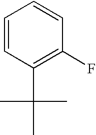 | 427.24 | 428.22 |
| 126 | 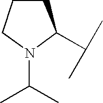 | R | 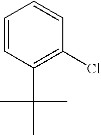 | 443.98 | 444.20 |
| 127 | 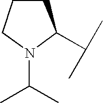 | R | 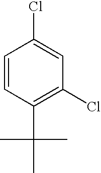 | 478.43 | 478.55 |
| 128 | 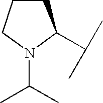 | R | 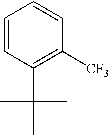 | 477.53 | 478.62 |
| 129 | 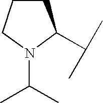 | R | 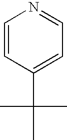 | 410.52 | 411.60 |
| 130 | 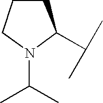 | R | 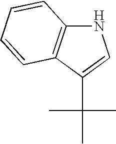 | 448.57 | 449.38 |
| 131 | 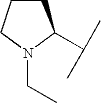 | R | 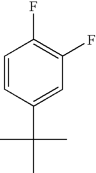 | 431.59 | 432.57 |
| 132 | 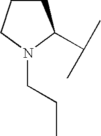 | R | 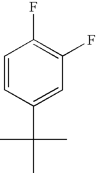 | 445.23 | 446.61 |
| 133 | 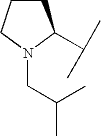 | R | 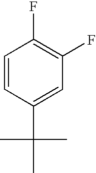 | 459.24 | 460.61 |
| 134 | 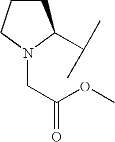 | R | 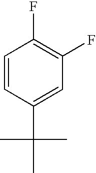 | 475.20 | 476.24 |
| 135 | 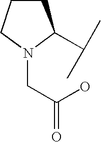 | R | 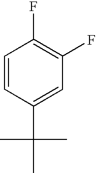 | 461.19 | 462.21 |
| 136 | 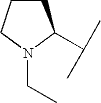 | R | 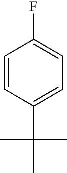 | 413.50 | 414.54 |
| 137 | 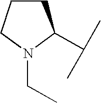 | R | 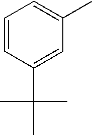 | 409.94 | 410.59 |
| 138 | 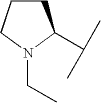 | R | 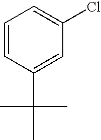 | 429.95 | 430.19 |
| 139 | 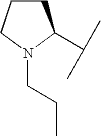 | R | 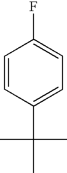 | 427.52 | 428.58 |
| 140 | 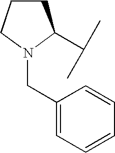 | R | 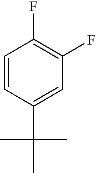 | 493.23 | 494.23 |
| 141 | 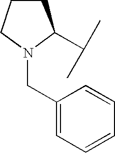 | R | 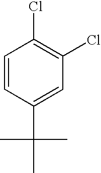 | 525.17 | 526.04, 528.02 |
| 142 | 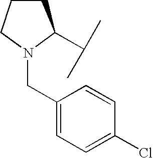 | R | 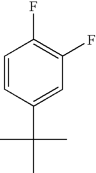 | 528.01 | 528.57 |
| 143 | 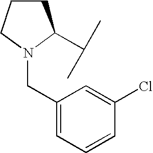 | R | 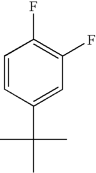 | 528.01 | 528.56 |
| 144 | 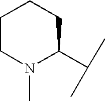 | R | 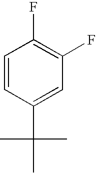 | 431.21 | 432.29 |
| 145 | 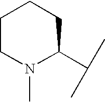 | R | 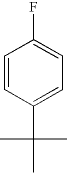 | 413.22 | 414.13 |
| 146 | 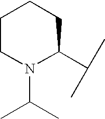 | R | 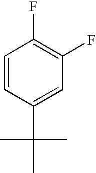 | 459.53 | 460.57 |
| 147 | 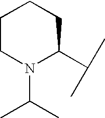 | R | 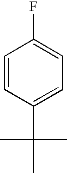 | 441.25 | 442.21 |
| 148 | 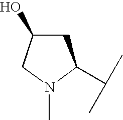 | R | 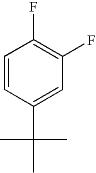 | 433.46 | 434.45 |
| 149 | 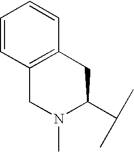 | R | 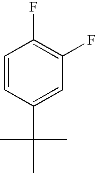 | 479.21 | 480.60 |
| 150 | 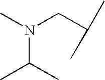 | | 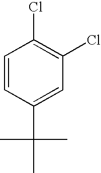 | 451.15 | 452.21, 454.20 |
| 151 | 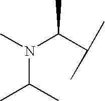 | R | 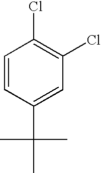 | 465.17 | 465.86 |
| 152 | 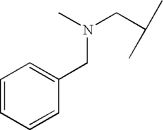 | | 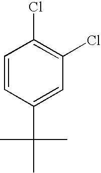 | 499.15 | 500.05 |
| 153 | 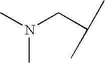 | | 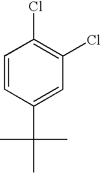 | 423.12 | 424.09 |
| 154 | 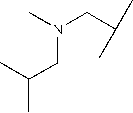 | | 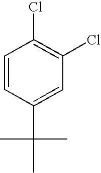 | 465.17 | 465.89 |
| 155 | 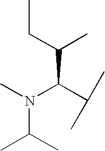 | R | 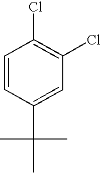 | 507.22 | 508.13 |
| 156 | 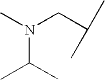 | | 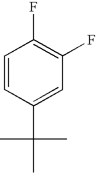 | 419.21 | 420.30 |
| 157 | 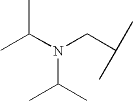 | | 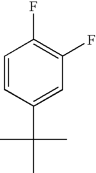 | 447.24 | 448.61 |
| 158 | 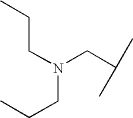 | | 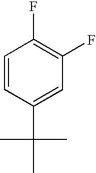 | 447.24 | 448.56 |
| 159 | 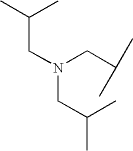 | | 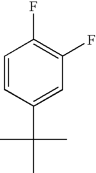 | 475.57 | 476.63 |
| 160 | 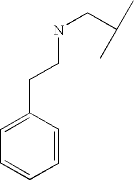 | | 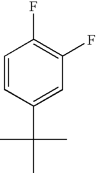 | 467.21 | 468.55 |
| 161 | 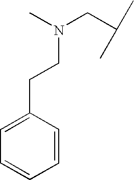 | | 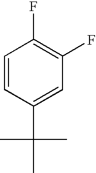 | 481.23 | 482.55 |
| 162 | 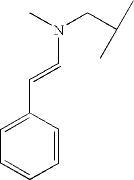 | | 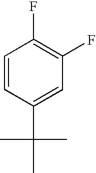 | 493.23 | 494.58 |
| 163 | 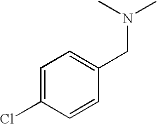 | | 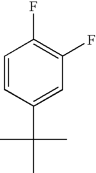 | 501.17 | 502.52 |
| 164 | 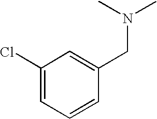 | | 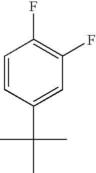 | 501.17 | 502.54 |
| 165 | 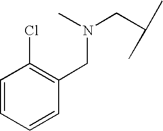 | | 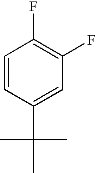 | 501.17 | 502.59 |
| 166 | 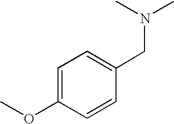 | | 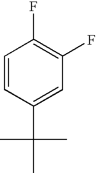 | 497.22 | 498.57 |
| 167 | 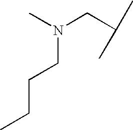 | | 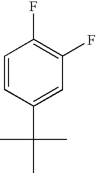 | 433.23 | 434.52 |
| 168 | 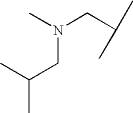 | | 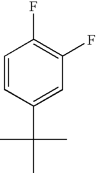 | 433.23 | 434.53 |
| 169 | 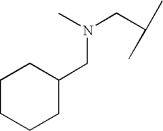 | | 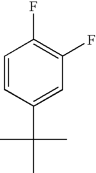 | 473.26 | 474.62 |
| 170 | 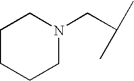 | | 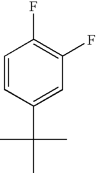 | 431.21 | 432.60 |
| 171 | 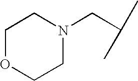 | | 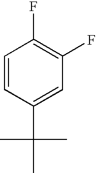 | 433.19 | 434.41 |
| 172 | 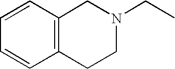 | | 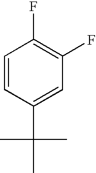 | 479.21 | 480.54 |
| 173 | 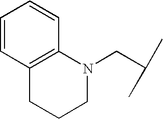 | | 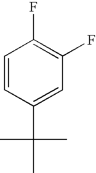 | 479.21 | 480.53 |
| 174 | 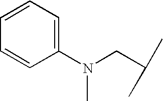 | | 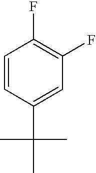 | 453.20 | 454.51 |
| 175 | 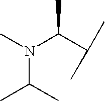 | R | 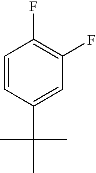 | 433.49 | 434.50 |
| 176 | 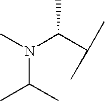 | S | 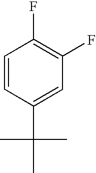 | 433.49 | 434.51 |
| 177 | 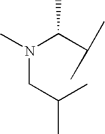 | S | 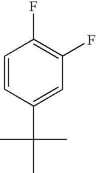 | 447.53 | 448.56 |
| 178 | 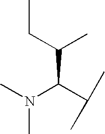 | R | 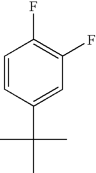 | 447.24 | 448.59 |
| 179 | 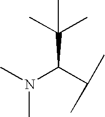 | R | 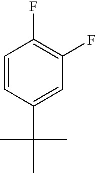 | 447.24 | 448.61 |
| 180 | 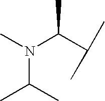 | R | 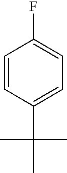 | 415.24 | 416.51 |
| 181 | 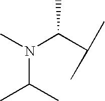 | S | 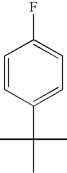 | 415.24 | 416.60 |
| 182 | 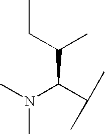 | R | 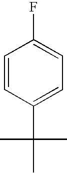 | 429.25 | 430.59 |
| 183 | 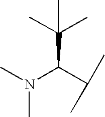 | R | 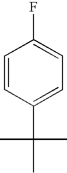 | 429.54 | 430.58 |
| 184 | 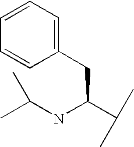 | R | 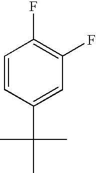 | 495.24 | 496.29 |
| 185 | 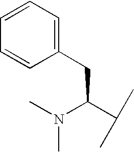 | R | 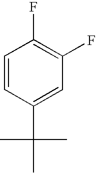 | 481.55 | 482.51 |

TABLE 3
Compounds were synthesised as described for Examples 1 to 7 and 9 and 10

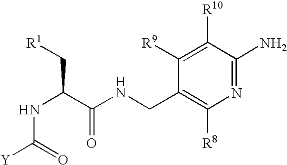

| | | Stereo | | | | | | |
|---|---|---|---|---|---|---|---|---|
| Example | | chem. | | | | | Mol | |
| No | Y | At Y | R¹ | R⁸ | R⁹ | R¹⁰ | Wt | [M + H]+ |
| 186 | 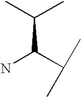 | R | 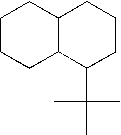 | Me | H | H | 443.33 | 444.3 |
| 187 | 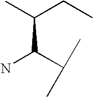 | R | 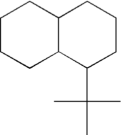 | Me | H | H | 457.34 | 458.4 |
| 188 | 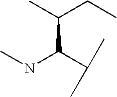 | R | 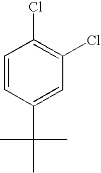 | Me | H | H | 479.19 | 480.20, 482.23 |
| 189 | 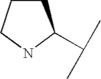 | R | 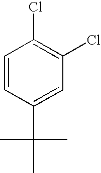 | Me | H | H | 449.14 | 450.7, 452.1 |
| 190 | 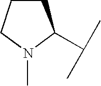 | R | 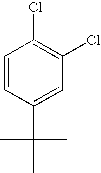 | Me | H | H | 463.15 | 464.11 |
| 191 | 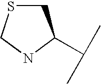 | R | 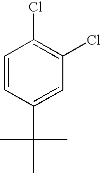 | Me | H | H | 467.09 | 468.1 |
| 192 | 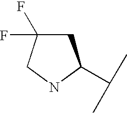 | R | 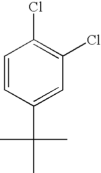 | Me | H | H | 485.12 | 486.13 |
| 193 | 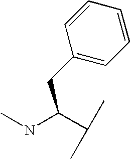 | R | 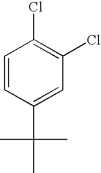 | Me | H | H | 513.17 | 514.1 |
| 194 | 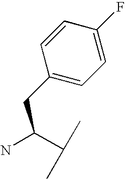 | R | 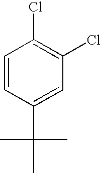 | Me | H | H | 517.14 | 518.1 |
| 195 | 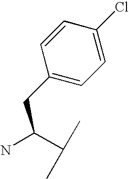 | R | 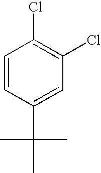 | Me | H | H | 533.12 | 534.0, 536.6 |
| 196 | 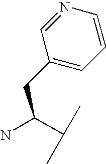 | R | 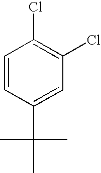 | Me | H | H | 500.15 | 501.03 |
| 197 | 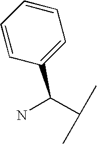 | R | 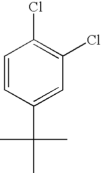 | Me | H | H | 485.14 | 486.18 |
| 198 | 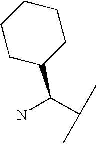 | R | 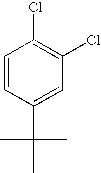 | Me | H | H | 491.19 | 492.2 |
| 199 | 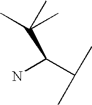 | R | 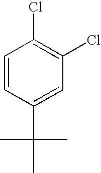 | Me | H | H | 465.17 | 466.2 |
| 200 | 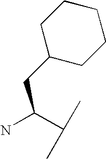 | R | 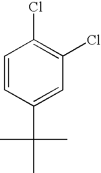 | Me | H | H | 505.20 | 506.24 |
| 201 | 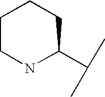 | R | 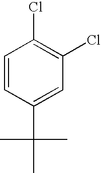 | Me | H | H | 463.15 | 464.16 |
| 202 | 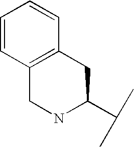 | R | 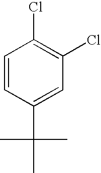 | Me | H | H | 511.15 | 512.16 |
| 203 | 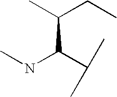 | R | 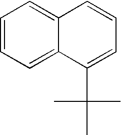 | Me | H | H | 461.28 | 462.23 |
| 204 | 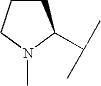 | R | 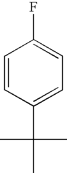 | Me | H | H | 413.22 | 414.90 |
| 205 | 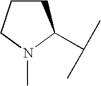 | R | 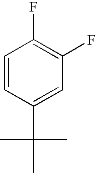 | Me | H | H | 431.21 | 432.16 |
| 206 | 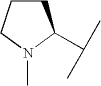 | R | 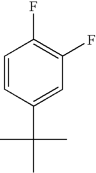 | Me | Me | H | 445.52 | 446.56 |
| 207 | 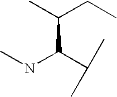 | R | 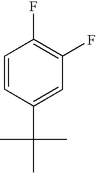 | Me | Me | H | 461.56 | 462.54 |
| 208 | 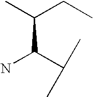 | R | 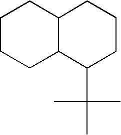 | Me | Me | H | 471.36 | 472.30 |
| 209 | 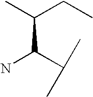 | R | 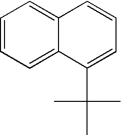 | H | H | Me | 447.26 | 448.11 |

TABLE 4
Compounds were synthesised as described for Examples 8

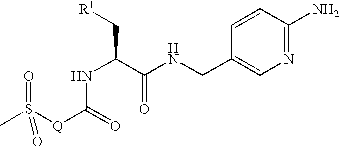

| Example | | Stereochemistry | | Mol | |
|---|---|---|---|---|---|
| No | Q | of Y | R¹ | Wt | [M + H]+ |
| 210 | 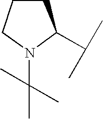 | R | 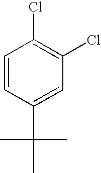 | 513.10 | 513.98 |
| 211 | 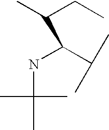 | R | 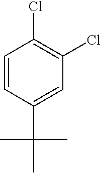 | 529.13 | 530.30 |
| 212 | 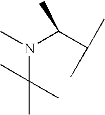 | R | 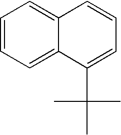 | 501.10 | 502.04 |
| 213 | 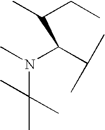 | R | 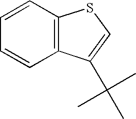 | 531.20 | 532.07 |

TABLE 5
Compounds were synthesised
as described for Examples 1 to 7 and 9 to 11

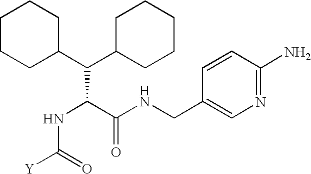

| Example | | Stereochemistry | Mol | |
|---|---|---|---|---|
| No | Y | of Y | Wt | [M + H]⁺ |
| 214 | 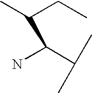 | R | 471.36 | 472.4 |
| 215 | 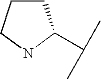 | S | 455.33 | 456.35 |
| 216 | 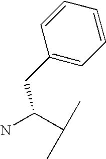 | S | 505.34 | 506.36 |
| 217 | 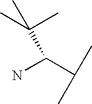 | S | 471.36 | 472.39 |
| 218 | 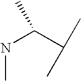 | S | 443.33 | 444.37 |
| 219 | 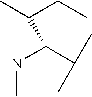 | S | 485.37 | 486.39 |
| 220 | 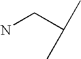 | | 415.29 | 416.28 |
| 221 | 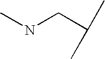 | | 429.31 | 430.34 |
| 222 | 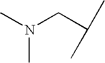 | | 443.33 | 444.36 |
| 223 | 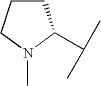 | S | 469.68 | 470.68 |

TABLE 6
Compounds were synthesised as described for Examples 1 to 11

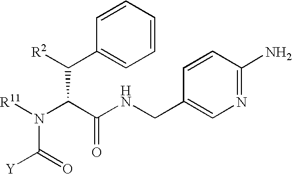

| Example | | Stereochemistry | | | Mol | |
|---|---|---|---|---|---|---|
| No | Y | of Y | R² | R¹¹ | Wt | [M + H]⁺ |
| 224 | 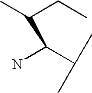 | R | OH | H | 399.49 | 400.3 |
| 225 | 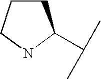 | R | H | Me | 395.5 | 396.2 |

### Example 226

### 3-Methyl-1H-pyrrole-2-carboxylic acid [(S)-1-[(6-amino-pyridin-3-ylmethyl)-carbamoyl]-2-(3,4-difluoro-phenyl)-ethyl]-amide

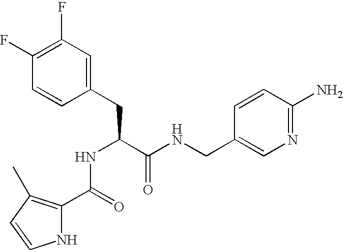

### A. 3-Methyl-1H-pyrrole-2-carboxylic acid [(S)-1-[(6-amino-pyridin-3-ylmethyl)-carbamoyl]-2-(3,4-difluoro-phenyl)-ethyl]-amide trifluoroacetate

((S)-2-Amino-N-(6-amino-pyridin-3-ylmethyl)-3-(3,4-difluoro-phenyl)-propionamide dihydrochloride (800 g, 2.11 mmol) was dissolved in CH₂Cl₂(100 mls). This solution was cooled to 0° C. 3-Methylpyrrole-2-carboxylic acid (264 mg, 2.11 mmol) was added followed by HOBt (399 mg, 2.95 mmol) and water soluble carbodiimide (486 mg, 2.53 mmol). After 15 mins triethylamine (640 g, 0.63 mmol) was added. After 18 hrs 0° C. to room temperature the reaction mixture was diluted with CHCl₃ (150 mls), this solution was washed with sat. NaHCO₃ (1×20 mls), water (1×50 mls), brine (1×50 mls), dried (Na₂SO₄) and filtered through PS paper and evaporated in vacuo. The residue was purified by flash chromatography (silica), eluent 7% MeOH, 93% CHCl₃, fractions combined and evaporated in vacuo to give an orange oil. The residue purified by Prep HPLC. (19×250 mm Sunfire C-18 Column) 10 to 90% 0.1% TFA/MeCN into 0.1% TFA/H2O over 35 min at 20 ml/min. Fractions combined and freeze dried to give a pale pink solid identified as the title compound.

Yield=368 mg, 0.70 mmol, 33%

[M+H]⁺=414.22

¹H NMR: (CD₃OD, 400 MHz) 2.30 (3H, s) 2.81-3.25 (2H, m) 4.17-4.35 (2H, m) 4.71 (1H, t, J=8.0 Hz) 5.25 (4H, br s) 6.00 (1H, s) 6.74-6.78 (1H, m) 7.08-7.14 (3H, m) 7.73-7.77 (2H, m) 8.73-8.79 (1H, m) 10.56 (1H, s).

TABLE 7
Compounds were synthesised as described for Example 226

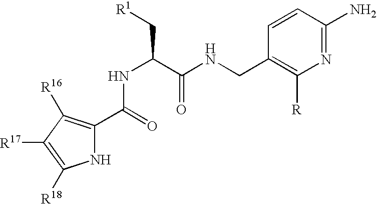

| Example | | | | | | Mol | |
|---|---|---|---|---|---|---|---|
| No | R¹⁶ | R¹⁷ | R¹⁸ | R | R¹ | Wt | [M + H]⁺ |
| 227 | H | H | H | H | 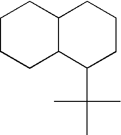 | 423.26 | 424.3 |
| 228 | H | H | H | H | 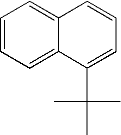 | 413.19 | 414.3 |
| 229 | H | H | H | H | 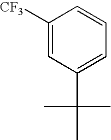 | 431.16 | 432.4 |
| 230 | Me | Me | Me | H | 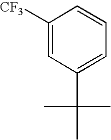 | 473.20 | 474.10 |
| 231 | Me | H | Me | H | 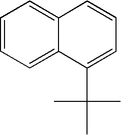 | 441.22 | 442.27 |
| 232 | Me | H | Me | H | 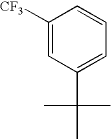 | 459.19 | 460.2 |
| 233 | Me | H | Me | H | 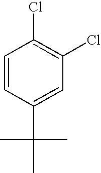 | 459.12 | 460.1, 462.1 |
| 234 | Me | H | Me | H | 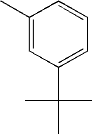 | 405.22 | 406.1 |
| 235 | Me | H | Me | H | 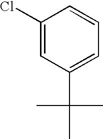 | 425.16 | 426.2, 428.2 |
| 236 | Me | H | Me | H | 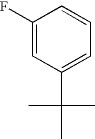 | 409.19 | 410.1 |
| 237 | Me | H | Me | H | 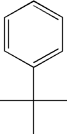 | 391.20 | 392.22 |
| 238 | Me | H | Me | H | 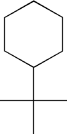 | 397.25 | 398.26 |
| 239 | Me | H | Me | H | 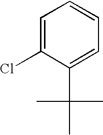 | 425.16 | 425.92 |
| 240 | Me | H | Me | H | 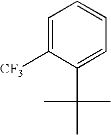 | 459.19 | 460.21 |
| 241 | Me | H | Me | H | 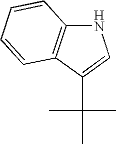 | 430.21 | 431.1 |
| 242 | Me | H | Me | H | 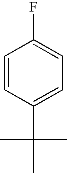 | 409.19 | 410.12 |
| 243 | Me | H | Me | H | 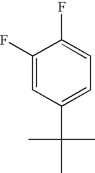 | 427.18 | 428.1 |
| 244 | Me | H | Me | H | 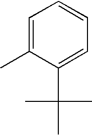 | 405.22 | 406.24 |
| 245 | Me | H | Me | H | 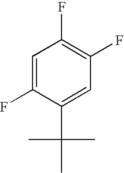 | 445.17 | 446.21 |
| 246 | Me | H | Me | H | 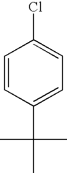 | 447.15 | 448.1, 450.1 |
| 247 | Me | H | Me | H | 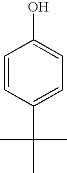 | 407.20 | 408.0 |
| 248 | Me | H | Me | H | 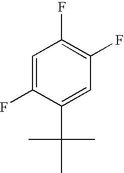 | 445.17 | 446.1 |
| 249 | Me | H | Me | H | 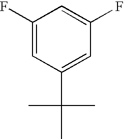 | 427.18 | 428.22 |
| 250 | Me | H | Me | H | 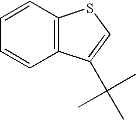 | 447.17 | 448.16 |
| 251 | Me | H | Me | H | 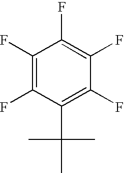 | 481.15 | 482.0 |
| 252 | Me | H | Me | H | 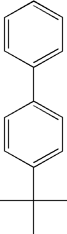 | 467.23 | 468.18 |
| 253 | Me | H | Me | H | 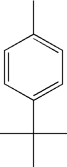 | 405.22 | 406.15 |
| 254 | Me | H | Me | H | 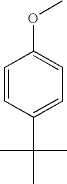 | 421.21 | 422.15 |
| 255 | Me | H | Me | H | 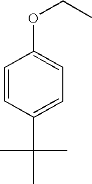 | 435.23 | 436.13 |
| 256 | Me | H | Me | H | 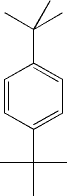 | 447.26 | 448.21 |
| 257 | Me | H | Me | H | 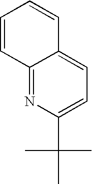 | 442.21 | 443.54 |
| 258 | Me | H | Me | H | 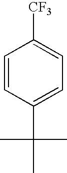 | 445.17 | 446.18 |
| 259 | H | Me | H | H | 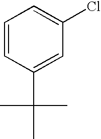 | 411.15 | 412.13 |
| 260 | H | Me | H | H | 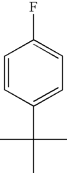 | 395.18 | 396.15 |
| 261 | Me | H | H | H | 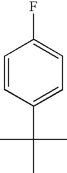 | 395.18 | 396.14 |
| 262 | Me | H | H | H | 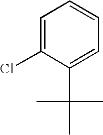 | 411.15 | 412.41 |
| 263 | Me | H | H | H | 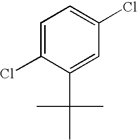 | 445.11 | 446.38, 448.1 |
| 264 | Me | H | H | H | 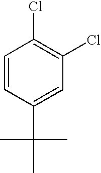 | 445.11 | 446.09, 448.1 |
| 265 | Me | H | H | H | 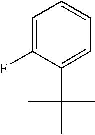 | 395.18 | 396.44 |
| 266 | Me | H | H | H | 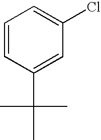 | 411.15 | 412.39, 414.3 |
| 267 | Me | H | H | H | 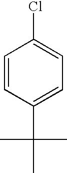 | 411.15 | 412.12 |
| 268 | Me | H | H | H | 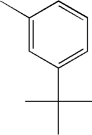 | 391.20 | 392.45 |
| 269 | Me | H | H | H | 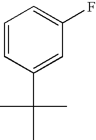 | 395.18 | 396.42 |
| 270 | Me | H | H | H | 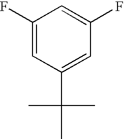 | 413.17 | 414.42 |
| 271 | Me | H | H | H | 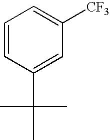 | 445.17 | 446.42 |
| 272 | Me | H | H | H | 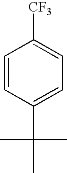 | 445.17 | 446.18 |
| 273 | Me | H | H | H | 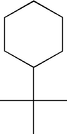 | 383.23 | 384.48 |
| 274 | H | H | CO₂Me | H | 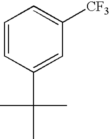 | 489.16 | 490.1 |
| 275 | H | H | CO₂H | H | 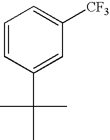 | 475.15 | 476.20 |
| 276 | Me | H | CO₂H | H | 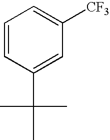 | 489.16 | 490.17 |
| 277 | Me | H | Me | Me | 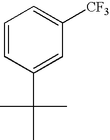 | 473.20 | 474.1 |
| 278 | Me | H | Me | Me | 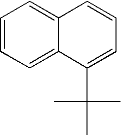 | 455.23 | 456.22 |
| 279 | Me | H | Me | Me | 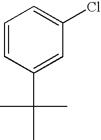 | 439.18 | 440.18 |
| 280 | Me | H | Me | Me | 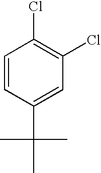 | 473.14 | 474.2 |
| 281 | Me | H | Me | Me | 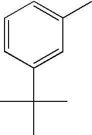 | 419.23 | 420.25 |
| 282 | Me | H | Me | Me | 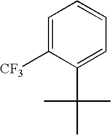 | 473.20 | 474.20 |
| 283 | Me | H | Me | Me | 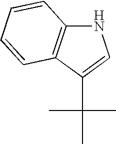 | 444.23 | 445.31 |
| 284 | Me | H | Me | Me | 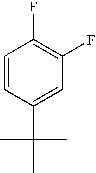 | 441.20 | 442.20 |
| 285 | Me | H | Me | Me | 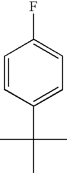 | 423.21 | 424.15 |
| 286 | Me | H | H | Me | 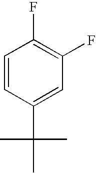 | 427.18 | 428.44 |

TABLE 8
Compounds were synthesised as described for Example 226

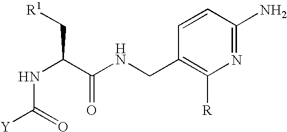

| Example | | | | | |
|---|---|---|---|---|---|
| No | Y | R | R¹ | Mol Wt | [M + H]⁺ |
| 287 | 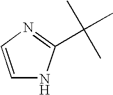 | H | 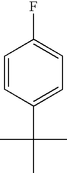 | 382.16 | 383.04 |
| 288 | 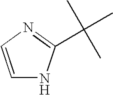 | H | 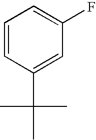 | 382.16 | 383.03 |
| 289 | 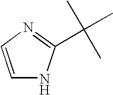 | H | 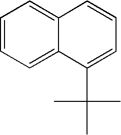 | 414.18 | 415.02 |
| 290 | 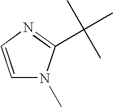 | H | 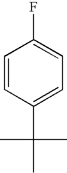 | 396.17 | 397.24 |
| 291 | 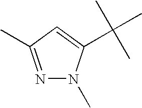 | H | 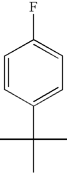 | 410.19 | 411.52 |
| 292 | 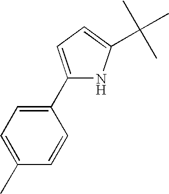 | H | 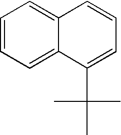 | 503.23 | 504.18 |
| 293 | 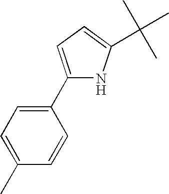 | H | 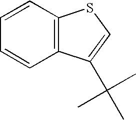 | 509.19 | 510.17 |
| 294 | 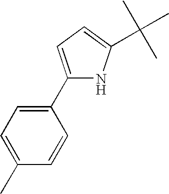 | H | 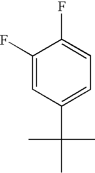 | 489.20 | 490.18 |
| 295 | 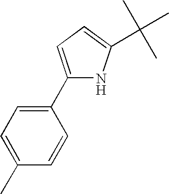 | H | 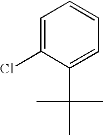 | 487.18 | 488.12 |
| 296 | 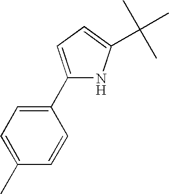 | H | 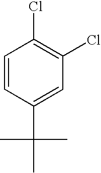 | 521.14 | 522.12, 524.12 |
| 297 | 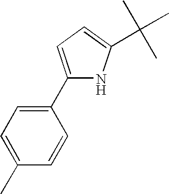 | H | 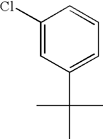 | 487.18 | 488.13 |
| 298 | 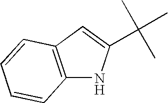 | H | 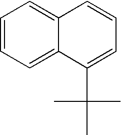 | 463.20 | 464.4 |
| 299 | 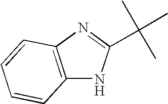 | H | 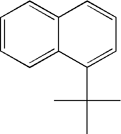 | 464.2 | 465.2 |
| 300 | 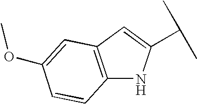 | H | 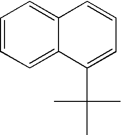 | 493.21 | 494.2 |
| 301 | 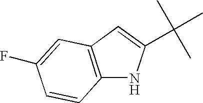 | H | 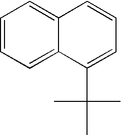 | 481.19 | 482.2 |
| 302 | 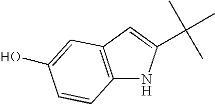 | H | 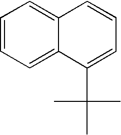 | 479.20 | 480.2 |
| 303 | 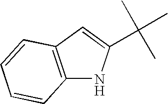 | Me | 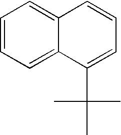 | 477.22 | 478.23 |
| 304 | 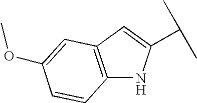 | Me | 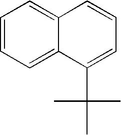 | 507.23 | 508.24 |
| 305 | 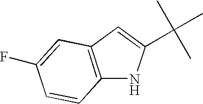 | Me | 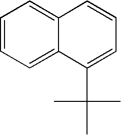 | 495.21 | 496.3 |
| 306 | 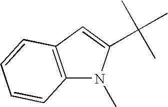 | H | 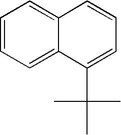 | 477.22 | 478.3 |
| 307 | 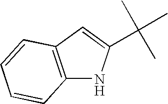 | H | 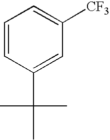 | 481.17 | 482.5 |
| 308 | 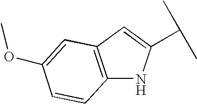 | H | 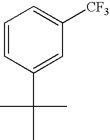 | 511.18 | 512.3 |
| 309 | 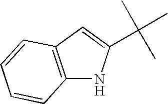 | Me | 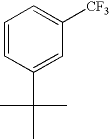 | 495.19 | 496.11 |
| 310 | 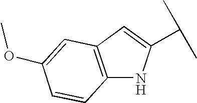 | Me | 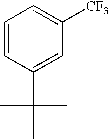 | 525.20 | 526.19 |
| 311 | 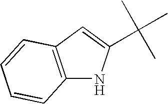 | H | 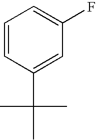 | 431.18 | 432.2 |
| 312 | 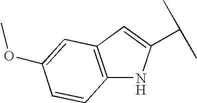 | H | 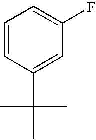 | 461.19 | 462.2 |
| 313 | 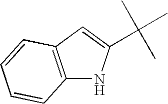 | H | 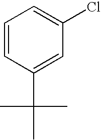 | 447.15 | 448.2, 550.2 |
| 314 | 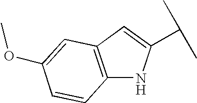 | H | 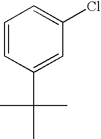 | 477.16 | 478.2, 480.2 |
| 315 | 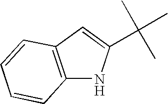 | Me | 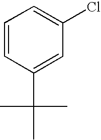 | 461.16 | 462.16 |
| 316 | 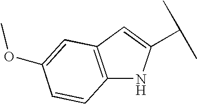 | Me | 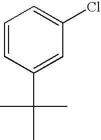 | 491.17 | 492.23 |
| 317 | 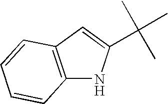 | H | 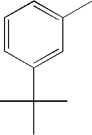 | 427.20 | 428.20 |
| 318 | 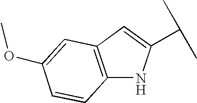 | H | 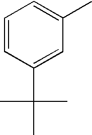 | 457.21 | 458.20 |
| 319 | 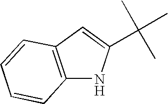 | Me | 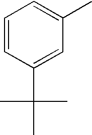 | 441.22 | 442.25 |
| 320 | 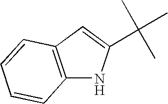 | Me | 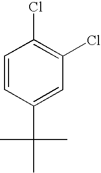 | 495.12 | 496.18 |
| 321 | 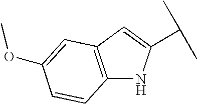 | Me | 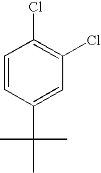 | 525.13 | 526.22 |
| 322 | 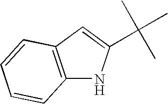 | Me | 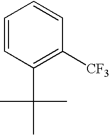 | 495.19 | 496.2 |
| 323 | 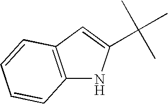 | H | 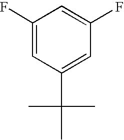 | 449.17 | 450.2 |
| 324 | 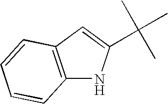 | H | 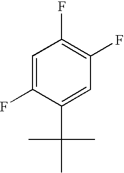 | 467.16 | 468.10 |
| 325 | 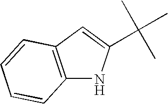 | H | 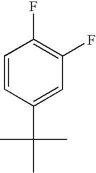 | 449.17 | 449.88, 451 |
| 326 | 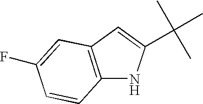 | H | 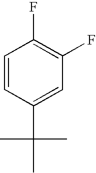 | 467.16 | 468.08 |
| 327 | 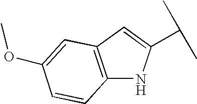 | H | 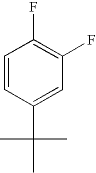 | 479.18 | 480.09 |
| 328 | 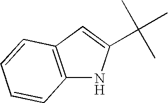 | H | 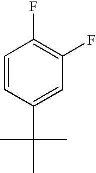 | 450.16 | 451.19 |
| 329 | 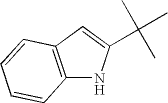 | H | 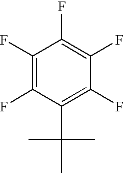 | 503.14 | 504.1 |
| 330 | 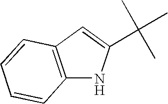 | H | 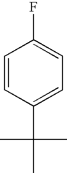 | 431.18 | 432.12 |
| 331 | 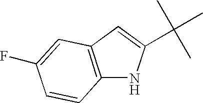 | H | 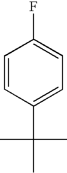 | 449.17 | 450.21 |
| 332 | 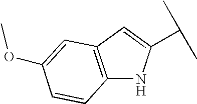 | H | 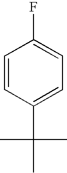 | 461.19 | 462.22 |
| 333 | 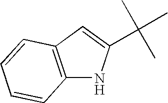 | H | 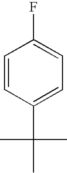 | 432.17 | 433.21 |
| 334 | 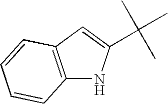 | H | 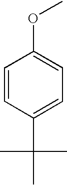 | 443.20 | 444.17 |
| 335 | 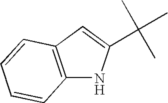 | H | 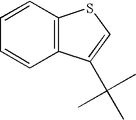 | 469.16 | 470.19 |
| 336 | 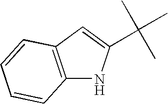 | Me | 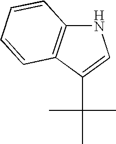 | 466.21 | 467.1 |
| 337 | 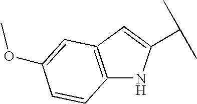 | Me | 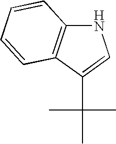 | 496.22 | 497.1 |
| 338 | 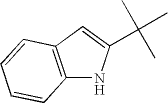 | H | 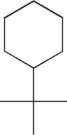 | 419.23 | 420.17 |

TABLE 9
Compounds were synthesised as described for Examples 1 to 4

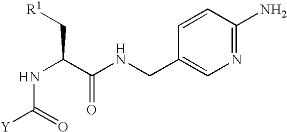

| Example | | Stereochemistry | | Mol | |
|---|---|---|---|---|---|
| No | Y | of Y | R¹ | Wt | [M + H]⁺ |
| 339 | 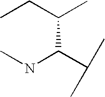 | S | 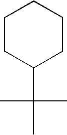 | 403.29 | 404.57 |
| 340 | 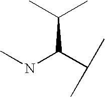 | R | 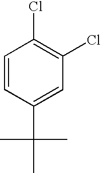 | 415.24 | 416.20 |
| 341 | 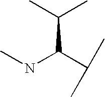 | R | 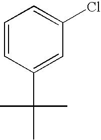 | 417.19 | 417.99 |
| 342 | 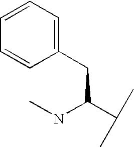 | R | 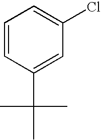 | 465.19 | 465.91 |

TABLE 10
Compounds were synthesised as described for Examples 1 to 7 and 9 and 10

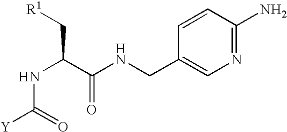

| Example | | Stereochem- | | Mol | |
|---|---|---|---|---|---|
| No | Y | istry of Y | R¹ | Wt | [M + H]⁺ |
| 343 | 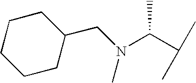 | S | 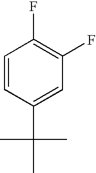 | 487.28 | 488.61 |
| 344 | 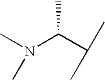 | S | 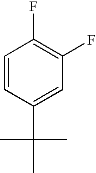 | 405.20 | 406.49 |
| 345 | 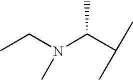 | S | 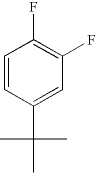 | 419.21 | 420.53 |
| 346 | 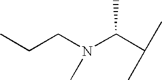 | S | 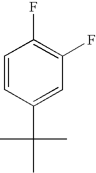 | 433.23 | 434.48 |
| 347 | 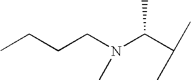 | S | 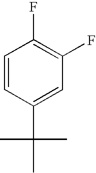 | 447.24 | 448.57 |
| 348 | 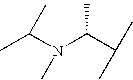 | S | 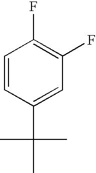 | 461.26 | 462.61 |
| 349 | 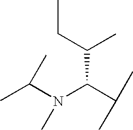 | S | 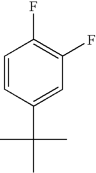 | 475.28 | 476.58 |
| 350 | 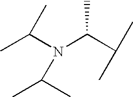 | S | 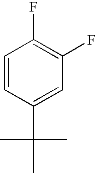 | 461.26 | 462.54 |
| 351 | 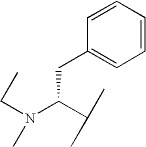 | S | 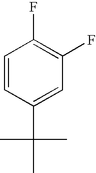 | 481.23 | 482.48 |
| 352 | 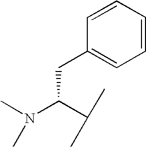 | S | 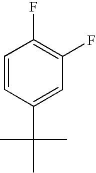 | 495.24 | 495.56 |
| 353 | 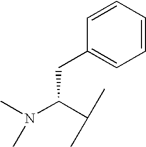 | S | 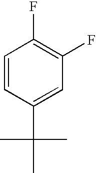 | 475.28 | 476.58 |
| 354 | 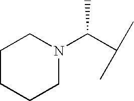 | S | 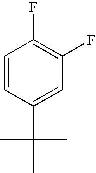 | 445.23 | 446.23 |
| 355 | 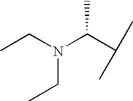 | S | 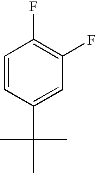 | 433.23 | 434.39 |
| 356 | 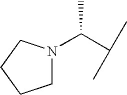 | S | 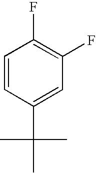 | 431.21 | 432.11 |
| 357 | 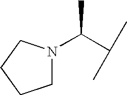 | R | 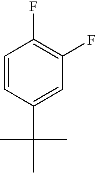 | 431.21 | 432.15 |
| 358 | 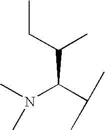 | R | 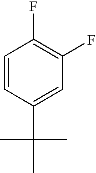 | 447.24 | 448.22 |
| 359 | 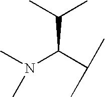 | R | 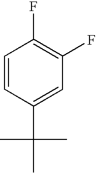 | 433.23 | 434.03 |
| 360 | 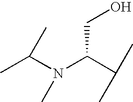 | S | 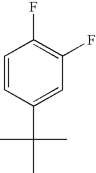 | 449.22 | 450.46 |
| 361 | 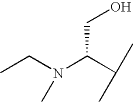 | S | 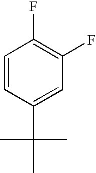 | 435.21 | 436.17 |
| 362 | 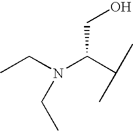 | S | 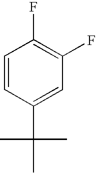 | 449.22 | 450.47 |
| 363 | 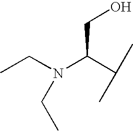 | R | 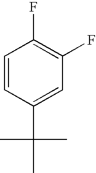 | 449.22 | 450.45 |
| 364 | 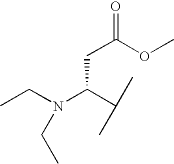 | S | 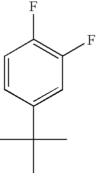 | 491.23 | 492.25 |
| 365 | 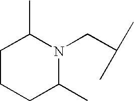 | | 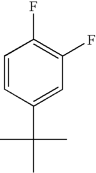 | 459.24 | 460.51 |
| 366 | 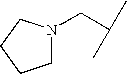 | | 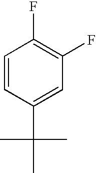 | 417.20 | 418.29 |
| 367 | 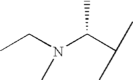 | S | 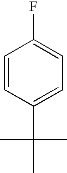 | 401.22 | 402.40 |
| 368 | 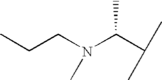 | S | 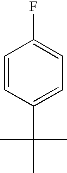 | 415.24 | 416.57 |
| 369 | 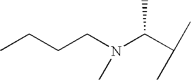 | S | 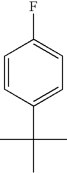 | 429.25 | 430.57 |
| 370 | 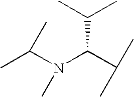 | S | 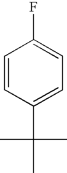 | 443.27 | 444.59 |
| 371 | 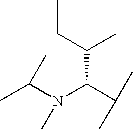 | S | 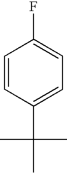 | 457.29 | 458.62 |
| 372 | 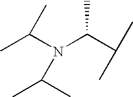 | S | 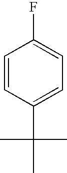 | 443.27 | 444.55 |
| 373 | 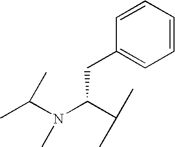 | S | 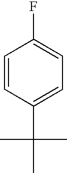 | 491.27 | 492.62 |
| 374 | 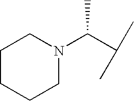 | S | 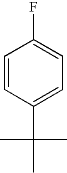 | 427.24 | 428.51 |
| 375 | 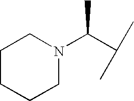 | R | 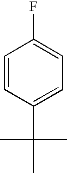 | 427.24 | 428.50 |
| 376 | 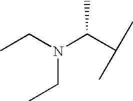 | S | 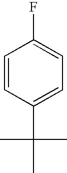 | 415.24 | 416.51 |
| 377 | 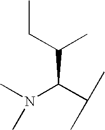 | R | 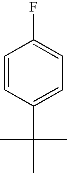 | 429.25 | 430.51 |
| 378 | 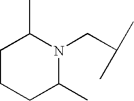 | | 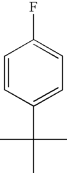 | 441.25 | 442.52 |
| 379 | 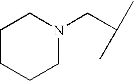 | | 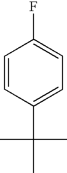 | 413.22 | 414.47 |
| 380 | 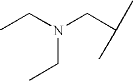 | | 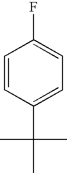 | 401.22 | 402.32 |
| 381 | 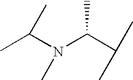 | S | 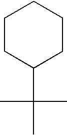 | 403.29 | 404.57 |
| 382 | 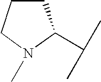 | S | 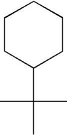 | 387.26 | 388.53 |
| 383 | 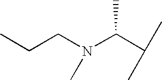 | S | 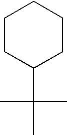 | 403.29 | 404.56 |
| 384 | 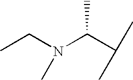 | S | 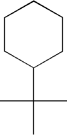 | 389.28 | 390.27 |
| 385 | 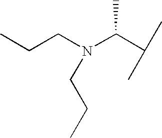 | S | 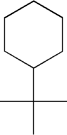 | 431.33 | 432.57 |
| 386 | 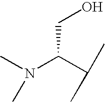 | S | 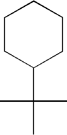 | 391.26 | 392.25 |
| 387 | 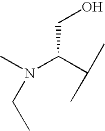 | S | 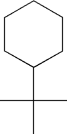 | 405.27 | 406.26 |
| 388 | 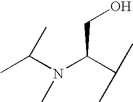 | S | 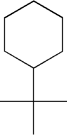 | 419.29 | 420.27 |
| 389 | 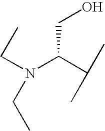 | S | 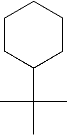 | 419.29 | 420.26 |
| 390 | 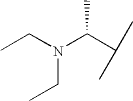 | S | 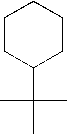 | 403.29 | 404.55 |
| 391 | 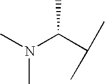 | S | 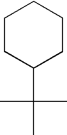 | 375.26 | 376.50 |
| 392 | 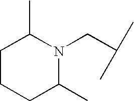 | | 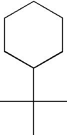 | 429.31 | 430.57 |
| 393 | 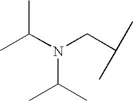 | | 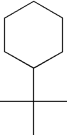 | 417.31 | 418.53 |
| 394 | 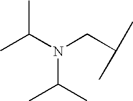 | | 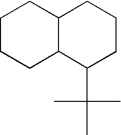 | 471.36 | 472.67 |
| 395 | 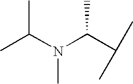 | S | 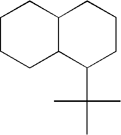 | 457.34 | 458.64 |
| 396 | 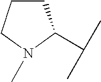 | R | 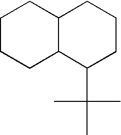 | 441.31 | 442.60 |
| 397 | 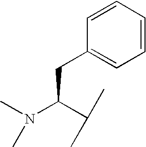 | R | 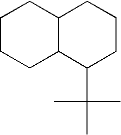 | 505.34 | 506.66 |
| 398 | 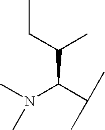 | R | 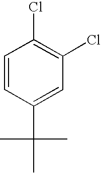 | 479.19 | 480.14, 482.12 |
| 399 | 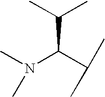 | R | 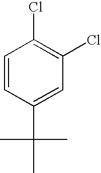 | 465.17 | 466.19 |
| 400 | 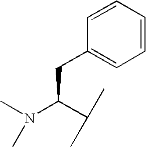 | R | 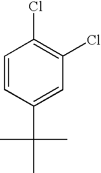 | 513.17 | 514.11, 516.10 |
| 401 | 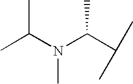 | S | 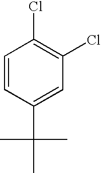 | 465.17 | 466.35 |
| 402 | 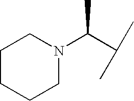 | R | 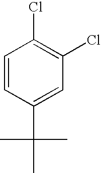 | 477.17 | 478.44 |
| 403 | 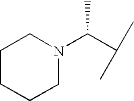 | S | 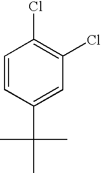 | 477.17 | 478.73 |
| 404 | 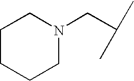 | | 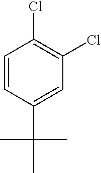 | 463.15 | 464.41 |
| 405 | 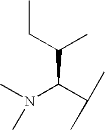 | R | 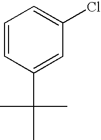 | 445.22 | 446.48, 448.40 |
| 406 | 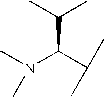 | R | 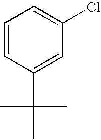 | 431.21 | 432.20 |
| 407 | 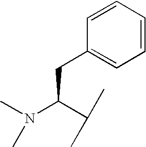 | R | 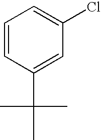 | 479.21 | 480.17 |
| 408 | 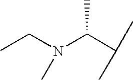 | S | 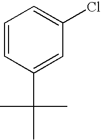 | 417.19 | 417.94 |
| 409 | 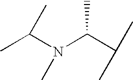 | S | 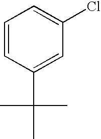 | 431.21 | 432.20 |
| 410 | 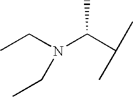 | S | 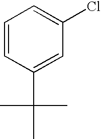 | 431.21 | 432.20 |
| 411 | 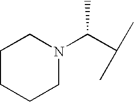 | S | 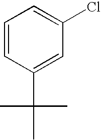 | 443.21 | 444.45 |
| 412 | 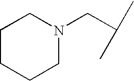 | | 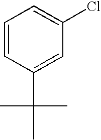 | 429.19 | 430.44 |
| 413 | 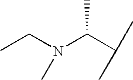 | S | 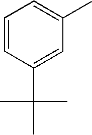 | 397.25 | 398.24 |
| 414 | 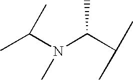 | S | 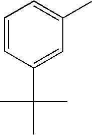 | 411.26 | 412.27 |
| 415 | 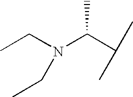 | S | 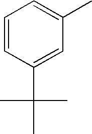 | 411.26 | 412.27 |
| 416 | 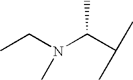 | S | 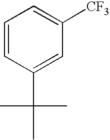 | 451.22 | 452.20 |
| 417 | 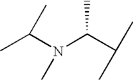 | S | 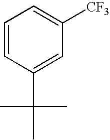 | 465.24 | 466.01 |
| 418 | 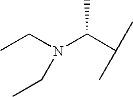 | S | 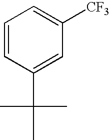 | 465.52 | 466.62 |
| 419 | 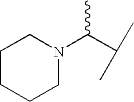 | R,S | 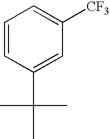 | 477.24 | 478.49 |
| 420 | 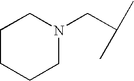 | | 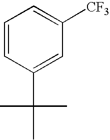 | 463.22 | 464.47 |
| 421 | 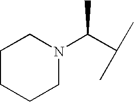 | R | 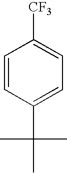 | 477.24 | 478.51 |
| 422 | 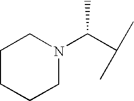 | S | 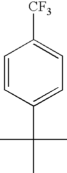 | 477.24 | 478.51 |
| 423 | 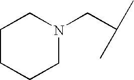 | | 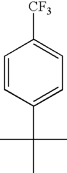 | 463.22 | 464.48 |
| 424 | 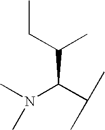 | R | 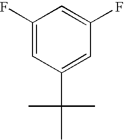 | 447.24 | 448.50 |
| 425 | 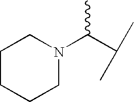 | R,S | 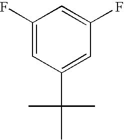 | 445.23 | 446.48, 448.40 |
| 426 | 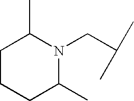 | | 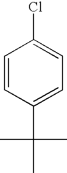 | 457.22 | 458.49, 460.48 |
| 427 | 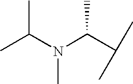 | S | 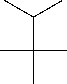 | 363.26 | 364.51 |

TABLE 11
Compounds were synthesised as described for Examples 1 to 7 and 9 and 10

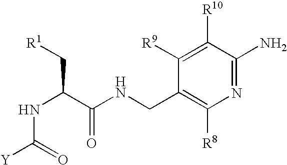

| | | Stereo- | | | | | | |
|---|---|---|---|---|---|---|---|---|
| Example | | chem. | | | | | Mol | |
| No | Y | At Y | R¹ | R⁸ | R⁹ | R¹⁰ | Wt | [M + H]⁺ |
| 428 | 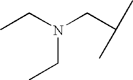 | | 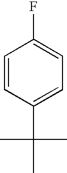 | Me | H | H | 415.24 | 416.20 |
| 429 | 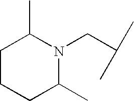 | | 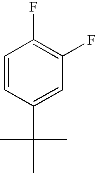 | Me | H | H | 473.26 | 474.53 |
| 430 | 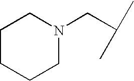 | | 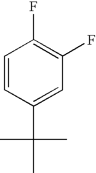 | Me | H | H | 445.23 | 446.49 |
| 431 | 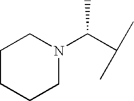 | R | 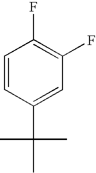 | Me | H | H | 459.24 | 460.50 |
| 432 | 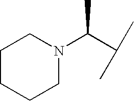 | S | 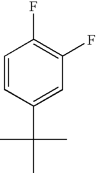 | Me | H | H | 429.50 | 460.50 |

TABLE 12
Compounds were synthesised as described for Examples 1 to 7
and 9 to 11

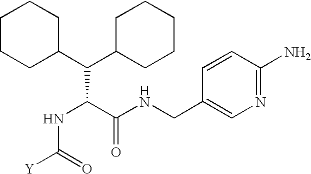

| | | Stereo- | | |
|---|---|---|---|---|
| Example | | chemistry | | |
| No | Y | of Y | Mol Wt | [M + H]⁺ |
| 433 | 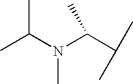 | S | 485.37 | 486.66 |
| 434 | 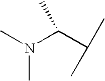 | S | 405.20 | 406.49 |

TABLE 13
Compounds were synthesised as described for Examples 1 to 11

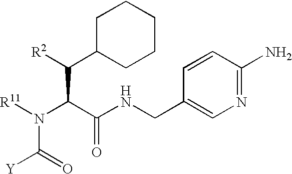

| | | Stereo- | | | | |
|---|---|---|---|---|---|---|
| Example | | chemistry | | | | |
| No | Y | of Y | R² | R¹¹ | Mol Wt | [M + H]⁺ |
| 435 | 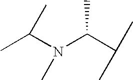 | S | OH | H | 419.29 | 420.57 |
| 436 | 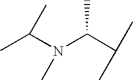 | S | Me | H | 417.31 | 418.50 |
| 437 | 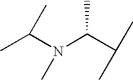 | S | H | Me | 417.31 | 418.49 |
| 438 | 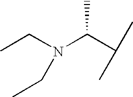 | S | H | Me | 417.31 | 418.46 |
| 439 | 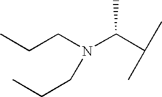 | S | H | Me | 431.33 | 432.57 |

TABLE 14
¹H NMR data for examples

| Example | | Frequency | |
|---|---|---|---|
| No | Solvent | (MHz) | Chemical Shifts |
| 12 | CD₃OD | 270 | 0.95-1.05 (6H, m), 1.10-2.10 (28H, m), |
| | | | 3.78-3.82 (1H, m), 4.25-4.34 (3H, m), 6.92-6.98 (1H, |
| | | | m), 7.61-7.84 (2H, m) |
| 13 | CD₃OD | 270 | 0.90-1.96 (22H, m), 2.70-2.98 (2H, m), |
| | | | 3.09-3.18 (4H, m), 4.05-4.20 (2H, m), 6.96 (1H, d, |
| | | | J = 9.1 Hz), 7.22-7.30 (4H, m), 7.72 (1H, s), |
| | | | 7.80-7.90 (2H, m) |
| 16 | CD₃OD | 270 | 1.00 (6H, d), 1.13-2.1 (28H, m), 3.75-3.95 (1H, |
| | | | m), 4.15-4.35 (2H, m), 6.97 (1H, d, J = 8.9 Hz), |
| | | | 7.72 (1H, s), 7.86 (1H, d, J = 8.7 Hz), |
| | | | 8.80-8.90 (1H, m) |
| 19 | CD₃OD | 270 | 1.19-1.78 (23H, m), 2.71 (3H, s), 3.83 (2H, s), |
| | | | 4.26-4.32 (3H, m), 6.97 (1H, d, J = 9.1 Hz), |
| | | | 7.74 (1H, s), 7.84-7.87 (1H, m), 8.85 (1H, s, br) |
| 20 | CD₃OD | 270 | 0.99-1.14 (8H, m), 1.21-1.32 (6H, m), |
| | | | 1.40-1.71 (6H, m), 3.19-3.21 (2H, m), 3.76 (1H, d, |
| | | | J = 5.2 Hz), 4.27-4.39 (3H, m), 4.91 (5H, s), |
| | | | 6.97 (1H, d, J = 9.4 Hz), 7.74 (1H, s), 7.86 (1H, d, |
| | | | J = 8.2 Hz), 8.70-8.90 (1H, m) |
| 21 | CD₃OD | 270 | 0.9-1.0 (7H, m), 1.4-1.8 (7H, m), 2.4-2.6 (5H, |
| | | | m), 3.3-3.7 (10H, m), 4.0-4.4 (3H, m) |
| | | | 6.9-7.0 (1H, d, J = 7 hz), 7.8-7.9 (2H, m), 8.7-8.9 (2H, m) |
| 22 | CD₃OD | 270 | 0.65 (3H, d, J = 6.9 Hz), 0.86 (3H, d, J = 6.9 Hz), |
| | | | 1.28-1.29 (1H, m), 1.88-1.91 (1H, m), |
| | | | 3.30-3.56 (3H, m), 3.66-3.68 (3H, m), 4.08-4.19 (2H, m), |
| | | | 4.63-4.85 (3H, m), 6.85-6.89 (1H, m), |
| | | | 7.33-7.37 (2H, m), 7.46-7.59 (4H, m), 7.75-7.71 (1H, m), |
| | | | 7.84-7.87 (1H, m), 8.18 (1H, d, J = 7.6 Hz) |
| 24 | CD₃OD | 270 | 0.81-0.83 (6H, m), 1.10-1.30 (2H, m), |
| | | | 2.95-3.20 (1H, m), 3.27-3.30 (3H, m), 3.71 (1H, d, |
| | | | J = 5.2 Hz), 4.21 (2H, s), 4.7-4.9 (1H, m), |
| | | | 4.91 (4H, s), 6.94 (1H, d, J = 9.1 Hz), 7.21-7.36 (4H, |
| | | | m), 7.68 (1H, s), 7.76 (1H, d, J = 9.4 Hz), |
| | | | 8.5-8.7 (1H, s, br) |
| 25 | CD₃OD | 270 | 0.81-0.87 (6H, m), 1.27-1.30 (2H, m), |
| | | | 3.19-3.29 (1H, m), 3.30-3.31 (3H, m), 3.73 (1H, d, |
| | | | J = 5.2 Hz), 4.21-4.22 (2H, m), 4.71-4.77 (1H, m), |
| | | | 4.91 (4H, s), 6.94 (1H, d, J = 9.1 Hz), |
| | | | 7.42-7.52 (3H, m), 7.66-7.69 (2H, s), 7.74-7.79 (1H, m), |
| | | | 8.5-8.7 (1H, s, br) |
| 26 | CD₃OD | 270 | 0.75-0.80 (6H, m), 1.10-1.30 (1H, m), |
| | | | 1.60-1.85 (1H, m), 2.83-2.92 (1H, m), 3.14-3.21 (1H, m), |
| | | | 3.68 (1H, d, J = 5.2 Hz), 4.22-4.26 (2H, m), |
| | | | 4.65-4.67 (1H, m), 4.90 (6H, s), 6.94 (1H, d, |
| | | | J = 8.8 Hz), 7.21-7.27 (5H, m), 7.68-7.76 (2H, m), |
| | | | 8.60-8.80 (1H, m) |
| 27 | CD₃OD | 270 | 0.77 (6H, t, J = 7.28 Hz), 1.10-1.40 (1H, m), |
| | | | 1.60-1.80 (1H, m), 2.29 (3H, s), 2.75-2.95 (1H, m) |
| | | | 3.12-3.28 (1H, m), 3.29-3.31 (2H, m), 3.68 (1H, |
| | | | d, J = 5.1 Hz), 4.22-4.26 (2H, m), 4.62-4.64 (1H, |
| | | | m), 4.90 (5H, s), 6.92-7.15 (4H, m), |
| | | | 7.69-7.75 (2H, m), 8.65-8.90 (1H, m) |
| 28 | CD₃OD | 270 | 0.83 (6H, t, J = 6.6 Hz), 0.90-1.10 (1H, m), |
| | | | 1.60-1.80 (1H, m), 2.34 (3H, s), 2.85-3.00 (1H, m), |
| | | | 3.12-3.28 (1H, m), 3.29-3.31 (2H, m), 3.71 (1H, |
| | | | d, J = 5.4 Hz), 4.16-4.23 (2H, m), 4.63-4.68 (1H, |
| | | | m), 4.90 (5H, s), 6.92 (1H, d, J = 9.2 Hz), |
| | | | 7.06-7.14 (3H, m), 7.63-7.69 (2H, m), 8.65-8.90 (1H, |
| | | | m) |
| 29 | CD₃OD | 270 | 0.77-0.82 (7H, m), 1.17-1.30 (1H, m), |
| | | | 1.6-1.8 (1H, m), 2.91-2.95 (1H, m), 3.29-3.30 (1H, m), |
| | | | 3.68 (1H, d, J = 5.2 Hz), 4.22-4.27 (2H, m), |
| | | | 4.62-4.66 (1H, m), 4.91 (4H, s), 6.94-7.09 (4H, m), |
| | | | 7.28-7.31 (2H, m), 7.71-7.79 (2H, m), |
| | | | 8.5-8.7 (1H, m) |
| 30 | CD₃OD | 270 | 0.72-0.77 (7H, m), 1.17-1.30 (1H, m), |
| | | | 1.6-1.8 (1H, m), 2.98-3.05 (1H, m), 3.25-3.30 (1H, m), |
| | | | 3.66 (1H, d, J = 5.2 Hz), 4.26 (2H, s), |
| | | | 4.71-4.77 (1H, m), 4.91 (4H, s), 6.95 (1H, d, J = 9.1 Hz), |
| | | | 7.51-7.59 (4H, m), 7.73 (1H, s), 7.75-7.80 (1H, |
| | | | m), 8.5-8.7 (1H, s, br) |
| 31 | CD₃OD | 270 | 0.70-1.05 (7H, m), 1.10-1.30 (1H, m), |
| | | | 1.60-1.80 (1H, m), 2.90-3.10 (1H, m), 3.20-3.40 (2H, m), |
| | | | 3.69 (1H, d, J = 5.18 Hz), 4.20-4.40 (2H, m), |
| | | | 4.65-4.75 (1H, m), 4.90-5.10 (4H, m), 6.97 (1H, d, |
| | | | J = 9.15 Hz), 7.40-7.90 (6H, m), 8.70-8.90 (1H, |
| | | | m) |
| 32 | CD₃OD | 270 | 0.75-1.30 (8H, m), 1.65-1.80 (1H, m), |
| | | | 2.80-2.95 (1H, m), 3.10-3.20 (1H, m), 3.25-3.35 (2H, m), |
| | | | 3.60-3.80 (2H, m), 4.15-4.35 (2H, m), |
| | | | 4.65-4.80 (1H, m), 4.90-5.10 (3H, m), 6.90-7.20 (4H, m), |
| | | | 7.70-7.85 (2H, m) |
| 33 | CD₃OD | 270 | 0.70-1.00 (6H, m), 1.10-1.30 (1H, m), |
| | | | 1.60-1.80 (1H, m), 2.80-2.95 (1H, m), 3.10-3.20 (1H, m), |
| | | | 3.25-3.35 (2H, m), 3.65-3.75 (1H, m), |
| | | | 4.15-4.40 (2H, m), 4.50-4.70 (1H, m), 4.80-5.00 (4H, m), |
| | | | 6.90-7.10 (3H, m), 7.20-7.30 (2H, m), |
| | | | 7.65-7.80 (2H, m), 8.70-8.90 (1H, m) |
| 34 | CD₃OD | 270 | 0.48 (3H, t, J = 7.13 Hz) 0.65-0.80 (3H, m), |
| | | | 1.00-1.20 (1H, m), 1.55-1.70 (1H, m), 3.00-3.10 (1H, |
| | | | m), 3.25-3.40 (2H, m), 3.60-3.80 (2H, m), |
| | | | 4.10-4.35 (2H, m), 4.80-4.95 (1H, m), 4.95-5.05 (4H, |
| | | | m), 6.74 (1H, d, J = 9.15 Hz), 7.30-7.85 (9H, m), |
| | | | 8.70-8.80 (1H, m) |
| 35 | CD₃OD | 270 | 0.70-0.90 (7H, m), 1.15-1.30 (1H, m), |
| | | | 1.65-1.80 (1H, m), 2.80-2.95 (1H, m), 3.10-3.20 (1H, |
| | | | m), 3.25-3.35 (2H, m), 3.60-3.80 (2H, m), |
| | | | 4.15-4.35 (2H, m), 4.70-4.80 (1H, m), 4.90-5.10 (3H, |
| | | | m), 6.90-7.00 (1H, m), 7.15-7.25 (1H, m), |
| | | | 7.35-7.50 (2H, m), 7.70-7.80 (2H, m) |
| 36 | CD₃OD | 270 | 0.80-0.89 (6H, m), 1.18 (1H, br, s), 1.71 (1H, br, |
| | | | s), 2.88-2.91 (1H, m), 3.14-3.21 (1H, m), |
| | | | 3.70 (1H, d, J = 4.7 Hz), 4.25 (1H, s), 4.63 (1H, t, J = 3.9 Hz), |
| | | | 4.91 (7H, s), 6.96 (1H, d, J = 8.9 Hz), |
| | | | 7.26 (4H, s), 7.72-7.78 (2H, m), 8.75 (1H, s) |
| 37 | CD₃OD | 270 | 0.81 (6H, s), 1.24 (1H, s), 1.73 (1H, s), |
| | | | 2.74-2.83 (1H, m), 3.06-3.11 (1H, m), 3.31 (1H, s), |
| | | | 3.71 (1H, s), 4.24 (1H, s), 4.57 (1H, br, s), 4.91 (7H, |
| | | | s), 6.69 (2H, d, J = 6.9 Hz), 6.94 (1H, d, J = 8.6 Hz), |
| | | | 7.05 (2H, d, J = 6.9 Hz), 7.73 (2H, d, J = 9.7 Hz) |
| 38 | CD₃OD | 270 | 0.84-0.86 (6H, m), 1.24-1.29 (1H, m), |
| | | | 1.77-1.79 (1H, m), 2.91-2.99 (1H, m), 3.14-3.19 (1H, m), |
| | | | 3.30 (1H, s), 3.73 (1H, d, J = 4.9 Hz), 4.24 (2H, |
| | | | s), 4.71 (1H, t, J = 3.5 Hz), 4.90 (5H, s), |
| | | | 6.95 (1H, d, J = 9.1 Hz), 7.09-7.19 (2H, m) |
| | | | 7.74-7.83 (2H, m), 8.75-8.78 (1H, m) |
| 39 | CD₃OD | 270 | 0.80-0.85 (6H, m), 1.25 (1H, s), 1.74 (1H, s), |
| | | | 2.92 (1H, s), 3.19-3.32 (2H, m), 3.71-3.73 (1H, |
| | | | m), 4.27 (2H, s), 4.69 (1H, t, J = 5.2 Hz), |
| | | | 4.93 (6H, s), 6.89-6.99 (4H, m), 7.75-7.84 (2H, m) |
| 40 | CD₃OD | 270 | 0.60-0.80 (6H, m), 1.10-1.30 (1H, m), |
| | | | 1.60-1.75 (1H, m), 3.00-3.15 (1H, m), 3.20-3.40 (2H, m), |
| | | | 3.69 (1H, d, J = 5.42 Hz), 4.10-4.30 (2H, m), |
| | | | 4.65-4.80 (2H, m), 4.90-5.10 (6H, m), 6.80-6.90 (1H, |
| | | | m), 6.95-7.20 (3H, m), 7.30-7.40 (1H, m), |
| | | | 7.50-7.60 (3H, m), 8.60-8.70 (1H, m) |
| 41 | CD₃OD | 270 | 0.77-0.91 (5H, m), 1.23 (1H, s), 1.71 (1H, s), |
| | | | 3.17-3.31 (2H, m), 3.41-3.46 (1H, m), 3.72 (1H, |
| | | | s), 4.22 (2H, s), 4.82-4.91 (7H, m), 6.88 (1H, d, J = 7.9 Hz), |
| | | | 7.37 (3H, s), 7.65 (2H, s), 7.87 (2H, |
| | | | s), 8.75 (1H, s) |
| 42 | CD₃OD | 270 | 0.65-0.85 (7H, m), 1.15-1.30 (1H, m), |
| | | | 1.65-1.80 (1H, m), 2.60 (3H, s), 2.90-3.10 (1H, m), |
| | | | 3.20-3.40 (3H, m), 3.61 (1H, d, J = 4.94 Hz), |
| | | | 4.20-4.40 (2H, m), 4.65-4.90 (1H, m), 4.90-5.10 (2H, m), |
| | | | 6.96 (1H, d, J = 9.15 Hz), 7.45-7.90 (6H, m), |
| | | | 8.75-8.90 (1H, m) |
| 43 | CD₃OD | 270 | 0.70-0.95 (7H, m), 1.20-1.40 (1H, m), |
| | | | 1.70-1.90 (1H, m), 2.50-2.70 (3H, m), 2.85-2.95 (1H, m), |
| | | | 3.05-3.20 (1H, m), 3.25-3.35 (2H, m), |
| | | | 3.60-3.70 (1H, m), 4.15-4.30 (2H, m), 4.55-4.70 (1H, m), |
| | | | 4.90-5.00 (2H, m), 6.90-7.30 (4H, m), |
| | | | 7.70-7.90 (2H, m), 8.70-8.80 (1H, m) |
| 44 | CD₃OD | 270 | 0.70-0.90 (7H, m), 1.20-1.35 (1H, m), |
| | | | 1.65-1.85 (1H, m), 2.60 (3H, s), 2.80-2.95 (1H, m), |
| | | | 3.05-3.20 (1H, m), 3.25-3.40 (2H, m), 3.63 (1H, d, |
| | | | J = 4.94 Hz), 4.20-4.35 (2H, m), 4.55-4.70 (1H, |
| | | | m), 4.90-5.10 (2H, m), 6.90-7.10 (3H, m), |
| | | | 7.20-7.35 (2H, m), 7.70-7.85 (2H, m), |
| | | | 8.70-8.85 (1H, m) |
| 45 | CD₃OD | 270 | 0.70-0.90 (7H, m), 1.20-1.40 (1H, m), |
| | | | 1.60-1.75 (1H, m), 2.60 (3H, s), 2.80-2.95 (1H, m), |
| | | | 3.10-3.20 (1H, m), 3.25-3.40 (2H, m), 3.63 (1H, d, |
| | | | J = 4.70 Hz), 4.20-4.40 (2H, m), 4.60-4.70 (1H, |
| | | | m), 4.90-5.10 (2H, m), 6.90-7.10 (1H, m), |
| | | | 7.15-7.40 (4H, m), 7.70-7.85 (2H, m), 8.70-8.90 (1H, |
| | | | m) |
| 46 | CD₃OD | 270 | 0.82 (6H, s), 1.28 (1H, s), 1.77 (1H, s), 2.60 (3H, |
| | | | s), 2.86-2.95 (1H, m), 3.11-3.18 (1H, m), |
| | | | 3.65 (1H, s), 4.25 (2H, s), 4.63 (1H, t, J = 6.2 Hz), |
| | | | 4.92 (5H, s), 6.97 (1H, d, J = 9.2 Hz), 7.27 (4H, |
| | | | s), 7.76 (2H, d, J = 9.4 Hz), 8.78 (1H, s) |
| 48 | CD₃OD | 270 | 0.84-0.96 (6H, m), 1.34-1.38 (1H, m), |
| | | | 1.78-1.83 (1H, m), 2.61 (3H, s), 2.95-3.03 (3H, m), |
| | | | 3.68 (1H, d, J = 4.7 Hz), 4.24 (2H, s), 4.64-4.70 (1H, |
| | | | m), 4.91 (5H, s), 6.95 (1H, d, J = 9.2 Hz), |
| | | | 7.11-7.21 (2H, m), 7.75-7.82 (2H, m) |
| 50 | CD₃OD | 270 | 0.8-0.9 (6H, m), 1.2-1.4 (1H, m), 1.6-1.9 (1H, |
| | | | m), 2.4-2.6 (7H, m), 2.9-3.2 (2H, m), |
| | | | 3.7-3.8 (1H, m), 4.0-4.4 (1H, m), 4.6-4.8 (1H, m), |
| | | | 6.85 (1H, d, J = 7 Hz), 7.8-8.0 (3H, m), 8.8-8.9 (1H, |
| | | | m), 9.15 (1H, d, J = 7 Hz) |
| 51 | CD₃OD | 270 | 0.78 (7H, s), 1.28 (1H, s), 1.76 (1H, s), 2.61 (3H, |
| | | | s), 3.21-3.44 (3H, m), 3.67 (1H, s), 4.22 (2H, s), |
| | | | 4.82-4.94 (4H, m), 6.89 (1H, d, J = 9.4 Hz), |
| | | | 7.39 (3H, s), 7.67 (2H, s), 7.88 (2H, s), 8.81 (1H, s) |
| 52 | CD₃OD | 270 | 0.6-0.8 (6H, m), 1.0-1.1 (1H, m), 1.3 (9H, s), |
| | | | 1.7-1.8 (1H, m), 2.4-2.6 (6H, m), 2.7-2.9 (1H, |
| | | | m), 3.0-3.2 (1H, m), 3.5-3.8 (1H, m), |
| | | | 4.1-4.2 (2H, m), 4.5-4.6 (1H, m), 6.95 (1H, d, J = 8 Hz), |
| | | | 7.1-7.3 (4H, m), 7.8-7.9 (2H, m), 8.3 (1H, m), |
| | | | 8.8-8.9 (2H, m) |
| 53 | CD₃OD | 400 | 0.85-1.00 (6H, m), 1.10-1.30 (1H, m), |
| | | | 1.50-1.70 (1H, m), 1.85-1.95 (1H, m), 2.47 (2H, s), |
| | | | 2.90-3.10 (2H, m), 3.25-3.35 (1H, m), 3.625 (1H. d, |
| | | | J = 4 Hz), 4.10-4.30 (2H, m), 4.67 (1H, t, J = 8 Hz), |
| | | | 4.85-5.00 (2H, m), 6.90-7.00 (2H, m), |
| | | | 7.05-7.30 (2H, m), 7.70-7.80 (2H, m), 8.70-8.80 (1H, m) |
| 54 | CD₃OD | 270 | 2.70-3.10 (4H, m), 3.20-3.40 (2H, m), |
| | | | 4.00-4.35 (3H, m), 4.40-4.45 (1H, m), 4.80-5.10 (3H, m), |
| | | | 6.90-7.50 (9H, m), 7.60-7.80 (2H, m), |
| | | | 8.60-8.80 (1H, m) |
| 55 | CD₃OD | 270 | 1.60-2.10 (3H, m), 2.20-2.40 (1H, m), |
| | | | 2.80-3.50 (5H, m), 4.10-4.35 (3H, m), 4.55-4.85 (1H, m), |
| | | | 4.80-5.10 (3H, m), 6.90-7.00 (1H, m), |
| | | | 7.10-7.25 (1H, m), 7.30-7.50 (2H, m), 7.70-7.90 (2H, m), |
| | | | 8.70-8.90 (1H, m) |
| 56 | CD₃OD | 270 | 2.90-3.00 (2H, m), 3.05-3.20 (1H, m), |
| | | | 3.25-3.40 (3H, m), 4.10-4.35 (4H, m), 4.40-4.50 (1H, m), |
| | | | 4.60-4.70 (1H, m), 4.90-5.10 (3H, m), |
| | | | 6.90-7.00 (1H, m), 7.10-7.20 (1H, m), 7.30-7.50 (2H, m), |
| | | | 7.70-7.80 (2H, m) |
| 57 | CD₃OD | 270 | 2.65-2.80 (1H, m), 3.00-3.20 (1H, m), |
| | | | 3.25-3.40 (3H, m), 4.10-4.40 (2H, m), 4.60-4.70 (1H, m), |
| | | | 4.90-5.00 (2H, m), 6.80-6.90 (1H, m), |
| | | | 6.95-7.05 (1H, m), 7.10-7.20 (2H, m), 7.25-7.50 (5H, m), |
| | | | 7.70-7.80 (2H, m), 8.70-8.80 (1H, m) |
| 58 | CD₃OD | 270 | 2.90-3.10 (2H, m), 3.25-3.40 (3H, m), |
| | | | 3.90-4.25 (2H, m), 4.50-4.65 (1H, m), 4.90-5.20 (2H, m), |
| | | | 6.85-7.00 (1H, m), 7.05-7.15 (1H, m), |
| | | | 7.20-7.80 (9H, m), 8.50-8.60 (1H, m) |
| 59 | CD₃OD | 270 | 2.70-3.10 (4H, m) 3.20-3.40 (2H, m), |
| | | | 4.05-4.35 (3H, m), 4.50-4.60 (1H, m), 4.80-5.00 (3H, m), |
| | | | 6.90-7.50 (8H, m), 7.65-7.80 (2H, m), |
| | | | 8.70-8.80 (1H, m) |
| 60 | CD₃OD | 270 | 2.64 (3H, s), 2.70-2.90 (2H, m), 2.95-3.15 (2H, |
| | | | m), 3.25-3.40 (2H, m), 3.95-4.40 (4H, m), |
| | | | 4.90-5.10 (2H, m), 6.90-7.30 (9H, m), 7.65-7.75 (2H, |
| | | | m), 8.70-8.80 (1H, m) |
| 61 | CD₃OD | 270 | 0.89 (9H, s), 2.85-3.00 (1H, m), 3.10-3.20 (1H, |
| | | | m), 3.25-3.35 (2H, m), 3.54 (1H, s), |
| | | | 4.10-4.25 (2H, m), 4.60-4.70 (1H, m), 4.90-5.10 (3H, m), |
| | | | 6.90-7.10 (1H, m), 7.15-7.25 (1H, m), |
| | | | 7.35-7.45 (2H, m), 7.70-7.80 (2H, m), 8.70-8.80 (1H, m) |
| 62 | CD₃OD | 270 | 1.35 (3H, d, J = 6.94 Hz), 2.62 (3H, s), |
| | | | 2.85-2.95 (1H, m), 3.05-3.15 (1H, m), 3.25-3.40 (2H, m), |
| | | | 3.70-3.90 (1H.m), 4.10-4.30 (2H, m), |
| | | | 4.50-4.70 (1H, m), 4.90-5.10 (2H, m), 6.90-7.00 (1H, m), |
| | | | 7.10-7.20 (1H, m), 7.25-7.50 (2H, m), |
| | | | 7.65-7.80 (2H, m), 8.70-8.80 (1H, m) |
| 63 | CD₃OD | 270 | 1.46-1.63 (2H, m) 1.83 (2H, d, J = 10.9 Hz) |
| | | | 2.00 (1H, d, J = 14.1 Hz) 2.83-2.97 (2H, m) |
| | | | 3.10-3.18 (1H, m) 3.30-3.37 (1H, m) 3.79 (1H, dd, J = 8.6, |
| | | | 3.0 Hz) 4.21 (2H, q, J = 7.7 Hz) 4.59-4.65 (1H, |
| | | | m) 4.93 (6H, s) 6.94 (1H, d, J = 9.2 Hz) |
| | | | 7.16 (1H, dd, J = 8.2, 2.0 Hz) 7.37-7.42 (2H, m) |
| | | | 7.71-7.76 (2H, m) |
| 64 | CD₃OD | 270 | 2.75-3.00 (2H, m), 3.16-3.25 (2H, m), |
| | | | 3.25-3.40 (2H, m), 4.10-4.40 (5H, m), 4.65-4.80 (1H, m), |
| | | | 4.90-5.00 (2H, m), 6.90-7.00 (1H, m), |
| | | | 7.15-7.50 (7H, m), 7.70-7.80 (2H, m), 8.70-8.85 (1H, m) |
| 65 | CD₃OD | 270 | 2.95-3.15 (2H, m), 3.25-3.35 (1H, m), |
| | | | 4.00-4.35 (4H, m), 4.55-4.70 (1H, m), 4.80-5.00 (3H, m), |
| | | | 5.05-5.15 (1H, m), 5.70-5.85 (1H, m), |
| | | | 5.95-6.10 (1H, m), 6.90-7.05 (1H, m), 7.10-7.20 (1H, m), |
| | | | 7.30-7.50 (2H, m), 7.65-7.80 (2H, m), |
| | | | 8.70-8.85 (1H, m) |
| 66 | CD₃OD | 270 | 2.20-2.50 (1H, m), 2.75-3.00 (2H, m), |
| | | | 3.05-3.20 (1H, m), 3.25-3.40 (2H, m), 3.65-3.80 (2H, m), |
| | | | 4.10-4.35 (2H, m), 4.50-4.75 (2H, m), |
| | | | 4.80-5.10 (2H, m), 6.90-7.05 (1H, m), 7.10-7.20 (1H, m), |
| | | | 7.30-7.50 (2H, m), 7.65-7.80 (2H, m), |
| | | | 8.70-8.85 (1H, m) |
| 67 | CD₃OD | 270 | 0.80-2.00 (2H, m), 2.30-2.50 (2H, m), |
| | | | 2.80-2.95 (1H, m), 3.10-3.20 (1H, m), 3.25-3.35 (2H, m), |
| | | | 3.85-3.95 (1H, m), 4.15-4.40 (2H, m), |
| | | | 4.60-4.70 (1H, m), 4.90-5.10 (3H, m), 6.90-7.50 (9H, m), |
| | | | 7.70-7.85 (2H, m), 8.70-8.85 (1H, m) |
| 68 | CD₃OD | 270 | 0.70-0.95 (2H, m), 1.00-1.30 (4H, m), |
| | | | 1.40-1.80 (7H, m), 2.80-2.95 (1H, m), 3.10-3.40 (3H, m), |
| | | | 3.80-3.90 (1H, m), 4.15-4.35 (2H, m), |
| | | | 4.60-4.70 (1H, m), 4.80-5.10 (3H, m), 6.90-7.00 (1H, m), |
| | | | 7.15-7.25 (1H, m), 7.35-7.50 (2H, m), |
| | | | 7.70-7.85 (2H, m), 8.70-8.85 (1H, m) |
| 70 | CD₃OD | 270 | 2.75-3.20 (4H, m), 3.05-3.15 (2H, m), |
| | | | 4.05-4.25 (3H, m), 4.50-4.65 (1H, m), 4.85-5.00 (4H, m), |
| | | | 6.90-7.75 (11H, m) |
| 72 | CD₃OD | 270 | 1.70-1.77 (1H, m), 1.85-1.94 (1H, m), |
| | | | 1.98-2.04 (1H, m), 2.29-2.38 (1H, m), 2.89-2.95 (1H, m), |
| | | | 3.11-3.17 (1H, m), 3.31-3.37 (2H, m), |
| | | | 4.16-4.20 (1H, m), 4.26-4.30 (2H, m), 4.64 (1H, dd, |
| | | | J = 6.56, 8.92), 4.86 (4H, s), 6.96 (1H, d, |
| | | | J = 9.1 Hz), 7.02-7.04 (1H, m), 7.11-7.20 (3H, m), |
| | | | 7.76-7.79 (1H, m), 8.60-8.75 (1H, m) |
| 73 | CD₃OD | 270 | 1.44-1.61 (3H, m), 1.81-2.03 (3H, m), |
| | | | 3.13-3.18 (1H, m), 3.30-3.37 (1H, m), 3.77 (1H, dd, J = 8.6, |
| | | | 3.2 Hz), 4.21 (2H, s), 4.56-4.62 (1H, m), |
| | | | 4.92 (3H, br, s), 6.92-7.01 (3H, m), |
| | | | 7.20-7.25 (2H, m), 7.69-7.76 (2H, m), 8.6 1-8.68 (2H, m) |
| 74 | CD₃OD | 270 | 1.37-1.94 (7H, m), 2.80-2.83 (3H, m), |
| | | | 2.87-2.91 (1H, m), 3.30-3.32 (1H, m), 3.76-3.80 (1H, m), |
| | | | 4.22 (2H, m), 4.60-4.65 (1H, m), 4.99 (2H, s) |
| | | | 6.93-7.19 (4H, m), 7.71-7.79 (2H, m), |
| | | | 8.66-8.69 (1H, m) |
| 75 | CD₃OD | 270 | 1.08-1.10 (2H, d, J = 6.5 Hz), 1.54-1.58 (2H, m), |
| | | | 1.78-1.94 (3H, m), 2.94 (1H, s), 3.28-3.33 (3H, |
| | | | m), 3.66-3.80 (3H, m), 4.12-4.22 (3H, m), |
| | | | 4.79-4.82 (1H, m), 6.84 (1H, d, J = 2.2 Hz), |
| | | | 7.34-7.36 (3H, m), 7.45-7.58 (4H, m), 7.59-7.61 (1H, m), |
| | | | 7.73-7.77 (1H, m), 8.16 (1H, d, J = 8.2 Hz) |
| 76 | CD₃OD | 270 | 1.36-1.61 (3H, m), 1.81-1.84 (2H, m), |
| | | | 1.97-2.02 (1H, m), 2.98-3.07 (2H, m), 3.30-3.38 (3H, m), |
| | | | 3.76-3.82 (1H, m), 4.21 (1H, s), 4.75-4.80 (1H, |
| | | | m), 4.92 (5H, s), 6.94 (1H, d, J = 9.02 Hz), |
| | | | 7.19-7.38 (4H, m), 7.68 (1H, s) 7.75-7.79 (1H, m) |
| 77 | CD₃OD | 270 | 1.39-1.84 (6H, m), 1.93-1.98 (1H, m), |
| | | | 2.83-3.01 (1H, m), 3.14-3.19 (1H, m), 3.21-3.30 (1H, m), |
| | | | 3.76-3.81 (1H, m) 4.23 (1H, s) 4.62-4.65 (1H, |
| | | | m) 4.93 (5H, br, s), 6.94 (1H, d, J = 8.9 Hz), |
| | | | 7.15-7.25 (4H, m), 7.69-7.77 (1H, m), |
| | | | 8.66-8.73 (1H, m) |
| 78 | CD₃OD | 270 | 1.36-1.95 (6H, m), 3.14-3.17 (1H, m), |
| | | | 3.19-3.22 (1H, m), 3.29-3.42 (2H, m), 3.76-3.80 (1H, m), |
| | | | 4.18 (2H, d, J = 8.2 Hz), 4.75-4.79 (1H, m), |
| | | | 4.95 (5H, br, s), 6.86 (1H, dd, J = 8.2, 1.0 Hz), |
| | | | 7.30-7.39 (3H, m), 7.61-7.66 (2H, m), 7.82-7.86 (2H, |
| | | | m) |
| 80 | CD₃OD | 400 | 2.70-2.80 (1H, m), 2.85-3.00 (2H, m), |
| | | | 3.05-3.15 (1H, m), 3.25-3.40 (3H, m), 4.05-4.30 (3H, m), |
| | | | 4.40-4.60 (1H, m), 4.80-5.10 (3H, m), |
| | | | 6.90-7.20 (6H, m), 7.25-7.40 (3H, m), 7.65-7.80 (2H, m) |
| 81 | CD₃OD | 400 | 2.70-2.80 (1H, m), 2.85-2.95 (3H, m), |
| | | | 3.00-3.15 (1H, m), 3.33 (3H, s), 4.05-4.30 (4H, m), |
| | | | 4.40-4.60 (1H, m), 4.80-5.00 (2H, m), 6.85-7.20 (6H, |
| | | | m), 7.25-7.45 (3H, m), 7.65-7.80 (2H, m), |
| | | | 8.65-8.80 (1H, m) |
| 82 | CD₃OD | 400 | 0.85-1.00 (6H, m), 1.10-1.30 (1H, m), |
| | | | 1.50-1.70 (1H, m), 1.85-1.95 (1H, m), 2.47 (2H, s), |
| | | | 2.90-3.10 (2H, m), 3.25-3.35 (1H, m), 3.625 (1H, d, |
| | | | J = 4 Hz), 4.10-4.30 (2H, m), 4.67 (1H, t, J = 8 Hz), |
| | | | 4.85-5.00 (2H, m), 6.90-7.00 (2H, m), |
| | | | 7.05-7.30 (2H, m), 7.70-7.80 (2H, m), 8.70-8.80 (1H, m) |
| 83 | CD₃OD | 400 | 0.90-1.10 (6H, m), 1.15-1.25 (1H, m), |
| | | | 1.45-1.60 (1H, m), 1.85-2.00 (1H, m), 2.90-3.10 (2H, m), |
| | | | 3.30-3.40 (2H, m), 3.70-3.80 (1H, m), |
| | | | 4.05-4.25 (2H, m), 4.56 (1H, t, J = 8 Hz), 4.80-5.10 (3H, m), |
| | | | 6.90-7.25 (4H, m), 7.60-7.80 (2H, m), |
| | | | 8.70-8.80 (1H, m) |
| 84 | CD₃OD | 400 | 1.34 (3H, d, J = 7.0 Hz), 2.65 (3H, s), |
| | | | 2.86-3.01 (1H, m), 3.06-3.19 (1H, m), 3.65-3.76 (1H, m), |
| | | | 4.21-4.30 (2H, m), 4.61-4.72 (1H, m), 4.85 (4H, |
| | | | s), 6.99 (1H, d, J = 9.3 Hz), 7.03-7.07 (1H, m), |
| | | | 7.13-7.20 (2H, m), 7.77 (1H, d, J = 1.5 Hz), |
| | | | 7.82-7.85 (1H, m), 8.72-8.76 (1H, m) |
| 85 | CD₃OD | 400 | 1.50 (3H, d, J = 6.96 Hz), 2.56 (3H, s), |
| | | | 2.96-3.01 (1H, m), 3.06-3.11 (1H, m), 3.84 (1H, q, |
| | | | J = 6.9 Hz), 4.15-4.28 (2H, m), 4.60 (1H, t, |
| | | | J = 2.0 Hz), 4.86 (4H, s), 6.95-6.98 (1H, m), |
| | | | 7.04-7.07 (1H, m), 7.11-7.20 (2H, m), 7.74-7.84 (2H, |
| | | | m), 8.56-8.68 (1H, m) |
| 86 | CD₃OD | 400 | 1.45 (3H, s), 1.59 (3H, s), 2.55 (3H, s), |
| | | | 2.97-3.03 (1H, m), 3.12-3.18 (1H, m), 4.22-4.28 (2H, m), |
| | | | 4.62-4.68 (1H, m), 8.86 (3H, s), 6.99 (1H, d, |
| | | | J = 9.2 Hz), 7.05-7.07 (1H, m), 7.13-7.20 (2H, m), |
| | | | 7.76 (1H, d, J = 7.95 Hz), 7.81 (1H, dd, J = 2.0, |
| | | | 9.2 Hz), 8.31 (1H, d, J = 7.6 Hz), 8.66-8.68 (1H, |
| | | | m) |
| 87 | CD₃OD | 400 | 1.12-1.16 (4H, m), 2.63 (3H, s), 2.88-2.97 (1H, |
| | | | m), 3.12-3.20 (1H, m), 3.87 (1H, q, J = 4.0 Hz), |
| | | | 4.22 (2H, s), 4.59-4.66 (1H, m), 5.01 (4H, br s), |
| | | | 6.95-7.01 (3H, m), 7.22-7.26 (2H, m), |
| | | | 7.69-7.77 (2H, m) |
| 88 | CD₃OD | 400 | 1.49 (3H, d, J = 7.0 Hz), 2.53 (3H, s), |
| | | | 2.92-3.00 (1H, m), 3.05-3.19 (1H, m), 3.31-3.33 (4H, m), |
| | | | 3.79-3.82 (1H, m), 4.11-4.17 (1H, m), |
| | | | 4.23-4.28 (1H, m), 4.59-4.70 (1H, m), 6.94-7.01 (3H, m), |
| | | | 7.23-7.28 (2H, m), 7.68-7.71 (2H, m), |
| | | | 8.62-8.72 (1H, m) |
| 90 | CD₃OD | 270 | 1.70-2.00 (2H, m), 2.05-2.20 (1H, m), |
| | | | 2.40-2.60 (1H, m), 2.88 (3H, s), 2.90-3.40 (4H, m), |
| | | | 3.60-3.70 (1H, m), 4.00-4.40 (3H, m), 4.60-4.70 (1H, |
| | | | m), 4.80-5.00 (2H, m), 6.90-7.00 (1H, m), |
| | | | 7.10-7.20 (1H, m), 7.30-7.50 (2H, m), 7.70-7.80 (2H, |
| | | | m), 8.70-8.80 (1H, m) |
| 91 | CD₃OD | 270 | 1.50-1.70 (1H, m), 1.75-1.90 (1H, m), |
| | | | 2.00-2.20 (1H, m), 2.30-2.50 (1H, m), 2.86 (3H, s), |
| | | | 2.90-3.40 (5H, m), 3.55-3.70 (1H, m), 4.00-4.10 (1H, |
| | | | m), 4.20-4.30 (2H, m), 4.70-4.80 (1H, m), |
| | | | 4.90-5.10 (1H, m), 6.95 (1H, d, J = 9.15 Hz), |
| | | | 7.40-7.60 (4H, m), 7.70-7.80 (2H, m), 8.75-8.90 (1H, m) |
| 92 | CD₃OD | 270 | 1.70-1.90 (2H, m), 1.95-2.20 (1H, m), |
| | | | 2.40-2.60 (1H, m), 2.85 (3H, s), 3.00-3.20 (1H, m), |
| | | | 3.30-3.33 (1H, m), 3.60-3.70 (2H, m), 4.03-4.08 (2H, |
| | | | m), 4.18-4.23 (1H, m), 4.82-4.93 (5H, m), |
| | | | 6.86 (1H, d, J = 8.9 Hz), 7.33-7.36 (2H, m), |
| | | | 7.48-7.57 (4H, m), 7.74-7.78 (1H, m), 7.84-7.87 (1H, m), |
| | | | 8.16 (1H, d, J = 7.9 Hz) |
| 93 | CD₃OD | 270 | 1.61-1.68 (1H, m), 1.82-1.85 (1H, m), |
| | | | 2.03-2.11 (1H, m), 2.43 (1H, br, s) 2.88-2.95 (3H, m), |
| | | | 3.13-3.18 (2H, m), 3.65 (1H, br, s), 4.08 (1H, t, J = 8.2 Hz), |
| | | | 4.21 (2H, s), 4.65 (1H, t, J = 8.9 Hz), |
| | | | 4.93 (5H, s), 6.96 (1H, d, J = 9.7 Hz), |
| | | | 7.19-7.27 (4H, m), 7.73 (2H, d, J = 8.7 Hz) |
| 94 | CD₃OD | 270 | 1.78-1.92 (1H, m), 2.13-2.16 (1H, m), |
| | | | 2.47-2.51 (1H, m), 2.88 (4H, s), 2.98-3.01 (2H, m), |
| | | | 3.14-3.19 (1H, m), 3.64-3.69 (1H, m), 4.23 (2H, s), |
| | | | 4.70 (1H, t, J = 7.4 Hz), 4.92 (5H, s), 6.96 (1H, |
| | | | d, J = 9.2 Hz), 7.12-7.23 (2H, m), 7.74-7.82 (2H, |
| | | | m) |
| 95 | CD₃OD | 270 | 1.68-1.83 (2H, m), 2.07 (1H, s), 2.39 (1H, s), |
| | | | 2.86 (3H, s), 3.15-3.39 (2H, m), 4.07-4.24 (3H, |
| | | | m), 4.83-4.92 (7H, m), 6.85 (1H, d, J = 5.9 Hz), |
| | | | 7.34 (3H, s), 7.62 (2H, s), 7.85 (2H, s) |
| 96 | CD₃OD | 270 | 3.85 (1H, s), 4.03 (1H, s), 4.59 (1H, s), 5.01 (3H, |
| | | | s), 5.29-5.43 (2H, m), 5.79 (1H, s), |
| | | | 6.81-7.03 (7H, m), 8.96-9.09 (4H, m), 9.87 (2H, s) |
| 97 | CD₃OD | 270 | 1.8-2.0 (4H, m), 2.1-2.3 (1H, m), 2.5-2.8 (1H, |
| | | | m), 2.9-3.0 (3H, s), 3.0-3.3 (4H, m) |
| | | | 3.6-3.8 (1H, m), 4.0-4.3 (4H, m), 4.8-5.0 (1H, |
| | | | m) 7.0 (1H, d, J = 7 Hz), 7.8-7.9 (2H, m) |
| 98 | CD₃OD | 270 | 1.60-2.20 (3H, m), 2.35-2.50 (1H, m), 2.87 (3H, |
| | | | s), 2.90-3.35 (5H, m), 3.60-3.70 (1H, m), |
| | | | 4.00-4.30 (3H, m), 4.60-4.70 (1H, m), 4.90-5.10 (1H, |
| | | | m), 6.90-7.10 (3H, m), 7.15-7.30 (2H, m), |
| | | | 7.70-7.80 (2H, m), 8.60-8.80 (1H, m) |
| 99 | CD₃OD | 270 | 1.65-1.70 (1H, m), 1.86-1.88 (1H, m), |
| | | | 2.09-2.12 (1H, m), 2.39-2.43 (1H, m), 2.88 (3H, s), |
| | | | 3.14-3.19 (2H, m), 3.30 (1H, s), 3.61-3.64 (1H, m), |
| | | | 4.06 (1H, t J = 8.4 Hz), 4.22-4.23 (2H, m), |
| | | | 4.69 (1H, t, J = 6.4 Hz), 4.92 (4H, br, s), |
| | | | 6.94-7.06 (4H, m), 7.24-7.29 (1H, m), 7.70-7.77 (2H, m) |
| 100 | d⁶DMSO | 270 | 1.7-1.8 (1H, m), 1.9-2.0 (1H, m), 2.2-2.4 (1H, |
| | | | m), 2.3-2.4 (1H, m), 2.5 (1H, s), 2.6-2.8 (4H, |
| | | | m), 2.9-3.2 (1H, m), 3.3-3.6 (6H, m), |
| | | | 3.9-4.4 (2H, m), 4.6-4.8 (1H, m), 6.8-6.9 (2H, m), |
| | | | 7.0-7.1 (1H, d, J = 7 Hz), 7.2-7.3 (2H, m) 7.6-7.7 (1H, |
| | | | d, J = 7 Hz), 7.8 (1H, s), 8.0-8.2 (1H, m), |
| | | | 8.8-8.9 (1H, m), 9.0-9.1 (1H, m) |
| 101 | d⁶DMSO | 270 | 2.5 (3H, s), 3.0-3.3 (3H, m), 3.6-3.9 (3H, m), |
| | | | 3.7-4.0 (7H, m), 6.9-7.0 (1H, d, J = 9 Hz), |
| | | | 7.2-7.4 (4H, m), 7.7-7.6 (1H, d, J = 9 Hz), 7.77-7.8 (1H, |
| | | | s), 8.2 (1H, s), 8.6 (2H, s), 9.3 (1H, s) |
| 102 | d⁶DMSO | 270 | 1.0-1.1 (9H, s), 1.3-1.4 (1H, m), 1.8-2.0 (1H, m), |
| | | | 2.1-2.2 (1H, m), 2.5-2.4 (6H, m), 2.6 (3H, s), |
| | | | 3.8-4.0 (1H, m), 4.1-4.2 (2H, m), 4.6-4.8 (1H, |
| | | | m), 6.5-6.8 (2H, m), 7.1-7.2 (2H, m), |
| | | | 7.3-7.35 (2H, m), 7.4-7.45 (1H, m), 7.9 (1H, s), 8.7 (1H, |
| | | | s), 8.9 (1H, s) |
| 103 | CD₃OD | 400 | 1.66-1.74 (1H, m), 1.84-1.92 (1H, m), |
| | | | 2.08-2.18 (1H, m), 2.43-2.50 (1H, m), 2.89-2.93 (5H, m), |
| | | | 3.13-3.19 (2H, m), 3.65-3.69 (1H, m), 4.09 (1H, |
| | | | t, J = 8.0 Hz), 4.24-4.30 (2H, m), 4.70-4.72 (1H, |
| | | | m), 4.90 (3H, br, s), 7.01 (1H, d, J = 8.0 Hz), |
| | | | 7.10-7.13 (1H, m), 7.22-7.37 (3H, m), 7.72 (1H, |
| | | | s), 7.76-7.89 (1H, m) |
| 104 | CD₃OD | 400 | 1.71-1.92 (2H, m), 2.09-2.15 (1H, m), 2.30 (3H, |
| | | | s), 2.35-2.51 (1H, m), 2.89 (3H, s), |
| | | | 2.92-2.98 (1H, m), 3.13-3.19 (2H, m), 3.63-3.66 (1H, m), |
| | | | 4.07 (1H, t, J = 8.0 Hz), 4.16-4.25 (2H, m), |
| | | | 4.71 (1H, t, J = 8.0 Hz), 4.91 (4H, br, s), 6.95 (1H, d, |
| | | | J = 12.0 Hz), 7.04-7.13 (4H, m), 7.65 (1H, s), |
| | | | 7.68-7.75 (1H, m) |
| 105 | CD₃OD | 400 | 1.63-1.70 (1H, m), 1.83-1.88 (1H, m), |
| | | | 2.06-2.16 (1H, m), 2.27 (3H, m), 2.37-2.46 (1H, m), |
| | | | 2.87-2.91 (3H, m), 3.12-3.19 (2H, m), 3.31-3.32 (1H, |
| | | | m), 3.62-3.68 (1H, m), 4.07 (1H, t, J = 8.0 Hz), |
| | | | 4.21-4.30 (2H, m), 4.67-4.70 (1H, m), 4.90 (4H, |
| | | | br, s), 6.89-7.05 (3H, m), 7.10-7.20 (1H, m), |
| | | | 7.64-7.79 (2H, m), 8.71-8.76 (1H, m) |
| 106 | CD₃OD | 400 | 1.64-1.73 (1H, m), 1.82-1.89 (1H, m), |
| | | | 2.04-2.15 (1H, m), 2.29 (3H, s), 2.37-2.47 (1H, m), |
| | | | 2.85-2.91 (4H, m), 3.10-3.19 (2H, m), 3.63-3.69 (1H, |
| | | | m), 4.08 (1H,, t J = 8.0 Hz), 4.18-4.27 (2H, m), |
| | | | 4.65-4.67 (1H, m), 4.90 (4H, br, s), |
| | | | 6.89-6.94 (1H, m), 7.06-7.30 (4H, m, 7.65-7.74 (2H, m) |
| 107 | CD₃OD | 400 | 1.67-1.76 (1H, m), 1.82-1.93 (1H, m), |
| | | | 2.07-2.17 (1H, m), 2.41-2.49 (1H, m), 2.86-2.89 (4H, m), |
| | | | 3.07-3.20 (2H, m), 3.64-3.68 (1H, m), |
| | | | 3.69-3.75 (3H, m), 4.09 (1H, t, J = 8.0 Hz), 4.18-4.29 (2H, |
| | | | m), 4.61-4.65 (1H, m), 4.92 (4H, br, s), |
| | | | 6.81 (2H, d, J = 8.0 Hz), 6.94-6.96 (1H, m), 7.13 (2H, |
| | | | d, J = 8.0 Hz), 7.71-7.77 (2H, m) |
| 108 | CD₃OD | 400 | 1.70-1.76 (1H, m), 1.83-1.90 (1H, m), |
| | | | 2.09-2.16 (1H, m), 2.40-2.48 (1H, m), 2.88 (3H, s), |
| | | | 2.98-3.03 (1H, m), 3.13-3.25 (2H, m), 3.63-3.68 (1H, |
| | | | m), 4.08 (1H, t, J = 8.0 Hz), 4.24-4.29 (2H, m), |
| | | | 4.74-4.82 (1H, m), 4.92 (4H, br, s), |
| | | | 7.03-7.08 (1H, m), 7.10-7.19 (2H, m), 7.23-7.33 (2H, m), |
| | | | 7.70-7.71 (1H, m), 7.77-7.84 (1H, m) |
| 109 | CD₃OD | 400 | 1.69-1.78 (1H, m), 1.81-1.92 (1H, m), |
| | | | 2.04-2.17 (1H, m), 2.42-2.51 (1H, m), 2.89 (3H, s), |
| | | | 3.05-3.20 (2H, m), 3.32-3.37 (1H, m), 3.62-3.68 (1H, |
| | | | m), 4.10 (1H, t, J = 8.0 Hz), 4.23-4.36 (2H, m), |
| | | | 4.82-4.85 (5H, m), 6.97 (1H, d, J = 12.0 Hz), |
| | | | 7.19-7.29 (3H, m), 7.32-7.40 (1H, m), 7.71 (1H, |
| | | | s), 7.79 (1H, dd, J = 8.0, 4.0 Hz) |
| 110 | CD₃OD | 400 | 1.76-1.83 (1H, m), 1.87-1.94 (1H, m), |
| | | | 2.11-2.19 (1H, m), 2.46-2.55 (1H, m), 2.89 (3H, s), |
| | | | 3.04-3.08 (1H, m), 3.10-3.19 (1H, m), 3.27-3.33 (1H, |
| | | | m), 3.64-3.70 (1H, m), 4.10 (1H, t, J = 8.0 Hz), |
| | | | 4.19-4.27 (2H, m), 4.81 (1H, t, J = 8.0 Hz), |
| | | | 4.91 (4H, br s), 6.98 (1H, d, J = 12.0 Hz), |
| | | | 7.18-7.28 (2H, m), 7.43 (1H, s), 7.71-7.73 (1H, m), |
| | | | 7.77-7.80 (1H, m) |
| 111 | CD₃OD | 400 | 1.74-1.81 (1H, m), 1.88-1.94 (1H, m), |
| | | | 2.11-2.18 (1H, m), 2.45-2.52 (1H, m), 2.89 (4H, s), |
| | | | 3.11-3.21 (2H, m), 3.33-3.42 (1H, m), 3.64-3.70 (1H, |
| | | | m), 4.11-4.30 (5H, m), 4.77 (2H, t, J = 8.0 Hz), |
| | | | 6.97 (1H, d, J = 8.0 Hz), 7.40-7.48 (2H, m), |
| | | | 7.51-7.61 (1H, m), 7.66-7.71 (2H, m), |
| | | | 7.78-7.83 (1H, m) |
| 112 | CD₃OD | 400 | 1.11 (2H, t, J = 4.0 Hz), 1.74-1.87 (1H, m), |
| | | | 1.90-1.95 (2H, m), 2.46-2.49 (1H, m), 2.88-2.90 (5H, |
| | | | m), 3.11-3.18 (3H, m), 3.22-3.30 (3H, m), |
| | | | 3.50-3.55 (1H, m), 3.65-3.69 (1H, m), 4.11-4.16 (1H, |
| | | | m), 4.22-4.37 (2H, m), 6.99 (2H, d, J = 8.0 Hz), |
| | | | 7.77 (1H, s), 7.87 (1H, dd, J = 8.0, 4.0 Hz), |
| | | | 7.93-8.01 (2H, m), 8.75-8.77 (2H, br, s), 8.87 (1H, t, J = 4.0 Hz) |
| 113 | CD₃OD | 400 | 1.54-1.59 (1H, m) 1.64-1.74 (1H, m) |
| | | | 1.92-1.99 (1H, m) 2.25-2.30 (1H, M) 2.72-2.76 (4H, m) |
| | | | 3.01-3.07 (3H, m) 3.18-3.23 (2H, m) |
| | | | 3.49-3.55 (1H, m) 3.94-4.12 (5H, m) 4.62 (1H, t, J = 4.0 Hz) |
| | | | 6.67 (1H, d, J = 8.0 Hz) 6.81-6.94 (1H, m) |
| | | | 6.99-7.07 (2H, m) 7.23 (1H, d, J = 8.0 Hz) |
| | | | 7.36-7.53 (3H, m) |
| 114 | CD₃OD | 400 | 1.69-1.80 (2H, m), 2.04-2.11 (1H, m), |
| | | | 2.43-2.48 (1H, m), 2.88 (3H, s), 3.11-3.18 (1H, m), |
| | | | 3.31-3.33 (1H, m), 3.46-3.51 (1H, m), 3.57-3.60 (1H, |
| | | | m), 4.13 (1H, t, J = 8.0 Hz), 4.22-4.31 (2H, m), |
| | | | 4.92 (4H, br s), 5.11 (1H, t, J = 8.0 Hz), |
| | | | 6.85 (1H, d, J = 8.0 Hz), 7.71-7.81 (4H, m), 7.94 (1H, |
| | | | m), 8.07-8.19 (2H, m), 8.60-8.64 (1H, m) |
| 115 | CD₃OD | 270 | 0.9-1.0 (3H, m), 1.1-1.4 (5H, m), 1.5-1.8 (8H, |
| | | | m), 2.0-2.3 (3H, m) 2.6-2.7 (1H, m) 2.9 (3H, s), |
| | | | 3.1-3.3 (1H, m), 3.7-3.8 (1H, m) 4.0-4.5 (4H, |
| | | | m), 7.0-7.1 (1H, d, J = 7 Hz), 7.7-7.8 (2H, s) |
| | | | 7.9-8.0 (1H, m) |
| 116 | CD₃OD | 400 | 1.90-2.25 (3H, m), 2.50-2.65 (1H, m), 2.82 (3H, |
| | | | m), 2.90-3.25 (3H, m), 3.30-3.40 (2H, m), |
| | | | 3.65-3.80 (1H, m), 4.00-4.30 (3H, m), 4.55-4.70 (1H, |
| | | | m), 4.85-5.00 (1H, m), 6.90-7.25 (4H, m), |
| | | | 7.65-7.80 (2H, m), 8.65-8.80 (1H, m) |
| 117 | CD₃OD | 270 | 1.24-1.29 (7H, m), 1.76-1.83 (2H, m), |
| | | | 2.00-2.05 (1H, m), 2.40 (1H, br, s), 2.89-2.94 (1H, m), |
| | | | 3.08-3.15 (2H, m), 3.49-3.64 (2H, m), |
| | | | 4.20-4.27 (3H, m), 4.63 (1H, t, J = 8.6 Hz), 4.98 (3H, br, |
| | | | s), 6.95 (1H, d, J = 9.9 Hz), 7.16 (1H, d, J = 6.6 Hz), |
| | | | 7.38-7.41 (2H, m), 7.72-7.75 (2H, m) |
| 118 | CD₃OD | 270 | 1.20-1.35 (6H, m), 1.60-1.80 (2H, m), |
| | | | 1.90-2.10 (1H, m), 2.20-2.40 (1H, m), 3.15-3.70 (8H, m), |
| | | | 4.00-4.30 (3H, m), 4.75-4.95 (1H, m), 6.86 (1H, |
| | | | d, J = 9.40 Hz), 7.30-7.90 (8H, m), 8.15-8.20 (1H, |
| | | | m), 8.55-8.70 (1H, m) |
| 119 | CD₃OD | 270 | 1.20-1.30 (6H, m), 1.75-1.85 (2H, m), |
| | | | 2.03-2.15 (2H, m), 2.25-2.50 (1H, m), 2.88-2.96 (2H, m), |
| | | | 3.07-3.14 (2H, m), 3.49-3.70 (2H, m), |
| | | | 4.17-4.22 (3H, m), 4.61-4.65 (1H, m), 4.90-5.10 (1H, m), |
| | | | 6.92-7.02 (3H, m), 7.20-7.25 (2H, m), |
| | | | 7.70-7.74 (2H, m), 8.65-8.80 (1H, m) |
| 120 | CD₃OD | 400 | 1.26-1.30 (6H, m), 1.72-1.84 (2H, m), |
| | | | 2.00-2.09 (1H, m), 2.33-2.41 (1H, m), 2.92-2.98 (1H, m), |
| | | | 3.14-3.27 (2H, m), 3.50-3.63 (2H, m), |
| | | | 4.23-4.36 (3H, m), 4.67 (1H, t, J = 8.0 Hz), 4.93 (4H, br, |
| | | | s), 6.75 (1H, d, J = 8.0 Hz), 7.18-7.35 (4H, m), |
| | | | 7.71-7.78 (2H, m) |
| 121 | CD₃OD | 400 | 1.26-1.30 (6H, m), 1.74-1.83 (2H, m), |
| | | | 2.00-2.08 (1H, m), 2.36-2.40 (3H, m), 2.95-3.00 (1H, m), |
| | | | 3.15-3.26 (2H, m), 3.50-3.64 (2H, m), |
| | | | 4.14-4.25 (3H, m), 4.68 (1h, t, J = 8.0 Hz), 4.91 (5H, br, s), |
| | | | 6.72 (1H, d, J = 8.0 Hz), 7.04-7.16 (4H, m), |
| | | | 7.64-7.79 (2H, m) |
| 122 | CD₃OD | 400 | 1.14-1.19 (6H, m), 1.57-1.69 (2H, m), |
| | | | 1.89-1.94 (1H, m), 2.18 (3H, s), 2.76-2.82 (1H, m), |
| | | | 2.99-3.12 (2H, m), 4.11-4.13 (3H, m), 4.53-4.57 (1H, |
| | | | m), 4.92 (6H, br, s) 6.84 (1H, d, J = 8.0 Hz), |
| | | | 6.90-6.95 (3H, m), 6.99-7.08 (1H, m), |
| | | | 7.57-7.62 (2H, m), 8.75 (1H, d, J = 8.0 Hz) |
| 123 | CD₃OD | 400 | 1.11-1.30 (6H, m), 1.71-1.79 (2H, m), |
| | | | 2.01-2.05 (1H, m), 2.28 (3H, s), 2.87-2.93 (1H, m), |
| | | | 3.08-3.13 (1H, m), 3.19-3.25 (1H, m), 3.31-3.32 (1H, |
| | | | m), 3.50-3.65 (2H, m), 4.14-4.35 (3H, m), |
| | | | 4.63 (1H, t, J = 8.0 Hz), 4.96 (4H, br, s), 6.95 (1H, d, |
| | | | J = 8.0 Hz), 7.05-7.11 (4H, m), 7.71-7.75 (2H, |
| | | | m) |
| 124 | CD₃OD | 400 | 1.26-1.30 (6H, m), 1.73-1.84 (1H, m), |
| | | | 2.00-2.08 (1H, m), 2.34-2.39 (1H, m), 2.86-2.91 (1H, m), |
| | | | 3.05-3.10 (1H, m), 3.19-3.26 (1H, m), |
| | | | 3.50-3.63 (2H, m), 3.75 (3H, s), 4.20-4.26 (3H, m), |
| | | | 4.60 (1H, t, J = 8.0 Hz), 4.94 (4H, br, s), |
| | | | 6.80-6.82 (2H, m), 6.94 (1H, dd, J = 8.0, 4.0 Hz), |
| | | | 7.13 (2H, d, J = 8.0 Hz), 7.70-7.72 (2H, m) |
| 125 | CD₃OD | 400 | 1.25-1.30 (6H, m), 1.73-1.84 (2H, m), |
| | | | 2.01-2.09 (1H, m), 2.34-2.43 (1H, m), 3.00-3.06 (1H, m), |
| | | | 3.19-3.26 (2H, m), 3.50-3.62 (2H, m), |
| | | | 4.22-4.36 (3H, m), 4.71-4.76 (1H, m), 4.98 (4H, br, s), |
| | | | 7.01 (1H, d, J = 8.0 Hz), 7.04-7.09 (2H, m), |
| | | | 7.23-7.36 (2H, m), 7.70 (1H, s), 7.77 (1H, dd, J = 8.0, |
| | | | 4.0 Hz) |
| 126 | CD₃OD | 400 | 1.26-1.30 (6H, m), 1.76-1.84 (2H, m), |
| | | | 2.01-2.10 (1H, m), 2.37-2.42 (1H, m), 3.09-3.14 (1H, m), |
| | | | 3.20-3.33 (2H, m), 3.50-3.63 (2H, m), |
| | | | 4.21-4.32 (3H, m), 4.80 (1H, t, J = 8.0 Hz), 4.88 (4H, br, s) |
| | | | 6.96 (1H, d, J = 8.0 Hz), 7.16-7.28 (2H, m), |
| | | | 7.37-7.47 (1H, m), 7.70 (1H, s) 7.75-7.83 (1H, |
| | | | m), 8.69 (1H, t, J = 8.0 Hz) |
| 127 | CD₃OD | 400 | 1.07-1.17 (6H, m), 1.66-1.73 (2H, m), |
| | | | 1.93-2.00 (1H, m), 2.30-2.35 (1H, m), 2.96-3.04 (1H, m), |
| | | | 3.14-3.21 (2H, m), 3.40-3.52 (2H, m), |
| | | | 4.04-4.24 (3H, m), 4.65 (1H, t, J = 8.0 Hz), 4.88 (4H, br, |
| | | | s), 6.90 (1H, d, J = 12.0 Hz), 7.05-7.18 (2H, m), |
| | | | 7.31 (1H, s), 7.62-7.73 (2H, m) |
| 128 | CD₃OD | 400 | 1.25-1.30 (6H, m), 1.76-1.89 (2H, m), |
| | | | 2.04-2.11 (1H, m), 2.37-2.44 (1H, m), 3.14-3.26 (2H, m), |
| | | | 3.31-3.38 (1H, m), 3.50-3.65 (2H, m), |
| | | | 4.15-4.37 (3H, m), 4.75 (1H, t, J = 8.0 Hz), 4.99 (4H, br, |
| | | | s), 6.96 (1H, d, J = 8.0 Hz), 7.40-7.46 (2H, m), |
| | | | 7.51-7.55 (1H, m), 7.67-7.71 (2H, m), 7.78 (1H, |
| | | | dd, J = 8.0, 4.0 Hz) |
| 129 | CD₃OD | 400 | 1.26-1.30 (5H, m), 1.78-1.87 (1H, m), |
| | | | 2.04-2.09 (1H, m), 2.39-2.44 (1H, m), 3.24-3.33 (3H, m), |
| | | | 3.44-3.63 (3H, m), 4.24-4.35 (3H, m), |
| | | | 4.82-4.89 (7H, m), 6.99 (1H, d, J = 12.0 Hz), 7.77 (1H, s), |
| | | | 7.84-7.89 (2H, m) |
| 130 | CD₃OD | 400 | 1.22-1.26 (6H, m), 1.67-1.75 (2H, m), |
| | | | 1.92-2.01 (1H, m), 2.26-2.32 (1H, m), 3.11-3.17 (2H, m), |
| | | | 3.30-3.33 (1H, m), 3.44-3.58 (2H, m), |
| | | | 4.09-4.14 (2H, m), 4.17-4.24 (1H, m), 4.71 (1H, t, J = 8.0 Hz), |
| | | | 4.93 (5H, br s), 6.85 (1H, d, J = 12.0 Hz), |
| | | | 6.92-7.01 (1H, m), 7.07-7.13 (2H, m), 7.34 (1H, |
| | | | d, J = 8.0 Hz), 7.47-7.61 (3H, m) |
| 131 | CD₃OD | 400 | 1.27 (3H, t, J = 7.3 Hz), 1.72-1.81 (1H, m), |
| | | | 1.85-1.96 (1H, m), 2.06-2.19 (1H, m), 2.42-2.49 (1H, |
| | | | m), 2.89-2.96 (2H, m), 3.09-3.29 (4H, m), |
| | | | 3.65-3.73 (1H, m), 4.10 (1H, t, J = 8.4 Hz), 4.16 (1H, d, |
| | | | J = 15.1 Hz), 4.31 (1H, d, J = 15.1 Hz), 4.65 (1H, |
| | | | dd, J = 7.0, 8.7 Hz), 4.86 (2H, s), 6.96 (1H, d, |
| | | | J = 9.1 Hz), 7.01-7.04 (1H, m), 7.10-7.24 (2H, m), |
| | | | 7.74-7.78 (2H, m), 8.65-8.75 (1H, m) |
| 132 | CD₃OD | 400 | 0.95 (3H, t, J = 7.3 Hz), 1.55-1.61 (3H, m), |
| | | | 1.62-1.68 (1H, m), 1.70-1.87 (1H, m), 2.00-2.12 (1H, |
| | | | m), 2.34-2.44 (1H, m), 2.89-3.33 (6H, m), |
| | | | 3.58-3.71 (2H, m), 3.97 (1H, dd, J = 7.2, 9.0 Hz), |
| | | | 4.21 (2H, s), 4.64 (1H, dd, J = 6.4, 9.0 Hz), 4.86 (1H, s), |
| | | | 6.72 (1H, d, J = 8.7 Hz), 7.00-7.04 (1H, m), |
| | | | 7.12-7.19 (2H, m), 7.54 (1H, dd, J = 2.2, 8.9 Hz), |
| | | | 7.80 (1H, d, J = 1.7 Hz) |
| 133 | CD₃OD | 400 | 0.97 (3H, d, J = 6.6 Hz), 1.03 (3H, d, J = 6.6 Hz), |
| | | | 1.71-1.79 (1H, m), 1.90-2.04 (2H, m), |
| | | | 2.07-2.23 (1H, m), 2.39-2.48 (1H, m), 2.85-3.04 (4H, m), |
| | | | 3.11-3.21 (2H, m), 3.74-3.79 (1H, m), 4.13 (1H, |
| | | | t, J = 8.5 Hz), 4.18-4.34 (2H, m), 4.65 (1H, dd, |
| | | | J = 6.7, 8.85 Hz), 4.87 (2H, s), 6.97 (1H, d, |
| | | | J = 9.4 Hz), 7.01-7.05 (1H, m), 7.11-7.20 (2H, m), |
| | | | 7.76 (1H, d, J = 10.1 Hz), 7.78-7.81 (1H, m), |
| | | | 8.73-8.76 (1H, m) |
| 135 | CD₃OD | 400 | 1.70-1.80 (1H, m), 1.81-2.00 (1H, m), |
| | | | 2.05-2.20 (1H, m), 2.30-2.50 (1H, m), 2.80-2.95 (1H, m), |
| | | | 3.10-3.40 (6H, m), 3.70-3.85 (1H, m), |
| | | | 3.95-4.00 (1H, m), 4.05-4.15 (1H, m), 4.20-4.50 (3H, m), |
| | | | 4.60-4.70 (1H, m), 6.90-7.10 (1H, m), |
| | | | 7.11-7.30 (1H, m), 7.65-7.75 (1H, m), 7.80-7.90 (1H, m), |
| | | | 8.65-8.80 (1H, m) |
| 136 | CD₃OD | 400 | 1.27 (3H, t, J = 7.2 Hz), 1.71-1.80 (1H, m), |
| | | | 1.83-1.94 (1H, m), 2.07-2.17 (1H, m), 2.37-2.44 (1H, m), |
| | | | 2.89-2.98 (2H, m), 3.07-3.32 (6H, m), |
| | | | 3.66-3.72 (1H, m), 4.09 (1H, t, J = 8.4 Hz), |
| | | | 4.15-4.36 (2H, m), 4.63-4.67 (1H, m), 6.95-7.09 (3H, m), |
| | | | 7.22-7.30 (2H, m), 7.72-7.86 (2H, m), |
| | | | 8.69-8.72 (1H, m) |
| 137 | CD₃OD | 400 | 1.25-1.28 (3H, m), 1.66-1.70 (1H, m), |
| | | | 1.82-1.87 (1H, m), 2.04-2.12 (1H, m), 2.29 (3H, s), |
| | | | 2.36-2.41 (1H, m), 2.86-2.92 (1H, m), 3.10-3.27 (5H, |
| | | | m), 3.31-3.33 (1H, m), 3.65-3.71 (1H, m), |
| | | | 4.10 (1H, t, J = 8.0 Hz), 4.18-4.28 (2H, m), |
| | | | 4.65-4.69 (1H, m), 4.87 (2H, br, s), 6.88 (1H, d, J = 12.0 Hz), |
| | | | 6.96-7.06 (3H, m), 7.10-7.17 (1H, m), |
| | | | 7.61-7.73 (2H, m) |
| 138 | CD₃OD | 400 | 1.25-1.28 (3H, m), 1.69-1.74 (1H, m), |
| | | | 1.83-1.97 (1H, m), 2.08-2.14 (1H, m), 2.40-2.45 (1H, m), |
| | | | 2.91-2.96 (1H, m), 3.11-3.33 (5H, m), |
| | | | 3.616-3.72 (1H, m), 4.12 (1H, t, J = 8.0 Hz), |
| | | | 4.19-4.28 (2H, m), 4.69 (1H, t, J = 4.0 Hz), 4.89 (3H, br, |
| | | | s), 6.97 (1H, d, J = 8.0 Hz), 7.17-7.19 (1H, m), |
| | | | 7.22-7.29 (3H, m), 7.72 (1H, s), 7.77 (1H, dd, J = 8.0, |
| | | | 4.0 Hz) |
| 139 | CD₃OD | 400 | 0.97 (3H, t, J = 7.4 Hz), 1.65-1.77 (4H, m), |
| | | | 1.85-1.92 (1H, m), 2.07-2.15 (1H, m), 2.37-2.44 (1H, |
| | | | m), 2.90-2.96 (2H, m), 3.03-3.20 (4H, m), |
| | | | 3.68-3.73 (1H, m), 4.10 (1H, t, J = 8.2 Hz), |
| | | | 4.20-4.29 (2H, m), 4.65 (1H, dd, J = 6.98, 8.61 Hz), |
| | | | 4.86 (1H, s), 6.95-7.02 (3H, m), 7.22-7.25 (2H, m), |
| | | | 7.72-7.76 (2H, m), 8.69-8.72 (1H, m) |
| 140 | CD₃OD | 400 | 1.65-1.70 (1H, m), 1.85-2.00 (1H, m), |
| | | | 2.10-2.25 (1H, m), 2.35-2.50 (1H, m), 2.80-2.90 (1H, m), |
| | | | 3.00-3.10 (1H, m), 3.25-3.40 (4H, m), |
| | | | 3.45-3.60 (1H, m), 4.10-4.40 (5H, m), 4.50-4.60 (1H, m), |
| | | | 6.90-7.00 (1H, m), 7.05-7.20 (2H, m), |
| | | | 7.40-7.65 (5H, m), 7.70-7.80 (2H, m), 8.60-8.80 (1H, m) |
| 141 | CD₃OD | 270 | 1.69-2.03 (4H, m), 2.89-2.94 (1H, m), |
| | | | 3.12-3.32 (4H, m), 4.17-4.28 (5H, m), 4.30-4.35 (1H, m), |
| | | | 4.96 (3H, br, s), 6.95 (1H, d, J = 8.9 Hz), |
| | | | 7.15-7.19 (2H, m), 7.33-7.42 (6H, m), |
| | | | 7.73-7.76 (2H, m) |
| 143 | CD₃OD | 400 | 1.71-1.76 (1H, m), 1.89-1.98 (1H, m), |
| | | | 2.12-2.18 (1H, m), 2.43-2.50 (1H, m), 2.84-2.90 (1H, m), |
| | | | 3.02-3.07 (1H, m), 3.31-3.33 (1H, m), |
| | | | 3.52-3.57 (1H, m), 4.11-4.18 (2H, m), 4.27-4.38 (3H, m), |
| | | | 4.56 (1H, dd, J = 7.2, 8.3 Hz), 4.86 (3H, s), |
| | | | 6.96-6.99 (2H, m), 7.05-7.17 (2H, m), 7.39-7.48 (3H, |
| | | | m), 7.53 (1H, s), 7.75-7.77 (2H, m), |
| | | | 8.63-8.66 (1H, m) |
| 144 | CD₃OD | 400 | 1.50-2.00 (6H, m), 2.78 (3H, s), 2.85-2.95 (1H, |
| | | | m), 3.05-3.15 (2H, m), 3.25-3.35 (2H, m), |
| | | | 3.40-3.50 (1H, m), 3.55-3.70 (1H, m), 4.10-4.30 (2H, |
| | | | m), 4.50-4.60 (1H, m), 4.80-5.00 (1H, m), |
| | | | 6.90-7.20 (4H, m), 7.65-7.80 (2H, m), |
| | | | 8.70-8.80 (1H, m) |
| 145 | CD₃OD | 400 | 1.52-1.63 (2H, m), 1.72-1.93 (4H, m), 2.78 (3H, |
| | | | s), 2.86-2.97 (2H, m), 3.08-3.14 (2H, m), |
| | | | 3.46-3.54 (1H, m), 3.65-3.69 (1H, m), 4.16 (1H, d, |
| | | | J = 15.1 Hz), 4.25 (1H, d, J = 15.1 Hz), 4.59 (1H, |
| | | | dd, J = 7.3, 8.4 Hz), 4.86 (3H, s), 6.92-7.02 (3H, |
| | | | m), 7.21-7.24 (2H, m), 7.69-7.71 (2H, m) |
| 146 | CD₃OD | 400 | 1.32 (6H, dd, J = 6.66, 25.1), 1.56-1.61 (1H, m), |
| | | | 1.68-1.97 (4H, m), 2.90-2.98 (2H, m), |
| | | | 3.00-3.14 (1H, m), 3.44 (1H, d, J = 13.05 Hz), |
| | | | 3.54-3.74 (2H, m), 3.94-3.97 (1H, m), 4.14-4.30 (2H, m), |
| | | | 4.55-4.61 (1H, m), 4.86 (2H, m), 6.98 (1H, d, |
| | | | J = 9.2 Hz), 7.06-7.12 (1H, m), 7.13-7.21 (2H, m), |
| | | | 7.76-7.81 (2H, m), 8.69-8.72 (1H, m), |
| | | | 8.86-8.92 (1H, m) |
| 147 | CD₃OD | 270 | 1.25-1.40 (6H, m), 1.44-1.92 (5H, m), |
| | | | 2.80-2.97 (2H, m), 3.30-3.36 (2H, m), 3.74-3.90 (2H, m), |
| | | | 4.11-4.28 (2H, m), 4.55-4.60 (1H, m), 4.90 (5H, |
| | | | br, s), 6.92-7.00 (2H, m), 7.19-7.24 (2H, m), |
| | | | 7.66-7.69 (2H, m), 8.67-8.69 (1H, m) |
| 148 | CD₃OD | 400 | 1.82-1.88 (1H, m), 2.62-2.79 (1H, m), |
| | | | 2.91 (3H, s), 2.94-3.00 (1H, m), 3.08-3.18 (1H, m), |
| | | | 3.28-3.32 (1H, m), 3.57 (1H, d, J = 11.6 Hz), |
| | | | 4.07-4.34 (3H, m), 4.49 (1H, s, br), 4.62-4.69 (1H, m), |
| | | | 4.92 (3H, s), 6.96 (1H, d, J = 9.1 Hz), |
| | | | 7.00-7.03 (1H, m), 7.11-7.24 (2H, m), 7.73-7.81 (2H, m), |
| | | | 8.69-8.11 (1H, m) |
| 149 | CD₃OD | 400 | 2.87-2.98 (1H, m), 3.00 (3H, s), 3.04-3.15 (2H, |
| | | | m), 3.20-3.26 (1H, m), 4.16-4.18 (2H, m), |
| | | | 4.20-4.34 (1H, m), 4.43 (1H, d, J = 15.4 Hz), 4.57 (1H, |
| | | | d, J = 15.5 Hz), 4.65 (1H, dd, J = 7.05, 8.61 Hz), |
| | | | 4.86 (4H, s), 6.97 (1H, d, J = 9.1 Hz), |
| | | | 7.04-7.07 (1H, m), 7.12-7.36 (4H, m), 7.74-7.79 (2H, m), |
| | | | 8.72-8.75 (2H, m) |
| 150 | CD₃OD | 270 | 1.28-1.30 (6H, m), 2.75 (3H, s), 2.89-2.97 (2H, |
| | | | m), 3.07-3.30 (2H, m), 3.40-3.45 (1H, m), |
| | | | 3.83-3.95 (2H, m), 4.12-4.27 (2H, m), 4.65 (1H, t, J = 6.9 Hz), |
| | | | 4.95 (2H, br, s), 6.93-6.96 (1H, m), |
| | | | 7.17 (1H, dd, J = 8.2, 1.5 Hz), 7.40 (2H, d, J = 8.2 Hz), |
| | | | 7.71 (2H, br, s) |
| 151 | CD₃OD | 270 | 1.25-1.50 (9H, m), 2.59 (3H, s), 2.85-3.15 (2H, |
| | | | m), 3.25-3.35 (2H, m), 3.75-3.95 (2H, m), |
| | | | 4.05-4.35 (2H, m), 4.55-4.70 (1H, m), 4.90-5.10 (2H, |
| | | | m), 6.85-6.95 (1H, m), 7.10-7.20 (1H, m), |
| | | | 7.35-7.45 (2H, m), 7.60-7.75 (2H, m) |
| 152 | CD₃OD | 270 | 2.79 (3H, s), 2.85-3.30 (3H, m), 3.80-3.95 (2H, |
| | | | m), 4.10-4.30 (4H, m), 4.60-4.70 (1H, m), |
| | | | 4.90-5.10 (1H, m), 6.90-7.00 (1H, m), 7.10-7.20 (1H, |
| | | | m), 7.30-7.50 (8H, m), 7.60-7.80 (2H, m), |
| | | | 8.60-8.80 (1H, m) |
| 153 | CD₃OD | 270 | 2.86 (6H, s), 2.90-310 (2H, m), 3.25-3.40 (2H, |
| | | | m), 3.85-4.30 (2H, m), 4.55-4.70 (1H, m), |
| | | | 4.80-5.00 (1H, m), 6.90-7.00 (1H, m), 7.10-7.20 (1H, |
| | | | m), 7.30-7.50 (2H, m), 7.60-7.80 (2H, m), |
| | | | 8.65-8.80 (1H, m) |
| 154 | CD₃OD | 270 | 0.98 (6H, d, J = 6.43 Hz), 1.95-2.15 (1H, m), |
| | | | 2.70-3.15 (7H, m), 3.25-3.40 (1H, m), 3.80-4.05 (2H, |
| | | | m), 4.10-4.35 (2H, m), 4.60-4.70 (1H, m), |
| | | | 4.90-5.20 (2H, m), 6.90-7.00 (1H, m), 7.10-7.20 (1H, |
| | | | m), 7.35-7.50 (2H, m), 7.65-7.80 (2H, m), |
| | | | 8.70-8.80 (1H, m) |
| 155 | CD₃OD | 270 | 0.75-0.90 (7H, m), 1.25-1.40 (7H, m), |
| | | | 1.60-1.80 (1H, m), 2.60 (3H, s), 2.65-3.05 (5H, m), |
| | | | 3.60-3.95 (2H, m), 4.10-4.40 (2H, m), 4.60-4.70 (1H, |
| | | | m), 6.90-7.10 (1H, m), 7.15-7.25 (1H, m), |
| | | | 7.40-7.50 (2H, m), 7.60-7.80 (2H, m), 8.70-8.90 (1H, |
| | | | m) |
| 156 | CD₃OD | 270 | 1.20-1.40 (6H, m), 2.75 (3H, s), 2.85-3.15 (2H, |
| | | | m), 3.25-3.35 (1H, m), 3.40-4.00 (1H, m), |
| | | | 4.10-4.30 (2H, m), 4.55-4.70 (1H, m), 4.80-5.10 (4H, |
| | | | m), 6.90-7.40 (4H, m) 7.65-7.90 (2H, m), |
| | | | 8.70-8.80 (1H, m) |
| 157 | CD₃OD | 400 | 1.16 (3H, d, J = 6.1 Hz), 1.27 (6H, d, J = 6.5 Hz), |
| | | | 1.34 (3H, d, J = 6.1 Hz), 2.90-3.16 (2H, m), |
| | | | 3.65-3.73 (2H, m), 3.81-3.97 (2H, m), 4.17-4.28 (2H, |
| | | | m), 4.59-4.69 (1H, m), 6.95-6.98 (1H, m), |
| | | | 7.0-7.05 (1H, m), 7.12-7.19 (2H, m), 7.73-7.79 (2H, |
| | | | m), 8.73-8.76 (1H, m). |
| 158 | CD₃OD | 400 | 0.95 (6H, t, J = 7.3 Hz), 1.65 (4H, br s), |
| | | | 2.89-2.95 (1H, m), 3.03-3.16 (6H, m), 3.88-4.03 (2H, m), |
| | | | 4.22-4.24 (2H, m), 4.63-4.67 (1H, m), 6.97 (1H, |
| | | | d, J = 9.2 Hz), 7.03-7.06 (1H, m), 7.12-7.19 (2H, |
| | | | m), 7.73 (1H, d, J = 1.2 Hz), 7.78 (1H, dd, J = 9.2, |
| | | | 2.1 Hz), 8.74 (1H, t, J = 5.9 Hz). |
| 159 | CD₃OD | 400 | 1.00 (12H, dd, J = 6.6, 3.0 Hz), 2.00-2.04 (2H, m), |
| | | | 2.89-3.18 (6H, m), 3.96-4.12 (2H, m), |
| | | | 4.24-4.25 (2H, m), 4.66-4.71 (1H, m), 6.97 (1H, d, J = 9.1 Hz), |
| | | | 7.04-7.07 (1H, m), 7.12-7.19 (2H, m), |
| | | | 7.74 (1H, d, J = 1.3 Hz), 7.79 (1H, dd, J = 9.2, |
| | | | 2.1 Hz). |
| 160 | CD₃OD | 400 | 2.91-3.10 (4H, m), 3.21-3.33 (3H, m), |
| | | | 3.78-3.89 (2H, m), 4.12-4.28 (2H, m), 4.60 (1H, s, J = 7.6 Hz), |
| | | | 6.95 (1H, d, J = 9.1 Hz), 7.00-7.03 (1H, |
| | | | m), 7.09-7.16 (2H, m), 7.25-7.37 (5H, m), |
| | | | 7.71-7.75 (2H, m), 8.69 (1H, t, J = 5.8 Hz). |
| 161 | CD₃OD | 400 | 2.92-3.13 (7H, m), 3.32-3.38 (2H, m), |
| | | | 3.98-4.09 (2H, m), 4.17-4.27 (2H, m), 4.61-4.65 (1H, m), |
| | | | 6.95 (1H, d, J = 9.1 Hz), 7.01-7.04 (1H, m), |
| | | | 7.08-7.17 (2H, m), 7.24-7.35 (5H, m), 7.72 (1H, s), |
| | | | 7.75 (1H, dd, J = 9.2, 2.0 Hz), 8.73 (1H, t, J = 5.8 Hz). |
| 162 | CD₃OD | 400 | 2.86-2.95 (4H, m), 3.01-3.14 (1H, m), |
| | | | 3.87-4.04 (4H, m), 4.14-4.24 (2H, m), 4.58-4.66 (1H, m), |
| | | | 6.24-6.31 (1H, m), 6.87-7.03 (3H, m), |
| | | | 7.07-7.50 (7H, m), 7.71-7.77 (2H, m), 8.69-8.84 (1H, m). |
| 163 | CD₃OD | 400 | 2.83 (3H, s), 2.89-2.95 (1H, m), 3.02-3.11 (1H, |
| | | | m), 3.89-3.99 (2H, m), 4.15-4.30 (4H, m), |
| | | | 4.59-4.63 (1H, m), 6.95 (1H, d, J = 9.0 Hz), |
| | | | 7.00-7.04 (1H, m), 7.10-7.17 (2H, m), 7.46-7.51 (4H, m), |
| | | | 7.72 (1H, d, J = 1.3 Hz), 7.76 (1H, dd, J = 9.1, |
| | | | 2.1 Hz), 8.72 (1H, t, J = 5.9 Hz). |
| 164 | CD₃OD | 400 | 2.84 (3H, s), 2.90-2.95 (1H, m), 3.02-3.12 (1H, |
| | | | m), 3.90-4.01 (2H, m), 4.16-4.34 (4H, m), |
| | | | 4.60-4.64 (1H, m), 6.95 (1H, d, J = 9.1 Hz), |
| | | | 7.00-7.04 (1H, m), 7.10-7.17 (2H, m), 7.42-7.57 (4H, m), |
| | | | 7.72 (1H, d, J = 1.1 Hz), 7.76 (1H, dd, J = 9.1, |
| | | | 2.0 Hz), 8.72 (1H, t, J = 5.8 Hz). |
| 165 | CD₃OD | 400 | 2.87 (3H, s), 2.90-2.95 (1H, m), 3.02-3.12 (1H, |
| | | | m), 3.97-4.08 (2H, m), 4.19-4.27 (2H, m), |
| | | | 4.43-4.54 (2H, m), 4.60-4.64 (1H, m), 6.95 (1H, d, J = 9.2 Hz), |
| | | | 7.01-7.04 (1H, m), 7.10-7.17 (2H, m), |
| | | | 7.42-7.63 (4H, m), 7.72 (1H, s), 7.76 (1H, dd, J = 9.1, |
| | | | 2.1 Hz), 8.72 (1H, t, J = 5.8 Hz). |
| 166 | CD₃OD | 400 | 2.81 (3H, s), 2.89-2.94 (1H, m), 3.06-3.11 (1H, |
| | | | m), 3.84 (3H, s), 3.87-3.94 (2H, m), |
| | | | 4.15-4.27 (4H, m), 4.59-4.63 (1H, m), 6.95 (1H, d, J = 9.0 Hz), |
| | | | 6.99-7.03 (3H, m), 7.10-7.17 (2H, m), |
| | | | 7.38 (2H, d, J = 8.7 Hz), 7.72 (1H, d, J = 1.3 Hz), |
| | | | 7.75 (1H, dd, J = 9.1, 2.1 Hz), 8.71 (1H, t, J = 5.8 Hz). |
| 167 | CD₃OD | 400 | 1.55-1.85 (6H, m), 2.91-3.12 (4H, m), |
| | | | 3.35-3.45 (2H, m), 3.83-3.94 (2H, m), 4.16-4.30 (2H, m), |
| | | | 4.60-4.65 (1H, s), 6.96 (1H, d, J = 9.4 Hz), |
| | | | 7.01-7.04 (1H, m), 7.10-7.19 (2H, m), 7.72 (1H, d, J = 1.2 Hz), |
| | | | 7.76 (1H, dd, J = 9.2, 2.1 Hz), 8.71 (1H, t, |
| | | | J = 5.8 Hz). |
| 168 | CD₃OD | 400 | 2.87 (3H, s), 2.90-2.95 (1H, m), 3.02-3.12 (1H, |
| | | | m), 3.97-4.08 (2H, m), 4.19-4.27 (2H, m), |
| | | | 4.43-4.54 (2H, m), 4.60-4.64 (1H, m), 6.95 (1H, d, J = 9.2 Hz), |
| | | | 7.01-7.04 (1H, m), 7.10-7.17 (2H, m), |
| | | | 7.42-7.63 (4H, m), 7.72 (1H, s), 7.76 (1H, dd, J = 9.1, |
| | | | 2.1 Hz), 8.72 (1H, t, J = 5.8 Hz). |
| 169 | CD₃OD | 400 | 0.94-1.02 (2H, m), 1.16-1.37 (3H, m), |
| | | | 1.68-1.77 (6H, m), 2.88 (3H, s), 2.92-2.95 (3H, m), |
| | | | 3.10-3.15 (1H, m), 3.87-4.00 (2H, m), 4.18-4.28 (2H, |
| | | | m), 4.64 (1H, t, J = 7.6 Hz), 6.96 (1H, d, J = 9.1 Hz), |
| | | | 7.02-7.05 (1H, m), 7.12-7.19 (2H, m), |
| | | | 7.73 (1H, s), 7.78 (1H, dd, J = 9.1, 2.0 Hz), |
| | | | 8.73 (1H, t, J = 5.8 Hz). |
| 170 | CD₃OD | 400 | 1.55-1.85 (6H, m), 2.91-3.12 (4H, m), |
| | | | 3.35-3.45 (2H, m), 3.83-3.94 (2H, m), 4.16-4.30 (2H, m), |
| | | | 4.60-4.65 (1H, s), 6.96 (1H, d, J = 9.4 Hz), |
| | | | 7.01-7.04 (1H, m), 7.10-7.19 (2H, m), 7.72 (1H, d, J = 1.2 Hz), |
| | | | 7.76 (1H, dd, J = 9.2, 2.1 Hz), 8.71 (1H, t, |
| | | | J = 5.8 Hz) |
| 171 | CD₃OD | 400 | 2.92-2.97 (1H, m), 3.07-3.14 (1H, m), |
| | | | 3.31-3.32 (4H, m), 3.92-4.04 (6H, m), 4.17-4.26 (2H, m), |
| | | | 4.59-4.65 (1H, m), 6.96 (1H, d, J = 9.2 Hz), |
| | | | 7.01-7.04 (1H, m), 7.10-7.17 (2H, m), 7.72 (1H, s), |
| | | | 7.76 (1H, dd, J = 9.1, 2.0 Hz), 8.73 (1H, t, J = 5.9 Hz). |
| 172 | CD₃OD | 400 | 2.93-2.99 (1H, m), 3.10-3.21 (3H, m), 3.56 (2H, |
| | | | t, J = 6.0 Hz), 4.05-4.28 (4H, m), 4.45 (2H, s), |
| | | | 4.64-4.69 (1H, m), 6.97 (1H, d, J = 9.1 Hz), |
| | | | 7.03-7.06 (1H, m), 7.13-7.20 (3H, m), 7.26-7.34 (3H, |
| | | | m), 7.73 (1H, d, J = 1.2 Hz), 7.78 (1H, dd, J = 9.2, |
| | | | 2.0 Hz), 8.76 (1H, t, J = 5.9 Hz). |
| 173 | CD₃OD | 400 | 1.85-1.94 (2H, m), 2.67 (2H, t, J = 6.3 Hz), |
| | | | 2.82-2.99 (2H, m), 3.13-3.25 (2H, m), 3.66-3.76 (2H, |
| | | | m), 4.09-4.13 (2H, m), 4.48-4.53 (1H, m), |
| | | | 6.09-6.12 (1H, m), 6.49-6.52 (1H, m), 6.77-6.97 (6H, |
| | | | m), 7.60 (1H, d, J = 1.4 Hz), 7.66 (1H, dd, J = 9.2, |
| | | | 2.1 Hz), 8.52 (1H, t, J = 5.8 Hz). |
| 174 | CD₃OD | 400 | 2.89-2.96 (1H, m), 2.99 (3H, s), 3.04-3.09 (1H, |
| | | | m), 3.91 (2H, d, J = 1.4 Hz), 4.20-4.21 (2H, m), |
| | | | 4.58-4.63 (1H, m), 6.65 (2H, d, J = 8.1 Hz), |
| | | | 6.75 (1H, t, J = 7.3 Hz), 6.88-6.93 (2H, m), |
| | | | 6.99-7.10 (2H, m), 7.12-7.18 (2H, m), 7.67 (1H, d, J = 1.2 Hz), |
| | | | 7.73 (1H, dd, J = 9.2, 2.1 Hz), 8.58 (1H, t, |
| | | | J = 5.8 Hz). |
| 175 | CD₃OD | 400 | 1.31 (6H, d, J = 6.5 Hz), 1.39 (3H, d, J = 6.6 Hz), |
| | | | 2.69 (3H, s), 2.93-3.03 (1H, m), 3.07-3.13 (1H, |
| | | | m), 3.52 (1H, s, br), 4.01 (1H, q, J = 6.6 Hz), |
| | | | 4.17-4.28 (2H, m), 4.57-4.61 (1H, m), 4.86 (3H, s), |
| | | | 6.97 (1H, d, J = 9.1 Hz), 7.02-7.05 (1H, m), |
| | | | 7.11-7.24 (2H, m), 7.74-7.79 (2H, m), 8.71-8.82 (1H, |
| | | | m) |
| 176 | CD₃OD | 400 | 1.26 (6H, d, J = 6.64 Hz), 1.51 (3H, d, J = 6.85), |
| | | | 2.64 (3H, s), 2.90-2.98 (2H, m), 3.07-3.17 (1H, |
| | | | m), 3.94 (1H, q, J = 6.8 Hz), 4.16-4.21 (1H, m), |
| | | | 4.27-4.31 (1H, m), 4.60-4.80 (1H, m), 4.86 (3H, |
| | | | s), 6.97 (1H, d, J = 9.2 Hz), 7.05-7.07 (1H, m), |
| | | | 7.14-7.22 (2H, m), 7.74-7.84 (2H, m), |
| | | | 8.70-8.89 (1H, m) |
| 178 | CD₃ OD | 400 | 0.43-0.54 (1H, m), 0.58-0.65 (6H, m), |
| | | | 0.97-1.10 (1H, m), 1.78-1.83 (1H, m), 2.64 (6H, s), |
| | | | 2.66-2.72 (1H, m), 2.89-2.99 (2H, m), 3.45-3.53 (2H, |
| | | | m), 3.96-4.08 (2H, m), 4.31-4.37 (1H, m), |
| | | | 6.76 (1H, d, J = 9.4 Hz), 6.86-6.88 (1H, m), |
| | | | 6.93-7.00 (2H, m), 7.55 (1H, d, J = 1.2 Hz), 7.60 (1H, dd, |
| | | | J = 2.1, 9.2 Hz), 8.51-8.59 (2H, m) |
| 179 | CD₃OD | 400 | 1.07 (9H, s), 3.00 (6H, s), 3.08-3.17 (2H, m), |
| | | | 3.62 (1H, s), 4.21-4.29 (2H, m), 4.52-4.58 (1H, |
| | | | m), 4.86 (2H, s), 6.97 (1H, d, J = 9.1 Hz), |
| | | | 7.06-7.09 (1H, m), 7.14-7.21 (2H, m), 7.76-7.79 (2H, |
| | | | m), 8.74-8.77 (1H, m), 8.94-8.96 (1H, m) |
| 180 | CD₃OD | 400 | 1.32 (6H, d, J = 8.0 Hz), 2.62-2.68 (5H, m), |
| | | | 2.84-2.95 (2H, m), 3.11-3.16 (2H, m), |
| | | | 3.81-3.86 (2H, m), 4.23 (3H, d, J = 4.0 Hz), 4.59-4.66 (2H, |
| | | | m), 6.95-7.04 (3H, m), 7.23-7.27 (2H, m), |
| | | | 7.69-7.77 (1H, m), 7.83 (1H, dd, J = 8.0, 4.0 Hz), |
| | | | 8.70 (1H, t, J = 8.0 Hz) |
| 181 | CD₃OD | 400 | 1.14-1.18 (2H, m), 1.23 (6H, d, J = 6.6 Hz), |
| | | | 1.28-1.39 (2H, m), 1.50 (3H, d, J = 6.9 Hz), 2.61 (3H, |
| | | | s), 2.85-2.94 (1H, m), 3.11-3.25 (1H, m), |
| | | | 3.91 (1H, t, J = 6.7 Hz), 4.18 (1H, d, J = 15.1 Hz), |
| | | | 4.28 (1H, d, J = 15.1 Hz), 4.58-4.78 (1H, m), 4.86 (1H, |
| | | | s), 6.92-6.96 (1H, m), 6.98-7.04 (2H, m), |
| | | | 7.25-7.28 (2H, m), 7.74-7.78 (2H, m) |
| 182 | CD₃OD | 400 | 0.67-0.76 (1H, m), 0.83-0.87 (6H, m), |
| | | | 1.18-1.24 (1H, m), 1.99-2.05 (1H, m), 2.87 (6H, s), |
| | | | 2.89-2.97 (1H, m), 3.12-3.17 (1H, m), 3.62 (1H, d, |
| | | | J = 5.0 Hz), 4.28 (2H, d, J = 5.7 Hz), 4.58 (1H, t, |
| | | | J = 4.2 Hz), 4.86 (3H, s), 6.96-7.06 (3H, m), |
| | | | 7.27-7.30 (2H, m), 7.75-7.80 (2H, m), 8.73-8.76 (1H, |
| | | | m) |
| 183 | CD₃OD | 400 | 1.05 (9H, s), 2.90-2.98 (1H, m), 3.00 (6H, s), |
| | | | 3.08-3.17 (1H, m), 3.59 (1H, s), 4.23 (2H, d, |
| | | | J = 5.8 Hz), 4.52-4.60 (1H, m), 4.86 (3H, s), |
| | | | 6.89-7.03 (3H, m), 7.26-7.29 (2H, m), 7.73-7.76 (2H, |
| | | | m), 8.71-8.73 (1H, m) |
| 184 | CD₃OD | 400 | 1.25-1.50 (6H, m), 2.40-2.60 (1H, m), |
| | | | 2.65-2.80 (1H, m), 3.00-3.20 (2H, m), 3.25-3.40 (3H, m), |
| | | | 4.05-4.40 (4H, m), 4.85-5.05 (2H, m), |
| | | | 6.70-7.00 (3H, m), 7.05-7.20 (3H, m), 7.25-7.35 (3H, m), |
| | | | 7.65-7.80 (2H, m), 8.60-8.70 (1H, m) |
| 185 | CD₃OD | 400 | 2.49-2.55 (1H, m), 2.70-2.81 (1H, m), 2.99 (6H, |
| | | | s), 3.02-3.08 (1H, m), 3.33-3.37 (1H, m), |
| | | | 3.99-4.03 (1H, m), 4.18-4.24 (3H, m), 4.87 (3H, s), |
| | | | 6.73-6.76 (1H, m), 6.82-6.90 (1H, m), 6.95 (1H, |
| | | | dd, J = 1.8, 8.3 Hz), 7.03-7.10 (1H, m), |
| | | | 7.15-7.18 (2H, m), 7.21-7.27 (3H, m), 7.72-7.75 (2H, m), |
| | | | 8.55-8.68 (1H, m) |
| 186 | CD₃OD | 270 | 0.8-2.1 (31H, m), 2.49 (3H, s), 3.6-3.9 (1H, m), |
| | | | 4.20-4.50 (3H, m), 6.7-6.9 (1H, m), |
| | | | 7.70-7.85 (1H, m), 7.9-8.1 (1H, m) |
| 187 | CD₃OD | 270 | 0.7-1.91 (33H, m), 2.56 (3H, s), 3.70-3.90 (1H, |
| | | | s), 4.20-4.55 (3H, m), 6.7-6.9 (1H, m), |
| | | | 7.7-7.9 (1H, m), 8.5-8.9 (1H, m) |
| 188 | d6DMSO | 270 | 0.8-1.0 (6H, m), 1.1-1.3 (1H, m), 1.5-1.8 (1H, |
| | | | m), 2.4-2.8 (9H, m), 2.8-2.9 (1H, m), |
| | | | 3.0-3.1 (1H, m) |
| | | | 3.2-3.7 (3H, m), 4.0-4.2 (1H, m), 4.7-4.8 (1H, |
| | | | m), 6.85 (1H, d, J = 7 Hz), 7.2-7.3 (1H, m), |
| | | | 7.5-7.6 (2H, m), 7.7-7.8 (1H, m), |
| | | | 8.5-8.6 (1H, m), 8.75-8.8 (1H, m). |
| 189 | CD₃OD | 270 | 3.6-4.0 (1H, m), 4.1-4.4 (6H, m), 4.7-5.2 (5H, |
| | | | m), 5.9-6.3 (5H, m), 6.4-6.6 (1H, m), |
| | | | 8.5-8.7 (1H, m), 8.9-9.1 (1H, m), 9.2-9.3 (2H, |
| | | | m), 9.4-9.7 (1H, m), 10.6-10.5 (1H, m) |
| 190 | CD₃OD | 270 | 1.7-2.3 (4H, m), 2.4-2.7 (5H, m), 2.8-3.4 (7H, |
| | | | m), 3.6-3.8 (1H, m), 4.0-4.4 (3H, m), |
| | | | 4.6-4.8 (1H, m) |
| | | | 6.7-6.9 (1H, m), 7.0-7.2 (1H, m), 7.3-7.4 (2H, |
| | | | m), 7.6-7.9 (1H, m) 8.6-8.9 (1H, m) |
| 191 | d6DMSO | 270 | 2.4-2.6 (3H, s), 2.6-2.8 (9H, m), 4.1 (3H, s), |
| | | | 4.4-4.6 (1H, s), |
| | | | 6.8-6.9 (1H, m), 7.2-7.4 (1H, m), 7.4-7.8 (5H, |
| | | | m) |
| 192 | d⁶DMSO | 270 | 2.43 (3H, s), 2.4 (3H, s), 2.5-2.9 (2H, m), |
| | | | 3.5-3.8 (3H, m), 4.0-4.4 (3H, m), 4.5-4.7 (2H, m), |
| | | | 6.8-6.9 (1H, m), 7.1-7.2 (1H, m), 7.4-7.6 (2H, m), |
| | | | 7.8-7.9 (1H, m), 8.8-8.9 (1H, m), 9.0-9.1 (1H, |
| | | | m) |
| 193 | CD₃OD | 270 | 2.3 (3H, s), 2.6 (4H, s), 2.7-2.8 (3H, m), |
| | | | 2.9-3.15 (3H, m), 3.3-3.4 (3H, m), 4.0-4.4 (5H, br, m), |
| | | | 6.7 (1H, d, J = 7 Hz), 6.9-7.0 (1H, m), |
| | | | 7.1-7.2 (3H, m), 7.25-7.3 (4H, m), 7.4-7.5 (1H, m), |
| | | | 7.6-7.7 (1H, m) |
| 194 | CD₃OD | 270 | 2.4-2.6 (3H, s), 2.7-3.2 (4H, m), 3.2-3.2 (5H, m), |
| | | | 4.0-4.1 (3H, m), 4.2-4.4 (2H, m), 4.5-4.6 (1H, |
| | | | m) |
| | | | 6.8-6.9 (1H, m), 7.0-7.3 (5H, m), 7.2-7.4 (2H, |
| | | | m), 7.6-7.8 (1H, m) |
| 195 | CD₃OD | 270 | 2.3-2.4 (4H, m), 2.6-3.1 (4H, m), 3.3-3.4 (5H, |
| | | | m), 4.0-4.1 (2H, m), 4.2-4.3 (1H, m), |
| | | | 4.1-4.5 (1H, m) |
| | | | 6.8-6.9 (1H, m), 7.0-7.3 (3H, m), 7.3-7.6 (4H, |
| | | | m), 7.7-7.9 (1H, s) |
| 196 | CD₃OD | 270 | 2.4-2.5 (4H, m), 2.8-3.1 (3H, m), 3.15-3.2 (1H, |
| | | | m), 3.2-3.3 (3H, m), 4.0-4.1 (1H, m), |
| | | | 4.2-4.4 (3H, m) 4.5-4.8 (1H, m), 6.85 (1H, d, J = 7 Hz), |
| | | | 7.1-7.2 (1H, m), 7.3-7.4 (2H, m), 7.45-7.5 (1H, |
| | | | m), 7.6-7.8 (1H, m), 8.0-8.1 (1H, m) |
| | | | 8.6-8 (3H, m) |
| 197 | CD₃OD | 270 | 3.0-3.1 (4H, m), 4.1-4.4 (3H, m), 4.7-4.9 (2H, |
| | | | m), 5.75 (2H, s), 6.25 (2H, d, J = 7 Hz), |
| | | | 6.45 (2H, d, J = 7H), 7.0-7.4 (9H, m), |
| | | | 9.8-9.9 (1H, m) |
| 198 | CD₃OD | 270 | 0.7-0.95 (2H, m), 1.0-1.25 (2H, m), 1.3-1.5 (3H, |
| | | | m), 1.6-2.0 (3H, m), 2.4-2.6 (3H, m), |
| | | | 2.8-3.0 (1H, m), 3.1-3.4 (8H, m) 3.5-3.8 (1H, m), |
| | | | 4.1-4.4 (3H, m), 4.6-4.8 (1H, m), 6.7-6.9 (1H, m), |
| | | | 7.1-7.3 (1H, m), 74-7.6 (2H, m), 7.7-7.8 (1H, m) |
| 199 | CD₃OD | 270 | 2.4-2.5 (4H, m), 2.8-3.1 (3H, m), 3.15-3.2 (1H, |
| | | | m), 3.2-3.3 (3H, m), 4.0-4.1 (1H, m), |
| | | | 4.2-4.4 (3H, m) 4.5-4.8 (1H, m), 6.85 (1H, d, J = 7 Hz), |
| | | | 7.1-7.2 (1H, m), 7.3-7.4 (2H, m), |
| | | | 7.45-7.5 (1H, m), 7.6-7.8 (1H, m), 8.0-8.1 (1H, m) |
| | | | 8.6-8.8 (3H, m) |
| 200 | d⁶DMSO | 270 | 1.0-1.3 (6H, m), 1.4-1.7 (5H, m), 2.4 (3H, s), |
| | | | 2.6 (2H, s), 2.7-2.9 (1H, m), 3.0-3.1 (1H, m), |
| | | | 3.7-3.8 (1H, m), 4.0-4.3 (2H, m), 4.7-4.8 (1H, m) |
| | | | 6.8-6.9 (1H, m), 7.1-7.2 (1H, m), 7.4-7.6 (2H m), |
| | | | 7.7 (1H, d, J = 8 Hz), 7.9-8.1 (4H, m), 8.6-8.7 (1H, m) |
| | | | 8.9-8.0 (1H, m) |
| 201 | CD₃OD | 270 | 1.8-1.9 (2H, m), 2.0-2.1 (1H, m), 2.4-2.5 (3H, s), |
| | | | 2.8-3.1 (3H, m), 3.2-3.3 (8H, m), 3.7-3.8 (1H, |
| | | | m), 4.9-5.1 (1H, m) 6.7-6.9 (1H, m), 7.1-7.2 (1H, |
| | | | m), 7.3-7.4 (2H, m), 7.6-7.8 (1H, m) |
| 202 | CD₃OD | 270 | 2.4 (3H, s), 2.7-3.0 (3H, m), 3.1-3.2 (2H, m), |
| | | | 3.3 (3H, s), 4.0-4.4 (5H, m), 4.6-4.8 (1H, m), |
| | | | 6.85 (1H, d, J = 7 Hz), 7.1-7.4 (8H, m), 7.75 (1H, |
| | | | d, J = 7 Hz) |
| 203 | CD₃OD | 270 | 0.8-0.9 (6H, m), 1.3-1.4 (1H, m), 1.8-1.7 (1H, |
| | | | m), 2.25 (3H, s), 2.6 (3H, s), 3.3-3.5 (3H, m), |
| | | | 3.6-3.7 (2H, m), 4.0-4.3 (2H, m), 4.8-4.9 (2H, |
| | | | m), 6.6 (1H, d, J = 7 Hz), 7.3-7.4 (5H, m), |
| | | | 7.5-7.6 (3H, m), 7.7-8.0 (2H, m), 8.2-8.3 (1H, m) |
| 204 | CD₃OD | 400 | 1.72-1.77 (1H, m), 1.87-1.96 (1H, m), |
| | | | 2.10-2.17 (1H, m), 2.45 (3H, s), 2.89 (3H, s), |
| | | | 2.91-2.95 (1H, m), 3.07-3.10 (1H, m), 3.12-3.21 (1H, m), |
| | | | 3.63-3.69 (1H, m), 4.05 (1H, t, J = 8.5 Hz), |
| | | | 4.13-4.18 (1H, m), 4.25-4.31 (1H, m), 4.64 (1H, t, |
| | | | J = 8.4 Hz), 4.86 (3H, s), 6.78 (1H, d, J = 9.0 Hz), |
| | | | 6.96-7.00 (2H, m), 7.19-7.28 (2H, m), 7.68 (2H, |
| | | | d, J = 9.1 Hz), 8.65-8.67 (1H, m) |
| 205 | CD₃OD | 400 | 1.75-1.82 (1H, m), 1.89-1.96 (1H, m), |
| | | | 2.14-2.18 (1H, m), 2.47 (3H, s), 2.89 (3H, s), |
| | | | 2.92-2.93 (1H, s), 3.06-3.11 (1H, m), 3.15-3.22 (1H, m), |
| | | | 3.64-3.70 (1H, m), 4.06 (1H, t, J = 8.6 Hz), |
| | | | 4.13-4.18 (1H, m), 4.27-4.32 (1H, m), 4.64 (1H, t, |
| | | | J = 8.1 Hz), 4.86 (3H, s), 6.79 (1H, d, J = 9.1 Hz), |
| | | | 7.00-7.07 (1H, m), 7.09-7.19 (2H, m), 7.71 (2H, |
| | | | d, J = 9.1 Hz), 8.67-8.69 (1H, m) |
| 206 | CD₃OD | 400 | 1.71-1.80 (1H, m), 1.86-1.98 (1H, m), |
| | | | 2.10-2.20 (1H, m), 2.34 (3H, s), 2.42-2.48 (1H, m), |
| | | | 2.51 (3H, s), 2.88 (3H, s), 2.91-2.95 (1H, m), |
| | | | 3.05-3.10 (1H, m), 3.14-3.21 (1H, m), 3.64-3.70 (1H, |
| | | | m), 4.09 (1H, t, J = 8.4 Hz), 4.21-4.33 (2H, m), |
| | | | 4.60-4.65 (1H, m), 6.66 (1H, s), 7.00-7.03 (1H, |
| | | | m), 7.09-7.17 (2H, m), 8.47 (1H, t, J = 4.9 Hz), |
| | | | 8.87 (1H, d, J = 7.6 Hz) |
| 207 | CD₃OD | 400 | 0.80 (3H, d, J = 6.8 Hz), 0.83-0.89 (4H, m), |
| | | | 1.27-1.36 (1H, m), 1.76-1.82 (1H, m), 2.36 (3H, s), |
| | | | 2.52 (3H, s), 2.60 (3H, s), 2.88-2.94 (1H, m), |
| | | | 3.08-3.13 (1H, m), 3.66 (1H, d, J = 4.9 Hz), |
| | | | 4.30 (2H, d, J = 4.8 Hz), 4.56-4.62 (1H, m), 6.67 (1H, |
| | | | s), 7.05-7.08 (1H, m), 7.12-7.19 (2H, m), |
| | | | 8.49 (1H, t, J = 4.9 Hz), 8.79 (1H, d, J = 7.5 Hz) |
| 209 | CD₃OD | 270 | 0.70-0.94 (7H, m), 1.16-1.32 (1H, m), |
| | | | 1.65-1.74 (1H, m,) 2.20 (3H, s), 3.30-3.40 (1H, m), |
| | | | 3.62-3.72 (2H, m), 4.05-4.28 (2H, m), 4.78-4.84 (1H, |
| | | | m), 7.31-7.38 (2H, m), 7.45-7.57 (4H, m), |
| | | | 7.74-7.78 (1H, m), 7.84-7.87 (1H, m), 8.16 (1H, d, J = 8.2 Hz), |
| | | | 8.62 (1H, m) |
| 210 | CD₃OD | 270 | 1.70-1.90 (3H, m), 2.05-2.30 (1H, m), |
| | | | 2.80-3.05 (4H, m), 3.10-3.80 (5H, m), 4.10-4.40 (2H, m), |
| | | | 4.45-4.60 (1H, m), 6.90-7.05 (1H, m), |
| | | | 7.10-7.25 (1H, m), 7.30-7.50 (2H, m), 7.65-7.85 (2H, m), |
| | | | 8.25-8.40 (2H, m) |
| 211 | CD₃OD | 270 | 0.50-0.70 (3H, m), 0.75-0.90 (3H, m), |
| | | | 0.95-1.15 (1H, m), 1.40-1.70 (2H, m), 1.95-2.10 (1H, m), |
| | | | 2.80-2.95 (4H, m), 3.15-3.40 (4H, m), |
| | | | 3.50-3.70 (1H, m), 4.10-4.40 (2H, m), 4.50-4.70 (1H, m), |
| | | | 6.90-7.05 (1H, m), 7.15-7.30 (1H, m), |
| | | | 7.35-7.50 (1H, m), 7.65-7.90 (2H, m), 8.30-8.40 (1H, m), |
| | | | 8.50-8.70 (1H, m) |
| 212 | CD₃OD | 270 | 1.25-1.40 (3H, m), 2.80-3.15 (8H, m), |
| | | | 3.25-3.35 (2H, m), 4.10-4.60 (4H, m), 6.90-7.00 (1H, m), |
| | | | 7.10-7.20 (1H, m), 7.30-7.50 (2H, m), |
| | | | 7.60-7.80 (2H, m), 8.15-8.30 (1H, m), 8.40-8.55 (1H, m) |
| 213 | CD₃OD | 270 | 0.58 (2H, d, J = 6.7 Hz), 0.89 (2H, t, J = 7.2 Hz), |
| | | | 1.04-1.09 (1H, m), 1.53-1.56 (1H, m), |
| | | | 1.77-1.83 (1H, m), 2.86 (3H, s), 3.16-3.41 (3H, m), |
| | | | 3.86-3.90 (1H, m), 4.11-4.21 (2H, m), 4.90 (7H, s), |
| | | | 6.87 (1H, d, J = 9.2 Hz), 7.32-7.41 (3H, m), |
| | | | 7.63 (2H, d, J = 9.9 Hz), 7.86 (2H, t, J = 5.5 Hz), |
| | | | 8.39 (1H, t, J = 5.7 Hz), 8.71 (1H, d, J = 7.2 Hz) |
| 214 | CD₃OD | 270 | 0.87-1.27 (18H, m), 1.30-1.80 (14H, m), |
| | | | 3.78-3.80 (1H, m), 4.17-4.23 (2H, m), 4.58 (1H, d, |
| | | | J = 9.1 Hz), 4.91 (4H, s), 6.98 (1H, d, J = 8.9 Hz), |
| | | | 7.81 (1H, s), 7.91 (1H, d, J = 9.2 Hz), 8.70-8.90 (1H, |
| | | | m) |
| 215 | CD₃OD | 270 | 0.95-1.23 (10H, m), 1.53-1.76 (16H, m), |
| | | | 2.02-2.07 (4H, m), 2.40-2.60 (1H, m), 3.32-3.41 (2H, |
| | | | m), 4.22-4.26 (2H, m), 4.40-4.42 (1H, m), |
| | | | 4.61 (1H, d, J = 7.0 Hz), 6.97 (1H, d, J = 9.1 Hz), |
| | | | 7.76 (1H, d, J = 1.5 Hz), 7.90 (1H, dd, J = 2.9 Hz, |
| | | | 9.2 Hz), 8.70-8.90 (1H, m) |
| 217 | CD₃OD | 270 | 1.11-1.22 (17H, m), 1.56-1.70 (14H, m), |
| | | | 3.81 (1H, s), 4.24-4.29 (2H, m), 4.59 (1H, d, |
| | | | J = 7.2 Hz), 4.86-4.89 (5H, m), 6.99 (1H, d, |
| | | | J = 9.2 Hz), 7.82 (1H, d), 7.93 (1H, dd, J = 2.0 Hz, |
| | | | 9.2 Hz), 8.60-8.70 (1H, m) |
| 218 | CD₃OD | 270 | 0.98-1.28 (8H, m), 1.52 (3H, d, J = 6.9 Hz), |
| | | | 1.63-1.81 (14H, m), 2.65 (3H, s), 3.93-3.95 (1H, m), |
| | | | 4.17-4.20 (1H, m), 4.28-4.30 (1H, m), 4.60 (1H, |
| | | | d, J = 7.6 Hz), 4.96 (5H, s), 6.98 (1H, d, J = 9.1 Hz), |
| | | | 7.78 (1H, d), 7.919 (1H, dd, J = 1.9 Hz, 9.1 Hz), |
| | | | 8.70-8.80 (1H, m) |
| 219 | CD₃OD | 270 | 0.97-1.28 (18H, m), 1.54-1.70 (14H, m), |
| | | | 2.59 (3H, s), 3.3 (1H, d, J = 4.7 Hz), 4.23-4.29 (2H, m), |
| | | | 4.58 (1H, d, J = 7.7 Hz), 4.90 (4H, s), 6.98 (1H, d, |
| | | | J = 9.1 Hz), 7.82 (1H, d, J = 1.5 Hz), 7.91 (1H, |
| | | | dd, J = 2.3 Hz, 9.2 Hz), 8.70-8.90 (1H, m) |
| 220 | CD₃OD | 270 | 0.95-1.23 (8H, m), 1.52-1.73 (14H, m), |
| | | | 3.29-3.32 (2H, m), 3.67-3.86 (2H, m), 4.21-4.26 (1H, |
| | | | m), 4.63 (1H, d, J = 6.4 Hz), 4.49 (5H, s), |
| | | | 6.98 (1H, d, J = 9.4 Hz), 7.76 (1H, d), 7.89 (1H, dd, |
| | | | J = 2 Hz, 9.2 Hz), 8.60-8.70 (1H, m) |
| 221 | CD₃OD | 270 | 1.10-1.28 (8H, m), 1.52-1.73 (14H, m), |
| | | | 2.72 (3H, s), 3.29-3.32 (2H, m), 3.86 (2H, dd, J = 8.9, |
| | | | 15.8 Hz), 4.20-4.27 (2H, m), 4.63 (1H, |
| | | | d, J = 6.5 Hz), 4.94 (3H, s), 6.97 (1H, d, J = 9.2 Hz), |
| | | | 7.76 (1H, d), 7.89 (1H, dd, J = 2.2 Hz, 7.3 Hz), |
| | | | 8.60-8.70 (1H, m) |
| 222 | CD₃OD | 270 | 1.10-1.28 (8H, m), 1.52-1.73 (14H, m), |
| | | | 2.93 (6H, s), 3.29-3.32 (2H, m), 4.01 (2H, d), |
| | | | 4.20-4.27 (2H, m), 4.66 (1H, d), 4.94 (2H, s), |
| | | | 7.01 (1H, d, J = 9.2 Hz), 7.77 (1H, s), 7.88 (1H, d), |
| | | | 8.60-8.70 (1H, m) |
| 223 | | 400 | 1.09-1.30 (12H, m), 1.64-1.76 (14H, m), |
| | | | 2.00-2.10 (2H, m), 2.19-2.26 (1H, m), 2.57-2.63 (1H, |
| | | | m), 2.93 (3H, s), 3.20-3.27 (1H, m), |
| | | | 3.71-3.77 (1H, m), 4.17-4.24 (2H, m), 4.29-4.39 (1H, m), |
| | | | 4.64 (1H, d, J = 7.2 Hz), 6.99 (1H, d, J = 9.1 Hz), |
| | | | 7.80 (1H, d, J = 1.5 Hz), 7.92 (1H, dd, |
| | | | J = 2.1, 9.2 Hz), 8.74-8.77 (1H, m) |
| 224 | CD₃OD | 270 | 0.87-0.96 (6H, m), 1.30-1.60 (1H, m), |
| | | | 1.70-2.10 (1H, m), 3.22-3.26 (2H, m), 3.71-3.76 (2H, m), |
| | | | 3.86-4.06 (1H, m), 4.51-4.53 (1H, m), 4.86 (6H, |
| | | | s), 6.71-6.80 (1H, m), 7.18-7.45 (7H, m), |
| | | | 8.35-8.55 (1H, m) |
| 225 | CD₃OD | 270 | 0.8-0.9 (1H, m), 1.5-1.8 (1H, m), 1.9-2.1 (1H, |
| | | | m), 2.2-2.4 (1H, m), 2.9 (3H, s), 2.5 (3H, s), |
| | | | 3.0-3.1 (3H, m), 3.3 (2H, s), 3.4-3.5 (2H, m), |
| | | | 3.6-3.8 (1H, m), 4.2-4.4 (3H, m), 5.65 (1H, dd, J = 7 Hz), |
| | | | 7.0-7.1 (1H, d, J = 9 Hz), 7.15-7.3 (4H, m), |
| | | | 7.7-7.9 (1H, m), 8.7-8.8 (1H, m) |
| 227 | CD₃OD | 270 | 1.19-1.78 (22H, m), 4.24-4.27 (2H, m), |
| | | | 4.50-4.60 (1H, m), 6.19 (1H, s), 6.89-6.97 (3H, m), |
| | | | 7.73 (1H, s), 6.86 (1H, dd, J = 2 Hz, 9 Hz), |
| | | | 7.85-7.88 (1H, m), 11.0 (1H, s, br) |
| 228 | CD₃OD | 270 | 3.47-3.54 (1H, m), 3.64-3.72 (1H, m), |
| | | | 4.04-4.11 (2H, m), 4.79-4.85 (1H, m), 4.89-4.91 (3H, m), |
| | | | 6.16-6.19 (1H, m), 6.81-6.93 (3H, m), |
| | | | 7.30-7.37 (2H, m), 7.46-7.56 (4H, m), 7.72-7.76 (1H, m), |
| | | | 7.84-7.88 (1H, m), 8.20 (1H, d, J = 7.9 Hz), |
| | | | 8.40-8.60 (1H, m), 10.8-11.10 (1H, s, br) |
| 229 | CD₃OD | 270 | 3.05-3.40 (3H, m), 4.15-4.30 (1H, m), |
| | | | 4.60-4.80 (1H, m), 4.90-5.00 (3H, m), 6.10-6.30 (1H, m), |
| | | | 6.80-7.00 (3H, m), 7.40-7.80 (3H, m), |
| | | | 8.55-8.70 (1H, m) |
| 231 | CD₃OD | 270 | 2.18 (3H, s), 2.20 (3H, s), 3.54-3.60 (1H, m), |
| | | | 3.64-3.72 (1H, m), 4.03-4.13 (2H, m), 4.80-4.86 (1H, m), |
| | | | 4.89-4.91 (2H, m), 5.71 (1H, d, J = 2.5 Hz), |
| | | | 6.16-6.19 (1H, m), 6.83 (1H, d, J = 8.9 Hz), |
| | | | 7.33-7.367 (2H, m), 7.476-7.54 (4H, m), |
| | | | 7.75-7.78 (1H, m), 7.85-7.886 (1H, m), |
| | | | 8.21 (1H, d, J = 7.7 Hz), 8.40-8.60 (1H, m), |
| | | | 10.8-11.10 (1H, s, br) |
| 232 | CD₃OD | 270 | 2.17 (3H, s), 2.19 (3H, s), 3.10-3.40 (4H, m), |
| | | | 4.15-4.30 (1H, m), 4.60-4.80 (1H, m), |
| | | | 4.90-5.10 (1H, m0, 5.71 (1H, s), 6.93 (1H, d, J = 9.15 Hz), |
| | | | 7.40-7.80 (6H, m), 8.60-8.80 (1H, m), |
| | | | 10.30-10.50 (1H, m) |
| 233 | CD₃OD | 270 | 2.10-2.30 (6H, m), 3.00-3.20 (2H, m), |
| | | | 3.25-3.40 (2H, m), 4.05-4.30 (2H, m), 4.70-4.85 (1H, m), |
| | | | 4.90-5.10 (1H, m), 5.72 (1H, s), |
| | | | 6.90-7.00 (1H, m), 7.10-7.25 (1H, m), 7.30-7.50 (2H, m), |
| | | | 7.65-7.80 (2H, m), 8.60-8.70 (1H, m0, |
| | | | 10.40-10.55 (1H, m) |
| 234 | CD₃OD | 270 | 2.18 (3H, s), 2.19 (3H, s), 2.27 (3H, s), |
| | | | 2.90-3.10 (2H, m), 3.25-3.35 (2H, m), 3.50-3.70 (1H, m), |
| | | | 4.15-4.30 (2H, m), 4.60-4.75 (1H, m), |
| | | | 5.71 (1H, s), 6.85-7.30 (5H, m), 7.60-7.80 (2H, m), |
| | | | 8.50-8.70 (1H, m), 10.30-40.50 (1H, m) |
| 235 | CD₃OD | 270 | 2.20 (6H, s), 3.00-3.35 (4H, m), |
| | | | 4.10-4.30 (2H, m), 4.60-4.75 (1H, m), 4.90-5.10 (1H, m), |
| | | | 5.72 (1H, s), 6.94 (1H, d, J = 9.15 Hz), |
| | | | 7.10-7.30 (4H, m), 7.60-7.80 (2H, m), 8.60-8.75 (1H, m), |
| | | | 10.30-10.50 (1H, m) |
| 236 | CD₃OD | 270 | 2.19 (6H, m), 3.00-3.40 (3H, m), |
| | | | 4.10-4.40 (2H, m), 4.60-4.80 (1H, m), 4.90-5.10 (2H, m), |
| | | | 5.72 (1H, s), 6.85-7.40 (5H, m), |
| | | | 7.60-7.80 (2H, m), 8.55-8.70 (1H, m), 10.40-10.50 (1H, m) |
| 237 | CD₃OD | 270 | 2.19 (6H, s), 3.07-3.20 (2H, m), |
| | | | 3.25-3.40 (3H, m), 4.05-4.40 (2H, m), 4.60-4.80 (1H, m), |
| | | | 5.71 (1H, s), 6.91-6.94 (1H, m), 7.23 (4H, s), |
| | | | 7.65-7.71 (3H, m), 8.50-8.70 (1H, m), |
| | | | 10.40-10.60 (1H, m) |
| 238 | d6- | 270 | 1.3-1.8 (7H, m), 2.1-2.3 (6H, m), 2.5-2.6 (4H, |
| | DMSO | | m), 3.1-3.4 (3H, m), 4.2-4.3 (1H, d, J = 7 Hz), |
| | | | 6.7 (1H, s), 6.9-7.0 (1H, m), 7.2-7.3 (1H, m), |
| | | | 7.7-8.1 (3H, m), 8.6-8.7 (1H, m), 10.9-11 (1H, s) |
| 239 | CD₃OD | 270 | 2.19 (3H, s), 2.20 (3H, s), 3.20-3.30 (2H, m), |
| | | | 4.20-4.21 (2H, m), 4.78 (1H, t, J = J = 7.7 Hz), |
| | | | 5.71 (1H, s), 6.93 (1H, d, J = 9.1 Hz), 7.18-7.38 (7H, m), |
| | | | 7.68 (1H, s), 7.73-7.77 (1H, m), 8.50-8.70 (1H, m), |
| | | | 10.35-10.55 (1H, m) |
| 240 | CD₃OD | 270 | 2.19 (6H, s), 3.25-3.36 (2H, m), 4.21-4.23 (2H, m), |
| | | | 4.75 (1H, t, J = J = 7.4 Hz), 4.89 (2H, s), |
| | | | 5.71 (1H, s), 6.93 (1H, d, J = 9.2 Hz), 7.40-7.51 (3H, m), |
| | | | 7.66-7.78 (4H, m), 8.50-8.70 (1H, m), |
| | | | 10.35-10.55 (1H, m) |
| 241 | CD₃OD | 270 | 2.14 (3H, s), 2.18 (3H, s), 3.20-3.40 (4H, m), |
| | | | 4.00-4.30 (2H, m), 4.65-4.90 (1H, m), |
| | | | 4.95-5.05 (2H, m), 5.70 (1H, s), 6.70-7.60 (8H, m), |
| | | | 8.35-8.50 (1H, m), 10.30-10.50 (1H, m) |
| 242 | CD₃OD | 270 | 2.10-2.30 (6H, m), 2.80-3.50 (5H, m), |
| | | | 4.20-4.40 (2H, m), 4.50-4.70 (1H, m), 5.71 (1H, s), |
| | | | 6.80-7.10 (3H, m), 7.10-7.30 (2H, m), |
| | | | 7.60-7.80 (2H, m), 8.50-8.70 (1H, m), 10.30-10.50 (1H, m) |
| 243 | CD₃OD | 270 | 2.00-2.10 (1H, m), 2.15-2.30 (6H, m), |
| | | | 2.95-3.20 (2H, m), 3.25-3.35 (2H, m), 4.10-4.30 (2H, m), |
| | | | 4.60-4.70 (1H, m), 5.71 (1H, s), |
| | | | 6.90-7.20 (4H, m), 7.70-7.80 (2H, m), 8.60-7.70 (1H, m), |
| | | | 10.40-40.50 (1H, m) |
| 244 | CD₃OD | 270 | 2.21 (6H, s), 2.35 (3H, s), 3.12-3.15 (2H, m), |
| | | | 4.14-4.20 (2H, m), 4.60-4.75 (1H, m), 4.89 (3H, s), |
| | | | 5.72 (1H, s), 6.90 (1H, d, J = 9.2 Hz), 7.11-7.13 (5H, m), |
| | | | 7.61-7.65 (2H, m), 8.50-8.70 (1H, m) |
| 245 | CD₃OD | 270 | 2.20 (3H, s), 2.22 (3H, s), 3.08-3.15 (2H, m), |
| | | | 4.22 (1H, s), 4.68-4.735 (1H, m), 4.89 (3H, s), |
| | | | 5.72 (1H, s), 6.95 (1H, d, J = 8.4 Hz), 7.11-7.20 (3H, m), |
| | | | 7.74-7.81 (2H, m), 8.60-8.80 (1H, m), |
| | | | 10.43 (1H, s, br) |
| 246 | CD₃OD | 270 | 2.19 (6H, s) 3.03-3.10 (2H, m) 4.09-4.26 (2H, |
| | | | m) 4.67 (1H, t, J = 7.4 Hz) 4.92 (5H, s) |
| | | | 5.71 (1H, s) 6.92 (1H, d, J = 9.9 Hz) 7.16-7.24 (4H, |
| | | | m) 7.67 (2H, d, J = 7.4 Hz) |
| 247 | CD₃OD | 270 | 2.19 (6H, s) 3.00 (2H, br s) 3.31 (1H, s) |
| | | | 4.11-4.27 (2H, m) 4.60 (1H, t, J = 7.2 Hz) 4.91 (5H, |
| | | | s) 5.71 (1H, s) 6.07 (2H, d, J = 7.9 Hz) 6.92 (1H, |
| | | | d, J = 9.4 Hz) 7.02 (2H, d, J = 7.6 Hz) 8.55 (1H, |
| | | | s) 10.45 (1H, s) |
| 248 | CD₃OD | 270 | 2.19 (3H, s) 2.21 (3H, s) 3.08-3.13 (2H, m) |
| | | | 3.30 (1H, s) 4.22-4.24 (2H, m) 4.69 (1H, t, J = 7.2 Hz) |
| | | | 4.90 (2H, s) 5.72 (1H, s) 6.95 (1H, d, J = 9.2 Hz) |
| | | | 7.05-7.17 (2H, m) 7.73-7.81 (2H, m) |
| | | | 8.69 (1H, t, J = 5.6 Hz) 10.43 (1H, s) |
| 249 | CD₃OD | 270 | 2.20 (6H, s) 3.08-3.12 (2H, m) 3.30 (1H, s) |
| | | | 4.18-4.25 (2H, m) 4.69 (1H, t, J = 7.2 Hz) 4.90 (2H, |
| | | | s) 5.72 (1H, s) 6.79-6.83 (4H, m) 7.72-7.78 (2H, |
| | | | m) 8.67 (1H, s) 10.44 (1H, s) |
| 250 | CD₃OD | 270 | 2.19 (6H, s) 3.31-3.42 (2H, m) 4.03-4.18 (2H, m) |
| | | | 4.90 (3H, s) 5.71 (1H, s) 6.81 (1H, d, J = 8.4 Hz) |
| | | | 7.34 (2H, s) 7.57 (2H, s) 7.86 (2H, s) 8.57 (1H, |
| | | | s) 10.46 (1H, s) |
| 251 | d6- | 270 | 2.2 (6H, s), 2.5 (3H, s), 3.0-3.3 (1H, m), |
| | DMSO | | 4.0-4.2 (1H, m), 4.7-4.8 (1H, m), 5.7 (1H, s), |
| | | | 6.9-7.0 (1H, d, j = 7 Hz), 7.3 (1H, d, j = 7 Hz), 7.7-7.8 (2H, |
| | | | m), 8.0-8.1 (1H, m), 8.6-8.8 (1H, m), |
| | | | 10.9-11.0 (1H, m). |
| 252 | d6- | 270 | 2.1-2.3 (7, m), 2.5-2.6 (3H, m), 2.8-3.1 (2H, m), |
| | DMSO | | 4.0-4.2 (2H, m), 4.5-4.6 (1H, m), 5.7 (1H, s), |
| | | | 6.9-7.0 (1H, m) 7.0-7.2 (4H, m), 7.3-7.4 (1H, |
| | | | m), 7.6-7.8 (2H, m), 8.0-8.1 (1H, m), |
| | | | 8.6-8.7 (1H, m), 10.9-11 (1H, m) |
| 253 | d6- | 270 | 1.2 (9H, s), 2.1-2.15 (4H, d, j = 7 Hz), 2.5 (6H, s), |
| | DMSO | | 2.8-3.1 (2H, m), 4.1-4.2 (2H, m), 4.6 (1H, m), |
| | | | 5.6 (1H, s), 6.9-7.0 (1H, d, j = 9 Hz), 7.1-7.4 (4H, |
| | | | m), 7.8-7.9 (1H, m), 8.0 (1H, s), 8.6 (1H, m) |
| 254 | d6- | 270 | 2.1 (6H, s), 2.5 (3H, s), 2.8-3.1 (3H, m), |
| | DMSO | | 4.1-4.2 (2H, m), 4.6-4.8 (1H, m), 5.7 (1H, s), |
| | | | 6.7-6.8 (2H, m), |
| | | | 6.9-7.0 (1H, d, j = 7 Hz), 7.1-7.2 (2H, m), |
| | | | 7.25-7.30 (1H, d, j = 7 Hz). 7.7-7.9 (2H, m), |
| | | | 8.0-8.1 (1H, m), 8.6-8.7 (1H, m), 10.9 (1H, s) |
| 255 | d6- | 270 | 1.4-1.5 (3H, t, j = 7 Hz), 2.2 (6H, s), 2.9-3.1 (1H, |
| | DMSO | | m), 3.3 (5H, s), 3.9-4.1 (2H, q, j = 7 Hz), |
| | | | 4.1-4.3 (1H, m), 4.6-4.8 (1H, m), 5.8 (1H, s), |
| | | | 6.8-6.9 (2H, d, j = 9 Hz), 6.91-6.94 (1H, d, j = 9 Hz), |
| | | | 7.0-7.11 (2H, d, j = 9 Hz), 7.7-7.8 (2H, m) |
| | | | 8.5-8.6 (1H, m), 10.5 (1H, s) |
| 256 | CD₃OD | 270 | 1.2 (9H, s), 2.1-2.15 (4H, d, j = 7 Hz), 2.5 (6H, s), |
| | | | 2.8-3.1 (2H, m), 4.1-4.2 (2H, m), 4.6 (1H, m), |
| | | | 5.6 (1H, s), 6.9-7.0 (1H, d, j = 9 Hz), 7.1-7.4 (4H, |
| | | | m), 7.8-7.9 (1H, m), 8.0 (1H, s), 8.6 (1H, m) |
| 257 | CD₃OD | 400 | 2.07 (3H, s) 2.09 (3H, s) 3.22-3.24 (1H, m) |
| | | | 3.52-3.64 (1H, m) 3.69-3.75 (1H, m) |
| | | | 4.13-4.37 (2H, m) 4.81 (3H, br s) 5.02-5.05 (1H, M) |
| | | | 5.61 (1H, m) 6.76 (1H, d, J = 8.0 Hz) 7.40 (1H, s) |
| | | | 7.52-7.75 (4H, m) 7.80-7.90 (1H, m) |
| | | | 7.92-8.02 (2H, m) 8.66 (1H, t, J = 8.0 Hz) |
| 258 | CD₃OD | 400 | 2.24 (3H, s) 3.20-3.25 (1H, m) 3.30-3.36 (1H, |
| | | | m) 4.23-4.33 (2H, m) 4.81 (1H, t, J = 8.0 Hz) |
| | | | 4.92 (4H, br s) 6.05-6.06 (1H, m) 6.85 (1H, d, J = 4.0 Hz) |
| | | | 6.99 (1H, d, J = 8.0 Hz) 7.47 (2H, d, J = 8.0 Hz) |
| | | | 7.60 (2H, d, J = 8.0 Hz) |
| | | | 7.79-7.82 (2H, m) 10.86 (1H, s) |
| 259 | CD₃OD | 270 | 2.06 (3H, s) 3.04-3.13 (3H, m) 4.16-4.18 (2H, |
| | | | m) 4.66 (1H, t, J = 7.7 Hz) 4.93 (2H, s) |
| | | | 6.66 (2H, d, J = 6.9 Hz) 6.89 (1H, d, J = 9.9 Hz) |
| | | | 7.14-7.24 (4H, m) 10.6 (1H, s) |
| 260 | CD₃OD | 270 | 2.06 (3H, s) 3.01-3.18 (3H, m) 4.17 (2H, s) |
| | | | 4.63 (1H, t, J = 7.8 Hz) 4.91 (3H, br s) 6.65 (2H, d, J = 7.4 Hz) |
| | | | 6.88-6.98 (3H, m) 7.20-7.25 (2H, m) |
| | | | 7.68 (2H, d, J = 6.4 Hz) 10.6 (1H s) |
| 261 | CD₃OD | 400 | 2.25 (3H, s) 3.16-3.30 (3H, m) 4.17-4.26 (2H, |
| | | | m) 4.68 (1H, t, J = 8.0 Hz) 4.94-4.99 (4H, m) |
| | | | 6.00-6.01 (1H, m) 6.99-7.01 (3H, m) |
| | | | 7.21-7.25 (2H, m) 7.72-7.75 (2H, m) 10.72 (1H, s) |
| 262 | CD₃OD | 400 | 2.30 (3H, s) 3.26-3.46 (2H, m) 4.30 (2H, d, J = 4.0 Hz) |
| | | | 4.86 (1H, t, J = 8.0 Hz) 4.91 (4H, br s) |
| | | | 6.05 (1H, d, J = 4.0 Hz) 6.83 (1H, s) 6.95 (1H, d, |
| | | | J = 8.0 Hz) 7.20-7.27 (2H, m) 7.32-7.46 (2H, m0 |
| | | | 7.72-7.77 (2H, m) 10.75 (1H, s) |
| 263 | CD₃OD | 400 | 2.29 (3H, s) 3.12-3.37 (2H, m) 4.22 (2H, d, J = 4.0 Hz) |
| | | | 4.84 (1H, t, J = 8.0 Hz) 4.99-5.08 (5H, |
| | | | m 0 6.05 (1H, d, J = 4.0 Hz) 6.78-6.85 (1H, m) |
| | | | 6.98-7.07 (1H, m) 7.22-7.24 (1H, m) 7.43 (1H, t, |
| | | | J = 4.0 Hz) 7.75-7.80 (2H, m) 10.76 (1H, s) |
| 264 | CD₃OD | 400 | 2.32 (3H, s) 3.12-3.19 (2H, m) 4.19-4.35 (2H, |
| | | | m) 4.76 (1H, t, J = 4.0 Hz) 4.93 (4H, br s) |
| | | | 6.06 (1H, s) 6.858 (1H, t, J = 4.0 Hz) 7.00 (1H, dd, J = 8.0, |
| | | | 4.0 Hz) 7.20-7.29 (1H, m) 7.41-7.48 (2H, |
| | | | m) 7.74-7.78 (2H, m) 10.75 (1H, br s) |
| 265 | CD₃OD | 400 | 2.30 (3H, s) 3.12-3.31 (2H, m) 4.19-4.39 (2H, |
| | | | m) 4.82 (1H, t, J = 4.0 Hz) 5.00 (4H, br s) |
| | | | 6.04 (1H, d, J = 4.0 Hz) 6.83 (1H, s) 6.98-7.04 (3H, |
| | | | m) 7.08-7.10 (1H, m) 7.25-7.32 (1H, m) |
| | | | 7.72-7.85 (2H, m) 10.80 (1H, s) |
| 266 | CD₃OD | 400 | 2.27 (3H, s) 3.12-3.30 (2H, m) 4.20-4.37 (2H, |
| | | | m) 4.77 (1H, t, J = 8.0 Hz) 5.01 (4H, s) |
| | | | 6.05 (1H, t, J = 4.0 Hz) 6.84 (1H, t, J = 4.0 Hz) |
| | | | 6.97 (1H, d, J = 8.0 Hz) 7.21-7.24 (1H, m) |
| | | | 7.27-7.33 (3H, m) 7.74-7.77 (2H, m) 10.79 (1H, s) |
| 267 | CD₃OD | 400 | 2.28 (3H, s) 3.07-3.20 (2H, m) 4.19-4.35 (2H, |
| | | | m) 4.75 (1H, t, J = 8.0 Hz) 5.07 (4H, s) |
| | | | 6.06 (1H, d, J = 4.0 Hz) 6.85 (1H, d, J = 4.0 Hz) |
| | | | 7.00 (1H, d, J = 8.0 Hz) 7.22-7.38 (4H, m) |
| | | | 7.73-7.79 (2H, m) 10.77 (1H, s) |
| 268 | CD₃OD | 400 | 2.27 (3H, s) 2.30 (3H, s) 3.10-3.29 (2H, m) |
| | | | 4.19-4.27 (2H, m) 4.77 (1H, t, J = 8.0 Hz) 5.24 (4H, s) |
| | | | 6.04 (1H, s) 6.82 (1H, s) 6.92-6.95 (1H, m) |
| | | | 7.04-7.09 (3H, m) 7.15-7.27 (1H, m) 7.68-7.74 (2H, |
| | | | m) 10.80 (1H, s) |
| 269 | CD₃OD | 400 | 2.23 (3H, s) 3.12-3.26 (2H, m) 4.22-4.46 (2H, |
| | | | m) 4.78 (1H, t, J = 4.0 Hz) 4.93 (4H, s) |
| | | | 6.05 (1H, t, J = 4.0 Hz) 6.84 (1H, t, J = 4.0 Hz) |
| | | | 6.91-7.04 (3H, m) 7.11-7.17 (1H, m) 7.26-7.34 (1H, |
| | | | m) 7.75-7.80 (2H, m) 10.86 (1H, s) |
| 270 | CD₃OD | 400 | 2.31 (3H, s) 3.05-3.15 (2H, m) 4.26-4.39 (2H, |
| | | | m) 4.60 (1H, t, J = 8.0 Hz) 4.93 (4H, br s) |
| | | | 6.06 (1H, s) 6.82-6.89 (4H, m) 7.00 (1H, d, J = 8.0 Hz) |
| | | | 7.78 (1H, s) 7.83 (1H, dd, J = 8.0, 4.0 Hz) |
| | | | 10.76 (1H, s) |
| 271 | CD₃OD | 400 | 2.27 (3H, s) 3.15-3.25 (1H, m) 3.33-3.37 (1H, |
| | | | m) 4.32 (2H, s) 4.81 (1H, t, J = 8.0 Hz) |
| | | | 4.95 (4H, br s) 6.04 (1H, s) 6.83 (1H, s) 7.08 (1H, d, J = 8.0 Hz) |
| | | | 7.48-7.61 (4H, m) 7.71-7.88 (2H, m) |
| | | | 10.86 (1H, s) |
| 272 | CD₃OD | 400 | 2.24 (3H, s) 3.20-3.25 (1H, m) 3.30-3.36 (1H, |
| | | | m) 4.23-4.33 (2H, m) 4.81 (1H, t, J = 8.0 Hz) |
| | | | 4.92 (4H, br s) 6.05-6.06 (1H, m) 6.85 (1H, d, J = 4.0 Hz) |
| | | | 6.99 (1H, d, J = 8.0 Hz) 7.47 (2H, d, J = 8.0 Hz) |
| | | | 7.60 (2H, d, J = 8.0 Hz) |
| | | | 7.79-7.82 (2H, m) 10.86 (1H, s) |
| 273 | CD₃OD | 400 | 0.96-1.08 (2H, m), 1.20-1.30 (3H, m), |
| | | | 1.43-1.51 (1H, m), 1.71-1.89 (7H, m), 2.40 (3H, s), |
| | | | 4.29-4.39 (2H, m), 4.55-4.61 (1H, m), 4.95 (4H, s), |
| | | | 6.08 (1, d, J = 2.3 Hz), 6.86 (1H, t, J = 2.7 Hz), 7.03 (1H, |
| | | | d, J = 9.5 Hz) 7.82 (1H, d, J = 1.04 Hz) 7.9 (1H, dd, J = 9.24, |
| | | | 2.12 Hz) 8.70-8.81 (1H, m) |
| 274 | CD₃OD | 270 | 3.26-3.30 (1H, m) 3.82 (3H, s) 4.17 (2H, s) |
| | | | 4.75-4.89 (7H, m) 6.53 (1H, d, J = 8.4 Hz) 6.79 (2H, |
| | | | s) 7.31 (1H, dd, J = 6.5, 2.2 Hz) 7.41-7.48 (3H, |
| | | | m) 7.60 (1H, s) 7.75 (1H, d, J = 1.7 Hz) |
| 275 | CD₃OD | 270 | 3.25-3.32 (2H, m) 4.11-4.23 (1H, m) |
| | | | 4.68-4.75 (1H, m) 4.90 (7H, br s) 6.83-6.84 (2H, m) |
| | | | 6.92 (1H, d, J = 9.2 Hz) 7.48-7.57 (3H, m) |
| | | | 7.71-7.74 (2H, m) 8.68 (1H, d, J = 5.3 Hz) |
| 276 | CD₃OD | 270 | 2.21 (3H, s) 3.18-3.30 (3H, m) 4.21 (2H, s) |
| | | | 4.74 (1H, t, J = 7.4 Hz) 4.93 (5H, br s) 6.63 (1H, s) |
| | | | 6.91 (1H, d, J = 9.1 Hz) 7.48-7.56 (3H, m) |
| | | | 7.69-7.71 (2H, m) 8.73 (1Hs) |
| 277 | CD₃OD | 270 | 1.8-1.9 (2H, m), 2.2-2.4 (6H, m), 3.2-3.3 (2H, |
| | | | m), 4.1-4.4 (4H, m), 4.7-4.9 (1H, m), |
| | | | 5.7-5.8 (1H, m) 6.2-6.4 (2H, m), 6.7-6.8 (1H, m), |
| | | | 7.1-7.2 (1H, m) 7.3-7.4 (4H, m), 7.5-7.6 (2H, m) |
| 278 | CD₃OD | 270 | 2.1 (4H, s), 2.2 (1H, s), 2.5 (5H, s), 4.0-4.1 (1H, |
| | | | m), 4.7-4.9 (1H, m), 5.6 (1H, s), 6.5-6.7 (1H, |
| | | | m), 7.4-7.6 (8H, m), 7.9-8.1 (2H, m), |
| | | | 8.3-8.4 (1H, m), 8.6-8.7 (1H, m), 9.9-10 (1H, m), |
| | | | 11 (1H, s) |
| 279 | CD₃OD | 270 | 1.3-1.5 (6, m), 3.0 (5H, m), 4.1-4.4 (2H, m), |
| | | | 4.7-4.9 (2H, m), 5.8 (1H, s), 6.2 (1H, d, J = 7 Hz), |
| | | | 6.5 (1H, d, J = 7 Hz), 7.0-7.4 (7H, m), 9.8 (1H, m) |
| 280 | CD₃OD | 270 | 1.3-1.5 (6, m), 3.0 (5H, m), 4.1-4.4 (2H, m), |
| | | | 4.7-4.9 (2H, m), 5.8 (1H, s), 6.2 (1H, d, J = 7 Hz), |
| | | | 6.5 (1H, d, J = 7 Hz), 7.0-7.4 (7H, m), 9.8 (1H, m) |
| 281 | CD₃OD | 270 | 2.2 (6H, s), 2.3 (2H, s), 2.4 (3H, s), 3.0-3.1 (2H, |
| | | | m), 3.3 (3H, s), 4.0-4.4 (2H, m), 4.7-4.8 (1H, m), |
| | | | 5.7 (1H, s), 6.7 (1H, d, J = 7 Hz), 7.0-7.3 (5H, |
| | | | m), 7.6 (1H, d, J = 7 Hz), 8.5 (1H, m), |
| | | | 10.5-10.8 (1H, m) |
| 282 | CD₃OD | 270 | 2.17 (6H, d, J = 3.2 Hz) 2.37 (3H, s) |
| | | | 3.24-3.33 (2H, m) 4.14-4.18 (2H, m) 4.78 (1H, t, J = 7.7 Hz) |
| | | | 4.98 (1H, s) 5.69 (1H, s) 6.71 (1H, d, J = 9.2 Hz) |
| | | | 7.44-7.48 (4H, m) 7.65-7.68 (3H, m) |
| | | | 8.57 (1H, t, J = 5.4 Hz) 10.46 (1H, s) |
| 286 | CD₃OD | 400 | 2.36 (3H, s) 2.49 (3H, s) 3.10-3.23 (2H, m) |
| | | | 4.17-4.22 (1H, m) 4.29-4.38 (1H, m) 4.76 (1H, t, J = 8.0 Hz) |
| | | | 4.93 (3H, br s) 6.05 (1H, s) |
| | | | 6.79-6.84 (2H, m) 7.05-7.07 (1H, m) 7.11-7.20 (2H, m) |
| | | | 7.74 (1H, d, J = 8.0 Hz) 8.67 (1H, t, J = 4.0 Hz) |
| | | | 10.89 (1H, br s) |
| 287 | CD₃OD | 270 | 3.10-3.15 (1H, m) 3.29-3.32 (1H, m) 4.19 (1H, |
| | | | q, J = 6.7 Hz) 4.72 (1H, t, J = 7.7 Hz) 4.91 (5H, |
| | | | br s) 6.92-6.99 (3H, m) 7.21-7.26 (2H, m) |
| | | | 7.40-7.41 (2H, m) 7.68 (2H, br s) 7.71 (1H, d, J = 2.2 Hz) |
| 288 | CD₃OD | 270 | 3.11-3.19 (1H, m) 3.30 (1H, s) 4.20 (1H, q, J = 8.9 Hz) |
| | | | 4.76 (1H, t, J = 7.4 Hz) 4.95 (6H, br s) |
| | | | 6.91-6.98 (3H, m) 7.05 (1H, d, J = 7.7 Hz) |
| | | | 7.22-7.27 (1H, m) 7.47 (2H, s) 7.68-7.73 (2H, m) |
| 289 | CD₃OD | 270 | 3.30 (1H, br s) 3.57-3.68 (1H, m) 3.97-4.18 (1H, |
| | | | m) 4.91 (7H, br s) 6.82 (1H, d, J = 9.2 Hz) |
| | | | 7.32-7.39 (4H, m) 7.45-7.56 (4H, m) 7.75 (1H, d, J = 6.9 Hz) |
| | | | 7.86 (1H, d, J = 7.4 Hz) 8.21 (1H, d, J = 8.2 Hz) |
| 291 | CD₃OD | 270 | 2.22 (3H, s) 2.88-3.06 (1H, m) 3.11-3.19 (1H, |
| | | | m) 3.94 (3H, s) 4.22 (2H, s) 4.65 (1H, t, J = 8.0 Hz) |
| | | | 4.88 (4H, S) 6.55 (1h, S) 6.94-7.06 (3H, m) |
| | | | 7.24-7.27 (2H, m) 7.72-7.76 (2H, m) |
| 292 | CD₃OD | 270 | 2.32 (3H, s) 2.79 (2H, s) 4.03-4.09 (2H, m) |
| | | | 4.88-4.94 (6H, m) 6.47 (2H, s) 6.78-6.81 (3H, s) |
| | | | 6.91-7.08 (2H, m) 7.16-7.53 (6H, m) 7.72-7.75 (1H, |
| | | | m) 7.83-7.85 (1H, m) 8.18 (1H, s) |
| 293 | CD₃OD | 270 | 2.33 (3H, s) 3.30-3.48 (3H, m) 4.03-4.14 (2H, |
| | | | m) 4.83-4.91 (5H, m) 6.82 (1H, s) |
| | | | 7.17-7.20 (2H, m) 7.31-7.56 (8H, m) 7.83-7.88 (2H, m) |
| | | | 11.06 (1H, br s) |
| 294 | CD₃OD | 270 | 1.27-1.37 (5H, m) 2.32 (2H, s) 3.13-3.15 (1H, |
| | | | m) 3.17-3.23 (3H, m) 4.20 (1H, s) 4.30 (1H, s) |
| | | | 6.91 (1H, d, J = 3.7 Hz) 6.94-7.07 (1H, m) |
| | | | 7.16-7.25 (8H, m) 7.53 (1H, d, J = 8.2 Hz) |
| | | | 7.71-7.73 (1H, m) |
| 295 | CD₃OD | 270 | 1.32-1.36 (2H, m) 2.30 (3H, s) 3.20-3.38 (2H, |
| | | | m) 4.16 (1H, s) 4.82-4.85 (1H, m) 4.92 (4H, br |
| | | | s) 6.45 (1H, d, J = 3.9 Hz) 6.85-6.90 (2H, m) |
| | | | 7.12-7.35 (6H, m) 7.50 (2H, d, J = 7.9 Hz) |
| | | | 7.62-7.71 (2H, m) |
| 296 | CD₃OD | 270 | 1.33-1.37 (1H, m) 2.33 (3H, s) 3.01-3.20 (2H, |
| | | | m) 3.30 (1H, s) 4.11-4.26 (2H, m) 4.70 (1H, t, J = 7.7 Hz) |
| | | | 4.91 (3H, s) 6.46 (1H, d, J = 3.9 Hz) |
| | | | 6.90-6.94 (2H, m) 7.17-7.25 (3H, m) |
| | | | 7.35-7.40 (2H, m) 7.53 (2H, d, J = 7.9 Hz) 7.68-7.70 (2H, |
| | | | m) |
| 297 | CD₃OD | 270 | 1.27-1.32 (1H, m) 2.32 (3H, s) 3.07-3.17 (3H, |
| | | | m) 4.17 (2H, d, J = 4.5 Hz) 4.69-4.72 (1H, m) |
| | | | 4.91 (2H, s) 6.46 (1H, s) 6.88-6.91 (2H, m) |
| | | | 7.16-7.26 (5H, m) 7.50-7.53 (2H, m) 7.65-7.69 (2H, |
| | | | m) 8.61 (1H, d, J = 4.9 Hz) 11.03 (1H, s) |
| 298 | CD₃OD | 270 | 3.29-3.31 (1H, m), 3.58-3.73 (1H, m), |
| | | | 4.05-4.13 (2H, m), 4.89-4.91 (4H, m), |
| | | | 6.83 (1H, d, J = 9.1 Hz), 7.06-7.09 (1H, m), |
| | | | 7.15 (1H, s), 7.19-7.25 (1H, m), 7.34-7.62 (9H, m), |
| | | | 7.73-7.76 (1H, m), 7.84-7.88 (1H, m), |
| | | | 8.22 (1H, d, J = 7.6 Hz), 8.40-8.60 (1H, m) |
| 299 | CD₃OD | 270 | 3.61-3.71 (2H, m), 4.06-4.143 (2H, m), |
| | | | 4.90-4.97 (4H, m), 6.84 (1H, d, J = 9.1 Hz), |
| | | | 7.33-7.40 (4H, m), 7.49-7.55 (4H, m), 7.64-7.68 (2H, m), |
| | | | 7.74-7.77 (1H, m), 7.84-7.88 (1H, m), |
| | | | 8.26 (1H, d, J = 8.46 Hz), 8.40-8.60 (1H, m) |
| 300 | CD₃OD | 270 | 3.57-3.60 (1H, m), 3.67-3.71 (1H, m), 3.72 (3H, s), |
| | | | 4.05-4.13 (2H, m), 4.86-4.92 (4H, m), |
| | | | 6.81-6.91 (2H, m), 7.07 (1H, s), 7.29-7.33 (3H, m), |
| | | | 7.37-7.54 (6H, m), 7.73-7.77 (1H, m), 7.85-7.88 (1H, m), |
| | | | 8.22 (1H, d, J = 8.2 Hz), 8.45-8.47 (1H, m) |
| 301 | CD₃OD | 270 | 3.57-3.60 (1H, m), 3.707-3.73 (1H, m), |
| | | | 4.06-4.13 (2H, m), 4.86-4.92 (4H, m), |
| | | | 6.82 (1H, d, J = 9.1 Hz), 6.98-7.05 (1H, m), |
| | | | 7.12 (1H, s), 7.26-7.33 (3H, m), 7.36-7.55 (6H, m), |
| | | | 7.74-7.77 (1H, m), 7.85-7.88 (1H, m), |
| | | | 8.22 (1H, d, J = 8.2 Hz), 8.45-8.47 (1H, m) |
| 302 | CD₃OD | 270 | 3.56-3.59 (1H, m), 3.69-3.72 (1H, m), |
| | | | 4.05-4.13 (2H, m), 4.86-4.90 (6H, m), 6.78-6.84 (2H, m), |
| | | | 6.95-6.99 (2H, m), 7.23-7.27 (1H, m), |
| | | | 7.31-7.40 (2H, m), 7.48-7.54 (4H, m), 7.74-7.77 (1H, m), |
| | | | 7.85-7.88 (1H, m), 8.21 (1H, d, J = 8.2 Hz), |
| | | | 8.45-8.47 (1H, m) |
| 303 | CD₃OD | 270 | 2.1 (1H, s), 2.4 (2H, s), 2.6 (2H, s), 3.0-3.3 (2H, |
| | | | m), 4.1-4.2 (2H, m), 4.7-4.8 (1H, m), 6.6 (1H, |
| | | | m), 7.0-7.1 (1H, m), 7.2-7.3 (6H, m), |
| | | | 7.6-7.8 (4H, m), 8.8-8.9 (2H, m), 11.6 (1H, s) |
| 304 | CD₃OD | 270 | 2.0 (3H, s), 3.4-3.7 (3H, m), 3.8 (3H, s), |
| | | | 4.1-4.3 (2H, m), 4.8-5.2 (3H, m), 5.5 (1H, s), |
| | | | 6.2-6.3 (1H, m), 6.8-6.9 (1H, m), |
| | | | 7.0-7.1 (3H, m), 7.2-7.4 (3H, m), 7.4-7.6 (3H, |
| | | | m), 7.8-7.9 (2H, m), 8.3 (1H, m) |
| 305 | CD₃OD | 270 | 2.17 (3H, s), 3.55-3.58 (1H, m), 3.65-3.68 (1H, m), |
| | | | 3.96-3.98 (1H, m), 4.115-4.13 (1H, m), |
| | | | 4.89-4.95 (4H, m), 6.63 (1H, d, J = 9.1 Hz), |
| | | | 6.98-6.84 (2H, m), 6.98-7.05 (1H, m), 7.13 (1H, s), |
| | | | 7.26-7.52 (6H, m), 7.74-7.76 (1H, m), 7.84-7.88 (1H, m), |
| | | | 8.17-8.19 (1H, m), 8.45-8.47 (1H, m) |
| 306 | CD₃OD | 270 | 3.40-3.60 (1H, m), 3.70-3.80 (1H, m), |
| | | | 3.89 (3H, d, J = 2.8 Hz), 4.05-4.11 (2H, m), |
| | | | 4.13-4.16 (1H, m), 4.89-4.91 (2H, m), |
| | | | 6.85 (1H, d, J = 8.9 Hz), 7.03 (1H, s), |
| | | | 7.07-7.12 (1H, m), 7.26-7.64 (9H, m), 7.76-7.78 (1H, m), |
| | | | 7.85-7.88 (1H, m), 8.22 (1H, d, J = 7.6 Hz), |
| | | | 8.50-8.59 (1H, m) |
| 307 | CD₃OD | 270 | 3.05-3.10 (1H, m), 3.25-3.40 (2H, m), |
| | | | 4.10-4.25 (2H, m), 4.85-4.95 (1H, m), 4.95-5.10 (4H, m), |
| | | | 6.50 (1H, d, J = 8.15 Hz), 7.00-7.80 (11H, m) |
| 308 | CD₃OD | 270 | 3.05-3.20 (1H, m), 3.25-3.40 (2H, m), |
| | | | 3.80 (3H, s), 4.10-4.25 (2H, m), 4.75-4.85 (1H, m), |
| | | | 4.95-5.05 (4H, m), 6.45-6.55 (1H, m), |
| | | | 6.85-6.90 (1H, m), 7.00-7.10 (2H, m), 7.25-7.80 (7H, m) |
| 309 | CD₃OD | 270 | 1.7-1.8 (3H, m), 2.1-2.2 (3H, s), 4.1-4.2 (2H, m), |
| | | | 4.3 (1H, s), 4.8-5.0 (1H, m), 6.1-6.2 (1H, d, |
| | | | J = 7 Hz), 6.5-6.6 (1H, m), 7.0 (1H, s), |
| | | | 7.1-7.7 (10H, m), 9.4-9.5 (1H, m) |
| 310 | CD₃OD | 270 | 1.2-1.3 (2H, m), 1.8-2.1 (3H, m), 2.2-2.3 (2H, |
| | | | m), 3.2-3.3 (2H, m), 3.8 (3H, s), 4.1-4.2 (2H, m), |
| | | | 4.8-4.9 (1H, m), 6.1-6.2 (1H, m) 7.1-7.4 (10H, |
| | | | m) |
| 311 | CD₃OD | 270 | 3.00-3.40 (3H, m), 4.10-4.30 (2H, m), |
| | | | 4.70-4.80 (1H, m), 4.90-5.10 (3H, m), 6.80-7.80 (12H, m), |
| | | | 8.60-8.80 (1H, m) |
| 312 | CD₃OD | 270 | 3.00-3.40 (4H, m), 3.80 (3H, s), |
| | | | 4.15-4.30 (2H, m), 4.75-4.90 (1H, m), 4.95-5.10 (2H, m), |
| | | | 6.80-7.40 (9H, m), 7.60-7.80 (2H, m), |
| | | | 8.40-8.70 (1H, m) |
| 313 | CD₃OD | 270 | 3.00-3.40 (4H, m), 4.10-4.30 (2H, m), |
| | | | 4.80-4.90 (1H, m), 4.95-5.10 (3H, m), 6.90-7.80 (12H, m), |
| | | | 8.60-8.80 (1H, m), 10.90-11.10 (1H, m) |
| 314 | CD₃OD | 270 | 3.05-3.40 (4H, m), 3.80 (3H, s), |
| | | | 4.10-4.30 (2H, m), 4.70-4.90 (1H, m), 4.95-5.10 (2H, m), |
| | | | 6.80-7.40 (9H, m), 7.60-7.80 (2H, m), |
| | | | 8.60-8.75 (1H, m) |
| 315 | d6- | 270 | 2.1 (2H, s), 2.4 (2H, s), 2.6 (3H, s), 3.0-3.3 (2H, |
| | DMSO | | m), 4.2-4.3 (2H, m), 4.8 (1H, br s), 6.8 (1H, br |
| | | | m), 7.0-7.1 (1H, m), |
| | | | 7.1 (6H, m), 7.6-7.8 (2H, m), 8.8-8.9 (1H, m), |
| | | | 11.4 (1H, m) |
| 316 | CD₃OD | 270 | 2.4 (3H, s), 2.6 (3H, s), 2.9-3.2 (2H, m), |
| | | | 4.1-4.2 (2H, m), 4.6-4.7 (1H, s), 6.8-6.9 (1H, m), |
| | | | 7.1-7.4 (8H, m), 8.2-8.3 (3H, m), 8.8 (2H, s), 11.4 (1H, |
| | | | s) |
| 317 | CD₃OD | 270 | 2.69 (3H, s), 3.00-3.25 (2H, m), |
| | | | 3.28-3.35 (1H, m), 4.15-4.30 (2H, m), 4.65-4.80 (1H, m), |
| | | | 4.90-5.10 (3H, m), 6.85-7.70 (12H, m), |
| | | | 8.55-8.70 (1H, m) |
| 318 | CD₃OD | 270 | 2.27 (3H, s) 3.00-3.25 (2H, m), 3.28-3.35 (1H, m), |
| | | | 3.80 (3H, s) 4.10-4.30 (2H, m), 4.75-4.85 (1H, m), |
| | | | 4.90-5.10 (4H, m), 6.80-7.50 (9H, m), |
| | | | 7.60-7.70 (2H, m), 8.50-8.70 (1H, m) |
| 319 | CD₃OD | 270 | 2.2 (3H, s), 2.4 (3H, s), 3.0-3.2 (2H, m), |
| | | | 3.2-3.4 (3H, m), 4.0-4.3 (2H, m), 4.7-4.8 (1H, m), |
| | | | 6.7-6.8 (1H, m), 7.0-7.3 (8H, m), 7.4-7.5 (1H, m), |
| | | | 7.6-7.7 (2H, m), 8.4-8.6 (1H, m) |
| 320 | CD₃OD | 270 | 2.4 (3H, s), 3.0-3.2 (3H, m), 3.3 (3H, s), |
| | | | 4.0-4.2 (2H, m), 4.7-4.8 (1H, m), 6.7 (1H, d, J = 7 Hz), |
| | | | 7.0-7.2 (4H, m), 7.3-7.5 (3H, m), 7.6-7.8 (2H, |
| | | | m), 8.6-8.7 (1H, m) |
| 321 | CD₃OD | 270 | 2.4 (3H, s), 3.0-3.2 (2H, m), 3.3-3.5 (3H, s), |
| | | | 3.7 (3H, s), 4.0-4.4 (2H, m), 4.8-4.9 (2H, m), |
| | | | 6.6-6.8 (1H, m), 6.9-7.0 (1H, m), 7.1 (2H, s), |
| | | | 7.2-7.6 (4H, m), 7.7-7.8 (1H, m), 8.7-8.6 (1H, m) |
| 322 | d6- | 270 | 2.38 (3H, s) 3.39-3.44 (2H, m) 4.12 (2H, s) |
| | DMSO | | 4.79 (1H, d, J = 4.9 Hz) 6.76 (1H, d, J = 8.7 Hz) |
| | | | 7.04-7.07 (1H, m) 7.18-7.26 (2H, m) 7.37-7.50 (4H, |
| | | | m) 7.62-7.72 (3H, m) 7.93 (2H, br s) 8.59 (1H, |
| | | | s) 8.83 (1H, d, J = 7.9 Hz) 11.54 (1H, br s) |
| 324 | CD₃OD | 270 | 3.10-3.15 (1H, m) 3.22-3.30 (2H, m) 4.21 (2H, |
| | | | s) 4.78 (1H, t, J = 7.7 Hz) 4.92 (4H, s) 6.91 (1H, |
| | | | d, J = 8.4 Hz) 7.05-7.24 (5H, m) 7.41 (1H, d, J = 8.4 Hz) |
| | | | 7.58 (1H, d, J = 7.9 Hz) 7.71-7.78 (2H, |
| | | | m) |
| 325 | CD₃OD | 270 | 3.12-3.33 (2H, m) 4.24 (1H, s) 4.76 (1H, s) |
| | | | 4.93 (6H, s) 7.10-7.24 (7H, m) 7.43 (1H, s) 7.62 (1H, |
| | | | s) 7.74-7.78 (2H, m) |
| 326 | CD₃OD | 270 | 3.09-3.30 (2H, m) 4.21 (2H, s) 4.71-4.76 (1H, s) |
| | | | 4.91 (5H, s) 6.91-7.10 (7H, m) 7.72 (2H, s) |
| | | | 8.68 (1H, s) |
| 327 | CD₃OD | 270 | 1.36 (1H, s) 3.03-3.30 (2H, m) 3.79 (3H, s) |
| | | | 4.19 (2H, s) 4.70-4.76 (1H, m) 4.91 (4H, s) |
| | | | 6.88-7.27 (8H, m) 7.69 (2H, s) |
| 328 | CD₃OD | 270 | 3.11-3.21 (2H, m) 4.23 (2H, s) 4.73-4.79 (1H, t) |
| | | | 4.98 (5H, s) 6.93-7.16 (4H, m) 7.74-7.99 (4H, |
| | | | m) 8.26 (1H, s) 9.33 (1H, s) |
| 329 | d6- | 270 | 3.0-3.2 (1H, m) 3.3-3.4 (2H, m), 4.0-4.2 (3H, |
| | DMSO | | m), 4.8-4.9 (1H, m), |
| | | | 7-7.8 (8H, m), 8.0-8.1 (1H, m), 8.7-9.0 (2H, m) |
| 330 | CD₃OD | 270 | 3.12-3.19 (2H, m) 4.18 (2H, s) 4.73 (1H, br s) |
| | | | 4.91 (5H, s) 6.95-7.26 (8H, m) 7.60-7.66 (3H, |
| | | | m) 8.64 (1H, br s) |
| 331 | CD₃OD | 270 | 3.09-3.30 (3H, m) 4.20 (2H, s) 4.71 (1H, br s) |
| | | | 4.89 (4H, br s) 6.96-7.10 (5H, m) 7.27-7.40 (4H, |
| | | | m) 7.69 (2H, s) |
| 332 | CD₃OD | 270 | 3.09-3.30 (3H, m) 3.79 (3H, s) 4.19 (2H, s) |
| | | | 4.71 (1H, brs) 4.91 (4H, s) 6.91-7.05 (7H, m) |
| | | | 7.27-7.28 (2H, m) 7.67 (2H, s) |
| 333 | CD₃OD | 270 | 4.09-4.18 (2H, m) 5.19 (2H, s) 5.70-5.75 (1H, |
| | | | m) 5.98 (5H, s) 7.89-7.97 (3H, m) |
| | | | 8.23-8.25 (2H, m) 8.85-9.00 (4H, m) 9.22 (1H, s) |
| | | | 10.34 (1H, s) |
| 334 | d6- | 270 | 2.5 (3H, s), 2.9-3.1 (2H, m), 3.7 (3H, s), |
| | DMSO | | 4.1-4.2 (2H, m), 4.5-4.6 (1H, m), 6.77-6.8 (1H, d, |
| | | | J = 8 Hz), 6.9-6.93 (1H, d, J = 9 Hz), 7.00-7.06 (1H, |
| | | | m), 7.14-7.3 (4H, m), 7.38-7.41 (1H, d, J = 8 Hz), |
| | | | 7.6-7.65 (1H, d, J = 8 Hz), 7.7-7.71 (1H, m), |
| | | | 7.91-8.0 (1H, m), 8.7-8.8 (1H, m), 11.5 (1H, s) |
| 335 | d6- | 270 | 3.39 (7H, s) 4.19 (1H, s) 4.85 (1H, s) |
| | DMSO | | 6.91-7.02 (2H, m) 7.18-7.23 (4H, m) 7.40-7.53 (4H, m) |
| | | | 7.80-8.01 (2H, m) 8.77 (1H, s) 11.52 (1H, s) |
| 336 | CD₃OD | 270 | 1.3-1.6 (1H, m), 2.2-2.3 (3H, m), 2.8-2.9 (1H, |
| | | | m), 3.2-3.4 (6H, m), 4.0-4.4 (2H, m), |
| | | | 4.7-4.9 (1H, m), 6.5-6.6 (1H, m), 7.0-7.3 (6H, m), |
| | | | 7.4-7.6 (4H, m), 7.7-7.9 (1H, m) |
| 337 | CD₃OD | 270 | 2.2 (3H, s), 3.3-3.4 (6H, m), 3.8 (3H, s), |
| | | | 3.9-4.2 (2H, m), 4.8-5.0 (1H, m), 6.5-6.6 (1H, d, J = Hz), |
| | | | 6.9-7.0 (3, m), 7.1-7.2 (4H, m), 7.3-7.5 (3H, |
| | | | m), 7.6-7.8 (1H, m), 8.3-8.4 (1H, m) |
| 338 | CD₃OD | 270 | 1.1-1.0 (2H, m), 1.5-1.8 (10H, m), 2.5 (3H, s), |
| | | | 4.1-4.2 (2H, m), 4.5-4.6 (1H, m), 6.9-7.1 (2H, |
| | | | m), 7.2-7.3 (2H, m), 7.4-7.5 (1H, m), |
| | | | 7.6-7.7 (1H, m), 7.8-7.9 (1H, m), 8.0-8.1 (1H, s), |
| | | | 8.5-8.7 (2H, m), 11.6 (1H, s) |
| 339 | CD₃OD | 400 | 0.99-1.14 (10H, m), 1.22-1.39 (6H, m), |
| | | | 1.65-1.84 (9H, m), 1.98-2.09 (2H, m), 2.71 (3H, s), |
| | | | 4.24-4.44 (2H, m), 4.53 (1H, dd, J = 6.64, 8.8 Hz), |
| | | | 7.06 (1H, d, J = 9.16 Hz) 7.85 (1H, s), |
| | | | 7.95 (1H, dd, J = 1.96, 9.12 Hz), 8.79-8.82 (1H, m) |
| 340 | CD₃OD | 400 | 0.58-0.89 (5H, m) 1.15-1.25 (3H, m) |
| | | | 1.88-1.99 (1H, m) 2.51-2.54 (3H, m) 2.76-2.88 (2H, m) |
| | | | 3.01-3.22 (3H, m) 3.47-3.55 (1H, m) |
| | | | 4.11-4.21 (1H, m) 4.52-4.64 (1H, m) 6.87 (1H, d, J = 8.0 Hz) |
| | | | 7.05-7.19 (1H, m) 7.31-7.48 (2H, m) |
| | | | 7.65-7.77 (2H, m) 8.66 (1H, d, J = 4.0 Hz) |
| 341 | CD₃OD | 400 | 0.70 (3H, d, J = 8.0 Hz) 0.76 (3H, d, J = 8.0 Hz) |
| | | | 1.88-1.96 (1H, m) 2.46-2.54 (3H, m) |
| | | | 2.80-2.92 (1H, m) 3.03-3.17 (2H, m) 3.20-3.22 (1H, m) |
| | | | 3.49 (1H, t, J = 4.0 Hz) 4.12-4.16 (2H, m) |
| | | | 4.53-4.57 (1H, m) 4.77 (3H, br s) 6.89 (1H, d, J = 8.0 Hz) |
| | | | 7.10-7.22 (4H, m) 7.60 (1H, d, J = 4.0 Hz) |
| | | | 7.68 (1H, dd, J = 8.0, 4.0 Hz) |
| 342 | CD₃OD | 400 | 2.53 (3H, s) 2.55-2.62 (1H, m) 2.71-2.77 (1H, |
| | | | m) 2.93-3.01 (2H, m) 3.95 (1H, t, J = 4.0 Hz) |
| | | | 4.09 (2H, d, J = 4.0 Hz) 4.34 (1H, t, J = 4.0 Hz) |
| | | | 4.82 (5H, br s) 6.83 (1H, dd, J = 8.0, 4.0 Hz) |
| | | | 6.92-6.99 (3H, m) 7.04 (1H, s) 7.09-7.20 (5H, |
| | | | m) 7.58-7.61 (2H, m) |
| 343 | CD₃OD | 400 | 0.76-0.93 (2H, m), 1.15-1.38 (3H, m), |
| | | | 1.54 (3H, d, J = 6.6 Hz), 1.71-1.86 (6H, m), |
| | | | 2.41 (1H, br s), 2.83 (3H, s), 2.90-2.94 (2H, m), |
| | | | 3.09-3.14 (1H, m), 3.88 (1H, br s), |
| | | | 4.18-4.30 (2H, m), 4.63-4.68 (1H, m), 6.97 (1H, d, J = 9.1 Hz), |
| | | | 7.05-7.08 (1H, m), 7.14-7.21 (2H, m), |
| | | | 7.73 (1H, s), 7.78 (1H, d, J = 9.2 Hz), |
| | | | 8.70-8.73 (1H, m). |
| 344 | CD₃OD | 400 | 1.58 (3H, d, J = 8.9 Hz), 2.83 (6H, s), |
| | | | 2.90-3.05 (1H, m), 3.10-3.21 (1H, m), |
| | | | 3.90 (1H, q, J = 7 Hz), 4.18-4.23 (1H, m), |
| | | | 4.28-4.39 (1H, m), 4.67 (1H, t, J = 7.6 Hz), 4.91 (3H, s), |
| | | | 7.00 (1H, d, J = 9.0 Hz), 7.08-7.10 (1H, m), |
| | | | 7.16-7.30 (2H, m), 7.77 (1H, d, J = 3.96 Hz), |
| | | | 7.80 (1H, t, J = 7.04 Hz), 8.72-8.74 (1H, m). |
| 345 | CD₃OD | 400 | 1.26 (3H, t, J = 7.2 Hz), 1.53 (3H, d, J = 7.0 Hz), |
| | | | 2.78 (3H, s), 2.93-3.13 (4H, m), 3.89-3.94 (1H, m), |
| | | | 4.16-4.29 (2H, m), 4.63-4.67 (1H, m), |
| | | | 6.96 (1H, d, J = 9.1 Hz), 7.03-7.06 (1H, m), |
| | | | 7.10-7.20 (2H, m), 7.73 (1H, s), 7.76 (1H, dd, J = 9.2, |
| | | | 2.1 Hz), 8.70 (1H, t, J = 5.9 Hz). |
| 346 | CD₃OD | 400 | 0.92-0.99 (3H, m), 1.54 (3H, d, J = 7.0 Hz), |
| | | | 1.66-1.72 (2H, m), 2.80 (3H, s), 2.93-3.13 (4H, m), |
| | | | 3.90-3.99 (1H, m), 4.16-4.29 (2H, m), 4.62-4.66 (1H, m), |
| | | | 6.96 (1H, d, J = 9.1 Hz), 7.03-7.06 (1H, m), |
| | | | 7.11-7.20 (2H, m), 7.73 (1H, s), 7.77 (1H, dd, J = 9.1, |
| | | | 2.0 Hz), 8.71 (1H, t, J = 5.8 Hz). |
| 347 | CD₃OD | 400 | 0.94-0.97 (3H, m), 1.54 (3H, d, J = 7.0 Hz), |
| | | | 1.60-1.68 (2H, m), 2.80 (3H, s), 2.82-3.13 (6H, m), |
| | | | 3.91-3.96 (1H, m), 4.16-4.29 (2H, m), 4.62-4.66 (1H, m), |
| | | | 6.96 (1H, d, J = 9.2 Hz), 7.03-7.06 (1H, m), |
| | | | 7.11-7.20 (2H, m), 7.73 (1H, s), 7.74-7.78 (1H, m), |
| | | | 8.71 (1H, t, J = 5.9 Hz). |
| 348 | CD₃OD | 400 | 0.99-1.20 (9H, m), 1.30 (3H, d, J = 6.6 Hz), |
| | | | 1.39 (1H, s), 2.41 (1H, s), 2.60-2.70 (3H, m), |
| | | | 2.79-2.84 (1H, m), 2.95-3.01 (1H, m), 3.15-3.20 (1H, m), |
| | | | 3.81 (1H, s), 4.22-4.33 (2H, m), 7.02 (1H, d, J = 9.1 Hz), |
| | | | 7.06-7.09 (1H, m), 7.21-7.28 (2H, m), |
| | | | 7.80-7.83 (2H, m), 8.79-8.82 (1H, m). |
| 349 | CD₃OD | 400 | 0.98-1.05 (6H, m), 1.11-1.24 (3H, m), |
| | | | 1.30 (3H, d, J = 6.6 Hz), 1.37-1.40 (1H, m), |
| | | | 1.63 (1H, s), 2.07-2.09 (1H, m), 2.56-2.69 (3H, m), |
| | | | 2.83-2.86 (1H, m), 2.96-3.01 (1H, m), |
| | | | 3.14-3.19 (1H, m), 3.81-3.82 (1H, m), 4.22-4.33 (2H, m), |
| | | | 7.02 (1H, d, J = 9.1 Hz), 7.11-7.14 (1H, m), |
| | | | 7.21-7.28 (2H, m), 7.80-7.85 (2H, m), 8.80 (1H, s). |
| 350 | CD₃OD | 400 | 1.04-1.17 (6H, m) 1.30-1.47 (6H, m) 1.53 (3H, |
| | | | d, J = 8.0 Hz) 2.94-3.04 (2H, m) 3.07-3.17 (1H, |
| | | | m) 3.34-3.47 (1H, m) 3.90-4.06 (2H, m) |
| | | | 4.20 (1H, dd, J = 8.0, 4.0 Hz) 4.31-4.41 (1H, m) |
| | | | 4.63-4.78 (1H, m) 4.91-5.01 (2H, br m) |
| | | | 7.00-7.08 (1H, m) 7.11-7.17 (1H, m) 7.19-7.32 (2H, m) |
| | | | 7.74-7.77 (2H, m) 8.78 (1H, t, J = 4.0 Hz) |
| 351 | CD₃OD | 400 | 2.88-2.92 (7H, m), 2.99-3.04 (1H, m), |
| | | | 3.11-3.17 (1H, m), 3.39-3.42 (1H, m), 4.03-4.11 (2H, m), |
| | | | 4.16-4.22 (1H, m), 4.59 (1H, t, J = 7.8 Hz), |
| | | | 6.99-7.04 (2H, m), 7.10-7.35 (7H, m), 7.69-7.70 (1H, m), |
| | | | 7.72 (1H, s), 8.29 (1H, t, J = 5.8 Hz). |
| 352 | CD₃OD | 400 | 1.33 (3H, t, J = 6.9 Hz), 2.84-3.21 (9H, m), |
| | | | 4.03-4.23 (3H, m), 4.59-4.63 (1H, m), 6.99-7.05 (2H, m), |
| | | | 7.11-7.35 (7H, m), 7.70-7.71 (1H, m), 7.73 (1H, s), |
| | | | 8.30 (1H, t, J = 5.8 Hz). |
| 353 | CD₃OD | 400 | 0.98-1.05 (6H, m), 1.11-1.24 (3H, m), |
| | | | 1.30 (3H, d, J = 6.6 Hz), 1.37-1.40 (1H, m), |
| | | | 1.63 (1H, s), 2.07-2.09 (1H, m), 2.56-2.69 (3H, m), |
| | | | 2.83-2.86 (1H, m), 2.96-3.01 (1H, m), |
| | | | 3.14-3.19 (1H, m), 3.81-3.82 (1H, m), 4.22-4.33 (2H, m), |
| | | | 7.02 (1H, d, J = 9.1 Hz), 7.11-7.14 (1H, m), |
| | | | 7.21-7.28 (2H, m), 7.80-7.85 (2H, m), 8.80 (1H, s). |
| 354 | CD₃OD | 400 | 1.31 (2H, d, J = 8.0 Hz) 1.41 (2H, d, J = 8.0 Hz) |
| | | | 1.52-1.84 (6H, br m) 2.76-2.87 (3H, m) |
| | | | 2.93-3.08 (2H, m) 3.12-3.25 (1H, m) 3.45 (1H, br s) |
| | | | 3.67-3.79 (1H, m) 4.10-4.23 (2H, m) |
| | | | 4.45-4.49 (1H, m) 4.55-4.61 (1H, m) 6.86 (1H, d, J = 8.0 Hz) |
| | | | 6.90-6.95 (1H, m) 7.01-7.12 (2H, m) |
| | | | 7.57-7.68 (2H, m 08.64 (1H, t, J = 4.0 Hz) |
| 355 | CD₃OD | 400 | 1.28-1.49 (6H, m) 1.56 (3H, d, J = 8.0 Hz) |
| | | | 2.93-3.03 (2H, m) 3.14-3.29 (3H, m) 3.36-3.37 (1H, |
| | | | m) 4.04 (1H, q, J = 8.0 Hz) 4.23-4.33 (2H, m) |
| | | | 4.65 (1H, q, J = 8.0 Hz) 5.00 (3H, m) 7.02 (1H, |
| | | | d, J = 8.0 Hz) 7.10-7.12 (1H, m) 7.18-7.25 (2H, |
| | | | m) 7.78-7.84 (2H, m) 8.81 (1H, t, J = 4.0 Hz) |
| 356 | CD₃OD | 400 | 1.22 (2H, d, J = 8.0 Hz) 1.36 (2H, d, J = 8.0 Hz) |
| | | | 1.91-1.93 (5H, br m) 2.71 (1H, br s) |
| | | | 2.78-2.88 (1H, m) 2.97-3.13 (3H, m) 3.21-3.24 (1H, m) |
| | | | 3.46 (1H, br s) 3.76-3.88 (1H, m) 4.07-4.15 (2H, |
| | | | m) 4.42-4.54 (1H, m) 4.83 (1H, s) 6.87 (1H, dd, |
| | | | J = 8.0, 4.0 Hz) 6.93-6.95 (1H, m) |
| | | | 7.02-7.08 (2H, m) 7.61-7.69 (2H, m) |
| 357 | CD₃OD | 400 | 1.46 (2H, d, J = 8.0 Hz) 1.59 (2H, d, J = 8.0 Hz) |
| | | | 2.10 (5H, br s) 2.96-3.03 (1H, m) 3.11-3.16 (1H, |
| | | | m) 3.32-3.37 (5H, m) 3.87-3.96 (1H, m) |
| | | | 4.18-4.24 (1H, m) 4.30 (1H, s) 4.33 (1H, s) |
| | | | 4.63-4.70 (1H, m) 7.01 (1H, d, J = 8.0 Hz) 7.09 (1H, br s) |
| | | | 7.15-7.25 (2H, m) 7.77-7.83 (2H, m) |
| 359 | CD₃OD | 400 | 0.90 (6H, dd, J = 2.22, 6.72 Hz) 2.09-2.42 (1H, |
| | | | m) 2.93 (6H, s) 2.97-3.03 (1H, m) |
| | | | 3.11-3.20 (1H, m) 3.66 (1H, d, J = 6.0 Hz) 4.28-4.31 (2H, |
| | | | m) 4.61-4.65 (1H, m) 4.92 (4H, br s) 7.02 (1H, |
| | | | d, J = 9.08 Hz), 7.11-7.15 (1H, m), 7.21-7.26 (2H, |
| | | | m) 7.81-7.86 (2H, m), 8.79-8.82 (1H, m) |
| 360 | CD₃OD | 400 | 1.21 (3H, s), 1.27 (3H, d, J = 6.6 Hz), 2.78 (3H, s), |
| | | | 2.93-3.08 (2H, m), 3.23-3.28 (1H, m), |
| | | | 4.03-4.11 (3H, m), 4.25-4.41 (2H, m), 4.82 (1H, s), |
| | | | 7.02 (1H, d, J = 9.1 Hz), 7.11-7.14 (1H, m), |
| | | | 7.20-7.27 (2H, m), 7.79 (1H, d, J = 1.3 Hz), 7.85 (1H, dd, J = 9.2, |
| | | | 2.0 Hz), 8.72 (1H, t, J = 5.8 Hz). |
| 361 | CD₃OD | 400 | 1.31 (3H, t, J = 7.3 Hz), 2.90 (3H, s), |
| | | | 2.96-3.01 (1H, m), 3.14-3.23 (3H, m), 4.00 (1H, t, J = 4.5 Hz), |
| | | | 4.05-4.12 (2H, m), 4.23-4.34 (2H, m), |
| | | | 4.73-4.77 (1H, m), 7.01 (1H, d, J = 9.1 Hz), |
| | | | 7.09-7.12 (1H, m), 7.19-7.25 (2H, m), 7.77 (1H, d, J = 1.2 Hz), |
| | | | 7.82 (1H, dd, J = 9.1, 2.1 Hz), 8.70 (1H, t, J = 5.8 Hz). |
| 362 | CD₃OD | 400 | 1.26 (6H, t, J = 7.2 Hz), 2.95-3.01 (1H, m), |
| | | | 3.08-3.45 (5H, m), 4.03-4.09 (3H, m), 4.24-4.36 (2H, m), |
| | | | 4.75-4.79 (1H, m), 7.02 (1H, d, J = 9.2 Hz), |
| | | | 7.09-7.12 (1H, m), 7.19-7.26 (2H, m), 7.78 (1H, d, J = 1.2 Hz), |
| | | | 7.83 (1H, dd, J = 9.2, 2.1 Hz). |
| 363 | CD₃OD | 400 | 1.36 (6H, t, J = 7.3 Hz), 3.01-3.15 (2H, m), |
| | | | 3.31-3.38 (4H, m), 3.92-4.01 (2H, m), 4.10-4.12 (1H, m), |
| | | | 4.19-4.33 (2H, m), 4.61 (1H, t, J = 7.7 Hz), |
| | | | 7.00-7.02 (1H, m), 7.06-7.09 (1H, m), 7.15-7.25 (2H, m), |
| | | | 7.78-7.81 (2H, m), 8.77 (1H, t, J = 5.8 Hz). |
| 364 | CD₃OD | 400 | 1.26 (6H, t, J = 7.2 Hz), 1.37-1.43 (1H, m), |
| | | | 2.96-3.25 (7H, m), 3.76 (3H, s), 4.25-4.38 (3H, m), |
| | | | 4.75-4.79 (1H, m), 7.02 (1H, d, J = 9.5 Hz), |
| | | | 7.10-7.13 (1H, m), 7.20-7.28 (2H, m), 7.79 (1H, d, J = 1.7 Hz), |
| | | | 7.84 (1H, dd, J = 9.2, 2.2 Hz), 8.62 (1H, t, J = 5.8 Hz). |
| 365 | CD₃OD | 400 | 1.07 (3H, d, J = 8.0 Hz) 1.25 (3H, d, J = 4.0 Hz) |
| | | | 1.33-1.40 (1H, m) 1.50-1.72 (4H, m) |
| | | | 1.79-1.94 (4H, m) 2.92-3.01 (1H, m) 3.16-3.21 (1H, m) |
| | | | 3.53-3.65 (2H, m) 3.78-3.93 (2H, m) |
| | | | 4.16-4.27 (2H, m) 4.72 (1H, q, J = 4.0 Hz) 7.02 (1H, d, J = 12.0 Hz) |
| | | | 7.10 (1H, br s) 7.17-7.23 (2H, m) |
| | | | 7.75-7.84 (2H, m) 8.82-8.87 (1H, m) |
| 366 | CD₃OD | 400 | 2.09-2.12 (5H, m) 2.92-3.06 (2H, m) |
| | | | 3.12-3.20 (3H, m) 3.69-3.71 (3H, br m) 4.03-4.14 (2H, m) |
| | | | 4.26 (2H, s) 4.65 (1H, t, J = 8.0 Hz) 7.01 (2H, d, |
| | | | J = 8.0 Hz) 7.06-7.09 (1H, m) 7.15-7.26 (2H, m) |
| | | | 7.77-7.86 (2H, m) |
| 367 | CD₃OD | 400 | 1.08-1.12 (3H, m), 1.39 (3H, d, J = 6.9 Hz), |
| | | | 2.61 (3H, s), 2.78-3.01 (4H, m), 3.74-3.79 (1H, m), |
| | | | 4.03-4.14 (2H, m), 4.52-4.58 (1H, m), |
| | | | 6.81-6.90 (3H, m), 7.10-7.15 (2H, m), 7.57 (1H, s), |
| | | | 7.61 (1H, dd, J = 9.2, 2.1 Hz), 8.56 (1H, t, J = 5.8 Hz). |
| 368 | CD₃OD | 400 | 0.78-0.84 (3H, m), 1.39 (3H, d, J = 7.0 Hz), |
| | | | 1.52-1.56 (2H, m), 2.63 (3H, s), 2.68-3.00 (4H, m), |
| | | | 3.74-3.79 (1H, m), 4.02-4.17 (2H, m), 4.49-4.53 (1H, m), |
| | | | 6.80-6.88 (3H, m), 7.10-7.14 (2H, m), 7.56 (1H, s), |
| | | | 7.58-7.61 (1H, m), 8.71 (1H, t, J = 5.6 Hz). |
| 369 | CD₃OD | 400 | 0.93-0.97 (3H, m), 1.32-1.40 (2H, m), |
| | | | 1.53 (3H, d, J = 6.9 Hz), 1.61-1.70 (2H, m), |
| | | | 2.78 (3H, s), 2.83-3.14 (4H, m), 3.90-3.98 (1H, m), |
| | | | 4.17-4.28 (2H, m), 4.63-4.67 (1H, m), |
| | | | 6.95-7.02 (3H, m), 7.19-7.28 (2H, m), 7.71 (1H, s), |
| | | | 7.74 (1H, dd, J = 9.2, 2.0 Hz), 8.69 (1H, t, J = 5.7 Hz). |
| 370 | CD₃OD | 400 | 0.99-1.13 (9H, m), 1.27 (3H, d, J = 6.4 Hz), |
| | | | 1.37 (1H, s), 2.40-2.53 (2H, m), 2.68 (3H, s), |
| | | | 2.94-3.00 (1H, m), 3.16-3.21 (1H, m), 3.78-3.79 (1H, m), |
| | | | 4.27-4.38 (2H, m), 7.01 (1H, d, J = 9.2 Hz), |
| | | | 7.07 (2H, t, J = 8.6 Hz), 7.32-7.36 (2H, m), |
| | | | 7.79-7.84 (2H, m), 8.81-8.89 (1H, m). |
| 371 | CD₃OD | 400 | 0.98 (3H, d, J = 6.4 Hz), 1.03 (3H, t, J = 7.2 Hz), |
| | | | 1.15 (3H, d, J = 6.2 Hz), 1.27 (3H, d, J = 6.4 Hz), |
| | | | 1.36-1.39 (1H, m), 1.58-1.63 (1H, m), 2.05-2.13 (1H, m), |
| | | | 2.46-2.49 (1H, m), 2.67 (3H, s), 2.71-2.75 (1H, m), |
| | | | 2.94-3.00 (1H, m), 3.15-3.20 (1H, m), 3.79 (1H, s), |
| | | | 4.27-4.36 (2H, m), 7.01 (1H, d, J = 9.2 Hz), |
| | | | 7.07 (2H, t, J = 8.4 Hz), 7.32-7.35 (2H, m), |
| | | | 7.79-7.84 (2H, m), 8.80 (1H, s). |
| 372 | CD₃OD | 400 | 1.16-1.33 (6H, m) 1.38 (6H, d, J = 8.0 Hz) |
| | | | 1.57 (3H, d, J = 8.0 Hz) 2.87-3.00 (2H, m) |
| | | | 3.13-3.18 (1H, m) 3.31-3.37 (1H, m) 3.96-4.05 (2H, m) |
| | | | 4.21 (1H, d, J = 12.0, 4.0 Hz) 4.34-4.48 (1H, m) |
| | | | 4.72-4.79 (1H, m) 4.96-5.06 (2H, br m) |
| | | | 7.00-7.07 (3H, m) 7.28-7.32 (2H, m) 7.71-7.74 (2H, |
| | | | m) 8.76 (1H, t, J = 4.0 Hz) |
| 373 | CD₃OD | 400 | 1.19-1.26 (6H, m), 2.79-2.85 (5H, m), |
| | | | 3.00-3.08 (2H, m), 3.41-3.45 (1H, m), 4.03-4.08 (1H, m), |
| | | | 4.14-4.21 (2H, m), 4.70 (1H, s), 7.00-7.05 (3H, m), |
| | | | 7.23-7.34 (7H, m), 7.71-7.74 (2H, m), |
| | | | 8.16-8.19 (1H, m), 8.69 (1H, d, J = 8.6 Hz). |
| 374 | CD₃OD | 400 | 1.43 (3H, d, J = 8.0 Hz) 1.54-1.57 (1H, m) |
| | | | 1.85-1.99 (5H, m) 2.97-3.03 (3H, m) 3.12-3.17 (1H, |
| | | | m) 3.36-3.41 (2H, M) 3.60 (1h, D, j = 8.0 Hz) |
| | | | 3.90 (1H, q, J = 8.0 Hz) 4.21-4.31 (2H, m) |
| | | | 4.60-4.64 (1H, m) 5.02-5.07 (2H, m) 6.92-7.08 (3H, |
| | | | m) 7.28-7.31 (2H, m) 7.77-7.80 (2H, m) |
| | | | 8.77 (1H, t, J = 4.0 Hz) |
| 375 | CD₃OD | 400 | 1.40 (4H, d, J = 8.0 Hz) 1.51-1.57 (1H, m) |
| | | | 1.68 (3H, d, J = 12.0 Hz) 1.77-1.81 (1H, m) |
| | | | 2.57-2.71 (2H, m) 2.78-2.87 (2H, m) 3.00-3.05 (1H, m) |
| | | | 3.20-3.22 (1H, m) 3.40 (1H, d, J = 4.0 Hz) |
| | | | 3.61-3.69 (1H, m) 4.07-4.18 (2H, m) 4.58-4.62 (1H, |
| | | | m) 4.81 (2H, br s) 6.84-6.93 (3H, m) |
| | | | 7.14-7.18 (2H, m) 7.61-7.66 (2H, m) 8.62 (1H, t, J = 4.0 Hz) |
| 376 | CD₃OD | 400 | 1.23-1.29 (6H, m) 1.55 (3H, d, J = 8.0 Hz) |
| | | | 2.78 (1H, br s) 2.87-3.04 (2H, m) 3.16-3.21 (2H, m) |
| | | | 3.32-3.34 (1H, m) 4.02 (1H, q, J = 8.0 Hz) |
| | | | 4.28 (2H, d, J = 4.0 Hz) 4.74 (1H, q, J = 8.0 Hz) |
| | | | 5.03 (4H, br s) 7.03-7.15 (3H, m) 7.25-7.37 (2H, m) |
| | | | 7.71-7.81 (2H, m) |
| 377 | CD₃OD | 400 | 0.70-0.78 (1H, m) 0.86-0.91 (6H, m) |
| | | | 1.24-1.32 (1H, m) 2.01-2.07 (1H, m) 2.92 (6H, m) |
| | | | 2.97-3.03 (1H, m) 3.21-3.27 (1H, m) 3.72 (1H, d, J = 8.0 Hz) |
| | | | 4.23-4.44 (2H, m) 4.64-4.72 (1H, m) |
| | | | 4.95 (3H, s) 6.96-7.04 (2H, m) 7.09 (1H, d, J = 8.0 Hz) |
| | | | 7.17 (1H, d, J = 8.0 Hz) 7.34-7.39 (1H, |
| | | | m) 7.80 (1H, s) 7.86 (1H, dd, J = 8.0, 4.0 Hz) |
| | | | 8.84-8.92 (1H, m) |
| 378 | CD₃OD | 400 | 1.05 (2H, d, J = 8.0 Hz) 1.22-1.32 (4H, m) |
| | | | 1.45-1.50 (2H, m) 1.61-1.68 (1H, m) 1.78-1.95 (3H, |
| | | | m) 2.95-3.01 (1H, m) 3.16-3.21 (1H, m) |
| | | | 3.50-3.55 (2H, m) 3.77-3.92 (2H, m) 4.26 (2H, s) |
| | | | 4.71 (1H, q, J = 8.0 Hz) 4.94 (4H, s) 6.99-7.05 (3H, |
| | | | m) 7.28-7.36 (2H, m) 7.76-7.81 (2H, m) |
| 379 | CD₃OD | 400 | 1.56 (1H, s) 1.88 (6H, s) 2.88-3.00 (3H, m) |
| | | | 3.14-3.24 (1H, m) 3.34-3.37 (1H, m) 3.50 (1H, br s) |
| | | | 3.87-3.99 (2H, m) 4.26 (2H, s) 4.66-4.72 (2H, |
| | | | m) 5.02 (2H, s) 6.96-7.02 (3H, m) |
| | | | 7.27-7.30 (2H, m) 7.75-7.80 (2H, m) |
| 380 | CD₃OD | 400 | 1.34-1.41 (6H, m) 2.95-3.01 (1H, m) |
| | | | 3.14-3.33 (5H, m) 3.37-3.40 (1H, m) 3.92-4.08 (2H, m) |
| | | | 4.21-4.39 (2H, m) 4.64-4.71 (1H, m) |
| | | | 4.86-4.92 (2H, m) 6.96-7.10 (3H, m) 7.27-7.31 (2H, m) |
| | | | 7.73-7.82 (2H, m) 8.73-8.76 (1H, m) |
| 381 | CD₃OD | 400 | 0.96-1.06 (2H, m) 1.24-1.44 (10H, m) 1.52 (3H, |
| | | | d, J = 4.0 Hz) 1.79 (4H, d, J = 8.0 Hz) 2.79 (3H, |
| | | | s) 3.36-3.37 (1H, m) 3.55-3.58 (1H, m) |
| | | | 3.89 (1H, br s) 4.12 (1H, d, J = 8.0 Hz) |
| | | | 4.28-4.35 (2H, m) 4.40-4.50 (1H, m) 7.04 (1H, d, J = 8.0 Hz) |
| | | | 7.81 (1H, s) 7.92 (1H, dd, J = 8.0, 4.0 Hz) |
| | | | 8.83 (1H, t, J = 8.0 Hz) |
| 382 | CD₃OD | 400 | 0.99-1.12 (2H, m), 1.14-1.42 (4H, m), |
| | | | 1.62-1.95 (7H, m), 2.07-2.28 (4H, m), 2.61-2.69 (1H, m), |
| | | | 2.93, 2.97 (3H, 2xs), 3.15-3.29 (3H, m), |
| | | | 3.75-3.89 (1H, m), 4.14-4.19 (1H, m), 4.31-4.37 (2H, m), |
| | | | 4.46 (1H, t, J = 7.7 Hz), 7.03 (1H, d, J = 9.24 Hz) |
| | | | 7.81 (1H, s), 7.92 (1H, dd, J = 9.24, 2.15 Hz) |
| | | | 8.78-8.81 (1H, m) |
| 383 | CD₃OD | 400 | 0.88-1.08 (6H, m), 1.22-1.50 (5H, m), 1.62 (3H, d, |
| | | | J = 6.96 Hz), 1.64-1.69 (4H, m), 1.77-1.84 (8H, m), |
| | | | 2.94 (3H, s), 4.03 (1H, dd, J = 6.92, 13.81 Hz), |
| | | | 4.32-4.34 (2H, m), 4.47-4.53 (1H, m), 7.04 (1H, d, J = 9.09 Hz) |
| | | | 7.81 (1H, d, J = 1.05 Hz), |
| | | | 7.92 (1H, dd, J = 2.2, 9.28 Hz), 8.77-8.90 (1H, m) |
| 384 | CD₃OD | 400 | 0.96-1.08 (2H, m), 1.19-1.36 (4H, m), 1.40 (3H, t, J = 7.3 Hz), |
| | | | 1.61 (3H, d, J = 6.9 Hz), 1.65-1.83 (7H, m), |
| | | | 2.92 (3H, s), 3.22-3.28 (2H, m), 4.05 (1H, q, d, J = 6.9 Hz), |
| | | | 4.28-4.40 (2H, m), 4.46-4.50 (1H, m), |
| | | | 7.04 (1H, d, J = 9.6 Hz), 7.81 (1H, s), 7.93 (1H, dd, J = 9.2, |
| | | | 2.2 Hz), 8.79 (1H, t, J = 5.7 Hz). |
| 385 | CD₃OD | 400 | 0.99-1.00 (2H, m), 1.07 (6H, t, J = 7.24 Hz), |
| | | | 1.22-1.40 (6H, m), 1.61 (3H, d, J = 6.92 Hz), |
| | | | 1.66-1.87 (10H, m), 3.10-3.19 (2H, m, br), |
| | | | 3.21-3.28 (2H, m), 4.13 (1H, dd, J = 7.37, 13.84 Hz), |
| | | | 4.32 (2H, d, J = 5.72 Hz), 4.49 (1H, dd, J = 6.08, 9.2 Hz), |
| | | | 4.91 (2H, s), 7.04 (1H, d, J = 9.25 Hz) 7.81 (1H, s), |
| | | | 7.93 (1H, dd, J = 2.04, 9.16 Hz), 8.77-8.80 (1H, m) |
| 386 | CD₃OD | 400 | 0.84-0.95 (2H, m), 1.08-1.29 (4H, m), |
| | | | 1.52-1.70 (7H, m), 2.88 (6H, s), 3.91 (1H, t, J = 4.3 Hz), |
| | | | 3.96-4.04 (2H, m), 4.14-4.24 (2H, m), |
| | | | 4.35-4.39 (1H, m), 6.89 (1H, d, J = 9.1 Hz), 7.66 (1H, d, J = 1.4 Hz), |
| | | | 7.77 (1H, dd, J = 9.2, 2.1 Hz), 8.59 (1H, t, J = 5.8 Hz). |
| 387 | CD₃OD | 400 | 0.98-1.09 (2H, m), 1.19-1.33 (4H, m), 1.41 (3H, t, J = 7.3 Hz), |
| | | | 1.66-1.80 (7H, m), 3.00 (3H, s), |
| | | | 3.31-3.37 (2H, m), 4.10-4.17 (3H, m), 4.28-4.38 (2H, m), |
| | | | 4.49-4.53 (1H, m), 7.03 (1H, d, J = 9.4 Hz), |
| | | | 7.79 (1H, d, J = 1.2 Hz), 7.91 (1H, dd, J = 9.2, 2.2 Hz), |
| | | | 8.72 (1H, t, J = 5.8 Hz). |
| 388 | CD₃OD | 400 | 0.99-1.10 (2H, m), 1.21-1.33 (4H, m), 1.39 (3H, d, J = 6.3 Hz), |
| | | | 1.44 (3H, d, J = 6.6 Hz), 1.63-1.84 (7H, m), |
| | | | 2.89 (3H, s), 3.67 (1H, br s), 4.06-4.16 (3H, m), |
| | | | 4.32 (2H, s), 4.51-4.55 (1H, m), 7.03 (1H, d, J = 9.1 Hz), |
| | | | 7.80 (1H, d, J = 1.4 Hz), 7.91 (1H, dd, J = 9.2, |
| | | | 2.1 Hz), 8.71 (1H, t, J = 5.8 Hz). |
| 389 | CD₃OD | 400 | 0.95-1.10 (2H, m), 1.22-1.33 (4H, m), 1.40 (6H, t, J = 7.3 Hz), |
| | | | 1.63-1.84 (7H, m), 3.33-3.46 (4H, m), |
| | | | 4.07-4.19 (3H, m), 4.32 (2H, s), 4.50-4.54 (1H, m), |
| | | | 7.04 (1H, d, J = 9.0 Hz), 7.79 (1H, d, J = 1.4 Hz), |
| | | | 7.91 (1H, dd, J = 9.2, 2.1 Hz), 8.71 (1H, t, J = 5.8 Hz). |
| 390 | CD₃OD | 400 | 0.96-1.09 (3H, m), 1.13-1.29 (4H, m), 1.38 (6H, d, |
| | | | J = 7.32 Hz), 1.60 (3H, d, J = 6.84 Hz), |
| | | | 1.65-1.83 (8H, m), 3.21-3.26 (2H, m), |
| | | | 4.08 (1H, dd, J = 6.92, 13.84 Hz), 4.32-4.38 (2H, m), |
| | | | 4.48-4.58 (1H, m), 4.91 (3H, s), 7.04 (1H, d, J = 9.05 Hz) |
| | | | 7.81 (1H, d, J = 1.05 Hz), |
| | | | 7.92 (1H, dd, J = 2.2, 9.28 Hz), 8.77-8.80 (1H, m) |
| 391 | CD₃OD | 400 | 0.96-1.08 (3H, m), 1.22-1.40 (5H, m), 1.61 (3H, d, |
| | | | J = 6.96 Hz), 1.65-1.80 (7H, m), 2.95 (6H, s), |
| | | | 3.95-4.00 (1H, m), 4.32-4.38 (2H, m), |
| | | | 4.48-4.38 (1H, m), 4.46-4.54 (1H, m), 7.04 (1H, d, J = 9.09 Hz) |
| | | | 7.81 (1H, d, J = 1.36 Hz), |
| | | | 7.93 (1H, dd, J = 2.16, 9.28 Hz), 8.77-8.80 (1H, m) |
| 392 | CD₃OD | 400 | 0.94-1.09 (2H, m), 1.21-1.41 (10H, m), |
| | | | 1.49-1.99 (16H, m), 3.57-3.58 (2H, m, br), 3.91 (2H, s), |
| | | | 4.31-4.37 (2H, m), 4.49-4.51 (1H, m), 7.04 (1H, d, |
| | | | J = 9.24 Hz) 7.81 (1H, d, J = 1.36 Hz), |
| | | | 7.92 (1H, dd, J = 2.07, 9.16 Hz), 8.82-8.83 (1H, m) |
| 393 | CD₃OD | 400 | 0.94-1.08 (2H, m), 1.18-1.43 (16H, m), |
| | | | 1.65-1.83 (7H, m), 3.76-3.83 (2H, m), 4.02 (2H, s), |
| | | | 4.26-4.39 (2H, m), 4.47-4.52 (1H, m), 7.04 (1H, d, J = 9.1 Hz), |
| | | | 7.81 (1H, d, J = 1.4 Hz), 7.92 (1H, dd, J = 9.1, |
| | | | 2.1 Hz), 8.77 (1H, d, J = 7.2 Hz), 8.85 (1H, t, J = 5.8 Hz). |
| 394 | CD₃OD | 400 | 1.13-1.85 (31H, m), 3.77-3.85 (2H, m), |
| | | | 3.97-4.03 (2H, m), 4.26-4.47 (3H, m), 7.03-7.05 (1H, m), |
| | | | 7.81 (1H, s), 7.92 (1H, dd, J = 9.2, 2.0 Hz), |
| | | | 8.71-8.91 (2H, m). |
| 395 | CD₃OD | 400 | 1.18-1.86 (28H, m), 2.79 (3H, s), 3.56 (1H, s), |
| | | | 4.08-4.13 (1H, m), 4.32 (2H, s), 4.42-4.46 (1H, m), |
| | | | 7.04 (1H, d, J = 9.1 Hz), 7.81 (1H, s), 7.93 (1H, dd, J = 9.2, |
| | | | 2.1 Hz), 8.84-8.91 (1H, m). |
| 396 | CD₃OD | 400 | 1.15-1.85 (19H, m), 2.04-2.12 (2H, m), |
| | | | 2.22-2.31 (1H, m), 2.63-2.72 (1H, m), 2.96-2.97 (3H, m), |
| | | | 3.25-3.32 (1H, m), 3.75-3.82 (1H, m), |
| | | | 4.19-4.45 (4H, m), 7.05 (1H, d, J = 9.1 Hz), 7.82 (1H, s), |
| | | | 7.91-7.95 (1H, m), 8.84-8.95 (1H, m). |
| 397 | CD₃OD | 400 | 0.98-1.81 (19H, m), 3.04 (6H, s), 3.10-3.16 (1H, m), |
| | | | 3.41-3.45 (1H, m), 3.91-3.97 (1H, m), |
| | | | 4.07-4.13 (1H, m), 4.18-4.26 (1H, m), 4.39-4.43 (1H, m), |
| | | | 7.04 (1H, d, J = 9.1 Hz), 7.28-7.41 (5H, m), |
| | | | 7.84 (1H, s), 7.91 (1H, dd, J = 9.3, 2.0 Hz), |
| | | | 8.56-8.61 (1H, m) 8.79-8.81 (1H, m). |
| 398 | CD₃OD | 400 | 0.53 (3H, d, J = 8.0 Hz) 0.62 (4H, t, J = 4.0 Hz) |
| | | | 1.33-1.43 (1H, m) 1.58-1.64 (1H, m) 2.11 (6H, |
| | | | s) 2.57 (2H, d, J = 8.0 Hz) 2.89-2.97 (1H, m) |
| | | | 3.21 (1H, quintet, J = 4.0 Hz) 4.07 (2H, s) |
| | | | 4.53 (1H, dd, J = 16.0, 4.0 Hz) 4.75 (3H, s) 6.43 (1H, |
| | | | d, J = 8.0 Hz) 7.00-7.08 (1H, m) 7.20-7.42 (3H, |
| | | | m) 7.70 (1H, d, J = 8.0 Hz) |
| 399 | CD₃OD | 400 | 0.88-0.92 (6H, m) 2.31-2.42 (1H, m) 2.93 (6H, |
| | | | s) 2.98-3.04 (1H, m) 3.11-3.20 (1H, m) |
| | | | 3.65 (1H, d, J = 4.0 Hz) 4.29 (2H, s) 4.63-4.69 (1H, |
| | | | m) 4.96 (4H, br s) 7.02 (1H, dd, J = 8.0, 4.0 Hz) |
| | | | 7.27 (1H, dd, J = 8.0, 4.0 Hz) 7.35-7.49 (2H, m) |
| | | | 7.74-7.82 (2H, m) |
| 400 | CD₃OD | 400 | 2.23 (6H, s) 2.28-2.35 (1H, m) 2.48 (1H, dd, J = 8.0, |
| | | | 4.0 Hz) 2.68-2.89 (2H, m) 3.12-3.22 (2H, |
| | | | m) 4.01-4.08 (2H, m) 4.18 (1H, t, J = 4.0 Hz) |
| | | | 4.75 (3H, s) 6.42 (1H, d, J = 8.0 Hz) 6.75 (1H, |
| | | | dd, J = 8.0, 4.0 Hz) 7.00-7.09 (6H, m) |
| | | | 7.12-7.16 (2H, m) 7.66 (1H, d, J = 4.0 Hz) |
| 401 | CD₃OD | 400 | 0.94 (3H, d, J = 6.6 Hz) 0.99 (3H, d, J = 6.6 Hz) |
| | | | 1.17 (3H, d, J = 6.9 Hz) 2.16 (3H, s) |
| | | | 2.68-2.76 (1H, m) 2.78-2.90 (1H, m) 3.04-3.18 (1H, m) |
| | | | 3.36-3.43 (1H, m) 4.00-4.18 (2H, m) |
| | | | 4.56-4.61 (1H, m) 4.83 (4H, br s) 6.48 (1H, d, J = 8.0 Hz) |
| | | | 7.04 (1H, dd, J = 8.0, 4.0 Hz) 7.26 (1H, dd, J = 8.0, |
| | | | 4.0 Hz) 7.30-7.35 (2h, m) 7.73 (1H, d, J = 4.0 Hz) |
| 402 | CD₃OD | 400 | 1.33 (3H, d, J = 4.0 Hz) 1.37-1.44 (1H, m) |
| | | | 1.72-1.83 (6H, br m) 2.82-2.97 (3H, m) |
| | | | 3.00-3.07 (1H, m) 3.23-3.28 (1H, m) 3.47 (1H, br d, J = 4.0 Hz) |
| | | | 3.78-3.83 (1H, m) 4.07-4.19 (2H, m) |
| | | | 4.50-4.57 (1H, m) 4.81 (3H, br s) 6.87-6.94 (1H, |
| | | | m) 7.12 (1H, dd, J = 8.0, 4.0 Hz) 7.32-7.38 (2H, |
| | | | m) 7.61-7.67 (2H, m) |
| 403 | CD₃OD | 400 | 1.05 (1H, t, J = 8.0 Hz) 1.39 (4H, d, J = 4.0 Hz) |
| | | | 1.45-1.53 (1H, m) 1.69-1.77 (4H, m) |
| | | | 2.57-2.68 (2H, m) 2.76 (2H, s) 2.79-2.87 (2H, m) |
| | | | 2.97-3.07 (1H, m) 3.11-3.23 (2H, m) 3.67-3.78 (1H, |
| | | | m) 4.04-4.18 (2H, m) 4.59-4.65 (1H, m) |
| | | | 6.86 (1H, d, J = 12.0 Hz) 7.07-7.77 (1H, m) |
| | | | 7.29-7.33 (2H, m) 7.62-7.66 (2H, m) |
| 404 | CD₃OD | 400 | 1.47-1.57 (1H, br m) 1.85-2.04 (5H, br m) |
| | | | 2.82-3.10 (3H, m) 3.16-3.22 (1H, m) 3.36-3.37 (1H, |
| | | | m) 3.65 (1H, d, J = 4.0 Hz) 3.83-3.91 (2H, m) |
| | | | 4.20-4.32 (2H, m) 4.65-4.71 (1H, m) 4.99 (4H, |
| | | | br s) 7.01 (1H, dd, J = 8.0, 4.0 Hz) 7.22 (1H, dd, |
| | | | J = 8.0, 4.0 Hz) 7.44-7.48 (2H, m) |
| | | | 7.77-7.82 (2H, m) |
| 405 | CD₃OD | 400 | 0.55-0.66 (1H, m) 0.74-0.94 (6H, m) |
| | | | 1.07-1.18 (1H, m) 1.87-1.97 (1H, m) 2.77 (6H, s) |
| | | | 2.81-2.91 (1H, m) 3.13 (1H, dd, J = 8.0, 4.0 Hz) |
| | | | 3.22-3.23 (1H, m) 3.56 (1H, d, J = 4.0 Hz) |
| | | | 4.13-4.34 (2H, m) 4.50-4.60 (1H, m) 4.81 (3H, s) |
| | | | 6.94 (1H, d, J = 4.0 Hz) 7.11-7.24 (4H, m) 7.66 (1H, |
| | | | s) 8.66-8.80 (1H, m) |
| 406 | CD₃OD | 400 | 0.87-0.90 (7H, m) 2.34-2.41 (1H, m) 2.93 (7H, |
| | | | s) 3.00-3.03 (1H, m) 3.17-3.24 (1H, m) |
| | | | 3.69 (1H, d, J = 4.0 Hz) 4.22-4.31 (2H, m) |
| | | | 4.66-4.71 (1H, m) 4.98 (2H, br s) 7.02 (1H, d, J = 8.0 Hz) |
| | | | 7.21-7.39 (4H, m) 7.78 (1H, S) 7.83 (1H, dd, J = 8.0, |
| | | | 4.0 Hz) |
| 407 | CD₃OD | 400 | 2.55-2.58 (1H, m) 2.74-2.80 (1H, m) 3.03 (6H, |
| | | | s) 3.08-3.14 (1H, m) 3.31-3.46 (1H, m) |
| | | | 4.07-4.11 (1H, m) 4.21-4.29 (2H, m) 4.33 (1H, t, J = 8.0 Hz) |
| | | | 4.93 (4H, br s) 6.91-7.00 (2H, m) |
| | | | 7.06 (1H, s) 7.15-7.25 (4H, m) 7.27-7.34 (3H, m) |
| | | | 7.70-7.73 (2H, m) |
| 408 | CD₃OD | 400 | 1.12 (3H, t, J = 7.0 Hz), 1.40 (3H, d, J = 6.9 Hz), |
| | | | 2.63 (3H, s), 2.80-3.05 (5H, m), 3.73-3.78 (1H, m), |
| | | | 4.00-4.20 (2H, m), 4.58-4.62 (1H, m), |
| | | | 6.84 (1H, d, J = 9.1 Hz), 7.08-7.18 (4H, m), |
| | | | 7.59 (1H, s), 7.62 (1H, dd, J = 9.2, 2.0 Hz), |
| | | | 8.60 (1H, t, J = 5.7 Hz). |
| 409 | CD₃OD | 400 | 0.97 (2H, d, J = 3.1 Hz), 1.11 (3H, d, J = 6.6 Hz), |
| | | | 1.20-1.22 (1H, m), 1.38 (3H, d, J = 6.6 Hz), |
| | | | 2.52 (3H, s), 2.73-2.84 (2H, m), 3.04-3.08 (1H, m), |
| | | | 3.78-3.83 (1H, m), 4.08-4.21 (2H, m), |
| | | | 6.86 (1H, d, J = 9.2 Hz), 7.09-7.19 (4H, m), |
| | | | 7.60 (1H, s), 7.62 (1H, d, J = 8.9 Hz), 8.64 (1H, t, J = 5.7 Hz). |
| 410 | CD₃OD | 400 | 1.11-1.18 (6H, m), 1.39 (3H, d, J = 7.0 Hz), |
| | | | 2.66-2.71 (1H, m), 2.80-3.05 (5H, m), 3.77-3.83 (1H, m), |
| | | | 4.04-4.20 (2H, m), 4.59-4.63 (1H, m), |
| | | | 6.85 (1H, d, J = 9.6 Hz), 7.08-7.19 (4H, m), |
| | | | 7.59 (1H, d, J = 1.0 Hz), 7.63 (1H, dd, J = 9.2, 2.1 Hz), |
| | | | 8.61 (1H, t, J = 5.7 Hz). |
| 411 | CD₃OD | 400 | 1.32-1.38 (1H, m) 1.43 (2H, d, J = 8.0 Hz) |
| | | | 1.55 (3H, d, J = 8.0 Hz) 1.79-1.84 (3H, m) |
| | | | 1.93-2.00 (2H, m) 2.78-2.87 (1H, m) 2.93-3.04 (2H, m) |
| | | | 3.12-3.27 (2H, m) 3.54-3.64 (1H, m) |
| | | | 3.85-3.94 (1H, m) 4.22-4.34 (2H, m) 4.65-4.69 (1H, m) |
| | | | 4.77-4.82 (1H, m) 7.01 (1H, dd, J = 8.0, 4.0 Hz) |
| | | | 7.21-7.39 (5H, m) 7.76 (1H, d, J = 4.0 Hz) |
| | | | 7.79-7.87 (1H, m) |
| 412 | CD₃OD | 400 | 1.40-1.67 (1H, br m) 1.88-1.91 (5H, m) |
| | | | 2.93-3.01 (3H, m) 3.19-3.26 (1H, m) 3.34-3.37 (1H, |
| | | | m) 3.42-3.44 (1H, m) 3.52 (1H, d, J = 12.0 Hz) |
| | | | 3.86-3.99 (2H, m) 4.26 (2H, q, J = 8.0 Hz) |
| | | | 4.74 (1H, dd, J = 8.0, 4.0 Hz) 5.01 (3H, br s) |
| | | | 6.98 (1H, d, J = 8.0 Hz) 7.22-7.33 (4H, m) 7.67 (1H, |
| | | | s) 7.78 (1H, dd, J = 8.0, 4.0 Hz) |
| 413 | CD₃OD | 400 | 1.25 (3H, s), 1.56 (3H, d, J = 7.0 Hz), 2.35 (3H, s), |
| | | | 2.76 (5H, s), 2.92-2.97 (1H, m), 3.14-3.19 (1H, m), |
| | | | 3.88-3.93 (1H, m), 4.21-4.35 (2H, m), |
| | | | 4.75-4.79 (1H, m), 6.99 (1H, d, J = 9.3 Hz), |
| | | | 7.08-7.13 (3H, m), 7.19-7.23 (1H, m), 7.72 (1H, s), |
| | | | 7.62 (1H, dd, J = 9.2, 1.9 Hz), 8.74 (1H, t, J = 5.7 Hz). |
| 414 | CD₃OD | 400 | 0.91 (2H, s), 1.07 (3H, d, J = 6.4 Hz), |
| | | | 1.20-1.22 (1H, m), 1.37 (3H, d, J = 6.5 Hz), 2.19 (3H, s), |
| | | | 2.50 (3H, s), 2.62 (1H, s), 2.72-2.78 (1H, m), |
| | | | 3.00-3.05 (1H, m), 3.75-3.80 (1H, m), 4.07-4.20 (2H, m), |
| | | | 6.84 (1H, d, J = 9.2 Hz), 6.92-6.98 (3H, m), |
| | | | 7.03-7.07 (1H, m), 7.58 (1H, s), 7.63 (1H, d, J = 8.7 Hz), |
| | | | 8.61 (1H, t, J = 5.7 Hz). |
| 415 | CD₃OD | 400 | 1.05-1.09 (6H, m), 1.38 (3H, d, J = 6.8 Hz), |
| | | | 2.18 (3H, s), 2.58-2.67 (1H, m), 2.74-3.02 (5H, m), |
| | | | 3.77 (1H, q, J = 6.9 Hz), 4.04-4.20 (2H, m), |
| | | | 4.61-4.65 (1H, m), 6.83 (1H, d, J = 9.2 Hz), |
| | | | 6.91-6.97 (3H, m), 7.01-7.06 (1H, m), 7.56 (1H, s), |
| | | | 7.59 (1H, dd, J = 9.1, 2.1 Hz), 8.58 (1H, t, J = 5.8 Hz). |
| 416 | CD₃OD | 400 | 1.08 (3H, br s), 1.40 (3H, d, J = 7.0 Hz), 2.63 (6H, br |
| | | | s), 2.91-2.96 (1H, m), 3.11-3.16 (1H, m), |
| | | | 3.76 (1H, q, J = 6.6 Hz), 4.12 (2H, s), |
| | | | 4.62-4.66 (1H, m), 6.84 (1H, d, J = 9.1 Hz), |
| | | | 7.36-7.48 (4H, m), 7.59 (1H, d, J = 1.1 Hz), 7.64 (1H, dd, J = 9.2, |
| | | | 2.1 Hz), 8.64 (1H, t, J = 5.7 Hz). |
| 417 | CD₃OD | 400 | 1.08 (3H, br s), 1.25 (3H, d, J = 6.6 Hz), |
| | | | 1.35-1.42 (1H, m), 1.55 (3H, d, J = 6.8 Hz), 2.67 (3H, s), |
| | | | 2.88 (1H, s), 3.05-3.11 (1H, m), 3.30-3.35 (1H, m), |
| | | | 3.96 (1H, q, J = 6.8 Hz), 4.31 (2H, s), 7.02 (1H, d, J = 9.2 Hz), |
| | | | 7.53-7.67 (4H, m), 7.78 (1H, s), |
| | | | 7.84 (1H, d, J = 9.1 Hz), 8.83 (1H, t, J = 5.6 Hz). |
| 418 | CD₃OD | 400 | 1.22-1.30 (6H, m), 1.56 (3H, d, J = 6.9 Hz), |
| | | | 2.78-2.83 (1H, m), 3.06-3.33 (5H, m), 3.95 (1H, q, J = 6.9 Hz), |
| | | | 4.25-4.34 (2H, m), 4.81-4.85 (1H, m), |
| | | | 7.01 (1H, d, J = 9.5 Hz), 7.53-7.65 (4H, m), |
| | | | 7.77 (1H, d, J = 1.4 Hz), 7.81 (1H, dd, J = 9.2, 2.1 Hz), |
| | | | 8.81 (1H, t, J = 5.6 Hz),. |
| 419 | CD₃OD | 400 | 1.37-1.40 (2H, m) 1.55 (2H, d, J = 8.0 Hz) |
| | | | 1.62-1.94 (5H, m) 2.70-2.85 (1H, m) 2.91-2.99 (2H, |
| | | | m) 3.03-3.20 (2H, m) 3.28-3.42 (2H, m) |
| | | | 3.50-3.59 (1H, m) 3.83-3.88 (1H, m) 3.92-3.99 (1H, |
| | | | m) 4.20-4.33 (2H, m) 4.71-4.78 (1H, m) |
| | | | 4.84-4.89 (1H, m) 7.01 (1H, d, J = 8.0 Hz) |
| | | | 7.51-7.72 (4H, m) 7.77 (1H, s) 7.83 (1H, dd, J = 8.0, 4.0 Hz) |
| 420 | CD₃OD | 400 | 1.55 (1H, br s) 1.87 (5H, br s) 2.93-3.11 (3H, m) |
| | | | 3.28-3.37 (2H, m) 3.50 (1H, br s) 3.85-3.99 (2H, |
| | | | m) 4.24-4.32 (2H, m) 4.75-4.81 (1H, m) |
| | | | 5.00 (5H, br, s) 7.00 (1H, d, J = 8.0 Hz) |
| | | | 7.50-7.62 (3H, m) 7.75-7.86 (2H, m) |
| 421 | CD₃OD | 400 | 1.41 (2H, d, J = 8.0 Hz) 1.56 (1H, br t, J = 4.0 Hz) |
| | | | 1.88 (5H, br s) 2.92-3.13 (3H, m) |
| | | | 3.25-3.30 (1H, m) 3.36-3.57 (3H, m) 3.83-3.97 (1H, m) |
| | | | 4.22-4.41 (2H, m) 4.71-4.78 (1H, m) |
| | | | 4.91-4.96 (4H, m) 6.99-7.04 (1H, m) 7.49 (2H, d, J = 8.0 Hz) |
| | | | 7.63 (2H, d, J = 8.0 Hz) 7.79-7.82 (2H, m) |
| 422 | CD₃OD | 400 | 1.32-1.42 (1H, m) 1.56 (3H, d, J = 8.0 Hz) |
| | | | 1.72-1.92 (5H, m) 2.86-2.97 (3H, m) 3.04-3.14 (1H, |
| | | | m) 3.25-3.46 (2H, m) 3.56 (1H, br s) |
| | | | 3.79-3.84 (1H, m) 4.22-4.35 (2H, m) 4.82-4.96 (4H, m) |
| | | | 6.99-7.06 (1H, m) 7.48-7.55 (2H, m) 7.64 (2H, |
| | | | d, J = 8.0 Hz) 7.79-7.84 (2H, m) |
| 423 | CD₃OD | 400 | 1.36-1.57 (1H, m) 1.87 (5H, br s) 2.93-3.09 (3H, |
| | | | m) 3.20-3.37 (2H, m) 3.39 (1H, br s) |
| | | | 3.82-3.98 (2H, m) 4.27-4.31 (2H, m) 4.74-4.78 (1H, m) |
| | | | 4.94 (4H, br s) 7.00 (1H, d, J = 8.0 Hz) |
| | | | 7.50-7.57 (2H, m) 7.61-7.68 (2H, m) 7.78-7.83 (2H, |
| | | | m) |
| 424 | CD₃OD | 400 | 0.71-0.81 (1H, m) 0.85-0.93 (6H, m) |
| | | | 1.21-1.44 (1H, m) 2.04-2.11 (1H, m) 2.98 (6H, s) |
| | | | 3.01-3.08 (1H, m) 3.19-3.27 (1H, m) 3.78 (1H, d, J = 4.0 Hz) |
| | | | 4.31-4.39 (2H, m) 4.65-4.69 (1H, m) |
| | | | 5.00 (3H, br s) 6.84-6.89 (1H, m) 6.99 (2H, d, J = 4.0 Hz) |
| | | | 7.05 (1H, d, J = 12.0 Hz) 73.82 (1H, s) |
| | | | 7.88 (1H, dd, J = 8.0, 4.0 Hz) 9.01 (1H, d, J = 4.0 Hz) |
| 425 | CD₃OD | 400 | 1.45 (2H, d, J = 8.0 Hz) 1.57 (2H, d, J = 8.0 Hz) |
| | | | 1.86 (3H, br s) 1.95 (1H, d, J = 8.0 Hz) |
| | | | 2.84-2.93 (1H, m) 3.00-3.06 (1H, m) 3.14-3.27 (1H, |
| | | | m) 3.35-3.37 (1H, m) 3.60 (1H, br s) |
| | | | 3.87-3.96 (1H, m) 4.23-4.35 (2H, m) 4.61-4.71 (1H, m) |
| | | | 4.73-4.81 (1H, m) 4.97 (5H, s) 6.84-6.97 (3H, |
| | | | m) 6.98-7.08 (1H, m) 7.78-7.81 (1H, m) |
| | | | 7.85-7.94 (1H, m) |
| 426 | CD₃OD | 400 | 1.05 (2H, d, J = 8.0 Hz) 1.24 (3H, d, J = 8.0 Hz) |
| | | | 1.32 (1H, d, J = 4.0 Hz) 1.49-1.72 (6H, br m) |
| | | | 2.95-3.01 (1H, m) 3.15-3.21 (1H, m) 3.26 (1H, |
| | | | d, J = 6.4 Hz) 3.36-3.37 (2H, m) 3.75-3.87 (2H, |
| | | | m) 4.14-4.28 (2H, m) 4.71-4.77 (1H, m) |
| | | | 4.98 (2H, s) 7.01 (1H, d, J = 8.0 Hz) 7.21-7.31 (4H, |
| | | | m) 7.75-7.83 (2H, m) 8.79-8.89 (1H, m) |
| 427 | CD₃OD | 400 | 1.01 (6H, dd, J = 6.24, 14.0 Hz), 1.34 (3H, s, br), |
| | | | 1.43 (3H, s, br), 1.60 (3H, d, J = J = 6.83 Hz), |
| | | | 1.64-1.75 (4H, m), 2.79 (3H, s), 3.57 (1H, s, br), |
| | | | 4.10 = 4.12 (1H, m), 4.26-4.46 (3H, m), 4.91 (2H, s), |
| | | | 7.04 (1H, d, J = 8.17 Hz) 7.80 (1H, d, J = 1.4 Hz), |
| | | | 7.92 (1H, dd, J = 2.2, 9.28 Hz), 8.79-8.82 (1H, m) |
| 428 | CD₃OD | 400 | 1.19-1.21 (6H, br m) 2.50 (3H, s) 2.95-3.05 (1H, |
| | | | m) 3.11-3.27 (5H, m) 3.33-3.36 (1H, m) |
| | | | 3.89-4.09 (2H, m) 4.19-4.30 (2H, m) 4.68 (1H, t, J = 4.0 Hz) |
| | | | 4.94 (2H, br s) 6.82 (1H, d, J = 8.0 Hz) |
| | | | 7.00-7.05 (2H, m) 7.25-7.33 (2H, m) 7.72 (1H, |
| | | | d, J = 8.0 Hz) 8.70 (1H, t, J = 4.0 Hz) |
| 429 | CD₃OD | 400 | 0.99 (2H, d, J = 4.0 Hz) 1.24 (3H, d, J = 4.0 Hz) |
| | | | 1.33 (1H, d, J = 4.0 Hz) 1.47-1.59 (2H, m) |
| | | | 1.62-1.74 (2H, m) 1.78-1.93 (3H, m) 2.52 (3H, s) |
| | | | 2.92-3.01 (1H, m) 3.15-3.19 (1H, m) 3.53 (2H, t, |
| | | | J = 4.0 Hz) 3.79-3.92 (2H, m) 4.02-4.22 (1H, m) |
| | | | 4.26-4.32 (2H, m) 4.71 (1H, t, J = 8.0 Hz) |
| | | | 6.83 (1H, d, J = 8.0 Hz) 7.03-7.09 (1H, m) |
| | | | 7.12-7.27 (2H, m) 7.76 (2H, d, J = 8.0 Hz) 8.76 (1H, t, J = 4.0 Hz) |
| 430 | CD₃OD | 400 | 1.58 (1H, br s) 1.88-1.91 (6H, br m) 2.51 (3H, s) |
| | | | 2.91-3.03 (3H, m) 3.10-3.13 (1H, m) |
| | | | 3.37-3.42 (1H, m) 3.51-3.53 (1H, br d, J = 8.0 Hz) |
| | | | 3.89-3.99 (2H, m) 4.17-4.31 (2H, m) 4.66 (1H, dd, J = 8.0, |
| | | | 4.0 Hz) 4.96 (2H, br s) 6.82 (1H, d, J = 8.0 Hz) |
| | | | 7.01-7.08 (2H, m) 7.14-7.24 (2H, m) |
| | | | 7.75 (1H, d, J = 12.0 Hz) |
| 434 | CD₃OD | 400 | 1.02-1.47 (12H, m), 1.58-1.77 (17H, m), |
| | | | 2.94 (6H, s), 4.06 (1H, q, J = 6.89 Hz), |
| | | | 4.22-4.27 (1H, m), 4.34-4.40 (1H, m), |
| | | | 4.65 (1H, d, J = 7.6 Hz), 7.04 (1H, d, J = 9.1 Hz), |
| | | | 7.85 (1H, d, J = 1.5 Hz), 7.97 (1H, dd, J = 2.1, 9.2 Hz), |
| | | | 8.77-8.80 (1H, m). |
| 435 | CD₃OD | 400 | 1.07-1.51 (17H, m), 1.64 (3H, d, J = 6.89 Hz), |
| | | | 1.73-1.84 (3H, m), 2.08 (1H, d, J = 11.08 Hz), 2.79 (3H, s), |
| | | | 3.73-3.77 (2H, m), 4.22-4.27 (1H, m), |
| | | | 4.33-4.41 (2H, m), 4.60 (1H, d, J = 3 Hz), 7.04 (1H, d, J = 9.25 Hz), |
| | | | 7.82 (1H, d, J = 1.68 Hz), 7.95 (1H, dd, J = 6.0, |
| | | | 2.08 Hz), 8.71-8.79 (1H, m). |
| 436 | CD₃OD | 400 | 0.98-1.19 (3H, m), 1.24-1.44 (14H, m), |
| | | | 1.62 (3H, d, J = 6.8 Hz), 1.73-1.92 (3H, m), |
| | | | 2.10 (1H, d, J = 13.21 Hz), 2.78 (3H, s), |
| | | | 3.76 (2H, dd, J = 3.04 Hz, 8.24 Hz), |
| | | | 4.26-4.33 (1H, m), 4.37 (2H, d, J = 2.6 Hz), 4.60 (1H, d, |
| | | | J = 3 Hz), 7.04 (1H, d, J = 9.05 Hz), 7.82 (1H, d, J = 1.12 Hz), |
| | | | 7.95 (1H, dd, J = 9.17, 2.08 Hz), |
| | | | 8.71-8.79 (1H, m). |
| 437 | CD₃OD | 400 | 0.97-1.51 (14H, m), 1.58 (3H, d, J = 6.65 Hz), |
| | | | 1.73-1.95 (8H, m), 2.79 (3H, s), 3.13 (3H, s), |
| | | | 4.26-4.37 (2H, m), 4.59-4.73 (1H, m), 5.17-5.21 (1H, m), |
| | | | 7.04 (1H, d, J = 7.36 Hz) 7.79 (1H, d, J = 1.1 Hz), |
| | | | 7.92 (1H, d, J = 7.84 Hz), 7.93 (1H, dd, J = 9.2, 2.08 Hz) |
| | | | 8.58-8.87 (1H, m) |
| 438 | CD₃OD | 400 | 0.97-1.33 (7H, m), 1.38 (6H, t, J = 7.2 Hz), |
| | | | 1.58 (3H, d, J = 6.81 Hz), 1.72-1.96 (8H, m), |
| | | | 3.12 (3H, s), 4.25-4.36 (2H, m), |
| | | | 4.51 (1H, dd, J = 7.0, 13.49 Hz), 4.91 (4H, s), |
| | | | 5.20-5.24 (1H, m), 7.04 (1H, d, J = 9.13 Hz) |
| | | | 7.791 (1H, d, J = 1.2 Hz), 7.92 (1H, dd, J = 2.12, |
| | | | 9.21 Hz), 8.58-8.78 (1H, m) |
| 439 | CD₃OD | 400 | 0.99-1.00 (2H, m), 1.07 (6H, t, J = 7.24 Hz), |
| | | | 1.22-1.40 (6H, m), 1.61 (3H, d, J = 6.92 Hz), |
| | | | 1.66-1.87 (10H, m), 3.10-3.19 (2H, m, br), |
| | | | 3.21-3.28 (2H, m), 4.13 (1H, dd, J = 7.37, 13.84 Hz), |
| | | | 4.32 (2H, d, J = 5.72 Hz), 4.49 (1H, dd, J = 6.08, 9.2 Hz), |
| | | | 4.91 (2H, s), 7.04 (1H, d, J = 9.25 Hz) 7.81 (1H, s), |
| | | | 7.93 (1H, dd, J = 2.04, 9.16 Hz), 8.77-8.80 (1H, m) |

TABLE 15
Names of the examples.

| Example No. | Name |
|---|---|
| 12 | (S)-2-Amino-3-methyl-pentanoic acid [(S)-1-[(6-amino-pyridin-3- |
| | ylmethyl)-carbamoyl]-2-(decahydro-naphthalen-1-yl)-ethyl]-amide |
| 13 | (R)-2-Amino-N-[(S)-1-[(6-amino-pyridin-3-ylmethyl)carbamoyl]-2- |
| | (decahydro-naphthalen-1-yl)-ethyl]-3-(4-chloro-phenyl)-propionamide |
| 14 | (R)-2-Amino-N-[(S)-1-[(6-amino-pyridin-3-ylmethyl)-carbamoyl]-2- |
| | (decahydro-naphthalen-1-yl)-ethyl]-3-methyl-butyramide |
| 15 | (R)-Pyrrolidine-2-carboxylic acid [(S)-1-[(6-amino-pyridin-3-ylmethyl)- |
| | carbamoyl]-2-(decahydro-naphthalen-1-yl)-ethyl]-amide |
| 16 | (R)-2-Amino-4-methyl-pentanoic acid [(S)-1-[(6-amino-pyridin-3- |
| | ylmethyl)-carbamoyl]-2-(decahydro-naphthalen-1-yl)-ethyl]-amide |
| 17 | (S)-2-(2-Amino-acetylamino)-N-(6-amino-pyridin-3-ylmethyl)-3- |
| | (decahydro-naphthalen-1-yl)-propionamide |
| 18 | (R)-2-Amino-N-[(S)-1-[(6-amino-pyridin-3-ylmethyl)-carbamoyl]-2- |
| | (decahydro-naphthalen-1-yl)-ethyl]-propionamide |
| 19 | (S)—N-(6-Amino-pyridin-3-ylmethyl)-3-(decahydro-naphthalen-1-yl)-2- |
| | (2-methylamino-acetylamino)-propionamide |
| 20 | (R)-2-Amino-3-methyl-pentanoic acid {(S)-1-[(6-amino-pyridin-3- |
| | ylmethyl)-carbamoyl]-2-cyclohexyl-ethyl}-amide |
| 21 | (R)-3-Methyl-2-methylamino-pentanoic acid {(S)-1-[(6-amino-pyridin- |
| | 3-ylmethyl)-carbamoyl]-2-cyclohexyl-ethyl}-amide |
| 22 | (R)-2-Amino-N-{(S)-1-[(6-amino-pyridin-3-ylmethyl)-carbamoyl]-2- |
| | naphthalen-1-yl-ethyl}-3-methyl-butyramide |
| 23 | (R)-Pyrrolidine-2-carboxylic acid {(S)-1-[(6-amino-pyridin-3-ylmethyl)- |
| | carbamoyl]-2-naphthalen-1-yl-ethyl}-amide |
| 24 | (R)-2-Amino-3-methyl-pentanoic acid [(S)-1-[(6-amino-pyridin-3- |
| | ylmethyl)-carbamoyl]-2-(2-chloro-phenyl)-ethyl]-amide |
| 25 | (R)-2-Amino-3-methyl-pentanoic acid [(S)-1-[(6-amino-pyridin-3- |
| | ylmethyl)-carbamoyl]-2-(2-trifluoromethyl-phenyl)-ethyl]-amide |
| 26 | (R)-2-Amino-3-methyl-pentanoic acid {(S)-1-[(6-amino-pyridin-3- |
| | ylmethyl)-carbamoyl]-2-phenyl-ethyl}-amide |
| 27 | (R)-2-Amino-3-methyl-pentanoic acid {(S)-1-[(6-amino-pyridin-3- |
| | ylmethyl)-carbamoyl]-2-m-tolyl-ethyl}-amide |
| 28 | (R)-2-Amino-3-methyl-pentanoic acid {(S)-1-[(6-amino-pyridin-3- |
| | ylmethyl)-carbamoyl]-2-o-tolyl-ethyl}-amide |
| 29 | (R)-2-Amino-3-methyl-pentanoic acid [(S)-1-[(6-amino-pyridin-3- |
| | ylmethyl)-carbamoyl]-2-(3-fluoro-phenyl)-ethyl]-amide |
| 30 | (R)-2-Amino-3-methyl-pentanoic acid [(S)-1-[(6-amino-pyridin-3- |
| | ylmethyl)-carbamoyl]-2-(3-trifluoromethyl-phenyl)-ethyl]-amide |
| 31 | (R)-2-Amino-3-methyl-pentanoic acid [(S)-1-[(6-amino-pyridin-3- |
| | ylmethyl)-carbamoyl]-2-(3-cyano-phenyl)-ethyl]-amide |
| 32 | (R)-2-Amino-3-methyl-pentanoic acid [(S)-1-[(6-amino-pyridin-3- |
| | ylmethyl)-carbamoyl]-2-(3,4-difluoro-phenyl)-ethyl]-amide |
| 33 | (R)-2-Amino-3-methyl-pentanoic acid [(S)-1-[(6-amino-pyridin-3- |
| | ylmethyl)-carbamoyl]-2-(4-fluoro-phenyl)-ethyl]-amide |
| 34 | (R)-2-Amino-3-methyl-pentanoic acid {(S)-1-[(6-amino-pyridin-3- |
| | ylmethyl)-carbamoyl]-2-naphthalen-2-yl-ethyl}-amide |
| 35 | (R)-2-Amino-3-methyl-pentanoic acid [(S)-1-[(6-amino-pyridin-3- |
| | ylmethyl)-carbamoyl]-2-(3,4-dichloro-phenyl)-ethyl]-amide |
| 36 | (R)-2-Amino-3-methyl-pentanoic acid [(S)-1-[(6-amino-pyridin-3- |
| | ylmethyl)-carbamoyl]-2-(4-chloro-phenyl)-ethyl]-amide |
| 37 | (R)-2-Amino-3-methyl-pentanoic acid [(S)-1-[(6-amino-pyridin-3- |
| | ylmethyl)-carbamoyl]-2-(4-hydroxy-phenyl)-ethyl]-amide |
| 38 | (R)-2-Amino-3-methyl-pentanoic acid [(S)-1-[(6-amino-pyridin-3- |
| | ylmethyl)-carbamoyl]-2-(2,4,5-trifluoro-phenyl)-ethyl]-amide |
| 39 | (R)-2-Amino-3-methyl-pentanoic acid [(S)-1-[(6-amino-pyridin-3- |
| | ylmethyl)-carbamoyl]-2-(3,5-difluoro-phenyl)-ethyl]-amide |
| 40 | (R)-2-Amino-3-methyl-pentanoic acid [(S)-1-[(6-amino-pyridin-3- |
| | ylmethyl)-carbamoyl]-2-(1H-indol-3-yl)-ethyl]-amide |
| 41 | (R)-2-Amino-3-methyl-pentanoic acid {(S)-1-[(6-amino-pyridin-3- |
| | ylmethyl)-carbamoyl]-2-benzo[b]thiophen-3-yl-ethyl}-amide |
| 42 | (R)-3-Methyl-2-methylamino-pentanoic acid [(S)-1-[(6-amino-pyridin- |
| | 3-ylmethyl)-carbamoyl]-2-(3-trifluoromethyl-phenyl)-ethyl]-amide |
| 43 | (R)-3-Methyl-2-methylamino-pentanoic acid [(S)-1-[(6-amino-pyridin- |
| | 3-ylmethyl)-carbamoyl]-2-(3,4-difluoro-phenyl)-ethyl]-amide |
| 44 | (R)-3-Methyl-2-methylamino-pentanoic acid [(S)-1-[(6-amino-pyridin- |
| | 3-ylmethyl)-carbamoyl]-2-(4-fluoro-phenyl)-ethyl]-amide |
| 45 | (R)-3-Methyl-2-methylamino-pentanoic acid [(S)-1-[(6-amino-pyridin- |
| | 3-ylmethyl)-carbamoyl]-2-(3-chloro-phenyl)-ethyl]-amide |
| 46 | (R)-3-Methyl-2-methylamino-pentanoic acid [(S)-1-[(6-amino-pyridin- |
| | 3-ylmethyl)-carbamoyl]-2-(4-chloro-phenyl)-ethyl]-amide |
| 47 | (R)-3-Methyl-2-methylamino-pentanoic acid [(S)-1-[(6-amino-pyridin- |
| | 3-ylmethyl)-carbamoyl]-2-(4-hydroxy-phenyl)-ethyl]-amide |
| 48 | (R)-3-Methyl-2-methylamino-pentanoic acid [(S)-1-[(6-amino-pyridin- |
| | 3-ylmethyl)-carbamoyl]-2-(2,4,5-trifluoro-phenyl)-ethyl]-amide |
| 49 | (R)-3-Methyl-2-methylamino-pentanoic acid [(S)-1-[(6-amino-pyridin- |
| | 3-ylmethyl)-carbamoyl]-2-(3,5-difluoro-phenyl)-ethyl]-amide |
| 50 | (R)-3-Methyl-2-methylamino-pentanoic acid {(S)-1-[(6-amino-pyridin- |
| | 3-ylmethyl)-carbamoyl]-2-pentafluorophenyl-ethyl}-amide |
| 51 | (R)-3-Methyl-2-methylamino-pentanoic acid {(S)-1-[(6-amino-pyridin- |
| | 3-ylmethyl)-carbamoyl]-2-benzo[b]thiophen-3-yl-ethyl}-amide |
| 52 | (R)-3-Methyl-2-methylamino-pentanoic acid [(S)-1-[(6-amino-pyridin- |
| | 3-ylmethyl)-carbamoyl]-2-(4-tert-butyl-phenyl)-ethyl]-amide |
| 53 | (R)-3-Methyl-2-methylamino-pentanoic acid [(S)-1-[(6-amino-pyridin- |
| | 3-ylmethyl)-carbamoyl]-2-(3,4-difluoro-phenyl)-ethyl]-amide |
| 54 | (R)-2-Amino-N-[(S)-1-[(6-amino-pyridin-3-ylmethyl)-carbamoyl]-2- |
| | (3,4-dichloro-phenyl)-ethyl]-3-phenyl-propionamide |
| 55 | (R)-Pyrrolidine-2-carboxylic acid [(S)-1-[(6-amino-pyridin-3-ylmethyl)- |
| | carbamoyl]-2-(3,4-dichloro-phenyl)-ethyl]-amide |
| 56 | (S)-Thiazolidine-4-carboxylic acid [(S)-1-[(6-amino-pyridin-3- |
| | ylmethyl)-carbamoyl]-2-(3,4-dichloro-phenyl)-ethyl]-amide |
| 57 | (S)-2-((R)-2-Amino-2-phenyl-acetylamino)-N-(6-amino-pyridin-3- |
| | ylmethyl)-3-(3,4-dichloro-phenyl)-propionamide |
| 58 | (S)-2-((S)-2-Amino-2-phenyl-acetylamino)-N-(6-amino-pyridin-3- |
| | ylmethyl)-3-(3,4-dichloro-phenyl)-propionamide |
| 59 | (R)-2-Amino-N-[(S)-1-[(6-amino-pyridin-3-ylmethyl)-carbamoyl]-2- |
| | (3,4-dichloro-phenyl)-ethyl]-3-(4-chloro-phenyl)-propionamide |
| 60 | (R)—N-[(S)-1-[(6-Amino-pyridin-3-ylmethyl)-carbamoyl]-2-(3,4- |
| | dichloro-phenyl)-ethyl]-2-methylamino-3-phenyl-propionamide |
| 61 | (R)-2-Amino-N-[(S)-1-[(6-amino-pyridin-3-ylmethyl)-carbamoyl]-2- |
| | (3,4-dichloro-phenyl)-ethyl]-3,3-dimethyl-butyramide |
| 62 | (R)—N-[(S)-1-[(6-Amino-pyridin-3-ylmethyl)-carbamoyl]-2-(3,4- |
| | dichloro-phenyl)-ethyl]-2-methylamino-propionamide |
| 63 | (R)-Piperidine-2-carboxylic acid [(S)-1-[(6-amino-pyridin-3-ylmethyl)- |
| | carbamoyl]-2-(3,4-dichloro-phenyl)-ethyl]-amide |
| 64 | (R)-1,2,3,4-Tetrahydro-isoquinoline-3-carboxylic acid [(S)-1-[(6-amino- |
| | pyridin-3-ylmethyl)-carbamoyl]-2-(3,4-dichloro-phenyl)-ethyl]-amide |
| 65 | (R)-2,5-Dihydro-1H-pyrrole-2-carboxylic acid [(S)-1-[(6-amino-pyridin- |
| | 3-ylmethyl)-carbamoyl]-2-(3,4-dichloro-phenyl)-ethyl]-amide |
| 66 | (R)-4,4-Difluoro-pyrrolidine-2-carboxylic acid [(S)-1-[(6-amino- |
| | pyridin-3-ylmethyl)-carbamoyl]-2-(3,4-dichloro-phenyl)-ethyl]-amide |
| 67 | (R)-2-Amino-N-[(S)-1-[(6-amino-pyridin-3-ylmethyl)-carbamoyl]-2- |
| | (3,4-dichloro-phenyl)-ethyl]-4-phenyl-butyramide |
| 68 | (R)-2-Amino-N-[(S)-1-[(6-amino-pyridin-3-ylmethyl)-carbamoyl]-2- |
| | (3,4-dichloro-phenyl)-ethyl]-3-cyclohexyl-propionamide |
| 69 | (S)—N-(6-Amino-pyridin-3-ylmethyl)-3-(3,4-dichloro-phenyl)-2-(2- |
| | methylamino-acetylamino)-propionamide |
| 70 | (R)-2-Amino-N-[(S)-1-[(6-amino-pyridin-3-ylmethyl)-carbamoyl]-2-(3- |
| | trifluoromethyl-phenyl)-ethyl]-3-(4-chloro-phenyl)-propionamide |
| 71 | (R)-Pyrrolidine-2-carboxylic acid [(S)-1-[(6-amino-pyridin-3-ylmethyl)- |
| | carbamoyl]-2-(4-fluoro-phenyl)-ethyl]-amide |
| 72 | (R)-Pyrrolidine-2-carboxylic acid [(S)-1-[(6-amino-pyridin-3-ylmethyl)- |
| | carbamoyl]-2-(3,4-difluoro-phenyl)-ethyl]-amide |
| 73 | (R)-Piperidine-2-carboxylic acid [(S)-1-[(6-amino-pyridin-3-ylmethyl)- |
| | carbamoyl]-2-(4-fluoro-phenyl)-ethyl]-amide |
| 74 | (R)-Piperidine-2-carboxylic acid [(S)-1-[(6-amino-pyridin-3-ylmethyl)- |
| | carbamoyl]-2-(3,4-difluoro-phenyl)-ethyl]-amide |
| 75 | (R)-Piperidine-2-carboxylic acid {(S)-1-[(6-amino-pyridin-3-ylmethyl)- |
| | carbamoyl]-2-naphthalen-1-ylethyl}- |
| | amide |
| 76 | (R)-Piperidine-2-carboxylic acid [(S)-1-[(6-amino-pyridin-3-ylmethyl)- |
| | carbamoyl]-2-(2-chloro-phenyl)-ethyl]-amide |
| 77 | (R)-Piperidine-2-carboxylic acid [(S)-1-[(6-amino-pyridin-3-ylmethyl)- |
| | carbamoyl]-2-(3-chloro-phenyl)-ethyl]-amide |
| 78 | (R)-Piperidine-2-carboxylic acid {(S)-1-[(6-amino-pyridin-3-ylmethyl)- |
| | carbamoyl]-2-benzo[b]thiophen-3-yl-ethyl}-amide |
| 79 | (S)—N-(6-Amino-pyridin-3-ylmethyl)-3-(3,4-difluoro-phenyl)-2-(2- |
| | methylamino-acetylamino)-propionamide |
| 80 | (R)-2-Amino-N-[(S)-1-[(6-amino-pyridin-3-ylmethyl)-carbamoyl]-2- |
| | (3,4-difluoro-phenyl)-ethyl]-3-phenyl-propionamide |
| 81 | (R)—N-[(S)-1-[(6-Amino-pyridin-3-ylmethyl)carbamoyl]-2-(3,4-difluoro- |
| | phenyl)-ethyl]-2-methylamino-3-phenyl-propionamide |
| 82 | (S)-3-Methyl-2-methylamino-pentanoic acid [(S)-1-[(6-amino-pyridin-3- |
| | ylmethyl)-carbamoyl]-2-(3,4-difluoro-phenyl)-ethyl]-amide |
| 83 | (S)-2-Amino-3-methyl-pentanoic acid [(S)-1-[(6-amino-pyridin-3- |
| | ylmethyl)-carbamoyl]-2-(3,4-difluoro-phenyl)-ethyl]-amide |
| 84 | (R)—N-[(S)-1-[(6-Amino-pyridin-3-ylmethyl)-carbamoyl]-2-(3,4- |
| | difluoro-phenyl)-ethyl]-2-methylamino-propionamide |
| 85 | (S)—N-[(S)-1-[(6-Amino-pyridin-3-ylmethyl)-carbamoyl]-2-(3,4- |
| | difluoro-phenyl)-ethyl]-2-methylamino-propionamide |
| 86 | N-[(S)-1-[(6-Amino-pyridin-3-ylmethyl)-carbamoyl]-2-(3,4-difluoro- |
| | phenyl)-ethyl]-2-methyl-2-methylamino-propionamide |
| 87 | (R)—N-[(S)-1-[(6-Amino-pyridin-3-ylmethyl)-carbamoyl]-2-(4-fluoro- |
| | phenyl)-ethyl]-2-methylamino-propionamide |
| 88 | (S)—N-[(S)-1-[(6-Amino-pyridin-3-ylmethyl)-carbamoyl]-2-(4-fluoro- |
| | phenyl)-ethyl]-2-methylamino-propionamide |
| 89 | (R)—N-[(S)-1-[(6-Amino-pyridin-3-ylmethyl)-carbamoyl]-2-(4-fluoro- |
| | phenyl)-ethyl]-3-methyl-2-methylamino-butyramide |
| 90 | (R)-1-Methyl-pyrrolidine-2-carboxylic acid [(S)-1-[(6-amino-pyridin-3- |
| | ylmethyl)-carbamoyl]-2-(3,4-dichloro-phenyl)-ethyl]-amide |
| 91 | (R)-1-Methyl-pyrrolidine-2-carboxylic acid [(S)-1-[(6-amino-pyridin-3- |
| | ylmethyl)-carbamoyl]-2-(3-trifluoromethyl-phenyl)-ethyl]-amide |
| 92 | (R)-1-Methyl-pyrrolidine-2-carboxylic acid {(S)-1-[(6-amino-pyridin-3- |
| | ylmethyl)-carbamoyl]-2-naphthalen-1-yl-ethyl}-amide |
| 93 | (R)-1-Methyl-pyrrolidine-2-carboxylic acid [(S)-1-[(6-amino-pyridin-3- |
| | ylmethyl)-carbamoyl]-2-(4-chloro-phenyl)-ethyl]-amide |
| 94 | (R)-1-Methyl-pyrrolidine-2-carboxylic acid [(S)-1-[(6-amino-pyridin-3- |
| | ylmethyl)-carbamoyl]-2-(2,4,5-trifluoro-phenyl)-ethyl]-amide |
| 95 | (R)-1-Methyl-pyrrolidine-2-carboxylic acid {(S)-1-[(6-amino-pyridin-3- |
| | ylmethyl)-carbamoyl]-2-benzo[b]thiophen-3-yl-ethyl}-amide |
| 96 | (R)-1-Methyl-pyrrolidine-2-carboxylic acid [(S)-1-[(6-amino-pyridin-3- |
| | ylmethyl)-carbamoyl]-2-(3,5-difluoro-phenyl)-ethyl]-amide |
| 97 | (R)-1-Methyl-pyrrolidine-2-carboxylic acid {(S)-1-[(6-amino-pyridin-3- |
| | ylmethyl)-carbamoyl]-2-pentafluorophenyl-ethyl}-amide |
| 98 | (R)-1-Methyl-pyrrolidine-2-carboxylic acid [(S)-1-[(6-amino-pyridin-3- |
| | ylmethyl)-carbamoyl]-2-(4-fluoro-phenyl)-ethyl]-amide |
| 99 | (R)-1-Methyl-pyrrolidine-2-carboxylic acid [(S)-1-[(6-amino-pyridin-3- |
| | ylmethyl)-carbamoyl]-2-(3-fluoro-phenyl)-ethyl]-amide |
| 100 | (R)-1-Methyl-pyrrolidine-2-carboxylic acid [(S)-1-[(6-amino-pyridin-3- |
| | ylmethyl)-carbamoyl]-2-(4-methoxy-phenyl)-ethyl]-amide |
| 101 | (R)-1-Methyl-pyrrolidine-2-carboxylic acid {(S)-1-[(6-amino-pyridin-3- |
| | ylmethyl)-carbamoyl]-2-phenyl-ethyl}-amide |
| 102 | (R)-1-Methyl-pyrrolidine-2-carboxylic acid [(S)-1-[(6-amino-pyridin-3- |
| | ylmethyl)-carbamoyl]-2-(4-tert-butyl-phenyl)-ethyl]-amide |
| 103 | (R)-1-Methyl-pyrrolidine-2-carboxylic acid [(S)-1-[(6-amino-pyridin-3- |
| | ylmethyl)-carbamoyl]-2-(3-chloro-phenyl)-ethyl]-amide |
| 104 | (R)-1-Methyl-pyrrolidine-2-carboxylic acid {(S)-1-[(6-amino-pyridin-3- |
| | ylmethyl)-carbamoyl]-2-o-tolyl-ethyl}-amide |
| 105 | (R)-1-Methyl-pyrrolidine-2-carboxylic acid {(S)-1-[(6-amino-pyridin-3- |
| | ylmethyl)-carbamoyl]-2-m-tolyl-ethyl}-amide |
| 106 | (R)-1-Methyl-pyrrolidine-2-carboxylic acid {(S)-1-[(6-amino-pyridin-3- |
| | ylmethyl)-carbamoyl]-2-p-tolyl-ethyl}-amide |
| 107 | (R)-1-Methyl-pyrrolidine-2-carboxylic acid [(S)-1-[(6-amino-pyridin-3- |
| | ylmethyl)-carbamoyl]-2-(3-methoxy-phenyl)-ethyl]-amide |
| 108 | (R)-1-Methyl-pyrrolidine-2-carboxylic acid [(S)-1-[(6-amino-pyridin-3- |
| | ylmethyl)-carbamoyl]-2-(2-fluoro-phenyl)-ethyl]-amide |
| 109 | (R)-1-Methyl-pyrrolidine-2-carboxylic acid [(S)-1-[(6-amino-pyridin-3- |
| | ylmethyl)-carbamoyl]-2-(2-chloro-phenyl)-ethyl]-amide |
| 110 | (R)-1-Methyl-pyrrolidine-2-carboxylic acid [(S)-1-[(6-amino-pyridin-3- |
| | ylmethyl)-carbamoyl]-2-(2,4-dichloro-phenyl)-ethyl]-amide |
| 111 | (R)-1-Methyl-pyrrolidine-2-carboxylic acid [(S)-1-[(6-amino-pyridin-3- |
| | ylmethyl)-carbamoyl]-2-(2-trifluoromethyl-phenyl)-ethyl]-amide |
| 112 | (R)-1-Methyl-pyrrolidine-2-carboxylic acid {(S)-1-[(6-amino-pyridin-3- |
| | ylmethyl)-carbamoyl]-2-pyridin-4-yl-ethyl}-amide |
| 113 | (R)-1-Methyl-pyrrolidine-2-carboxylic acid [(S)-1-[(6-amino-pyridin-3- |
| | ylmethyl)-carbamoyl]-2-(1H-indol-3-yl)-ethyl]-amide |
| 114 | (R)-1-Methyl-pyrrolidine-2-carboxylic acid {(S)-1-[(6-amino-pyridin-3- |
| | ylmethyl)-carbamoyl]-2-quinolin-2-yl-ethyl}-amide |
| 115 | (R)-1-Methyl-pyrrolidine-2-carboxylic acid {(S)-1-[(6-amino-pyridin-3- |
| | ylmethyl)-carbamoyl]-2-cyclohexyl-ethyl}-amide |
| 116 | (S)-1-Methyl-pyrrolidine-2-carboxylic acid [(S)-1-[(6-amino-pyridin-3- |
| | ylmethyl)-carbamoyl]-2-(3,4-difluoro-phenyl)-ethyl]-amide |
| 117 | (R)-1-Isopropyl-pyrrolidine-2-carboxylic acid [(S)-1-[(6-amino-pyridin- |
| | 3-ylmethyl)-carbamoyl]-2-(3,4-dichloro-phenyl)-ethyl]-amide |
| 118 | (R)-1-Isopropyl-pyrrolidine-2-carboxylic acid {(S)-1-[(6-amino-pyridin- |
| | 3-ylmethyl)-carbamoyl]-2-naphthalen-1-yl-ethyl}-amide |
| 119 | (R)-1-Isopropyl-pyrrolidine-2-carboxylic acid [(S)-1-[(6-amino-pyridin- |
| | 3-ylmethyl)-carbamoyl]-2-(4-fluoro-phenyl)-ethyl]-amide |
| 120 | (R)-1-Isopropyl-pyrrolidine-2-carboxylic acid [(S)-1-[(6-amino-pyridin- |
| | 3-ylmethyl)-carbamoyl]-2-(3-chloro-phenyl)-ethyl]-amide |
| 121 | (R)-1-Isopropyl-pyrrolidine-2-carboxylic acid {(S)-1-[(6-amino-pyridin- |
| | 3-ylmethyl)-carbamoyl]-2-o-tolyl-ethyl}-amide |
| 122 | (R)-1-Isopropyl-pyrrolidine-2-carboxylic acid {(S)-1-[(6-amino-pyridin- |
| | 3-ylmethyl)-carbamoyl]-2-m-tolyl-ethyl}-amide |
| 123 | (R)-1-Isopropyl-pyrrolidine-2-carboxylic acid {(S)-1-[(6-amino-pyridin- |
| | 3-ylmethyl)-carbamoyl]-2-p-tolyl-ethyl}-amide |
| 124 | (R)-1-Isopropyl-pyrrolidine-2-carboxylic acid [(S)-1-[(6-amino-pyridin- |
| | 3-ylmethyl)-carbamoyl]-2-(3-methoxy-phenyl)-ethyl]-amide |
| 125 | (R)-1-Isopropyl-pyrrolidine-2-carboxylic acid [(S)-1-[(6-amino-pyridin- |
| | 3-ylmethyl)-carbamoyl]-2-(2-fluoro-phenyl)-ethyl]-amide |
| 126 | (R)-1-Isopropyl-pyrrolidine-2-carboxylic acid [(S)-1-[(6-amino-pyridin- |
| | 3-ylmethyl)-carbamoyl]-2-(2-chloro-phenyl)-ethyl]-amide |
| 127 | (R)-1-Isopropyl-pyrrolidine-2-carboxylic acid [(S)-1-[(6-amino-pyridin- |
| | 3-ylmethyl)-carbamoyl]-2-(2,4-dichloro-phenyl)-ethyl]-amide |
| 128 | (R)-1-Isopropyl-pyrrolidine-2-carboxylic acid [(S)-1-[(6-amino-pyridin- |
| | 3-ylmethyl)-carbamoyl]-2-(2-trifluoromethyl-phenyl)-ethyl]-amide |
| 129 | (R)-1-Isopropyl-pyrrolidine-2-carboxylic acid {(S)-1-[(6-amino-pyridin- |
| | 3-ylmethyl)-carbamoyl]-2-pyridin-4-yl-ethyl}-amide |
| 130 | (R)-1-Isopropyl-pyrrolidine-2-carboxylic acid [(S)-1-[(6-amino-pyridin- |
| | 3-ylmethyl)-carbamoyl]-2-(1H-indol-3-yl)-ethyl]-amide |
| 131 | (R)-1-Ethyl-pyrrolidine-2-carboxylic acid [(S)-1-[(6-amino-pyridin-3- |
| | ylmethyl)-carbamoyl]-2-(3,4-difluoro-phenyl)-ethyl]-amide |
| 132 | (R)-1-Propyl-pyrrolidine-2-carboxylic acid [(S)-1-[(6-amino-pyridin-3- |
| | ylmethyl)-carbamoyl]-2-(3,4-difluoro-phenyl)-ethyl]-amide |
| 133 | (R)-1-Isobutyl-pyrrolidine-2-carboxylic acid [(S)-1-[(6-amino-pyridin-3- |
| | ylmethyl)-carbamoyl]-2-(3,4-difluoro-phenyl)-ethyl]-amide |
| 134 | {(R)-2-[(S)-1-[(6-Amino-pyridin-3-ylmethyl)-carbamoyl]-2-(3,4- |
| | difluoro-phenyl)-ethylcarbamoyl]-pyrrolidin-1-yl}-acetic acid methyl |
| | ester |
| 135 | {(R)-2-[(S)-1-[(6-Amino-pyridin-3-ylmethyl)-carbamoyl]-2-(3,4- |
| | difluoro-phenyl)-ethylcarbamoyl]-pyrrolidin-1-yl}-acetic acid |
| 136 | (R)-1-Ethyl-pyrrolidine-2-carboxylic acid [(S)-1-[(6-amino-pyridin-3- |
| | ylmethyl)-carbamoyl]-2-(4-fluoro-phenyl)-ethyl]-amide |
| 137 | (R)-1-Ethyl-pyrrolidine-2-carboxylic acid {(S)-1-[(6-amino-pyridin-3- |
| | ylmethyl)-carbamoyl]-2-m-tolyl-ethyl}-amide |
| 138 | (R)-1-Ethyl-pyrrolidine-2-carboxylic acid [(S)-1-[(6-amino-pyridin-3- |
| | ylmethyl)-carbamoyl]-2-(3-chloro-phenyl)-ethyl]-amide |
| 139 | (R)-1-Propyl-pyrrolidine-2-carboxylic acid [(S)-1-[(6-amino-pyridin-3- |
| | ylmethyl)-carbamoyl]-2-(4-fluoro-phenyl)-ethyl]-amide |
| 140 | (R)-1-Benzyl-pyrrolidine-2-carboxylic acid [(S)-1-[(6-amino-pyridin-3- |
| | ylmethyl)-carbamoyl]-2-(3,4-difluoro-phenyl)-ethyl]-amide |
| 141 | (R)-1-Benzyl-pyrrolidine-2-carboxylic acid [(S)-1-[(6-amino-pyridin-3- |
| | ylmethyl)-carbamoyl]-2-(3,4-dichloro-phenyl)-ethyl]-amide |
| 142 | (R)-1-(4-Chloro-benzyl)-pyrrolidine-2-carboxylic acid [(S)-1-[(6-amino- |
| | pyridin-3-ylmethyl)-carbamoyl]-2-(3,4-difluoro-phenyl)-ethyl]-amide |
| 143 | (R)-1-(3-Chloro-benzyl)-pyrrolidine-2-carboxylic acid [(S)-1-[(6-amino- |
| | pyridin-3-ylmethyl)-carbamoyl]-2-(3,4-difluoro-phenyl)-ethyl]-amide |
| 144 | (R)-1-Methyl-piperidine-2-carboxylic acid [(S)-1-[(6-amino-pyridin-3- |
| | ylmethyl)-carbamoyl]-2-(3,4-difluoro-phenyl)-ethyl]-amide |
| 145 | (R)-1-Methyl-piperidine-2-carboxylic acid [(S)-1-[(6-amino-pyridin-3- |
| | ylmethyl)-carbamoyl]-2-(4-fluoro-phenyl)-ethyl]-amide |
| 146 | (R)-1-Isopropyl-piperidine-2-carboxylic acid [(S)-1-[(6-amino-pyridin- |
| | 3-ylmethyl)-carbamoyl]-2-(3,4-difluoro-phenyl)-ethyl]-amide |
| 147 | (R)-1-Isopropyl-piperidine-2-carboxylic acid [(S)-1-[(6-amino-pyridin- |
| | 3-ylmethyl)-carbamoyl]-2-(4-fluoro-phenyl)-ethyl]-amide |
| 148 | (2R,4R)-4-Hydroxy-1-methyl-pyrrolidine-2-carboxylic acid [(S)-1-[(6- |
| | amino-pyridin-3-ylmethyl)-carbamoyl]-2-(3,4-difluoro-phenyl)-ethyl]- |
| | amide |
| 149 | (R)-2-Methyl-1,2,3,4-tetrahydro-isoquinoline-3-carboxylic acid [(S)-1- |
| | [(6-amino-pyridin-3-ylmethyl)-carbamoyl]-2-(3,4-difluoro-phenyl)- |
| | ethyl]-amide |
| 150 | (S)—N-(6-Amino-pyridin-3-ylmethyl)-3-(3,4-dichloro-phenyl)-2-[2- |
| | (isopropyl-methyl-amino)-acetylamino]-propionamide |
| 151 | (R)—N-[(S)-1-[(6-Amino-pyridin-3-ylmethyl)-carbamoyl]-2-(3,4- |
| | dichloro-phenyl)-ethyl]-2-(isopropyl-methyl-amino)-propionamide |
| 152 | (S)—N-(6-Amino-pyridin-3-ylmethyl)-2-[2-(benzyl-methyl-amino)- |
| | acetylamino]-3-(3,4-dichloro-phenyl)-propionamide |
| 153 | (S)—N-(6-Amino-pyridin-3-ylmethyl)-3-(3,4-dichloro-phenyl)-2-(2- |
| | dimethylamino-acetylamino)-propionamide |
| 154 | (S)—N-(6-Amino-pyridin-3-ylmethyl)-3-(3,4-dichloro-phenyl)-2-[2- |
| | (isobutyl-methyl-amino)-acetylamino]-propionamide |
| 155 | (R)-2-(Isopropyl-methyl-amino)-3-methyl-pentanoic acid [(S)-1-[(6- |
| | amino-pyridin-3-ylmethyl)-carbamoyl]-2-(3,4-dichloro-phenyl)-ethyl]- |
| | amide |
| 156 | (S)—N-(6-Amino-pyridin-3-ylmethyl)-3-(3,4-difluoro-phenyl)-2-[2- |
| | (isopropyl-methyl-amino)-acetylamino]-propionamide |
| 157 | (S)—N-(6-Amino-pyridin-3-ylmethyl)-3-(3,4-difluoro-phenyl)-2-(2- |
| | diisopropylamino-acetylamino)-propionamide |
| 158 | (S)—N-(6-Amino-pyridin-3-ylmethyl)-3-(3,4-difluoro-phenyl)-2-(2- |
| | dipropylamino-acetylamino)-propionamide |
| 159 | (S)—N-(6-Amino-pyridin-3-ylmethyl)-3-(3,4-difluoro-phenyl)-2-(2- |
| | diisobutylamino-acetylamino)-propionamide |
| 160 | (S)—N-(6-Amino-pyridin-3-ylmethyl)-3-(3,4-difluoro-phenyl)-2-(2- |
| | phenethylamino-acetylamino)-propionamide |
| 161 | (S)—N-(6-Amino-pyridin-3-ylmethyl)-3-(3,4-difluoro-phenyl)-2-[2- |
| | (methyl-phenethyl-amino)-acetylamino]-propionamide |
| 162 | (S)—N-(6-Amino-pyridin-3-ylmethyl)-3-(3,4-difluoro-phenyl)-2-{2- |
| | [methyl-((E)-3-phenyl-allyl)-amino]-acetylamino}-propionamide |
| 163 | (S)—N-(6-Amino-pyridin-3-ylmethyl)-2-{2-[(4-chloro-benzyl)-methyl- |
| | amino]-acetylamino}-3-(3,4-difluoro-phenyl)-propionamide |
| 164 | (S)—N-(6-Amino-pyridin-3-ylmethyl)-2-{2-[(3-chloro-benzyl)-methyl- |
| | amino]-acetylamino}-3-(3,4-difluoro-phenyl)-propionamide |
| 165 | (S)—N-(6-Amino-pyridin-3-ylmethyl)-2-{2-[(2-chloro-benzyl)-methyl- |
| | amino]-acetylamino}-3-(3,4-difluoro-phenyl)-propionamide |
| 166 | (S)—N-(6-Amino-pyridin-3-ylmethyl)-3-(3,4-difluoro-phenyl)-2-{2-[(4- |
| | methoxy-benzyl)-methyl-amino]-acetylamino}-propionamide |
| 167 | (S)—N-(6-Amino-pyridin-3-ylmethyl)-2-[2-(butyl-methyl-amino)- |
| | acetylamino]-3-(3,4-difluoro-phenyl)-propionamide |
| 168 | (S)—N-(6-Amino-pyridin-3-ylmethyl)-3-(3,4-difluoro-phenyl)-2-[2- |
| | (isobutyl-methyl-amino)-acetylamino]-propionamide |
| 169 | (S)—N-(6-Amino-pyridin-3-ylmethyl)-2-[2-(cyclohexylmethyl-methyl- |
| | amino)-acetylamino]-3-(3,4-difluoro-phenyl)-propionamide |
| 170 | (S)—N-(6-Amino-pyridin-3-ylmethyl)-3-(3,4-difluoro-phenyl)-2-(2- |
| | piperidin-1-yl-acetylamino)-propionamide |
| 171 | (S)—N-(6-Amino-pyridin-3-ylmethyl)-3-(3,4-difluoro-phenyl)-2-(2- |
| | morpholin-4-yl-acetylamino)-propionamide |
| 172 | (S)—N-(6-Amino-pyridin-3-ylmethyl)-3-(3,4-difluoro-phenyl)-2-(2-3,4- |
| | dihydro-1H-isoquinolin-2-yl-acetylamino)-propionamide |
| 173 | (S)—N-(6-Amino-pyridin-3-ylmethyl)-3-(3,4-difluoro-phenyl)-2-(2-3,4- |
| | dihydro-2H-quinolin-1-yl-acetylamino)-propionamide |
| 174 | (S)—N-(6-Amino-pyridin-3-ylmethyl)-3-(3,4-difluoro-phenyl)-2-[2- |
| | (methyl-phenyl-amino)-acetylamino]-propionamide |
| 175 | (R)—N-[(S)-1-[(6-Amino-pyridin-3-ylmethyl)-carbamoyl]-2-(3,4- |
| | difluoro-phenyl)-ethyl]-2-(isopropyl-methyl-amino)-propionamide |
| 176 | (S)—N-[(S)-1-[(6-Amino-pyridin-3-ylmethyl)-carbamoyl]-2-(3,4- |
| | difluoro-phenyl)-ethyl]-2-(isopropyl-methyl-amino)-propionamide |
| 177 | (S)—N-[(S)-1-[(6-Amino-pyridin-3-ylmethyl)-carbamoyl]-2-(3,4- |
| | difluoro-phenyl)-ethyl]-2-(isobutyl-methyl-amino)-propionamide |
| 178 | (R)-2-Dimethylamino-3-methyl-pentanoic acid [(S)-1-[(6-amino- |
| | pyridin-3-ylmethyl)-carbamoyl]-2-(3,4-difluoro-phenyl)-ethyl]-amide |
| 179 | (R)—N-[(S)-1-[(6-Amino-pyridin-3-ylmethyl)-carbamoyl]-2-(3,4- |
| | difluoro-phenyl)-ethyl]-2-dimethylamino-3,3-dimethyl-butyramide |
| 180 | (R)—N-[(S)-1-[(6-Amino-pyridin-3-ylmethyl)-carbamoyl]-2-(4-fluoro- |
| | phenyl)-ethyl]-2-(isopropyl-methyl-amino)-propionamide |
| 181 | (S)—N-[(S)-1-[(6-Amino-pyridin-3-ylmethyl)-carbamoyl]-2-(4-fluoro- |
| | phenyl)-ethyl]-2-(isopropyl-methyl-amino)-propionamide |
| 182 | (R)-2-Dimethylamino-3-methyl-pentanoic acid [(S)-1-[(6-amino- |
| | pyridin-3-ylmethyl)-carbamoyl]-2-(4-fluoro-phenyl)-ethyl]-amide |
| 183 | (R)—N-[(S)-1-[(6-Amino-pyridin-3-ylmethyl)-carbamoyl]-2-(4-fluoro- |
| | phenyl)-ethyl]-2-dimethylamino-3,3-dimethyl-butyramide |
| 184 | (R)—N-[(S)-1-[(6-Amino-pyridin-3-ylmethyl)-carbamoyl]-2-(3,4- |
| | difluoro-phenyl)-ethyl]-2-isopropylamino-3-phenyl-propionamide |
| 185 | (R)—N-[(S)-1-[(6-Amino-pyridin-3-ylmethyl)-carbamoyl]-2-(3,4- |
| | difluoro-phenyl)-ethyl]-2-dimethylamino-3-phenyl-propionamide |
| 186 | (R)-2-Amino-N-[(S)-1-[(6-amino-2-methyl-pyridin-3-ylmethyl)- |
| | carbamoyl]-2-(decahydro-naphthalen-1-yl)-ethyl]-3-methyl-butyramide |
| 187 | (R)-2-Amino-3-methyl-pentanoic acid [(S)-1-[(6-amino-2-methyl- |
| | pyridin-3-ylmethyl)-carbamoyl]-2-(decahydro-naphthalen-1-yl)-ethyl]- |
| | amide |
| 188 | (2R)-3-Methyl-2-methylamino-pentanoic acid [(R)-1-[(6-amino-2- |
| | methyl-pyridin-3-ylmethyl)-carbamoyl]-2-(3,4-dichloro-phenyl)-ethyl]- |
| | amide |
| 189 | (R)-Pyrrolidine-2-carboxylic acid [(R)-1-[(6-amino-2-methyl-pyridin-3- |
| | ylmethyl)-carbamoyl]-2-(3,4-dichloro-phenyl)-ethyl]-amide |
| 190 | (R)-1-Methyl-pyrrolidine-2-carboxylic acid [(R)-1-[(6-amino-2-methyl- |
| | pyridin-3-ylmethyl)-carbamoyl]-2-(3,4-dichloro-phenyl)-ethyl]-amide |
| 191 | (S)-Thiazolidine-4-carboxylic acid [(R)-1-[(6-amino-2-methyl-pyridin- |
| | 3-ylmethyl)-carbamoyl]-2-(3,4-dichloro-phenyl)-ethyl]-amide |
| 192 | (R)-4,4-Difluoro-pyrrolidine-2-carboxylic acid [(S)-1-[(6-amino-2- |
| | methyl-pyridin-3-ylmethyl)-carbamoyl]-2-(3,4-dichloro-phenyl)-ethyl]- |
| | amide |
| 193 | (S)—N-[(R)-1-[(6-Amino-2-methyl-pyridin-3-ylmethyl)-carbamoyl]-2- |
| | (3,4-dichloro-phenyl)-ethyl]-2-methylamino-3-phenyl-propionamide |
| 194 | (R)-2-Amino-N-[(S)-1-[(6-amino-2-methyl-pyridin-3-ylmethyl)- |
| | carbamoyl]-2-(3,4-dichloro-phenyl)-ethyl]-3-(4-fluoro-phenyl)- |
| | propionamide |
| 195 | (R)-2-Amino-N-[(S)-1-[(6-amino-2-methyl-pyridin-3-ylmethyl)- |
| | carbamoyl]-2-(3,4-dichloro-phenyl)-ethyl]-3-(4-chloro-phenyl)- |
| | propionamide |
| 196 | (R)-2-Amino-N-[(S)-1-[(6-amino-2-methyl-pyridin-3-ylmethyl)- |
| | carbamoyl]-2-(3,4-dichloro-phenyl)-ethyl]-3-pyridin-3-yl-propionamide |
| 197 | (R)—N-(6-Amino-2-methyl-pyridin-3-ylmethyl)-2-((S)-2-amino-2- |
| | phenyl-acetylamino)-3-(3,4-dichloro-phenyl)-propionamide |
| 198 | (S)-2-((R)-2-Amino-2-cyclohexyl-acetylamino)-N-(6-amino-2-methyl- |
| | pyridin-3-ylmethyl)-3-(3,4-dichloro-phenyl)-propionamide |
| 199 | (R)-2-Amino-N-[(S)-1-[(6-amino-2-methyl-pyridin-3-ylmethyl)- |
| | carbamoyl]-2-(3,4-dichloro-phenyl)-ethyl]-3,3-dimethyl-butyramide |
| 200 | (R)-2-Amino-N-[(S)-1-[(6-amino-2-methyl-pyridin-3-ylmethyl)- |
| | carbamoyl]-2-(3,4-dichloro-phenyl)-ethyl]-3-cyclohexyl-propionamide |
| 201 | (R)-Piperidine-2-carboxylic acid [(S)-1-[(6-amino-2-methyl-pyridin-3- |
| | ylmethyl)-carbamoyl]-2-(3,4-dichloro-phenyl)-ethyl]-amide |
| 202 | (R)-1,2,3,4-Tetrahydro-quinoline-2-carboxylic acid [(S)-1-[(6-amino-2- |
| | methyl-pyridin-3-ylmethyl)-carbamoyl]- |
| | 2-(3,4-dichloro-phenyl)-ethyl]-amide |
| 203 | (R)-3-Methyl-2-methylamino-pentanoic acid {(S)-1-[(6-amino-2- |
| | methyl-pyridin-3-ylmethyl)-carbamoyl]-2-naphthalen-1-yl-ethyl}-amide |
| 204 | (R)-1-Methyl-pyrrolidine-2-carboxylic acid [(S)-1-[(6-amino-2-methyl- |
| | pyridin-3-ylmethyl)-carbamoyl]-2-(4-fluoro-phenyl)-ethyl]-amide |
| 205 | (R)-1-Methyl-pyrrolidine-2-carboxylic acid [(S)-1-[(6-amino-2-methyl- |
| | pyridin-3-ylmethyl)-carbamoyl]-2-(3,4-difluoro-phenyl)-ethyl]-amide |
| 206 | (R)-1-Methyl-pyrrolidine-2-carboxylic acid [(S)-1-[(6-amino-2,4- |
| | dimethyl-pyridin-3-ylmethyl)-carbamoyl]-2-(3,4-difluoro-phenyl)- |
| | ethyl]-amide |
| 207 | (R)-3-Methyl-2-methylamino-pentanoic acid [(S)-1-[(6-amino-2,4- |
| | dimethyl-pyridin-3-ylmethyl)-carbamoyl]-2-(3,4-difluoro-phenyl)- |
| | ethyl]-amide |
| 208 | (R)-2-Amino-3-methyl-pentanoic acid [(S)-1-[(6-amino-2,4-dimethyl- |
| | pyridin-3-ylmethyl)-carbamoyl]-2-(decahydro-naphthalen-1-yl)-ethyl]- |
| | amide |
| 209 | (R)-2-Amino-3-methyl-pentanoic acid {(S)-1-[(6-amino-5-methyl- |
| | pyridin-3-ylmethyl)-carbamoyl]-2-naphthalen-1-yl-ethyl}-amide |
| 210 | (R)-1-Methanesulfonyl-pyrrolidine-2-carboxylic acid [(S)-1-[(6-amino- |
| | pyridin-3-ylmethyl)-carbamoyl]-2-(3,4-dichloro-phenyl)-ethyl]-amide |
| 211 | (R)-2-Methanesulfonylamino-3-methyl-pentanoic acid [(S)-1-[(6-amino- |
| | pyridin-3-ylmethyl)-carbamoyl]-2-(3,4-dichloro-phenyl)-ethyl]-amide |
| 212 | (R)—N-[(S)-1-[(6-Amino-pyridin-3-ylmethyl)-carbamoyl]-2-(3,4- |
| | dichloro-phenyl)-ethyl]-2-(methanesulfonyl-methyl-amino)- |
| | propionamide |
| 213 | (R)-2-(Methanesulfonyl-methyl-amino)-3-methyl-pentanoic acid {(S)-1- |
| | [(6-amino-pyridin-3-ylmethyl)-carbamoyl]-2-benzo[b]thiophen-3-yl- |
| | ethyl}-amide |
| 214 | (R)-2-Amino-3-methyl-pentanoic acid {(R)-1-[(6-amino-pyridin-3- |
| | ylmethyl)-carbamoyl]-2,2-dicyclohexyl-ethyl}-amide |
| 215 | (S)-Pyrrolidine-2-carboxylic acid {(R)-1-[(6-amino-pyridin-3-ylmethyl)- |
| | carbamoyl]-2,2-dicyclohexyl-ethyl}-amide |
| 216 | (S)-2-Amino-N-{(R)-1-[(6-amino-pyridin-3-ylmethyl)-carbamoyl]-2,2- |
| | dicyclohexyl-ethyl}-3-phenyl-propionamide |
| 217 | (S)-2-Amino-N-{(R)-1-[(6-amino-pyridin-3-ylmethyl)-carbamoyl]-2,2- |
| | dicyclohexyl-ethyl}-3,3-dimethyl-butyramide |
| 218 | (S)—N-{(R)-1-[(6-Amino-pyridin-3-ylmethyl)-carbamoyl]-2,2- |
| | dicyclohexyl-ethyl}-2-methylamino-propionamide |
| 219 | (S)-3-Methyl-2-methylamino-pentanoic acid {(R)-1-[(6-amino-pyridin- |
| | 3-ylmethyl)-carbamoyl]-2,2-dicyclohexyl-ethyl}-amide |
| 220 | (R)-2-(2-Amino-acetylamino)-N-(6-amino-pyridin-3-ylmethyl)-3,3- |
| | dicyclohexyl-propionamide |
| 221 | (R)—N-(6-Amino-pyridin-3-ylmethyl)-3,3-dicyclohexyl-2-(2- |
| | methylamino-acetylamino)-propionamide |
| 222 | (R)—N-(6-Amino-pyridin-3-ylmethyl)-3,3-dicyclohexyl-2-(2- |
| | dimethylamino-acetylamino)-propionamide |
| 223 | (S)-1-Methyl-pyrrolidine-2-carboxylic acid {(R)-1-[(6-amino-pyridin-3- |
| | ylmethyl)-carbamoyl]-2,2-dicyclohexyl-ethyl}-amide |
| 224 | (S)-2-Amino-3-methyl-pentanoic acid {(1R,2R)-1-[(6-amino-pyridin-3- |
| | ylmethyl)-carbamoyl]-2-hydroxy-2-phenyl-ethyl}-amide |
| 225 | (R)-1-Methyl-pyrrolidine-2-carboxylic acid {(S)-1-[(6-amino-pyridin-3- |
| | ylmethyl)-carbamoyl]-2-phenyl-ethyl}-methyl-amide |
| 227 | 1H-Pyrrole-2-carboxylic acid [(S)-1-[(6-amino-pyridin-3-ylmethyl)- |
| | carbamoyl]-2-(decahydro-naphthalen-1-yl)-ethyl]-amide |
| 228 | 1H-Pyrrole-2-carboxylic acid {(S)-1-[(6-amino-pyridin-3-ylmethyl)- |
| | carbamoyl]-2-naphthalen-1-yl-ethyl}-amide |
| 229 | 1H-Pyrrole-2-carboxylic acid [(S)-1-[(6-amino-pyridin-3-ylmethyl)- |
| | carbamoyl]-2-(3-trifluoromethyl-phenyl)-ethyl]-amide |
| 230 | 3,4,5-Trimethyl-1H-pyrrole-2-carboxylic acid [(S)-1-[(6-amino-pyridin- |
| | 3-ylmethyl)-carbamoyl]-2-(3-trifluoromethyl-phenyl)-ethyl]-amide |
| 231 | 3,5-Dimethyl-1H-pyrrole-2-carboxylic acid {(S)-1-[(6-amino-pyridin-3- |
| | ylmethyl)-carbamoyl]-2-naphthalen-1-yl-ethyl}-amide |
| 232 | 3,5-Dimethyl-1H-pyrrole-2-carboxylic acid [(S)-1-[(6-amino-pyridin-3- |
| | ylmethyl)-carbamoyl]-2-(3-trifluoromethyl-phenyl)-ethyl]-amide |
| 233 | 3,5-Dimethyl-1H-pyrrole-2-carboxylic acid [(S)-1-[(6-amino-pyridin-3- |
| | ylmethyl)-carbamoyl]-2-(3,4-dichloro-phenyl)-ethyl]-amide |
| 234 | 3,5-Dimethyl-1H-pyrrole-2-carboxylic acid {(S)-1-[(6-amino-pyridin-3- |
| | ylmethyl)-carbamoyl]-2-m-tolyl-ethyl}-amide |
| 235 | 3,5-Dimethyl-1H-pyrrole-2-carboxylic acid [(S)-1-[(6-amino-pyridin-3- |
| | ylmethyl)-carbamoyl]-2-(3-chloro-phenyl)-ethyl]-amide |
| 236 | 3,5-Dimethyl-1H-pyrrole-2-carboxylic acid [(S)-1-[(6-amino-pyridin-3- |
| | ylmethyl)-carbamoyl]-2-(3-fluoro-phenyl)-ethyl]-amide |
| 237 | 3,5-Dimethyl-1H-pyrrole-2-carboxylic acid {(S)-1-[(6-amino-pyridin-3- |
| | ylmethyl)-carbamoyl]-2-phenyl-ethyl}-amide |
| 238 | 3,5-Dimethyl-1H-pyrrole-2-carboxylic acid {(S)-1-[(6-amino-pyridin-3- |
| | ylmethyl)-carbamoyl]-2-cyclohexyl-ethyl}-amide |
| 239 | 3,5-Dimethyl-1H-pyrrole-2-carboxylic acid [(S)-1-[(6-amino-pyridin-3- |
| | ylmethyl)-carbamoyl]-2-(2-chloro-phenyl)-ethyl]-amide |
| 240 | 3,5-Dimethyl-1H-pyrrole-2-carboxylic acid [(S)-1-[(6-amino-pyridin-3- |
| | ylmethyl)-carbamoyl]-2-(2-trifluoromethyl-phenyl)-ethyl]-amide |
| 241 | 3,5-Dimethyl-1H-pyrrole-2-carboxylic acid [(S)-1-[(6-amino-pyridin-3- |
| | ylmethyl)-carbamoyl]-2-(1H-indol-3-yl)-ethyl]-amide |
| 242 | 3,5-Dimethyl-1H-pyrrole-2-carboxylic acid [(S)-1-[(6-amino-pyridin-3- |
| | ylmethyl)-carbamoyl]-2-(4-fluoro-phenyl)-ethyl]-amide |
| 243 | 3,5-Dimethyl-1H-pyrrole-2-carboxylic acid [(S)-1-[(6-amino-pyridin-3- |
| | ylmethyl)-carbamoyl]-2-(3,4-difluoro-phenyl)-ethyl]-amide |
| 244 | 3,5-Dimethyl-1H-pyrrole-2-carboxylic acid {(S)-1-[(6-amino-pyridin-3- |
| | ylmethyl)-carbamoyl]-2-o-tolyl-ethyl}-amide |
| 245 | 3,5-Dimethyl-1H-pyrrole-2-carboxylic acid [(S)-1-[(6-amino-pyridin-3- |
| | ylmethyl)-carbamoyl]-2-(2,4,5-trifluoro-phenyl)-ethyl]-amide |
| 246 | 3,5-Dimethyl-1H-pyrrole-2-carboxylic acid [(S)-1-[(6-amino-pyridin-3- |
| | ylmethyl)-carbamoyl]-2-(4-chloro-phenyl)-ethyl]-amide |
| 247 | 3,5-Dimethyl-1H-pyrrole-2-carboxylic acid [(S)-1-[(6-amino-pyridin-3- |
| | ylmethyl)-carbamoyl]-2-(4-hydroxy-phenyl)-ethyl]-amide |
| 248 | 3,5-Dimethyl-1H-pyrrole-2-carboxylic acid [(S)-1-[(6-amino-pyridin-3- |
| | ylmethyl)-carbamoyl]-2-(2,4,5-trifluoro-phenyl)-ethyl]-amide |
| 249 | 3,5-Dimethyl-1H-pyrrole-2-carboxylic acid [(S)-1-[(6-amino-pyridin-3- |
| | ylmethyl)-carbamoyl]-2-(3,5-difluoro-phenyl)-ethyl]-amide |
| 250 | 3,5-Dimethyl-1H-pyrrole-2-carboxylic acid {(S)-1-[(6-amino-pyridin-3- |
| | ylmethyl)-carbamoyl]-2-benzo[b]thiophen-3-yl-ethyl}-amide |
| 251 | 3,5-Dimethyl-1H-pyrrole-2-carboxylic acid {(S)-1-[(6-amino-pyridin-3- |
| | ylmethyl)-carbamoyl]-2-pentafluorophenyl-ethyl}-amide |
| 252 | 3,5-Dimethyl-1H-pyrrole-2-carboxylic acid {(S)-1-[(6-amino-pyridin-3- |
| | ylmethyl)-carbamoyl]-2-biphenyl-4-yl-ethyl}-amide |
| 253 | 3,5-Dimethyl-1H-pyrrole-2-carboxylic acid {(S)-1-[(6-amino-pyridin-3- |
| | ylmethyl)-carbamoyl]-2-p-tolyl-ethyl}-amide |
| 254 | 3,5-Dimethyl-1H-pyrrole-2-carboxylic acid [(S)-1-[(6-amino-pyridin-3- |
| | ylmethyl)-carbamoyl]-2-(4-methoxy-phenyl)-ethyl]-amide |
| 255 | 3,5-Dimethyl-1H-pyrrole-2-carboxylic acid [(S)-1-[(6-amino-pyridin-3- |
| | ylmethyl)-carbamoyl]-2-(4-ethoxy-phenyl)-ethyl]-amide |
| 256 | 3,5-Dimethyl-1H-pyrrole-2-carboxylic acid [(S)-1-[(6-amino-pyridin-3- |
| | ylmethyl)-carbamoyl]-2-(4-tert-butyl-phenyl)-ethyl]-amide |
| 257 | 3,5-Dimethyl-1H-pyrrole-2-carboxylic acid {(S)-1-[(6-amino-pyridin-3- |
| | ylmethyl)-carbamoyl]-2-quinolin-2-yl-ethyl}-amide |
| 258 | 3-Methyl-1H-pyrrole-2-carboxylic acid [(S)-1-[(6-amino-pyridin-3- |
| | ylmethyl)-carbamoyl]-2-(4-trifluoromethyl-phenyl)-ethyl]-amide |
| 259 | 4-Methyl-1H-pyrrole-2-carboxylic acid [(S)-1-[(6-amino-pyridin-3- |
| | ylmethyl)-carbamoyl]-2-(3-chloro-phenyl)-ethyl]-amide |
| 260 | 4-Methyl-1H-pyrrole-2-carboxylic acid [(S)-1-[(6-amino-pyridin-3- |
| | ylmethyl)-carbamoyl]-2-(4-fluoro-phenyl)-ethyl]-amide |
| 261 | 3-Methyl-1H-pyrrole-2-carboxylic acid [(S)-1-[(6-amino-pyridin-3- |
| | ylmethyl)-carbamoyl]-2-(4-fluoro-phenyl)-ethyl]-amide |
| 262 | 3-Methyl-1H-pyrrole-2-carboxylic acid [(S)-1-[(6-amino-pyridin-3- |
| | ylmethyl)-carbamoyl]-2-(2-chloro-phenyl)-ethyl]-amide |
| 263 | 3-Methyl-1H-pyrrole-2-carboxylic acid [(S)-1-[(6-amino-pyridin-3- |
| | ylmethyl)-carbamoyl]-2-(2,5-dichloro-phenyl)-ethyl]-amide |
| 264 | 3-Methyl-1H-pyrrole-2-carboxylic acid [(S)-1-[(6-amino-pyridin-3- |
| | ylmethyl)-carbamoyl]-2-(3,4-dichloro-phenyl)-ethyl]-amide |
| 265 | 3-Methyl-1H-pyrrole-2-carboxylic acid [(S)-1-[(6-amino-pyridin-3- |
| | ylmethyl)-carbamoyl]-2-(2-fluoro-phenyl)-ethyl]-amide |
| 266 | 3-Methyl-1H-pyrrole-2-carboxylic acid [(S)-1-[(6-amino-pyridin-3- |
| | ylmethyl)-carbamoyl]-2-(3-chloro-phenyl)-ethyl]-amide |
| 267 | 3-Methyl-1H-pyrrole-2-carboxylic acid [(S)-1-[(6-amino-pyridin-3- |
| | ylmethyl)-carbamoyl]-2-(4-chloro-phenyl)-ethyl]-amide |
| 268 | 3-Methyl-1H-pyrrole-2-carboxylic acid {(S)-1-[(6-amino-pyridin-3- |
| | ylmethyl)-carbamoyl]-2-m-tolyl-ethyl}-amide |
| 269 | 3-Methyl-1H-pyrrole-2-carboxylic acid [(S)-1-[(6-amino-pyridin-3- |
| | ylmethyl)-carbamoyl]-2-(3-fluoro-phenyl)-ethyl]-amide |
| 270 | 3-Methyl-1H-pyrrole-2-carboxylic acid [(S)-1-[(6-amino-pyridin-3- |
| | ylmethyl)-carbamoyl]-2-(3,5-difluoro-phenyl)-ethyl]-amide |
| 271 | 3-Methyl-1H-pyrrole-2-carboxylic acid [(S)-1-[(6-amino-pyridin-3- |
| | ylmethyl)-carbamoyl]-2-(3-trifluoromethyl-phenyl)-ethyl]-amide |
| 272 | 3-Methyl-1H-pyrrole-2-carboxylic acid [(S)-1-[(6-amino-pyridin-3- |
| | ylmethyl)-carbamoyl]-2-(4-trifluoromethyl-phenyl)-ethyl]-amide |
| 273 | 3-Methyl-1H-pyrrole-2-carboxylic acid {(S)-1-[(6-amino-pyridin-3- |
| | ylmethyl)-carbamoyl]-2-cyclohexyl-ethyl}-amide |
| 274 | 5-[(S)-1-[(6-Amino-pyridin-3-ylmethyl)-carbamoyl]-2-(3- |
| | trifluoromethyl-phenyl)-ethylcarbamoyl]-1H-pyrrole-2-carboxylic acid |
| | methyl ester |
| 275 | 5-[(S)-1-[(6-Amino-pyridin-3-ylmethyl)-carbamoyl]-2-(3- |
| | trifluoromethyl-phenyl)-ethylcarbamoyl]-1H-pyrrole-2-carboxylic acid |
| 276 | 5-[(S)-1-[(6-Amino-pyridin-3-ylmethyl)-carbamoyl]-2-(3- |
| | trifluoromethyl-phenyl)-ethylcarbamoyl]-4-methyl-1H-pyrrole-2- |
| | carboxylic acid |
| 277 | 3,5-Dimethyl-1H-pyrrole-2-carboxylic acid [(S)-1-[(6-amino-2-methyl- |
| | pyridin-3-ylmethyl)-carbamoyl]-2-(3-trifluoromethyl-phenyl)-ethyl]- |
| | amide |
| 278 | 3,5-Dimethyl-1H-pyrrole-2-carboxylic acid {(S)-1-[(6-amino-2-methyl- |
| | pyridin-3-ylmethyl)-carbamoyl]-2-naphthalen-1-yl-ethyl}-amide |
| 279 | 3,5-Dimethyl-1H-pyrrole-2-carboxylic acid [(S)-1-[(6-amino-2-methyl- |
| | pyridin-3-ylmethyl)-carbamoyl]-2-(3-chloro-phenyl)-ethyl]-amide |
| 280 | 3,5-Dimethyl-1H-pyrrole-2-carboxylic acid [(S)-1-[(6-amino-2-methyl- |
| | pyridin-3-ylmethyl)-carbamoyl]-2-(3,4-dichloro-phenyl)-ethyl]-amide |
| 281 | 3,5-Dimethyl-1H-pyrrole-2-carboxylic acid {(S)-1-[(6-amino-2-methyl- |
| | pyridin-3-ylmethyl)-carbamoyl]-2-m-tolyl-ethyl}-amide |
| 282 | 3,5-Dimethyl-1H-pyrrole-2-carboxylic acid [(S)-1-[(6-amino-2-methyl- |
| | pyridin-3-ylmethyl)-carbamoyl]-2-(2-trifluoromethyl-phenyl)-ethyl]- |
| | amide |
| 283 | 3,5-Dimethyl-1H-pyrrole-2-carboxylic acid [(S)-1-[(6-amino-2-methyl- |
| | pyridin-3-ylmethyl)-carbamoyl]-2-(1H-indol-3-yl)-ethyl]-amide |
| 284 | 3,5-Dimethyl-1H-pyrrole-2-carboxylic acid [(S)-1-[(6-amino-2-methyl- |
| | pyridin-3-ylmethyl)-carbamoyl]-2-(3,4-difluoro-phenyl)-ethyl]-amide |
| 285 | 3,5-Dimethyl-1H-pyrrole-2-carboxylic acid [(S)-1-[(6-amino-2-methyl- |
| | pyridin-3-ylmethyl)-carbamoyl]-2-(4-fluoro-phenyl)-ethyl]-amide |
| 286 | 3-Methyl-1H-pyrrole-2-carboxylic acid [(S)-1-[(6-amino-2-methyl- |
| | pyridin-3-ylmethyl)-carbamoyl]-2-(3,4-difluoro-phenyl)-ethyl]-amide |
| 287 | 1H-Imidazole-2-carboxylic acid [(S)-1-[(6-amino-pyridin-3-ylmethyl)- |
| | carbamoyl]-2-(4-fluoro-phenyl)-ethyl]-amide |
| 288 | 1H-Imidazole-2-carboxylic acid [(S)-1-[(6-amino-pyridin-3-ylmethyl)- |
| | carbamoyl]-2-(3-fluoro-phenyl)-ethyl]-amide |
| 289 | 1H-Imidazole-2-carboxylic acid {(S)-1-[(6-amino-pyridin-3-ylmethyl)- |
| | carbamoyl]-2-naphthalen-1-yl-ethyl}-amide |
| 290 | 1-Methyl-1H-imidazole-2-carboxylic acid [(S)-1-[(6-amino-pyridin-3- |
| | ylmethyl)-carbamoyl]-2-(4-fluoro-phenyl)-ethyl]-amide |
| 291 | 2,5-Dimethyl-2H-pyrazole-3-carboxylic acid [(S)-1-[(6-amino-pyridin- |
| | 3-ylmethyl)-carbamoyl]-2-(4-fluoro-phenyl)-ethyl]-amide |
| 292 | 5-p-Tolyl-1H-pyrrole-2-carboxylic acid {(S)-1-[(6-amino-pyridin-3- |
| | ylmethyl)-carbamoyl]-2-naphthalen-1-yl-ethyl}-amide |
| 293 | 5-p-Tolyl-1H-pyrrole-2-carboxylic acid {(S)-1-[(6-amino-pyridin-3- |
| | ylmethyl)-carbamoyl]-2-benzo[b]thiophen-3-yl-ethyl}-amide |
| 294 | 5-p-Tolyl-1H-pyrrole-2-carboxylic acid [(S)-1-[(6-amino-pyridin-3- |
| | ylmethyl)-carbamoyl]-2-(3,4-difluoro-phenyl)-ethyl]-amide |
| 295 | 5-p-Tolyl-1H-pyrrole-2-carboxylic acid [(S)-1-[(6-amino-pyridin-3- |
| | ylmethyl)-carbamoyl]-2-(2-chloro-phenyl)-ethyl]-amide |
| 296 | 5-p-Tolyl-1H-pyrrole-2-carboxylic acid [(S)-1-[(6-amino-pyridin-3- |
| | ylmethyl)-carbamoyl]-2-(3,4-dichloro-phenyl)-ethyl]-amide |
| 297 | 5-p-Tolyl-1H-pyrrole-2-carboxylic acid [(S)-1-[(6-amino-pyridin-3- |
| | ylmethyl)-carbamoyl]-2-(3-chloro-phenyl)-ethyl]-amide |
| 298 | 1H-Indole-2-carboxylic acid {(S)-1-[(6-amino-pyridin-3-ylmethyl)- |
| | carbamoyl]-2-naphthalen-1-yl-ethyl}-amide |
| 299 | 1H-Benzoimidazole-2-carboxylic acid {(S)-1-[(6-amino-pyridin-3- |
| | ylmethyl)-carbamoyl]-2-naphthalen-1-yl-ethyl}-amide |
| 300 | 5-Methoxy-1H-indole-2-carboxylic acid {(S)-1-[(6-amino-pyridin-3- |
| | ylmethyl)-carbamoyl]-2-naphthalen-1-yl-ethyl}-amide |
| 301 | 5-Fluoro-1H-indole-2-carboxylic acid {(S)-1-[(6-amino-pyridin-3- |
| | ylmethyl)-carbamoyl]-2-naphthalen-1-yl-ethyl}-amide |
| 302 | 5-Hydroxy-1H-indole-2-carboxylic acid {(S)-1-[(6-amino-pyridin-3- |
| | ylmethyl)-carbamoyl]-2-naphthalen-1-yl-ethyl}-amide |
| 303 | 1H-Indole-2-carboxylic acid {(S)-1-[(6-amino-2-methyl-pyridin-3- |
| | ylmethyl)-carbamoyl]-2-naphthalen-1-yl-ethyl}-amide |
| 304 | 5-Methoxy-1H-indole-2-carboxylic acid {(S)-1-[(6-amino-2-methyl- |
| | pyridin-3-ylmethyl)-carbamoyl]-2-naphthalen-1-yl-ethyl}-amide |
| 305 | 5-Fluoro-1H-indole-2-carboxylic acid {(S)-1-[(6-amino-2-methyl- |
| | pyridin-3-ylmethyl)-carbamoyl]-2-naphthalen-1-yl-ethyl}-amide |
| 306 | 1-Methyl-1H-indole-2-carboxylic acid {(S)-1-[(6-amino-pyridin-3- |
| | ylmethyl)-carbamoyl]-2-naphthalen-1-yl-ethyl}-amide |
| 307 | 1H-Indole-2-carboxylic acid [(S)-1-[(6-amino-pyridin-3-ylmethyl)- |
| | carbamoyl]-2-(3-trifluoromethyl-phenyl)-ethyl]-amide |
| 308 | 5-Methoxy-1H-indole-2-carboxylic acid [(S)-1-[(6-amino-pyridin-3- |
| | ylmethyl)-carbamoyl]-2-(3-trifluoromethyl-phenyl)-ethyl]-amide |
| 309 | 1H-Indole-2-carboxylic acid [(S)-1-[(6-amino-2-methyl-pyridin-3- |
| | ylmethyl)-carbamoyl]-2-(3-trifluoromethyl-phenyl)-ethyl]-amide |
| 310 | 5-Methoxy-1H-indole-2-carboxylic acid [(S)-1-[(6-amino-2-methyl- |
| | pyridin-3-ylmethyl)-carbamoyl]-2-(3-trifluoromethyl-phenyl)-ethyl]- |
| | amide |
| 311 | 1H-Indole-2-carboxylic acid [(S)-1-[(6-amino-pyridin-3-ylmethyl)- |
| | carbamoyl]-2-(3-fluoro-phenyl)-ethyl]-amide |
| 312 | 5-Methoxy-1H-indole-2-carboxylic acid [(S)-1-[(6-amino-pyridin-3- |
| | ylmethyl)-carbamoyl]-2-(3-fluoro-phenyl)-ethyl]-amide |
| 313 | 1H-Indole-2-carboxylic acid [(S)-1-[(6-amino-pyridin-3-ylmethyl)- |
| | carbamoyl]-2-(3-chloro-phenyl)-ethyl]-amide |
| 314 | 5-Methoxy-1H-indole-2-carboxylic acid [(S)-1-[(6-amino-pyridin-3- |
| | ylmethyl)-carbamoyl]-2-(3-chloro-phenyl)-ethyl]-amide |
| 315 | 1H-Indole-2-carboxylic acid [(R)-1-[(6-amino-2-methyl-pyridin-3- |
| | ylmethyl)-carbamoyl]-2-(3-chloro-phenyl)-ethyl]-amide |
| 316 | 5-Methoxy-1H-indole-2-carboxylic acid [(S)-1-[(6-amino-2-methyl- |
| | pyridin-3-ylmethyl)-carbamoyl]-2-(3-chloro-phenyl)-ethyl]-amide |
| 317 | 1H-Indole-2-carboxylic acid {(S)-1-[(6-amino-pyridin-3-ylmethyl)- |
| | carbamoyl]-2-m-tolyl-ethyl}-amide |
| 318 | 5-Methoxy-1H-indole-2-carboxylic acid {(S)-1-[(6-amino-pyridin-3- |
| | ylmethyl)-carbamoyl]-2-m-tolyl-ethyl}-amide |
| 319 | 1H-Indole-2-carboxylic acid {(S)-1-[(6-amino-2-methyl-pyridin-3- |
| | ylmethyl)-carbamoyl]-2-m-tolyl-ethyl}-amide |
| 320 | 1H-Indole-2-carboxylic acid [(S)-1-[(6-amino-2-methyl-pyridin-3- |
| | ylmethyl)-carbamoyl]-2-(3,4-dichloro-phenyl)-ethyl]-amide |
| 321 | 5-Methoxy-1H-indole-2-carboxylic acid [(S)-1-[(6-amino-2-methyl- |
| | pyridin-3-ylmethyl)-carbamoyl]-2-(3,4-dichloro-phenyl)-ethyl]-amide |
| 322 | 1H-Indole-2-carboxylic acid [(S)-1-[(6-amino-2-methyl-pyridin-3- |
| | ylmethyl)-carbamoyl]-2-(2-trifluoromethyl-phenyl)-ethyl]-amide |
| 323 | 1H-Indole-2-carboxylic acid [(S)-1-[(6-amino-pyridin-3-ylmethyl)- |
| | carbamoyl]-2-(3,5-difluoro-phenyl)-ethyl]-amide |
| 324 | 1H-Indole-2-carboxylic acid [(S)-1-[(6-amino-pyridin-3-ylmethyl)- |
| | carbamoyl]-2-(2,4,5-trifluoro-phenyl)-ethyl]-amide |
| 325 | 1H-Indole-2-carboxylic acid [(S)-1-[(6-amino-pyridin-3-ylmethyl)- |
| | carbamoyl]-2-(3,4-difluoro-phenyl)-ethyl]-amide |
| 326 | 5-Fluoro-1H-indole-2-carboxylic acid [(S)-1-[(6-amino-pyridin-3- |
| | ylmethyl)-carbamoyl]-2-(3,4-difluoro-phenyl)-ethyl]-amide |
| 327 | 5-Methoxy-1H-indole-2-carboxylic acid [(S)-1-[(6-amino-pyridin-3- |
| | ylmethyl)-carbamoyl]-2-(3,4-difluoro-phenyl)-ethyl]-amide |
| 328 | 1H-Benzoimidazole-2-carboxylic acid [(S)-1-[(6-amino-pyridin-3- |
| | ylmethyl)-carbamoyl]-2-(3,4-difluoro-phenyl)-ethyl]-amide |
| 329 | 1H-Indole-2-carboxylic acid {(S)-1-[(6-amino-pyridin-3-ylmethyl)- |
| | carbamoyl]-2-pentafluorophenyl-ethyl}-amide |
| 330 | 1H-Indole-2-carboxylic acid [(S)-1-[(6-amino-pyridin-3-ylmethyl)- |
| | carbamoyl]-2-(4-fluoro-phenyl)-ethyl]-amide |
| 331 | 5-Fluoro-1H-indole-2-carboxylic acid [(S)-1-[(6-amino-pyridin-3- |
| | ylmethyl)-carbamoyl]-2-(4-fluoro-phenyl)-ethyl]-amide |
| 332 | 5-Methoxy-1H-indole-2-carboxylic acid [(S)-1-[(6-amino-pyridin-3- |
| | ylmethyl)-carbamoyl]-2-(4-fluoro-phenyl)-ethyl]-amide |
| 333 | 1H-Benzoimidazole-2-carboxylic acid [(S)-1-[(6-amino-pyridin-3- |
| | ylmethyl)-carbamoyl]-2-(4-fluoro-phenyl)-ethyl]-amide |
| 334 | 1H-Indole-2-carboxylic acid [(S)-1-[(6-amino-pyridin-3-ylmethyl)- |
| | carbamoyl]-2-(4-methoxy-phenyl)-ethyl]-amide |
| 335 | 1H-Indole-2-carboxylic acid {(S)-1-[(6-amino-pyridin-3-ylmethyl)- |
| | carbamoyl]-2-benzo[b]thiophen-3-yl-ethyl}-amide |
| 336 | 1H-Indole-2-carboxylic acid [(S)-1-[(6-amino-2-methyl-pyridin-3- |
| | ylmethyl)-carbamoyl]-2-(1H-indol-3-yl)-ethyl]-amide |
| 337 | 5-Methoxy-1H-indole-2-carboxylic acid [(S)-1-[(6-amino-2-methyl- |
| | pyridin-3-ylmethyl)-carbamoyl]-2-(1H-indol-3-yl)-ethyl]-amide |
| 338 | 1H-Indole-2-carboxylic acid {(S)-1-[(6-amino-pyridin-3-ylmethyl)- |
| | carbamoyl]-2-cyclohexyl-ethyl}-amide |
| 339 | (S)-3-Methyl-2-methylamino-pentanoic acid {(S)-1-[(6-amino-pyridin- |
| | 3-ylmethyl)-carbamoyl]-2-cyclohexyl-ethyl}-amide |
| 340 | (R)—N-[(S)-1-[(6-Amino-pyridin-3-ylmethyl)-carbamoyl]-2-(3,4- |
| | dichloro-phenyl)-ethyl]-3-methyl-2-methylamino-butyramide |
| 341 | (R)—N-[(S)-1-[(6-Amino-pyridin-3-ylmethyl)-carbamoyl]-2-(3-chloro- |
| | phenyl)-ethyl]-3-methyl-2-methylamino-butyramide |
| 342 | (R)—N-[(S)-1-[(6-Amino-pyridin-3-ylmethyl)-carbamoyl]-2-(3-chloro- |
| | phenyl)-ethyl]-2-methylamino-3-phenyl-propionamide |
| 343 | (S)—N-[(S)-1-[(6-Amino-pyridin-3-ylmethyl)-carbamoyl]-2-(3,4- |
| | difluoro-phenyl)-ethyl]-2-(cyclohexylmethyl-methyl-amino)- |
| | propionamide |
| 344 | (S)—N-[(S)-1-[(6-Amino-pyridin-3-ylmethyl)-carbamoyl]-2-(3,4- |
| | difluoro-phenyl)-ethyl]-2-dimethylamino-propionamide |
| 345 | (S)—N-[(S)-1-[(6-Amino-pyridin-3-ylmethyl)-carbamoyl]-2-(3,4- |
| | difluoro-phenyl)-ethyl]-2-(ethyl-methyl-amino)-propionamide |
| 346 | (S)—N-[(S)-1-[(6-Amino-pyridin-3-ylmethyl)-carbamoyl]-2-(3,4- |
| | difluoro-phenyl)-ethyl]-2-(methyl-propyl-amino)-propionamide |
| 347 | (S)—N-[(S)-1-[(6-Amino-pyridin-3-ylmethyl)-carbamoyl]-2-(3,4- |
| | difluoro-phenyl)-ethyl]-2-(butyl-methyl-amino)-propionamide |
| 348 | (S)—N-[(S)-1-[(6-Amino-pyridin-3-ylmethyl)-carbamoyl]-2-(3,4- |
| | difluoro-phenyl)-ethyl]-2-(isopropyl-methyl-amino)-3-methyl- |
| | butyramide |
| 349 | (S)-2-(Isopropyl-methyl-amino)-3-methyl-pentanoic acid [(S)-1-[(6- |
| | amino-pyridin-3-ylmethyl)-carbamoyl]-2-(3,4-difluoro-phenyl)-ethyl]- |
| | amide |
| 350 | (S)—N-[(S)-1-[(6-Amino-pyridin-3-ylmethyl)-carbamoyl]-2-(3,4- |
| | difluoro-phenyl)-ethyl]-2-diisopropylamino-propionamide |
| 351 | (S)—N-[(S)-1-[(6-Amino-pyridin-3-ylmethyl)-carbamoyl]-2-(3,4- |
| | difluoro-phenyl)-ethyl]-2-dimethylamino-3-phenyl-propionamide |
| 352 | (S)—N-[(S)-1-[(6-Amino-pyridin-3-ylmethyl)-carbamoyl]-2-(3,4- |
| | difluoro-phenyl)-ethyl]-2-(ethyl-methyl-amino)-3-phenyl-propionamide |
| 353 | (S)-2-(Isopropyl-methyl-amino)-3-methyl-pentanoic acid [(S)-1-[(6- |
| | amino-pyridin-3-ylmethyl)-carbamoyl]-2-(3,4-difluoro-phenyl)-ethyl]- |
| | amide |
| 354 | N-[(S)-1-[(6-Amino-pyridin-3-ylmethyl)-carbamoyl]-2-(3,4-difluoro- |
| | phenyl)-ethyl]-2-piperidin-1-yl-propionamide |
| 355 | (S)—N-[(S)-1-[(6-Amino-pyridin-3-ylmethyl)-carbamoyl]-2-(3,4- |
| | difluoro-phenyl)-ethyl]-2-diethylamino-propionamide |
| 356 | (R)—N-[(S)-1-[(6-Amino-pyridin-3-ylmethyl)-carbamoyl]-2-(3,4- |
| | difluoro-phenyl)-ethyl]-2-pyrrolidin-1-yl-propionamide |
| 357 | (S)—N-[(S)-1-[(6-Amino-pyridin-3-ylmethyl)-carbamoyl]-2-(3,4- |
| | difluoro-phenyl)-ethyl]-2-pyrrolidin-1-yl-propionamide |
| 358 | (R)-2-Dimethylamino-3-methyl-pentanoic acid [(S)-1-[(6-amino- |
| | pyridin-3-ylmethyl)-carbamoyl]-2-(3,4-difluoro-phenyl)-ethyl]-amide |
| 359 | (R)—N-[(S)-1-[(6-Amino-pyridin-3-ylmethyl)-carbamoyl]-2-(3,4- |
| | difluoro-phenyl)-ethyl]-2-dimethylamino-3-methyl-butyramide |
| 360 | (S)—N-[(S)-1-[(6-Amino-pyridin-3-ylmethyl)-carbamoyl]-2-(3,4- |
| | difluoro-phenyl)-ethyl]-3-hydroxy-2-(isopropyl-methyl-amino)- |
| | propionamide |
| 361 | (S)—N-[(S)-1-[(6-Amino-pyridin-3-ylmethyl)-carbamoyl]-2-(3,4- |
| | difluoro-phenyl)-ethyl]-2-(ethyl-methyl-amino)-3-hydroxy- |
| | propionamide |
| 362 | (S)—N-[(S)-1-[(6-Amino-pyridin-3-ylmethyl)-carbamoyl]-2-(3,4- |
| | difluoro-phenyl)-ethyl]-2-diethylamino-3-hydroxy-propionamide |
| 363 | (R)—N-[(S)-1-[(6-Amino-pyridin-3-ylmethyl)-carbamoyl]-2-(3,4- |
| | difluoro-phenyl)-ethyl]-2-diethylamino-3-hydroxy-propionamide |
| 364 | (S)—N-[(S)-1-[(6-Amino-pyridin-3-ylmethyl)-carbamoyl]-2-(3,4- |
| | difluoro-phenyl)-ethyl]-3-diethylamino-succinamic acid methyl ester |
| 365 | (S)—N-(6-Amino-pyridin-3-ylmethyl)- |
| | 3-(3,4-difluoro-phenyl)-2-[2-(2,6-dimethyl-piperidin-1-yl)- |
| | acetylamino]-propionamide |
| 366 | (S)—N-(6-Amino-pyridin-3-ylmethyl)-3-(3,4-difluoro-phenyl)-2-(2- |
| | pyrrolidin-1-yl-acetylamino)-propionamide |
| 367 | (S)—N-[(S)-1-[(6-Amino-pyridin-3-ylmethyl)-carbamoyl]-2-(4-fluoro- |
| | phenyl)-ethyl]-2-(ethyl-methyl-amino)-propionamide |
| 368 | (S)—N-[(S)-1-[(6-Amino-pyridin-3-ylmethyl)-carbamoyl]-2-(4-fluoro- |
| | phenyl)-ethyl]-2-(methyl-propyl-amino)-propionamide |
| 369 | (S)—N-[(S)-1-[(6-Amino-pyridin-3-ylmethyl)-carbamoyl]-2-(4-fluoro- |
| | phenyl)-ethyl]-2-(butyl-methyl-amino)-propionamide |
| 370 | (S)—N-[(S)-1-[(6-Amino-pyridin-3-ylmethyl)-carbamoyl]-2-(4-fluoro- |
| | phenyl)-ethyl]-2-(isopropyl-methyl-amino)-3-methyl-butyramide |
| 371 | (S)-2-(Isopropyl-methyl-amino)-3-methyl-pentanoic acid [(S)-1-[(6- |
| | amino-pyridin-3-ylmethyl)-carbamoyl]-2-(4-fluoro-phenyl)-ethyl]- |
| | amide |
| 372 | (S)—N-[(S)-1-[(6-Amino-pyridin-3-ylmethyl)-carbamoyl]-2-(4-fluoro- |
| | phenyl)-ethyl]-2-diisopropylamino-propionamide |
| 373 | (S)—N-[(S)-1-[(6-Amino-pyridin-3-ylmethyl)-carbamoyl]-2-(4-fluoro- |
| | phenyl)-ethyl]-2-(isopropyl-methyl-amino)-3-phenyl-propionamide |
| 374 | (R)—N-[(S)-1-[(6-Amino-pyridin-3-ylmethyl)-carbamoyl]-2-(4-fluoro- |
| | phenyl)-ethyl]-2-piperidin-1-yl-propionamide |
| 375 | (S)—N-[(S)-1-[(6-Amino-pyridin-3-ylmethyl)-carbamoyl]-2-(4-fluoro- |
| | phenyl)-ethyl]-2-piperidin-1-yl-propionamide |
| 376 | (S)—N-[(S)-1-[(6-Amino-pyridin-3-ylmethyl)-carbamoyl]-2-(4-fluoro- |
| | phenyl)-ethyl]-2-diethylamino-propionamide |
| 377 | (R)-2-Dimethylamino-3-methyl-pentanoic acid [(S)-1-[(6-amino- |
| | pyridin-3-ylmethyl)-carbamoyl]-2-(3-fluoro-phenyl)-ethyl]-amide |
| 378 | (S)—N-(6-Amino-pyridin-3-ylmethyl)-2-[2-(2,6-dimethyl-piperidin-1-yl)- |
| | acetylamino]-3-(4-fluoro-phenyl)-propionamide |
| 379 | (S)—N-(6-Amino-pyridin-3-ylmethyl)-3-(4-fluoro-phenyl)-2-(2- |
| | piperidin-1-yl-acetylamino)-propionamide |
| 380 | (S)—N-(6-Amino-pyridin-3-ylmethyl)-2-(2-diethylamino-acetylamino)-3- |
| | (4-fluoro-phenyl)-propionamide |
| 381 | (S)—N-{(S)-1-[(6-Amino-pyridin-3-ylmethyl)-carbamoyl]-2-cyclohexyl- |
| | ethyl}-2-(isopropyl-methyl-amino)-propionamide |
| 382 | (S)-1-Methyl-pyrrolidine-2-carboxylic acid {(S)-1-[(6-amino-pyridin-3- |
| | ylmethyl)-carbamoyl]-2-cyclohexyl-ethyl}-amide |
| 383 | (S)—N-{(S)-1-[(6-Amino-pyridin-3-ylmethyl)-carbamoyl]-2-cyclohexyl- |
| | ethyl}-2-(methyl-propyl-amino)-propionamide |
| 384 | (S)—N-{(S)-1-[(6-Amino-pyridin-3-ylmethyl)-carbamoyl]-2-cyclohexyl- |
| | ethyl}-2-(ethyl-methyl-amino)-propionamide |
| 385 | (S)—N-{(S)-1-[(6-Amino-pyridin-3-ylmethyl)-carbamoyl]-2-cyclohexyl- |
| | ethyl}-2-dipropylaminopropionamide |
| 386 | (S)—N-{(S)-1-[(6-Amino-pyridin-3-ylmethyl)-carbamoyl]-2-cyclohexyl- |
| | ethyl}-2-dimethylamino-3-hydroxy-propionamide |
| 387 | (S)—N-{(S)-1-[(6-Amino-pyridin-3-ylmethyl)-carbamoyl]-2-cyclohexyl- |
| | ethyl}-2-(ethyl-methyl-amino)-3-hydroxy-propionamide |
| 388 | (S)—N-{(S)-1-[(6-Amino-pyridin-3-ylmethyl)-carbamoyl]-2-cyclohexyl- |
| | ethyl}-3-hydroxy-2-(isopropyl-methyl-amino)-propionamide |
| 389 | (S)—N-{(S)-1-[(6-Amino-pyridin-3-ylmethyl)-carbamoyl]-2-cyclohexyl- |
| | ethyl}-2-diethylamino-3-hydroxy-propionamide |
| 390 | (S)—N-{(S)-1-[(6-Amino-pyridin-3-ylmethyl)-carbamoyl]-2-cyclohexyl- |
| | ethyl}-2-diethylamino-propionamide |
| 391 | (S)—N-{(S)-1-[(6-Amino-pyridin-3-ylmethyl)-carbamoyl]-2-cyclohexyl- |
| | ethyl}-2-dimethylamino-propionamide |
| 392 | (S)—N-(6-Amino-pyridin-3-ylmethyl)-3-cyclohexyl-2-[2-(2,6-dimethyl- |
| | piperidin-1-yl)-acetylamino]-propionamide |
| 393 | (S)—N-(6-Amino-pyridin-3-ylmethyl)-3-cyclohexyl-2-(2- |
| | diisopropylamino-acetylamino)-propionamide |
| 394 | (S)—N-(6-Amino-pyridin-3-ylmethyl)-3-(decahydro-naphthalen-1-yl)-2- |
| | (2-diisopropylamino-acetylamino)-propionamide |
| 395 | (S)—N-[(S)-1-[(6-Amino-pyridin-3-ylmethyl)-carbamoyl]-2-(decahydro- |
| | naphthalen-1-yl)-ethyl]-2-(isopropyl-methyl-amino)-propionamide |
| 396 | (R)-1-Methyl-pyrrolidine-2-carboxylic acid [(S)-1-[(6-amino-pyridin-3- |
| | ylmethyl)-carbamoyl]-2-(decahydro-naphthalen-1-yl)-ethyl]-amide |
| 397 | (R)—N-[(S)-1-[(6-Amino-pyridin-3-ylmethyl)-carbamoyl]-2-(decahydro- |
| | naphthalen-1-yl)-ethyl]-2-dimethylamino-3-phenyl-propionamide |
| 398 | (R)-2-Dimethylamino-3-methyl-pentanoic acid [(S)-1-[(6-amino- |
| | pyridin-3-ylmethyl)-carbamoyl]-2-(3,4-dichloro-phenyl)-ethyl]-amide |
| 399 | (R)—N-[(S)-1-[(6-Amino-pyridin-3-ylmethyl)-carbamoyl]-2-(3,4- |
| | dichloro-phenyl)-ethyl]-2-dimethylamino-3-methyl-butyramide |
| 400 | (R)—N-[(S)-1-[(6-Amino-pyridin-3-ylmethyl)-carbamoyl]-2-(3,4- |
| | dichloro-phenyl)-ethyl]-2-dimethylamino-3-phenyl-propionamide |
| 401 | (S)—N-[(S)-1-[(6-Amino-pyridin-3-ylmethyl)-carbamoyl]-2-(3,4- |
| | dichloro-phenyl)-ethyl]-2-(isopropyl-methyl-amino)-propionamide |
| 402 | (R)—N-[(S)-1-[(6-Amino-pyridin-3-ylmethyl)-carbamoyl]-2-(3,4- |
| | dichloro-phenyl)-ethyl]-2-piperidin-1-yl-propionamide |
| 403 | (S)—N-[(S)-1-[(6-Amino-pyridin-3-ylmethyl)-carbamoyl]-2-(3,4- |
| | dichloro-phenyl)-ethyl]-2-piperidin-1-yl-propionamide |
| 404 | (S)—N-(6-Amino-pyridin-3-ylmethyl)-3-(3,4-dichloro-phenyl)-2-(2- |
| | piperidin-1-yl-acetylamino)-propionamide |
| 405 | (R)-2-Dimethylamino-3-methyl-pentanoic acid [(S)-1-[(6-amino- |
| | pyridin-3-ylmethyl)-carbamoyl]-2-(3-chloro-phenyl)-ethyl]-amide |
| 406 | (R)—N-[(S)-1-[(6-Amino-pyridin-3-ylmethyl)-carbamoyl]-2-(3-chloro- |
| | phenyl)-ethyl]-2-dimethylamino-3-methyl-butyramide |
| 407 | (R)—N-[(S)-1-[6-Amino-pyridin-3-ylmethyl)-carbamoyl]-2-(3-chloro- |
| | phenyl)-ethyl]-2-dimethylamino-3-phenyl-propionamide |
| 408 | (S)—N-[(S)-1-[6-Amino-pyridin-3-ylmethyl)-carbamoyl]-2-(3-chloro- |
| | phenyl)-ethyl]-2-(ethyl-methyl-amino)-propionamide |
| 409 | (S)—N-[(S)-1-[6-Amino-pyridin-3-ylmethyl)-carbamoyl]-2-(3-chloro- |
| | phenyl)-ethyl]-2-(isopropyl-methyl-amino)-propionamide |
| 410 | (S)—N-[(S)-1-[(6-Amino-pyridin-3-ylmethyl)-carbamoyl]-2-(3-chloro- |
| | phenyl)-ethyl]-2-diethylamino-propionamide |
| 411 | N-[(S)-1-[(6-Amino-pyridin-3-ylmethyl)-carbamoyl]-2-(3-chloro- |
| | phenyl)-ethyl]-2-piperidin-1-yl-propionamide |
| 412 | (S)—N-(6-Amino-pyridin-3-ylmethyl)-3-(3-chloro-phenyl)-2-(2- |
| | piperidin-1-yl-acetylamino)-propionamide |
| 413 | (S)—N-{(S)-1-[(6-Amino-pyridin-3-ylmethyl)-carbamoyl]-2-m-tolyl- |
| | ethyl}-2-(ethyl-methyl-amino)-propionamide |
| 414 | (S)—N-{(S)-1-[(6-Amino-pyridin-3-ylmethyl)-carbamoyl]-2-m-tolyl- |
| | ethyl}-2-(isopropyl-methyl-amino)-propionamide |
| 415 | (S)—N-{(S)-1-[(6-Amino-pyridin-3-ylmethyl)-carbamoyl]-2-m-tolyl- |
| | ethyl}-2-diethylamino-propionamide |
| 416 | (S)—N-[(S)-1-[(6-Amino-pyridin-3-ylmethyl)-carbamoyl]-2-(3- |
| | trifluoromethyl-phenyl)-ethyl]-2-(ethyl-methyl-amino)-propionamide |
| 417 | (S)—N-[(S)-1-[(6-Amino-pyridin-3-ylmethyl)-carbamoyl]-2-(3- |
| | trifluoromethyl-phenyl)-ethyl]-2-(isopropyl-methyl-amino)- |
| | propionamide |
| 418 | (S)—N-[(S)-1-[(6-Amino-pyridin-3-ylmethyl)-carbamoyl]-2-(3- |
| | trifluoromethyl-phenyl)-ethyl]-2-diethylamino-propionamide |
| 419 | N-[(S)-1-[(6-Amino-pyridin-3-ylmethyl)-carbamoyl]-2-(3- |
| | trifluoromethyl-phenyl)-ethyl]-2-piperidin-1-yl-propionamide |
| 420 | (S)—N-(6-Amino-pyridin-3-ylmethyl)-2-(2-piperidin-1-yl-acetylamino)- |
| | 3-(3-trifluoromethyl-phenyl)-propionamide |
| 421 | (R)—N-[(S)-1-[(6-Amino-pyridin-3-ylmethyl)-carbamoyl]-2-(4- |
| | trifluoromethyl-phenyl)-ethyl]-2-piperidin-1-yl-propionamide |
| 422 | (S)—N-[(S)-1-[(6-Amino-pyridin-3-ylmethyl)-carbamoyl]-2-(4- |
| | trifluoromethyl-phenyl)-ethyl]-2-piperidin-1-yl-propionamide |
| 423 | (S)—N-(6-Amino-pyridin-3-ylmethyl)-2-(2-piperidin-1-yl-acetylamino)- |
| | 3-(4-trifluoromethyl-phenyl)-propionamide |
| 424 | (R)-2-Dimethylamino-3-methyl-pentanoic acid [(S)-1-[(6-amino- |
| | pyridin-3-ylmethyl)-carbamoyl]-2-(3,5-difluoro-phenyl)-ethyl]-amide |
| 425 | N-[(S)-1-[(6-Amino-pyridin-3-ylmethyl)-carbamoyl]-2-(3,5-difluoro- |
| | phenyl)-ethyl]-2-piperidin-1-yl-propionamide |
| 426 | (S)—N-(6-Amino-pyridin-3-ylmethyl)-3-(4-chloro-phenyl)-2-[2-(2,6- |
| | dimethyl-piperidin-1-yl)-acetylamino]-propionamide |
| 427 | (S)-2-[(S)-2-(Isopropyl-methyl-amino)-propionylamino]-4-methyl- |
| | pentanoic acid (6-amino-pyridin-3-ylmethyl)-amide |
| 428 | (S)—N-(6-Amino-2-methyl-pyridin-3-ylmethyl)-2-(2-diethylamino- |
| | acetylamino)-3-(4-fluoro-phenyl)-propionamide |
| 429 | (S)—N-(6-Amino-2-methyl-pyridin-3-ylmethyl)-3-(3,4-difluoro-phenyl)- |
| | 2-[2-(2,6-dimethyl-piperidin-1-yl)-acetylamino]-propionamide |
| 430 | (S)—N-(6-Amino-2-methyl-pyridin-3-ylmethyl)-3-(3,4-difluoro-phenyl)- |
| | 2-(2-piperidin-1-yl-acetylamino)-propionamide |
| 431 | (R)—N-[(S)-1-[(6-Amino-2-methyl-pyridin-3-ylmethyl)-carbamoyl]-2- |
| | (3,4-difluoro-phenyl)-ethyl]-2-piperidin-1-yl-propionamide |
| 432 | (S)—N-[(S)-1-[(6-Amino-2-methyl-pyridin-3-ylmethyl)-carbamoyl]-2- |
| | (3,4-difluoro-phenyl)-ethyl]-2-piperidin-1-yl-propionamide |
| 433 | (S)—N-{(R)-1-[(6-Amino-pyridin-3-ylmethyl)-carbamoyl]-2,2- |
| | dicyclohexyl-ethyl}-2-(isopropyl-methyl-amino)-propionamide |
| 434 | (S)—N-{(R)-1-[(6-Amino-pyridin-3-ylmethyl)-carbamoyl]-2,2- |
| | dicyclohexyl-ethyl}-2-dimethylamino-propionamide |
| 435 | (S)—N-{(R)-1-[(6-Amino-pyridin-3-ylmethyl)-carbamoyl]-2-cyclohexyl- |
| | 2-hydroxy-ethyl}-2-(isopropyl-methyl-amino)-propionamide |
| 436 | (R)—N-(6-Amino-pyridin-3-ylmethyl)-3-cyclohexyl-2-[(S)-2-(isopropyl- |
| | methyl-amino)-propionylamino]-butyramide |
| 437 | (S)—N-{(S)-1-[(6-Amino-pyridin-3-ylmethyl)-carbamoyl]-2-cyclohexyl- |
| | ethyl}-2-(isopropyl-methyl-amino)-N-methyl-propionamide |
| 438 | S)—N-{(S)-1-[(6-Amino-pyridin-3-ylmethyl)-carbamoyl]-2-cyclohexyl- |
| | ethyl}-2-diethylamino-N-methyl-propionamide |
| 439 | (S)—N-{(S)-1-[(6-Amino-pyridin-3-ylmethyl)-carbamoyl]-2-cyclohexyl- |
| | ethyl}-2-dipropylamino-propionamide |

Biological Methods

The ability of the compounds of formula (I) to inhibit KLK1 may be determined using the following biological assays:

Determination of the IC₅₀ for KLK1

KLK1 inhibitory activity in vitro was determined using standard published methods (see e.g. Johansen et al., Int. J. Tiss. Reac. 1986, 8, 185; Shori et al., Biochem. Pharmacol., 1992, 43, 1209; Stürzebecher et al., Biol. Chem. Hoppe-Seyler, 1992, 373, 1025). Human KLK1 (Callbiochem) was incubated at 37° C. with the fluorogenic substrate H-DVal-Leu-Arg-AFC and various concentrations of the test compound. Residual enzyme activity (initial rate of reaction) was determined by measuring the change in optical absorbance at 410 nm and the IC₅₀ value for the test compound was determined.

Determination of Enzyme Selectivity

Selected compounds were further screened for inhibitory activity against other trypsin-like serine proteases using the appropriate enzyme and chromogenic substrate (Chromogenix AB). The activity against the following human enzymes was tested (substrate in brackets):—plasma kallikrein (S-2302), thrombin (S-2238), plasmin (S-2390) and trypsin (S-2222). The enzyme was incubated at 37° C. with the chromogenic substrate. Residual enzyme activity (initial rate of reaction) was determined by measuring the change in optical absorbance at 405 nm.

Data acquired from these assays are shown in Tables 16 and 17 below:

TABLE 16
(In vitro activity)

| Example No | IC₅₀ vs KLK1 (nM) |
|---|---|
| 1 | 0.96 |
| 2 | 1.0 |
| 3 | 0.22 |
| 4 | 0.7 |
| 5 | 1.3 |
| 6 | 2.0 |
| 7 | 10.5 |
| 8 | 1.7 |
| 9 | 6.3 |
| 10 | 13.7 |
| 11 | 4.9 |
| 12 | 6.3 |
| 13 | 9.8 |
| 14 | 0.96 |
| 15 | 1.2 |
| 16 | 1.1 |
| 17 | 211 |
| 18 | 5.0 |
| 19 | 21.6 |
| 20 | 4.3 |
| 21 | 22.8 |
| 22 | 2.2 |
| 24 | 27.8 |
| 25 | 9.8 |
| 26 | 20.5 |
| 27 | 2.5 |
| 28 | 32.1 |
| 29 | 10.2 |
| 30 | 1.1 |
| 31 | 18.1 |
| 32 | 2.8 |
| 33 | 5.3 |
| 34 | 3.3 |
| 35 | 0.47 |
| 36 | 1.6 |
| 37 | 626 |
| 38 | 9.2 |
| 39 | 5.8 |
| 40 | 6.6 |
| 41 | 0.76 |
| 42 | 1.4 |
| 43 | 2.9 |
| 44 | 5.4 |
| 45 | 3.1 |
| 46 | 1.2 |
| 47 | 316 |
| 48 | 8.0 |
| 49 | 4.2 |
| 50 | 29.1 |
| 51 | 0.26 |
| 52 | 24.5 |
| 53 | 11.2 |
| 54 | 0.29 |
| 55 | 0.70 |
| 56 | 7.1 |
| 57 | 4.6 |
| 58 | 48.6 |
| 59 | 0.56 |
| 60 | 0.71 |
| 61 | 0.4 |
| 62 | 2.4 |
| 63 | 0.66 |
| 64 | 3.1 |
| 65 | 2.7 |
| 66 | 20.7 |
| 67 | 3.1 |
| 68 | 9.2 |
| 69 | 42.2 |
| 70 | 1.1 |
| 72 | 7.2 |
| 73 | 1.1 |
| 74 | 1.8 |
| 75 | 2.2 |
| 76 | 21.8 |
| 77 | 2.9 |
| 78 | 1.2 |
| 80 | 16.1 |
| 81 | 5.0 |
| 82 | 11.2 |
| 83 | 158 |
| 84 | 8.0 |
| 85 | 13.9 |
| 86 | 15.5 |
| 87 | 6.4 |
| 88 | 6.7 |
| 89 | 11.0 |
| 90 | 0.86 |
| 91 | 2.1 |
| 92 | 3.6 |
| 93 | 0.85 |
| 94 | 12.5 |
| 95 | 1.8 |
| 96 | 5.5 |
| 97 | 45.2 |
| 98 | 1.3 |
| 99 | 17.1 |
| 100 | 85.8 |
| 101 | 24.1 |
| 102 | 258 |
| 103 | 5.4 |
| 104 | 24.4 |
| 105 | 3.8 |
| 106 | 3.9 |
| 107 | 71.2 |
| 108 | 34.0 |
| 109 | 21.7 |
| 110 | 4.0 |
| 111 | 15.0 |
| 112 | 257 |
| 113 | 14.5 |
| 114 | 28.4 |
| 115 | 19.5 |
| 116 | 16.1 |
| 117 | 0.45 |
| 118 | 2.1 |
| 119 | 1.8 |
| 120 | 1.6 |
| 121 | 11.0 |
| 122 | 1.8 |
| 123 | 1.4 |
| 124 | 36.8 |
| 125 | 8.8 |
| 126 | 9.3 |
| 127 | 0.9 |
| 128 | 7.6 |
| 129 | 215 |
| 130 | 5.0 |
| 131 | 0.54 |
| 132 | 6.4 |
| 133 | 16.5 |
| 134 | 13.5 |
| 135 | 17.3 |
| 136 | 8.6 |
| 137 | 288 |
| 138 | 39.8 |
| 139 | 3.7 |
| 140 | 4.3 |
| 141 | 13.7 |
| 142 | 15.0 |
| 143 | 21.0 |
| 144 | 20.9 |
| 145 | 10.5 |
| 146 | 102 |
| 147 | 49.6 |
| 148 | 55.7 |
| 149 | 36.8 |
| 150 | 12.0 |
| 151 | 17.7 |
| 152 | 5.7 |
| 153 | 69.9 |
| 154 | 216 |
| 155 | 63.3 |
| 156 | 80.6 |
| 157 | 123.3 |
| 158 | 114.4 |
| 159 | 165.1 |
| 160 | 184.8 |
| 161 | 93.5 |
| 162 | 38.1 |
| 163 | 56.5 |
| 164 | 345 |
| 165 | 83.5 |
| 166 | 726 |
| 167 | 258 |
| 168 | 291 |
| 169 | 334 |
| 170 | 16.6 |
| 171 | 1.3 |
| 172 | 12.7 |
| 173 | 78.3 |
| 174 | 90.0 |
| 175 | 2.0 |
| 176 | 4.0 |
| 177 | 1.9 |
| 178 | 15.9 |
| 179 | 3.01 |
| 180 | 7.9 |
| 181 | 1.1 |
| 182 | 21.2 |
| 183 | 5.1 |
| 184 | 44.4 |
| 185 | 14.5 |
| 186 | 0.81 |
| 187 | 0.71 |
| 188 | 1.2 |
| 189 | 4.2 |
| 190 | 1.8 |
| 191 | 33.3 |
| 192 | 60.4 |
| 193 | 10.9 |
| 194 | 5.1 |
| 195 | 5.4 |
| 196 | 23.2 |
| 197 | 84.6 |
| 198 | 28.0 |
| 199 | 1.4 |
| 200 | 22.1 |
| 201 | 1.3 |
| 202 | 22.5 |
| 203 | 4.5 |
| 204 | 5.9 |
| 205 | 6.7 |
| 208 | 32.1 |
| 209 | 36.0 |
| 210 | 39.0 |
| 211 | 0.29 |
| 212 | 29.8 |
| 213 | 2.8 |
| 214 | 16.9 |
| 215 | 12.6 |
| 216 | 24.4 |
| 217 | 29.3 |
| 218 | 35.3 |
| 219 | 3.6 |
| 220 | 55.5 |
| 221 | 50.5 |
| 222 | 39.7 |
| 223 | 17.37 |
| 224 | 61.6 |
| 225 | 51.1 |
| 226 | 10.1 |
| 227 | 5.3 |
| 228 | 32.1 |
| 229 | 3.4 |
| 230 | 20.3 |
| 231 | 3.1 |
| 232 | 1.5 |
| 233 | 2.1 |
| 234 | 3.2 |
| 235 | 2.4 |
| 236 | 9.0 |
| 237 | 10.9 |
| 238 | 35.2 |
| 239 | 6.5 |
| 240 | 1.5 |
| 241 | 3.8 |
| 242 | 4.4 |
| 243 | 3.4 |
| 244 | 7.8 |
| 245 | 13.7 |
| 246 | 3.5 |
| 247 | 200 |
| 248 | 12.7 |
| 249 | 6.9 |
| 250 | 0.8 |
| 251 | 128 |
| 252 | 43.2 |
| 253 | 12.5 |
| 254 | 37.2 |
| 255 | 105 |
| 256 | 122 |
| 257 | 12.1 |
| 258 | 10.3 |
| 259 | 85.4 |
| 260 | 150 |
| 261 | 13.2 |
| 262 | 28.3 |
| 263 | 20.5 |
| 264 | 3.1 |
| 265 | 21.6 |
| 266 | 5.7 |
| 267 | 8.2 |
| 268 | 7.7 |
| 269 | 26.1 |
| 270 | 20.9 |
| 271 | 3.3 |
| 272 | 10.3 |
| 273 | 375 |
| 274 | 4.8 |
| 275 | 147 |
| 276 | 30.5 |
| 277 | 0.9 |
| 278 | 3.2 |
| 279 | 4.5 |
| 280 | 1.9 |
| 281 | 1.8 |
| 282 | 1.3 |
| 283 | 5.1 |
| 284 | 5.2 |
| 285 | 6.5 |
| 286 | 4.3 |
| 287 | 75.6 |
| 288 | 93.2 |
| 289 | 30.5 |
| 290 | 626 |
| 291 | 165 |
| 292 | 31.5 |
| 293 | 36.3 |
| 294 | 35.6 |
| 295 | 192 |
| 296 | 40.9 |
| 297 | 40.1 |
| 298 | 9.4 |
| 299 | 8.1 |
| 300 | 7.4 |
| 301 | 11.0 |
| 302 | 7.2 |
| 303 | 240 |
| 304 | 148 |
| 305 | 119 |
| 306 | 163 |
| 307 | 22.0 |
| 308 | 15.6 |
| 309 | 42.3 |
| 310 | 48.9 |
| 311 | 46.5 |
| 312 | 38.4 |
| 313 | 8.7 |
| 314 | 15.6 |
| 315 | 68.6 |
| 316 | 76.7 |
| 317 | 14.7 |
| 318 | 12.1 |
| 319 | 110 |
| 320 | 98.5 |
| 321 | 85.4 |
| 322 | 12.4 |
| 323 | 48.1 |
| 324 | 68.4 |
| 325 | 21.3 |
| 326 | 25.4 |
| 327 | 17.8 |
| 328 | 179 |
| 329 | 299 |
| 330 | 23.6 |
| 331 | 32.0 |
| 332 | 21.5 |
| 333 | 184 |
| 334 | 235 |
| 335 | 15.8 |
| 338 | 881 |
| 339 | 39.8 |
| 340 | 0.38 |
| 341 | 2.2 |
| 342 | 2.7 |
| 343 | 91.0 |
| 344 | 21.3 |
| 345 | 7.7 |
| 346 | 21.3 |
| 347 | 24.5 |
| 348 | 11.1 |
| 349 | 7.8 |
| 350 | 28.5 |
| 351 | 8.8 |
| 352 | 13.7 |
| 353 | 7.8 |
| 354 | 11.6 |
| 355 | 2.5 |
| 356 | 3.8 |
| 357 | 3.0 |
| 358 | 10.4 |
| 359 | 5.0 |
| 360 | 20.2 |
| 361 | 55.6 |
| 362 | 25.9 |
| 363 | 84.4 |
| 364 | 43.7 |
| 365 | 2.5 |
| 366 | 54.5 |
| 367 | 10.8 |
| 368 | 19.4 |
| 369 | 29.1 |
| 370 | 11.3 |
| 371 | 7.3 |
| 372 | 34.3 |
| 373 | 25.7 |
| 374 | 52.7 |
| 375 | 4.9 |
| 376 | 2.7 |
| 377 | 32.2 |
| 378 | 2.5 |
| 379 | 128 |
| 380 | 13.9 |
| 381 | 1.1 |
| 382 | 48.6 |
| 383 | 45.9 |
| 384 | 9.7 |
| 385 | 107 |
| 386 | 260 |
| 387 | 170 |
| 388 | 22.2 |
| 389 | 42.9 |
| 390 | 2.7 |
| 391 | 49.4 |
| 392 | 47.2 |
| 393 | 26.9 |
| 394 | 8.4 |
| 395 | 0.17 |
| 396 | 1.4 |
| 397 | 44.2 |
| 398 | 0.82 |
| 399 | 0.74 |
| 400 | 1.7 |
| 401 | 0.16 |
| 402 | 13.0 |
| 403 | 1.1 |
| 404 | 33.4 |
| 405 | 7.1 |
| 406 | 4.7 |
| 407 | 17.0 |
| 408 | 4.8 |
| 409 | 0.63 |
| 410 | 2.6 |
| 411 | 12.7 |
| 412 | 9.3 |
| 413 | 7.9 |
| 414 | 0.77 |
| 415 | 5.3 |
| 416 | 6.2 |
| 417 | 0.83 |
| 418 | 2.0 |
| 419 | 16.3 |
| 420 | 117 |
| 421 | 67.4 |
| 422 | 3.0 |
| 423 | 129 |
| 424 | 28.0 |
| 425 | 14.9 |
| 426 | 2.7 |
| 427 | 106 |
| 428 | 9.9 |
| 429 | 1.7 |
| 430 | 59.8 |
| 431 | 50.6 |
| 432 | 17.0 |
| 433 | 100 |
| 434 | 43.9 |
| 435 | 5.0 |
| 436 | 3.6 |
| 437 | 4.2 |
| 438 | 5.4 |
| 439 | 107 |

TABLE 17
(Selectivity data)

IC₅₀ (nM)

| Example | Plasma | | | |
|---|---|---|---|---|
| No | Kallikrein | Thrombin | Trypsin | Plasmin |
| 1 | >10000 | >10000 | >10000 | >10000 |
| 2 | >10000 | >10000 | >10000 | >10000 |
| 3 | >10000 | >10000 | >10000 | >10000 |
| 4 | >10000 | >10000 | >10000 | >10000 |
| 5 | >10000 | >10000 | >10000 | >10000 |
| 11 | >10000 | >10000 | >10000 | >10000 |
| 13 | >10000 | >10000 | >10000 | >10000 |
| 20 | >10000 | >10000 | >10000 | >10000 |
| 25 | >10000 | >10000 | >10000 | >10000 |
| 26 | >10000 | >10000 | >10000 | >10000 |
| 27 | >10000 | >10000 | >10000 | >10000 |
| 29 | >10000 | >10000 | >10000 | >10000 |
| 30 | >10000 | >10000 | >10000 | >10000 |
| 54 | >10000 | >10000 | >10000 | >10000 |
| 55 | >10000 | >10000 | >10000 | >10000 |
| 59 | >10000 | >10000 | >10000 | >10000 |
| 60 | >10000 | >10000 | >10000 | >10000 |
| 61 | >10000 | >10000 | >10000 | >10000 |
| 62 | >10000 | >10000 | >10000 | >10000 |
| 90 | >10000 | >10000 | >10000 | >10000 |
| 91 | >10000 | >10000 | >10000 | >10000 |
| 92 | >10000 | >10000 | >10000 | >10000 |
| 213 | >10000 | >10000 | >10000 | >10000 |
| 131 | >10000 | >10000 | >10000 | >10000 |
| 145 | >10000 | >10000 | >10000 | >10000 |
| 157 | >10000 | >10000 | >10000 | >10000 |
| 176 | >10000 | >10000 | >10000 | >10000 |
| 226 | >10000 | >10000 | >10000 | >10000 |
| 227 | >10000 | >10000 | >10000 | >10000 |
| 228 | >10000 | >10000 | >10000 | >10000 |
| 229 | >10000 | >10000 | >10000 | >10000 |
| 231 | >10000 | >10000 | >10000 | >10000 |
| 232 | >10000 | >10000 | >10000 | >10000 |
| 233 | >10000 | >10000 | >10000 | >10000 |
| 234 | >10000 | >10000 | >10000 | >10000 |
| 235 | >10000 | >10000 | >10000 | >10000 |
| 242 | >10000 | >10000 | >10000 | >10000 |
| 243 | >10000 | >10000 | >10000 | >10000 |
| 277 | >10000 | >10000 | >10000 | >10000 |
| 355 | >10000 | >10000 | >10000 | >10000 |
| 365 | >10000 | >10000 | >10000 | >10000 |
| 381 | >10000 | >10000 | >10000 | >10000 |
| 390 | >10000 | >10000 | >10000 | >10000 |
| 398 | >10000 | >10000 | >10000 | >10000 |
| 400 | >10000 | >10000 | >10000 | >10000 |
| 437 | >10000 | >10000 | >10000 | >10000 |

1. A compound of formula (I): 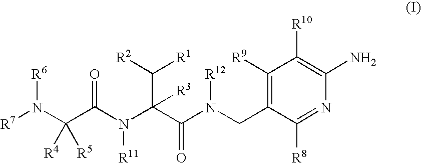
wherein:
R¹ and R² are independently selected from H, OH, (C₁-C₁₀)alkyl, (C₁-C₆)alkoxy, (C₂-C₆)alkenyl, (C₃-C₁₀)cycloalkyl, heterocycloalkyl, aryl, heteroaryl, aryl(C₁-C₄)alkyl- and heteroaryl(C₁-C₄)alkyl-;
R³ is selected from H, (C₁-C₁₀)alkyl and (C₂-C₆)alkenyl;
R⁴ and R⁵ are selected from H, (C₁-C₁₀)alkyl, (C₂-C₆)alkenyl, (C₃-C₁₀)cycloalkyl, heterocycloalkyl, aryl, heteroaryl, aryl(C₁-C₄)alkyl- and heteroaryl(C₁-C₄)alkyl-;
R⁶ and R⁷ are selected from H, (C₁-C₁₀)alkyl, (C₂-C₆)alkenyl, (C₃-C₁₀)cycloalkyl, heterocycloalkyl, aryl, heteroaryl, aryl(C₁-C₄)alkyl-, aryl(C₂-C₄)alkenyl-, heteroaryl(C₁-C₄)alkyl-, —SO₂(C₁-C₆)alkyl, —SO₂aryl and —SO₂arylC₁-C₄)alkyl;
or R⁶ and R⁷ together with the nitrogen atom to which they are attached may form a 4-7 membered N-containing ring, optionally containing one further heteroatom selected from N, O and S, and optionally substituted with 1 or 2 substituents independently selected from (C₁-C₆)alkyl, (C₁-C₆)alkoxy, halo, CN and hydroxyl, said N-containing ring may also optionally be fused to an aryl group;
or R⁴ and R⁶ together with the atoms to which they are attached may form a saturated or partially unsaturated 4-7 membered N-containing ring, optionally containing one further heteroatom selected from N, O and S, and optionally substituted on carbon with 1 or 2 substituents independently selected from (C₁-C₆)alkyl, (C₁-C₆)alkoxy, halo, CN and hydroxyl;
or R⁵ is absent and R⁴ and R⁶ together with the atoms to which they are attached may form a 5, 6, 9 or 10 membered mono- or bi-cyclic N-containing aromatic ring, optionally containing one further heteroatom selected from N, O and S, and optionally substituted on carbon with 1, 2 or 3 substituents independently selected from (C₁-C₆)alkyl, (C₁-C₆)alkoxy, halo, CN, aryl, COOR¹⁴ and hydroxyl;
or R⁴ and R⁶ may together form a group according to formula II or formula III: 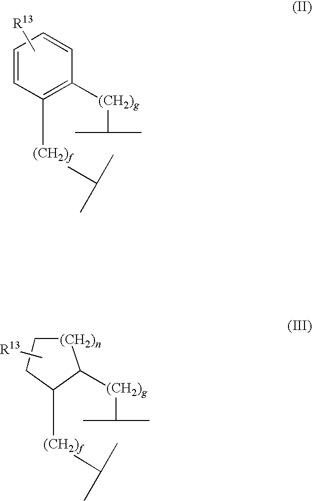
R⁸, R⁹ and R¹⁰ are independently selected from H, (C₁-C₁₀)alkyl, halo, hydroxyl and (C₁-C₆)alkoxy;
R¹¹ is selected from H and (C₁-C₆)alkyl;
R¹² is selected from H and (C₁-C₆)alkyl;
R¹³ is selected from H, (C₁-C₆)alkyl, (C₁-C₆)alkoxy, OH, CN, CF₃, COOR¹⁴, halo and NR¹⁴R¹⁵;
R¹⁴ and R¹⁵ are independently selected from H and (C₁-C₆)alkyl;
f and g are independently selected from 0, 1, 2 and 3, such that f+g=1, 2 or 3;
h is selected from 1 and 2;
wherein:
alkyl may optionally be substituted with 1 or 2 substituents independently selected from (C₃-C₁₀)cycloalkyl, (C₁-C₆)alkoxy, OH, CN, CF₃, COOR¹⁴, halo and NR¹⁴R¹⁵;
alkenyl may optionally be substituted with 1 or 2 substituents independently selected from (C₃-C₁₀)cycloalkyl, (C₁-C₆)alkoxy, OH, CN, CF₃, COOR¹⁴, halo and NR¹⁴R¹⁵;
alkoxy may optionally be substituted with 1 or 2 substituents independently selected from (C₃-C₁₀)cycloalkyl, OH, CN, CF₃, COOR¹⁴, halo and NR¹⁴_{R}¹⁵;
cycloalkyl is a non-aromatic mono- or bi-cylic hydrocarbon ring, optionally fused to an aryl group, wherein said cycloalkyl ring optionally contains, where possible, up to 2 double bonds; and wherein, unless otherwise stated, said cycloalkyl may optionally be substituted with 1 or 2 substituents independently selected from (C₁-C₆)alkyl, (C₁-C₆)alkoxy, OH, CN, CF₃, COOR¹⁴ , halo and NR¹⁴R¹⁵;
heterocycloalkyl is a C-linked or N-linked 3 to 10 membered non-aromatic, mono- or bi-cyclic ring, wherein said heterocycloalkyl ring contains, where possible, 1, 2 or 3 heteroatoms independently selected from N, NR¹⁴, S(O)_{q} and O; and said heterocycloalkyl ring optionally contains, where possible, 1 or 2 double bonds, and is optionally substituted on carbon with 1 or 2 substituents independently selected from (C₁-C₆)alkyl, (C₁-C₆)alkoxy, OH, CN, CF₃, halo, COOR¹⁴, NR¹⁴R¹⁵ and aryl;
aryl is a single or fused aromatic ring system containing 6 or 10 carbon atoms; wherein, unless otherwise stated, each occurrence of aryl may be optionally substituted with up to 5 substituents independently selected from (C₁-C₆)alkyl, (C₁-C₆)alkoxy, OH, halo, CN, COOR¹⁴, CF₃ and NR¹⁴R¹⁵;
heteroaryl is a 5, 6, 9 or 10 membered mono- or bi-cyclic aromatic ring, containing 1 or 2 N atoms and, optionally, an NR¹⁴ atom, or one NR¹⁴ atom and an S or an O atom, or one S atom, or one O atom; wherein, unless otherwise stated, said heteroaryl may be optionally substituted with 1, 2 or 3 substituents independently selected from (C₁-C₆)alkyl, (C₁-C₆)alkoxy, OH, halo, CN, COOR¹⁴, CF₃ and NR¹⁴R¹⁵;
q is 0, 1 or 2;
and tautomers, stereoisomers, pharmaceutically acceptable salts and solvates thereof.

2. A compound according to claim 1, or a tautomer, stereoisomer, pharmaceutically acceptable salt or solvate thereof, wherein R¹ is selected from (C₁-C₁₀)alkyl, (C₃-C₁₀)cycloalkyl, aryl, heteroaryl and aryl(C₁-C₄)alkyl- and R² is selected from H, (C₁-C₆)alkyl, (C₁-C₆)alkoxy, OH, (C₃-C₁₀)cycloalkyl and aryl.

3. A compound according to claim 1, or a tautomer, stereoisomer, pharmaceutically acceptable salt or solvate thereof, wherein R⁴ is selected from (C₁-C₁₀)alkyl, (C₃-C₁₀)cycloalkyl, aryl, heteroaryl, heteroaryl(C₁-C₄)alkyl- and aryl(C₁-C₄)alkyl- and R⁵ is selected from H and (C₁-C₆)alkyl.

4. A compound according to claim 1, or a tautomer, stereoisomer, pharmaceutically acceptable salt or solvate thereof, wherein:
R⁴ and R⁶ together with the atoms to which they are attached may form a 4-7 membered N-containing ring, optionally containing one carbon-carbon double bond, optionally containing one further heteroatom selected from N, O and S, and optionally substituted on carbon with 1 or 2 substituents independently selected from (C₁-C₆)alkyl, (C₁-C₆)alkoxy, halo, CN and hydroxyl; or
R⁵ is absent and R⁴ and R⁶ together with the atoms to which they are attached may form a 5, 6 or 9 membered mono- or bi-cyclic N-containing aromatic ring, optionally containing one further heteroatom selected from N and O, and optionally substituted on carbon with 1, 2 or 3 substituents independently selected from (C₁-C₆)alkyl, (C₁-C₆)alkoxy, halo, aryl, COOR¹⁴ and hydroxyl; or
R⁴ and R⁶ may together form a group according to formula II or formula III: 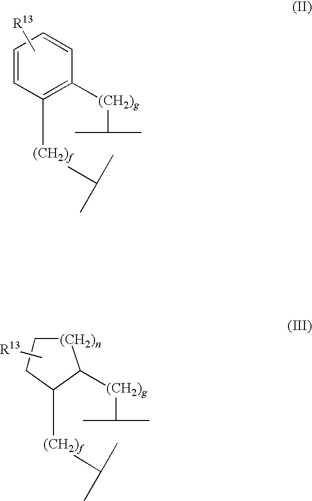
wherein R¹³ is H and f and g are independently selected from 0, 1, 2 and 3, such that f+g=1, 2 or 3; and h is selected from 1 and 2.

5. A compound according to claim 1, or a tautomer, stereoisomer, pharmaceutically acceptable salt or solvate thereof, wherein R⁶ is selected from H and (C₁-C₆)alkyl, and R⁷ is selected from H, (C₁-C₁₀)alkyl, (C₃-C₁₀)cycloalkyl, aryl, heteroaryl, aryl(C₁-C₄)alkyl-, aryl(C₂-C₄)alkenyl-, heteroaryl(C₁-C₄)alkyl-, —SO₂(C₁-C₆)alkyl and —SO₂aryl.

6. A compound according to claim 1, or a tautomer, stereoisomer, pharmaceutically acceptable salt or solvate thereof, wherein R⁶ and R⁷ together with the nitrogen atom to which they are attached may form a 5-6 membered N-containing ring, optionally containing one further heteroatom selected from N, O and S, and optionally substituted with 1 or 2 substituents independently selected from (C₁-C₆)alkyl, (C₁-C₆)alkoxy, halo, CN and hydroxyl, said N-containing ring may also optionally be fused to an aryl group.

7. A compound according to claim 1, or a tautomer, stereoisomer, pharmaceutically acceptable salt or solvate thereof, wherein R⁸, R⁹ and R¹⁹ are independently selected from H, (C₁-C₁₀)alkyl and halogen, and R¹¹ and R¹² are H.

8. A compound according to claim 1, selected from:
(R)-1-Methyl-pyrrolidine-2-carboxylic acid {(S)-1-[(6-amino-pyridin-3-ylmethyl)-carbamoyl]-2-naphthalen-1-yl-ethyl}-amide;
(R)-1-Methyl-pyrrolidine-2-carboxylic acid [(S)-1-[(6-amino-pyridin-3-ylmethyl)-carbamoyl]-2-(3-chloro-phenyl)-ethyl]-amide;
(R)-1-Methyl-pyrrolidine-2-carboxylic acid [(S)-1-[(6-amino-pyridin-3-ylmethyl)-carbamoyl]-2-(3,4-difluoro-phenyl)-ethyl]-amide;
(R)-3-Methyl-2-methylamino-pentanoic acid [(S)-1-[(6-amino-pyridin-3-ylmethyl)-carbamoyl]-2-(3,4-difluoro-phenyl)-ethyl]-amide;
(R)-1-Methyl-pyrrolidine-2-carboxylic acid [(S)-1-[(6-amino-pyridin-3-ylmethyl)-carbamoyl]-2-(3,5-difluoro-phenyl)-ethyl]-amide;
(R)-1-Methyl-pyrrolidine-2-carboxylic acid [(S)-1-[(6-amino-pyridin-3-ylmethyl)-carbamoyl]-2-(4-fluoro-phenyl)-ethyl]-amide;
(R)-1-Methyl-pyrrolidine-2-carboxylic acid [(S)-1-[(6-amino-pyridin-3-ylmethyl)-carbamoyl]-2-(3-fluoro-phenyl)-ethyl]-amide;
(R)-1-Isopropyl-pyrrolidine-2-carboxylic acid [(S)-1-[(6-amino-pyridin-3-ylmethyl)-carbamoyl]-2-(3,4-difluoro-phenyl)-ethyl]-amide;
(R)-1-Isopropyl-pyrrolidine-2-carboxylic acid [(S)-1-[(6-amino-pyridin-3-ylmethyl)-carbamoyl]-2-(4-fluoro-phenyl)-ethyl]-amide;
(R)-1-Ethyl-pyrrolidine-2-carboxylic acid [(S)-1-[(6-amino-pyridin-3-ylmethyl)-carbamoyl]-2-(3,4-difluoro-phenyl)-ethyl]-amide;
(R)-1-Propyl-pyrrolidine-2-carboxylic acid [(S)-1-[(6-amino-pyridin-3-ylmethyl)-carbamoyl]-2-(3,4-difluoro-phenyl)-ethyl]-amide;
(R)-1-Isobutyl-pyrrolidine-2-carboxylic acid [(S)-1-[(6-amino-pyridin-3-ylmethyl)-carbamoyl]-2-(3,4-difluoro-phenyl)-ethyl]-amide;
(R)-1-Ethyl-pyrrolidine-2-carboxylic acid [(S)-1-[(6-amino-pyridin-3-ylmethyl)-carbamoyl]-2-(4-fluoro-phenyl)-ethyl]-amide;
(S)—N-(6-Amino-pyridin-3-ylmethyl)-3-(3,4-difluoro-phenyl)-2-(2-diisopropylamino-acetylamino)-propionamide;
(R)-1-Methyl-piperidine-2-carboxylic acid [(S)-1-[(6-amino-pyridin-3-ylmethyl)-carbamoyl]-2-(3,4-difluoro-phenyl)-ethyl]-amide;
(R)-1-Methyl-piperidine-2-carboxylic acid [(S)-1-[(6-amino-pyridin-3-ylmethyl)-carbamoyl]-2-(4-fluoro-phenyl)-ethyl]-amide;
(S)—N—[(S)-1-[(6-Amino-pyridin-3-ylmethyl)-carbamoyl]-2-(3,4-difluoro-phenyl)-ethyl]-2-(isopropyl-methyl-amino)-propionamide;
(R)-2-Dimethylamino-3-methyl-pentanoic acid [(S)-1-[(6-amino-pyridin-3-ylmethyl)-carbamoyl]-2-(3,4-difluoro-phenyl)-ethyl]-amide;
(R)—N—[(S)-1-[(6-Amino-pyridin-3-ylmethyl)-carbamoyl]-2-(3,4-difluoro-phenyl)-ethyl]-2-dimethylamino-3,3-dimethyl-butyramide;
(R)-1-Methyl-pyrrolidine-2-carboxylic acid [(S)-1-[(6-amino-2-methyl-pyridin-3-ylmethyl)-carbamoyl]-2-(4-fluoro-phenyl)-ethyl]-amide;
(R)-1-Methyl-pyrrolidine-2-carboxylic acid [(S)-1-[(6-amino-2-methyl-pyridin-3-ylmethyl)-carbamoyl]-2-(3,4-difluoro-phenyl)-ethyl]-amide;
(S)-1-Methyl-pyrrolidine-2-carboxylic acid {(R)-1-[(6-amino-pyridin-3-ylmethyl)-carbamoyl]-2,2-dicyclohexyl-ethyl}-amide;
3-Methyl-1H-pyrrole-2-carboxylic acid [(S)-1-[(6-amino-pyridin-3-ylmethyl)-carbamoyl]-2-(3,4-difluoro-phenyl)-ethyl]-amide;
3,5-Dimethyl-1H-pyrrole-2-carboxylic acid [(S)-1-[(6-amino-pyridin-3-ylmethyl)-carbamoyl]-2-(3-fluoro-phenyl)-ethyl]-amide;
3,5-Dimethyl-1H-pyrrole-2-carboxylic acid [(S)-1-[(6-amino-pyridin-3-ylmethyl)-carbamoyl]-2-(4-fluoro-phenyl)-ethyl]-amide;
3,5-Dimethyl-1H-pyrrole-2-carboxylic acid [(S)-1-[(6-amino-pyridin-3-ylmethyl)-carbamoyl]-2-(3,4-difluoro-phenyl)-ethyl]-amide;
3-Methyl-1H-pyrrole-2-carboxylic acid [(S)-1-[(6-amino-pyridin-3-ylmethyl)-carbamoyl]-2-(4-fluoro-phenyl)-ethyl]-amide;
3,5-Dimethyl-1H-pyrrole-2-carboxylic acid [(S)-1-[(6-amino-2-methyl-pyridin-3-ylmethyl)-carbamoyl]-2-(3-trifluoromethyl-phenyl)-ethyl]-amide;
(R)—N—[(S)-1-[(6-Amino-pyridin-3-ylmethyl)-carbamoyl] -2-(3,4-dichloro-phenyl)-ethyl]-3-methyl-2-methylamino-butyramide;
(S)—N—[(S)-1-[(6-Amino-pyridin-3-ylmethyl)-carbamoyl]-2-(3,4-difluoro-phenyl)-ethyl]-2-(ethyl-methyl-amino)-propionamide;
(S)—N—[(S)-1-[(6-Amino-pyridin-3-ylmethyl)-carbamoyl]-2-(3,4-difluoro-phenyl)-ethyl]-2-diethylamino-propionamide;
(S)—N—{(S)-1-[(6-Amino-pyridin-3-ylmethyl)-carbamoyl]-2-cyclohexyl-ethyl}-2-(isopropyl-methyl-amino)-propionamide;
(S)—N—{(S)-1-[(6-Amino-pyridin-3-ylmethyl)-carbamoyl]-2-cyclohexyl-ethyl}-2-diethylamino-propionamide;
(S)—N—[(S)-1-[(6-Amino-pyridin-3-ylmethyl)-carbamoyl]-2-(decahydro-naphthalen-1-yl)-ethyl]-2-(isopropyl-methyl-amino)-propionamide;
(R)-2-Dimethylamino-3-methyl-pentanoic acid [(S)-1-[(6-amino-pyridin-3-ylmethyl)-carbamoyl]-2-(3,4-dichloro-phenyl)-ethyl]-amide;
(R)—N—[(S)-1-[(6-Amino-pyridin-3-ylmethyl)-carbamoyl]-2-(3,4-dichloro-phenyl)-ethyl]-2-dimethylamino-3-methyl-butyramide;
(S)—N—[(S)-1-[(6-Amino-pyridin-3-ylmethyl)-carbamoyl]-2-(3,4-dichloro-phenyl)-ethyl-2-(isopropyl-methyl-amino)-propionamide;
(S)—N—[(S)-1-[(6-Amino-pyridin-3-ylmethyl)-carbamoyl]-2-(3-chloro-phenyl)-ethyl]-2-(isopropyl-methyl-amino)-propionamide;
(S)—N—{(S)-1-[(6-Amino-pyridin-3-ylmethyl)-carbamoyl]-2-m-tolyl-ethyl}-2-(isopropyl-methyl-amino)-propionamide;
(S)—N—[(S)-1-[(6-Amino-pyridin-3-ylmethyl)-carbamoyl]-2-(3-trifluoromethyl-phenyl)-ethyl]-2-(isopropyl-methyl-amino)-propionamide;
and tautomers, stereoisomers, pharmaceutically acceptable salts and solvates thereof.

9. A method of treatment of a disease or condition in which KLK1 activity is implicated comprising administration to a subject in need thereof a therapeutically effective amount of a compound according to claim 1, or a tautomer, stereoisomer, pharmaceutically acceptable salt or solvate thereof.

10. The method of claim 9 wherein the disease or condition in which KLK1 activity is implicated is selected from an inflammatory or respiratory disorder or condition selected from asthma (allergic and non-allergic), chronic obstructive pulmonary disease (COPD), allergic rhinitis (hayfever), cough, exacerbations resulting from asthma and chronic obstructive pulmonary disease (COPD), multiple sclerosis, arthritis, rheumatoid arthritis, osteopathic arthritis, osteoarthritis, rhinitis, sinusitis, inflammatory bowel disease (such as Crohn's disease and ulcerative colitis), immune mediated diabetes, acute pancreatitis and interstitial cystitis, conjunctivitis, periodontal disease, chronic prostate inflammation, chronic recurrent parotitis, inflammatory skin disorders (e.g. psoriasis, eczema), and SIRS (systemic inflammatory response syndrome); smooth muscle spasm (e.g. asthma, angina), RDS (respiratory distress syndrome), rhino-conjunctivitis, rhinorrhoea, urticaria or a neoplastic disorder.

11. The method of claim 9 wherein the disease or condition in which KLK1 activity is implicated is selected from asthma (allergic and non-allergic), chronic obstructive pulmonary disease (COPD), allergic rhinitis (hayfever), cough, exacerbations resulting from asthma and chronic obstructive pulmonary disease (COPD),

12. The method of claim 9 wherein the disease or condition in which KLK1 activity is implicated is selected from asthma (allergic and non-allergic) and cough.

13. A pharmaceutical composition comprising a compound according to claim 1, or a tautomer, stereoisomer, pharmaceutically acceptable salt or solvate thereof, and a pharmaceutically acceptable carrier, diluent or excipient.
